(19) United States
(12) Patent Application Publication
(10) Pub. No.: US 2006/0019984 A1
(43) Pub. Date: Jan. 26, 2006

(54) Treatment of diseases with alpha-7nACh receptor full agonists

(75) Inventors: Vincent E. Groppi, Kalamazoo, MI (US); Bruce N. Rogers, Mystic, CT (US); Daniel Rudmann, Fishers, IN (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/761,914

(22) Filed: Jan. 21, 2004

Related U.S. Application Data
(60) Provisional application No. 60/441,801, filed on Jan. 22, 2003.

(51) Int. Cl. A61K 31/4745; A61K 31/407

(52) U.S. Cl. 514/305; 514/412

(57) ABSTRACT

The present invention relates to compositions and methods to treat diseases or conditions with alpha-7 nicotinic acetylcholine receptor (AChR) full agonists by decreasing levels of tumor necrosis factor-alpha and/or by stimulating vascular angiogenesis.

### CROSS REFERENCE TO RELATED APPLICATIONS

This application claims the benefit of U.S. provisional application Ser. No. 60/441,801, filed on 22 Jan. 2003, under 35 USC 119(e)(i), which is incorporated herein by reference in its entirety.

### FIELD OF INVENTION

The present invention relates to compositions and methods to treat diseases or conditions with alpha-7 nicotinic acetylcholine receptor (AChR) full agonists, relative to nicotine, by decreasing levels of tumor necrosis factor-alpha or by stimulating vascular angiogenesis.

### BACKGROUND OF THE INVENTION

Nicotinic acetylcholine receptors (nAChRs) play a large role in central nervous system (CNS) activity and in different tissues throughout the body. They are known to be involved in functions, including, but not limited to, cognition, learning, mood, emotion, and neuroprotection. There are several types of nicotinic acetylcholine receptors, and each one appears to have a different role. Some nicotinic receptors regulate CNS function; some regulate pain, inflammation, cancer, and diabetes by controlling tumor necrosis factor alpha (TNF-α); and some regulate vascular angiogenesis; for example, the binding of nicotine to the alpha-7 nAChR stimulates DNA synthesis and proliferation of vascular endothelial cells in vitro (Villablanca, A. C., 1998, J. Appl. Physiol., 84(6):2089-2098) and induces angiogenesis in vivo (Heeschen C., et al. 2002, J. Clin. Invest., 110:527-535; Heeschen, C., et al. 2001, Nature Medicine, 7(7): 833-839). Nicotine affects all such receptors, and has a variety of activities. Unfortunately, not all of the activities are desirable. In fact, undesirable properties of nicotine include its addictive nature and the low ratio between efficacy and safety.

Alpha 7 nAChR agonists are useful to treat, or used to prepare a medicament used to treat, diseases or conditions where a mammal receives symptomatic relief by decreasing levels of TNF-α. Alpha 7 nAChR agonists are also useful to treat, or are used to prepare a medicament to treat, diseases or conditions where a mammal receives symptomatic relief by stimulating vascular angiogenesis.

Cell surface receptors are, in general, excellent and validated drug targets. nAChRs comprise a large family of ligand-gated ion channels that control neuronal activity and brain function. These receptors have a pentameric structure. In mammals, this gene family is composed of nine alpha and four beta subunits that co-assemble to form multiple subtypes of receptors that have a distinctive pharmacology. Acetylcholine is the endogenous regulator of all of the subtypes, while nicotine non-selectively activates all nAChRs.

The α7 nAChR is one receptor system that has proved to be a difficult target for testing. Native α7 nAChR is not routinely able to be stably expressed in most mammalian cell lines (Cooper and Millar, J. Neurochem., 1997, 68(5):2140-51). Another feature that makes functional assays of α7 nAChR challenging is that the receptor is rapidly (100 milliseconds) inactivated. This rapid inactivation greatly limits the functional assays that can be used to measure channel activity.

Agonists of the α7 nAChR are assayed using a cell-based, calcium flux assay on FLIPR. SHEP-1 cells expressing a novel, mutated form of the α7 nAChR that permitted stable cell surface expression were used for these assays. The details of the mutated form of the α7 nAChR are described in WO 00/73431.

### SUMMARY OF THE INVENTION

The present invention claims a method of treating, or use of the any compound of the present invention to prepare a medicament to treat, a disease or condition in a mammal in need thereof to provide symptomatic relief by decreasing levels of tumor narcrosis factor alpha (TNF-α), and/or by stimulating vascular angiogenesis. By way of example but not limitation, some α7 nAChR full agonists are the compounds of Formula I as described herein.

Embodiments of the invention may include one or more or combination of the following.

Disease or conditions treated by decreasing levels of TNF-α, including, but are not limited to, any one or more or combination of the following: inflammation; pain; cancer; or diabetes. Types of inflammation and/or pain that are to be treated include, but are not limited to, any one or more of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, and HIV-3; cytomegalovirus (CMV); influenza; adenovirus; a herpes virus (including HSV-1, HSV-2); or herpes zoster. Types of cancer that are to be treated include, but are not limited to, any one or more of the following: multiple myeloma; acute and chronic myelogenous leukemia; or cancer-associated cachexia. Alpha-7 nAChR full agonists can be used to treat, or be used to prepare a medicament to treat, the TNF-α aspects associated with pancreatic beta cell destruction; or type I and type II diabetes. Diseases or conditions treated by stimulating vascular angiogenesis include, but are not limited to, any one or more of the following: wound healing (healing burns, and wounds in general including from surgery), bone fracture healing, ischemic heart disease, and stable angina pectoris.

Another aspect of the present invention includes α7 nAChR full agonists as described elsewhere: for example, but not by way of limitation, in any one or more of the following patents and published applications: WO 01/60821A1, WO 01/36417A1, WO 02/100857A1, WO 03/042210A1, and WO 03/029252A1. As meant herein, an α7 nAChR full agonist is a ligand that is a full agonist of the nicotinic acetylcholine receptor relative to nicotine. The use of the term α7 nAChR full agonist is used interchangeably with α7 nAChR agonists when discussing the compounds of the present invention.

Another aspect of the present invention includes the method or use of a compound of Formula I, where X is O, or X is S.

Another aspect of the present invention includes the method or use of a compound of Formula I, where Azabicyclo is any one or more of I, II, III, IV, V, VI, or VII. The method or use of a compound of Formula I, where R₁ is H, alkyl, cycloalkyl, haloalkyl, substituted phenyl, or substituted naphthyl; each R₂ is independently F, Cl, Br, I, alkyl, substituted alkyl, haloalkyl, cycloalkyl, aryl, or R₂ is absent; and R₂₋₃ is H, F, Cl, Br, I, alkyl, haloalkyl, substituted alkyl, cycloalkyl, or aryl. The method or use of a compound of Formula I, where the variables of formula I have any definition discussed herein.

Another aspect of the present invention includes the method or use of a compound of Formula I, where W is any one or more of (A), (B), (C), (D), (E), (F), (G), or (H). The method or use of a compound of Formula I, where W is any one or more of (A), (B), (C), (D), (E), (F), (G), or (H). The method or use of a compound of Formula I, where W is any one or more of (A), (B), (C), (D), (E), (F), (G), or (H), wherein the variables within each has any definition allowed. For example, and not by way of limitation, W includes any one or more of the following: 4-chlorophen-1-yl; dibenzo[b,d]thiophene-2-yl; isoquinoline-3-yl; furo[2,3-c]pyridine-5-yl; furo[3,2-c]pyridine-6; 1,3-benzodioxole-5-yl; 2,3-dihydro-1,4-benzodioxine-6-yl; 2,3-dihydro-1,4-benzodioxine-7-yl; 1,3-benzoxazole-5-yl; thieno[2,3-c]pyridine-5-yl; thieno[3,2-c]pyridine-6-yl; [1]benzothieno[3,2-c]pyridine-3-yl; 1,3-benzothiazole-6-yl; thieno[3,4-c]pyridine-6-yl; 2,3-dihydro-1-benzofuran-5-yl; 1-benzofuran-5-yl; furo[3,2-c]pyridine-6-yl; [1]benzothieno[2,3-c]pyridine-3-yl; dibenzo[b,d]furan-2-yl; 1-benzofuran-6-yl; 2-naphthyl; 1H-indole-6-yl; pyrrolo[1,2-c]pyrimidine-3-yl; 1-benzothiophene-5-yl; 1-benzothiophene-5-yl; 1-benzothiophene-6-yl; pyrrolo[1,2-a]pyrazine-3-yl; 1H-indole-6-yl; pyrazino[1,2-a]indole-3-yl; 1,3-benzothiazole-6-yl; [1]benzofuro[2,3-c]pyridine-3-yl; [1]benzofuro[2,3-c]pyridine-3-yl; 2H-chromene-6-yl; indolizine-6-yl; and [1,3]dioxolo[4,5-c]pyridine-6-yl; any of which is optionally substituted as allowed in formula I. One of ordinary skill in the art will recognize how the variables are defined by comparing the named radicals with the different values for W. When W is (D), it is preferred that one of R_{D-1} is the bond to C(X). Specific compounds within the scope of this invention include any one or more of the following as the free base or as a pharmaceutically acceptable salt thereof:
N-[(3R)-1-azabicyclo[2.2.2]oct-3-yl]-4-chlorobenzamide;
N-[(3R)-1-azabicyclo[2.2.2]oct-3-yl]dibenzo[b,d]thiophene-2-carboxamide;
N-[(3R)-1-azabicyclo[2.2.2]oct-3-yl]isoquinoline-3-carboxamide;
N-[(3R)-1-azabicyclo[2.2.2]oct-3-yl]furo[2,3-c]pyridine-5-carboxamide;
N-[(3R)-1-azabicyclo[2.2.2]oct-3-yl]-1,3-benzodioxole-5-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-2,3-dihydro-1,4-benzodioxine-6-carboxamide;
N-[(3R)-1-azabicyclo[2.2.2]oct-3-yl]-3-methylfuro[2,3-c]pyridine-5-carboxamide;
N-[(1S,2R,4R)-7-azabicyclo[2.2.1]hept-2-yl]isoquinoline-3-carboxamide;
N-[(1S,2R,4R)-7-azabicyclo[2.2.1]hept-2-yl]-3-methylfuro[2,3-c]pyridine-5-carboxamide;
N-[(3R)-1-azabicyclo[2.2.2]oct-3-yl]-1,3-benzoxazole-5-carboxamide;
N-[(3R)-1-azabicyclo[2.2.2]oct-3-yl]-2-methyl-1,3-benzoxazole-5-carboxamide;
N-[(1S,2R,4R)-7-azabicyclo[2.2.1]hept-2-yl]thieno[2,3-c]pyridine-5-carboxamide;
N-[(1S,2R,4R)-7-azabicyclo[2.2.1]hept-2-yl]thieno[3,2-c]pyridine-6-carboxamide;
N-[(1S,2R,4R)-7-azabicyclo[2.2.1]hept-2-yl]furo[2,3-c]pyridine-5-carboxamide;
N-[(3R)-1-azabicyclo[2.2.2]oct-3-yl]-3-ethylfuro[2,3-c]pyridine-5-carboxamide;
N-[(3R)-1-azabicyclo[2.2.2]oct-3-yl]-3-isopropylfuro[2,3-c]pyridine-5-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
5-{[(2R)-7-azoniabicyclo[2.2.1]hept-2-ylamino]carbonyl}-3-ethylfuro[2,3-c]pyridin-6-ium dichloride;
5-{[(2R)-7-azoniabicyclo[2.2.1]hept-2-ylamino]carbonyl}-3-isopropylfuro[2,3-c]pyridin-6-ium dichloride;
N-[(3R,4S)-1-azabicyclo[2.2.1]hept-3-yl]furo[2,3-c]pyridine-5-carboxamide;
N-1-azabicyclo[2.2.2]oct-3-yl[1]benzothieno[3,2-c]pyridine-3-carboxamide;
N-[(3R)-1-azabicyclo[2.2.2]oct-3-yl]-1,3-benzothiazole-6-carboxamide;
N-[(3R)-1-azabicyclo[2.2.2]oct-3-yl]-3-chlorofuro[2,3-c]pyridine-5-carboxamide;
N-1-azabicyclo[2.2.2]oct-3-ylfuro[2,3-c]pyridine-5-carboxamide;
N-[(3R)-1-azabicyclo[2.2.2]oct-3-yl]thieno[3,4-c]pyridine-6-carboxamide;
N-[(3R,5R)-1-azabicyclo[3.2.1]oct-3-yl]-3-methylfuro[2,3-c]pyridine-5-carboxamide;
N-[(3R,4S)-1-azabicyclo[2.2.1]hept-3-yl]-3-methylfuro[2,3-c]pyridine-5-carboxamide;
N-[(3R)-1-azabicyclo[2.2.2]oct-3-yl]-2,3-dihydro-1-benzofuran-5-carboxamide;
N-[(3R,4S)-1-azabicyclo[2.2.1]hept-3-yl]thieno[2,3-c]pyridine-5-carboxamide;
N-[(3R)-1-azabicyclo[2.2.2]oct-3-yl]-1-benzofuran-5-carboxamide;
N-[(3R)-1-azabicyclo[2.2.2]oct-3-yl]furo[3,2-c]pyridine-6-carboxamide;
N-[(3R,4S)-1-azabicyclo[2.2.1]hept-3-yl]thieno[3,2-c]pyridine-6-carboxamide;
N-[(3R,4S)-1-azabicyclo[2.2.1]hept-3-yl]3-ethylfuro[2,3-c]pyridine-5-carboxamide;
N-[(3R,4S)-1-azabicyclo[2.2.1]hept-3-yl]3-isopropylfuro[2,3-c]pyridine-5-carboxamide;
N-[(1S,2R,4R)-7-azabicyclo[2.2.1]hept-2-yl]-3-chlorofuro[2,3-c]pyridine-5-carboxamide;
N-[(3R,4S)-1-azabicyclo[2.2.1]hept-3-yl]3-chlorofuro[2,3-c]pyridine-5-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]furo[3,2-c]pyridine-6-carboxamide;
N-[(3R,5R)-1-azabicyclo[3.2.1]oct-3-yl]4-chlorobenzamide;
N-[(1S,2R,4R)-7-azabicyclo[2.2.1]hept-2-yl]thieno[3,4-c]pyridine-6-carboxamide;
N-[(1S,2R,4R)-7-azabicyclo[2.2.1]hept-2-yl]dibenzo[b,d]thiophene-2-carboxamide;
N-[(3R,4S)-1-azabicyclo[2.2.1]hept-3-yl]-1-benzofuran-5-carboxamide;
N-[(3R)-1-azabicyclo[2.2.2]oct-3-yl][1]benzothieno[2,3-c]pyridine-3-carboxamide;
N-[(1S,2R,4R)-7-azabicyclo[2.2.1]hept-2-yl][1]benzothieno[2,3-c]pyridine-3-carboxamide;
N-[(1S,2R,4R)-7-azabicyclo[2.2.1]hept-2-yl]-1-benzofuran-5-carboxamide;
N-[(1S,2R,4R)-7-azabicyclo[2.2.1]hept-2-yl]dibenzo[b,d]furan-2-carboxamide;
N-[(3R,5R)-1-azabicyclo[3.2.1]oct-3-yl]furo[2,3-c]pyridine-5-carboxamide;
N-[(3R,5R)-1-azabicyclo[3.2.1]oct-3-yl]furo[3,2-c]pyridine-6-carboxamide;
N-[(3R,5R)-1-azabicyclo[3.2.1]oct-3-yl]-1-benzofuran-5-carboxamide;
N-[(3R)-1-azabicyclo[2.2.2]oct-3-yl]-3-bromofuro[2,3-c]pyridine-5-carboxamide;
N-[(1S,2R,4R)-7-azabicyclo[2.2.1]hept-2-yl]-3-bromofuro[2,3-c]pyridine-5-carboxamide;
N-[(3R)-1-azabicyclo[2.2.2]oct-3-yl]-1-benzofuran-6-carboxamide;
N-[(2S,3R)-2-methyl-1-azabicyclo[2.2.2]oct-3-yl]-2-naphthamide;
N-[(3R)-1-azabicyclo[2.2.2]oct-3-yl]pyrrolo[1,2-c]pyrimidine-3-carboxamide;
N-[(3R,5R)-1-azabicyclo[3.2.1]oct-3-yl]thieno[2,3-c]pyridine-5-carboxamide;
N-[(3R,5R)-1-azabicyclo[3.2.1]oct-3-yl]thieno[3,2-c]pyridine-6-carboxamide;
N-[(3R,4S)-1-azabicyclo[2.2.1]hept-3-yl]-1H-indole-6-carboxamide;
N-[(2S,3R)-2-methyl-1-azabicyclo[2.2.2]oct-3-yl]thieno[2,3-c]pyridine-5-carboxamide;
3-methyl-N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]-1-benzofuran-5-carboxamide;
N-[(2S,3R)-2-methyl-1-azabicyclo[2.2.2]oct-3-yl]thieno[3,2-c]pyridine-6-carboxamide;
N-[(2S,3R)-2-methyl-1-azabicyclo[2.2.2]oct-3-yl]pyrrolo[1,2-c]pyrimidine-3-carboxamide;
N-[(2S,3R)-2-methyl-1-azabicyclo[2.2.2]oct-3-yl]-1,3-benzothiazole-6-carboxamide;
N-[(3R,5R)-1-azabicyclo[3.2.1]oct-3-yl]pyrrolo[1,2-c]pyrimidine-3-carboxamide;
N-[(3R)-1-azabicyclo[2.2.2]oct-3-yl]-1-benzothiophene-5-carboxamide;
N-[(1S,2R,4R)-7-azabicyclo[2.2.1]hept-2-yl]pyrrolo[1,2-c]pyrimidine-3-carboxamide;
N-[(3R,4S)-1-azabicyclo[2.2.1]hept-3-yl]pyrrolo[1,2-c]pyrimidine-3-carboxamide;
N-[(3R,4S)-1-azabicyclo[2.2.1]hept-3-yl]-3-bromofuro[2,3-c]pyridine-5-carboxamide;
N-[(3R,4S)-1-azabicyclo[2.2.1]hept-3-yl]-1,3-benzodioxole-5-carboxamide;
N-[(3R)-1-azabicyclo[2.2.2]oct-3-yl]-3-bromo-1-benzofuran-5-carboxamide;
N-[(1S,2R,4R)-7-azabicyclo[2.2.1]hept-2-yl]-3-bromo-1-benzofuran-5-carboxamide;
N-[(3R)-1-azabicyclo[2.2.2]oct-3-yl]-3-bromothieno[2,3-c]pyridine-5-carboxamide;
N-[(1S,2R,4R)-7-azabicyclo[2.2.1]hept-2-yl]-3-bromothieno[2,3-c]pyridine-5-carboxamide;
N-[(3R,4S)-1-azabicyclo[2.2.1]hept-3-yl]-1-benzothiophene-5-carboxamide;
N-[(3S)-1-azabicyclo[2.2.2]oct-3-yl]furo[2,3-c]pyridine-5-carboxamide;
N-[(3R)-1-azabicyclo[2.2.2]oct-3-yl]-3-methyl-1-benzofuran-5-carboxamide;
N-[(1S,2R,4R)-7-azabicyclo[2.2.1]hept-2-yl]-3-methyl-1-benzofuran-5-carboxamide;
N-[(3R)-1-azabicyclo[2.2.2]oct-3-yl]-2-methyl-1-benzofuran-6-carboxamide;
N-[(3R,5R)-1-azabicyclo[3.2.1]oct-3-yl]-1-benzofuran-6-carboxamide;
N-[(2S,3R)-2-methyl-1-azabicyclo[2.2.2]oct-3-yl]-1-benzofuran-6-carboxamide;
N-[(2S,3R)-2-methyl-1-azabicyclo[2.2.2]oct-3-yl]-1-benzothiophene-5-carboxamide;
N-[(3R)-1-azabicyclo[2.2.2]oct-3-yl]-1-benzothiophene-6-carboxamide;
N-[(3R)-1-azabicyclo[2.2.2]oct-3-yl]pyrrolo[1,2-a]pyrazine-3-carboxamide;
N-[(3R,4S)-1-azabicyclo[2.2.1]hept-3-yl]-1-benzothiophene-6-carboxamide;
N-[(3R)-1-azabicyclo[2.2.2]oct-3-yl]-1-methyl-1H-indole-6-carboxamide;
N-[(3S)-1-azabicyclo[2.2.2]oct-3-yl]-1-benzofuran-5-carboxamide;
N-[(3R)-1-azabicyclo[2.2.2]oct-3-yl]-3-isopropyl-1-benzofuran-5-carboxamide;
N-[(1S,2R,4R)-7-azabicyclo[2.2.1]hept-2-yl]-3-isopropyl-1-benzofuran-5-carboxamide;
N-[(3R)-1-azabicyclo[2.2.2]oct-3-yl]-3-ethynylfuro[2,3-c]pyridine-5-carboxamide;
N-[(3R)-1-azabicyclo[2.2.2]oct-3-yl]-1H-indazole-6-carboxamide;
N-[(3R)-1-azabicyclo[2.2.2]oct-3-yl]-2-methyl-1-benzofuran-5-carboxamide;
N-[(1S,2R,4R)-7-azabicyclo[2.2.1]hept-2-yl]-2-methyl-1-benzofuran-5-carboxamide;
N-[(3R)-1-azabicyclo[2.2.2]oct-3-yl]pyrazino[1,2-a]indole-3-carboxamide;
3-bromo-N-[(2S,3R)-2-methyl-1-azabicyclo[2.2.2]oct-3-yl]furo[2,3-c]pyridine-5-carboxamide;
N-[(3R,5R)-1-azabicyclo[3.2.1]oct-3-yl]pyrrolo[1,2-a]pyrazine-3-carboxamide;
N-[(3R)-1-azabicyclo[2.2.2]oct-3-yl]-7-methoxy-2-naphthamide;
N-[(1S,2R,4R)-7-azabicyclo[2.2.1]hept-2-yl]pyrrolo[1,2-a]pyrazine-3-carboxamide;
N-[(3R,5R)-1-azabicyclo[3.2.1]oct-3-yl]-1,3-benzothiazole-6-carboxamide;
N-[(3R,4S)-1-azabicyclo[2.2.1]hept-3-yl]-3-bromo-1-benzofuran-6-carboxamide;
N-[(3R)-1-azabicyclo[2.2.2]oct-3-yl][1]benzofuro[2,3-c]pyridine-3-carboxamide;
N-[(1S,2R,4R)-7-azabicyclo[2.2.1]hept-2-yl][1]benzofuro[2,3-c]pyridine-3-carboxamide;
N-[(3R)-1-azabicyclo[2.2.2]oct-3-yl]-3-ethynyl-1-benzofuran-5-carboxamide;
N-[(1S,2R,4R)-7-azabicyclo[2.2.1]hept-2-yl]-3-ethynyl-1-benzofuran-5-carboxamide;
N-[(3R)-1-azabicyclo[2.2.2]oct-3-yl]-2H-chromene-6-carboxamide;
N-[(3R)-1-azabicyclo[2.2.2]oct-3-yl]-3-prop-1-ynyl-1-benzofuran-5-carboxamide;
N-[(3R)-1-azabicyclo[2.2.2]oct-3-yl]-2-phenyl-1,3-benzodioxole-5-carboxamide;
N-[(3R)-1-azabicyclo[2.2.2]oct-3-yl]-6-bromopyrrolo[1,2-a]pyrazine-3-carboxamide;
N-[(3R)-1-azabicyclo[2.2.2]oct-3-yl]-3-prop-1-ynylfuro[2,3-c]pyridine-5-carboxamide;
N-[(2S,3R)-2-methyl-1-azabicyclo[2.2.2]oct-3-yl]pyrrolo[1,2-a]pyrazine-3-carboxamide;
N-[(3R)-1-azabicyclo[2.2.2]oct-3-yl]indolizine-6-carboxamide;
2-amino-N-[(3R)-1-azabicyclo[2.2.2]oct-3-yl]-1,3-benzothiazole-6-carboxamide;
N-[(3R)-1-azabicyclo[2.2.2]oct-3-yl]-6-ethynylpyrrolo[1,2-a]pyrazine-3-carboxamide;
N-[(3R)-1-azabicyclo[2.2.2]oct-3-yl]-8-methoxy-2-naphthamide;
N-[(2S,3R)-2-methyl-1-azabicyclo[2.2.2]oct-3-yl]indolizine-6-carboxamide;
N-[(3R)-1-azabicyclo[2.2.2]oct-3-yl][1,3]dioxolo[4,5-c]pyridine-6-carboxamide;
N-[(1S,2R,4R)-7-azabicyclo[2.2.1]hept-2-yl][1,3]dioxolo[4,5-c]pyridine-6-carboxamide;
N-[(3R)-1-azabicyclo[2.2.2]oct-3-yl]-3-cyano-1-benzofuran-5-carboxamide;
N-[(3R,4S)-1-azabicyclo[2.2.1]hept-3-yl][1,3]dioxolo[4,5-c]pyridine-6-carboxamide;
N-[(3R)-1-azabicyclo[2.2.2]oct-3-yl]-3-ethyl-2,3-dihydro-1,4-benzodioxine-6-carboxamide;
N-[(3R)-1-azabicyclo[2.2.2]oct-3-yl]-3-ethyl-2,3-dihydro-1,4-benzodioxine-7-carboxamide;
N-[(3R)-1-azabicyclo[2.2.2]oct-3-yl]-7-hydroxy-2-naphthamide;
N-[(1S,2R,4R)-7-azabicyclo[2.2.1]hept-2-yl]-3-ethynylfuro[2,3-c]pyridine-5-carboxamide;
N-[(1S,2R,4R)-7-azabicyclo[2.2.1]hept-2-yl]-6-chloroisoquinoline-3-carboxamide;
N-[(3R)-1-azabicyclo[2.2.2]oct-3-yl]-6-methylisoquinoline-3-carboxamide;
N-[(1S,2R,4R)-7-azabicyclo[2.2.1]hept-2-yl]-6-methylisoquinoline-3-carboxamide;
N-[(3R)-1-azabicyclo[2.2.2]oct-3-yl]-3-cyanofuro[2,3-c]pyridine-5-carboxamide;
N-[(3R)-1-azabicyclo[2.2.2]oct-3-yl]-2-naphthamide; and
N-[(3R)-1-azabicyclo[2.2.2]oct-3-yl]dibenzo[b,d]furan-2-carboxamide.

The compounds of Formula I (Azabicyclo I) have asymmetric centers on the quinuclidine ring. The compounds of the present invention include quinuclidines having 3R configuration, 2S, 3R configuration, or 3S configuration and also include racemic mixtures and compositions of varying degrees of streochemical purities. For example, and not by limitation, embodiments of the present invention include compounds of Formula I having the following stereospecificity and substitution: 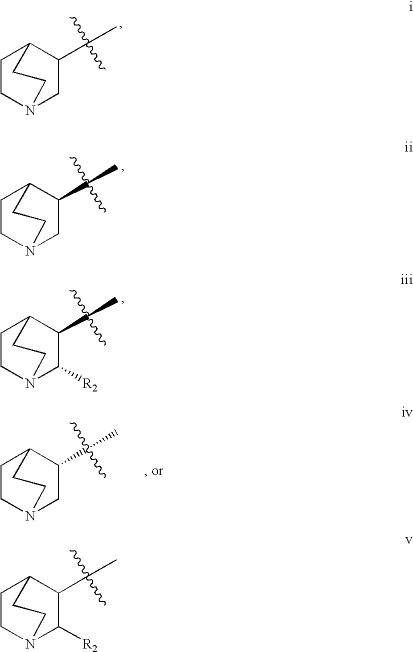
wherein the Azabicyclo
(i) is a racemic mixture;
(ii) has the stereochemistry of 3R at C3;
(iii) has the 3R,2S stereochemistry at C3 and C2, respectively;
(iv) has the stereochemistry of 3S at C3; or
(v) is a racemic mixture; and for (iii) and (v), R₂ has any definition or specific value discussed herein.

The compounds of Formula I (Azabicyclo VII) have asymmetric centers on the 7-azabicyclo[2.2.1]heptane ring which can exhibit a number of stereochemical configurations.

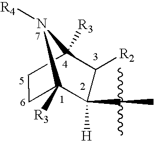

The terms exo and endo are stereochemical prefixes that describe the relative configuration of a substituent on a bridge (not a bridgehead) of a bicyclic system. If a substituent is oriented toward the larger of the other bridges, it is endo. If a substituent is oriented toward the smaller bridge it is exo. Depending on the substitution on the carbon atoms, the endo and exo orientations can give rise to different stereoisomers. For instance, when carbons 1 and 4 are substituted with hydrogen and carbon 2 is bonded to a nitrogen-containing species, the endo orientation gives rise to the possibility of a pair of enantiomers: either the 1S, 2S, 4R isomer or its enantiomer, the 1R, 2R, 4S isomer. Likewise, the exo orientation gives rise to the possibility of another pair of stereoisomers which are diastereomeric and C-2 epimeric with respect to the endo isomers: either the 1R, 2S, 4S isomer or its enantiomer, the 1S, 2R, 4R isomer. The compounds of this invention exist in the exo orientation. For example, when R₂ is absent (C3 is —CH₂—) and R₃=H, the absolute stereochemistry is exo-(1S, 2R, 4R).

The compounds of the present invention have the exo orientation at the C-2 carbon and S configuration at the C-1 carbon and the R configuration at the C-2 and the C-4 carbons of the 7-azabicyclo[2.2.1]heptane ring. Unexpectedly, the inventive compounds exhibit much higher activity relative to compounds lacking the exo 2R, stereochemistry. For example, the ratio of activities for compounds having the exo 2R configuration to other stereochemical configurations may be greater than about 100:1. Although it is desirable that the stereochemical purity be as high as possible, absolute purity is not required. For example, pharmaceutical compositions can include one or more compounds, each having an exo 2R configuration, or mixtures of compounds having exo 2R and other configurations. In mixtures of compounds, those species possessing stereochemical configurations other than exo 2R act as diluents and tend to lower the activity of the pharmaceutical composition. Typically, pharmaceutical compositions including mixtures of compounds possess a larger percentage of species having the exo 2R configuration relative to other configurations.

The compounds of Formula I (Azabicyclo II) have asymmetric center(s) on the [2.2.1] azabicyclic ring at C3 and C4. The scope of this invention includes the separate stereoisomers of Formula I being endo-4S, endo-4R, exo-4S, exo-4R: 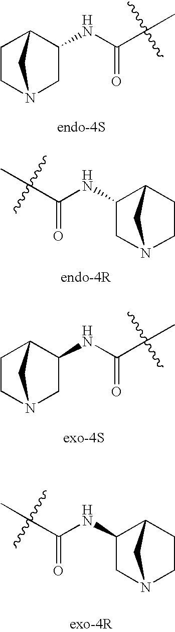
The endo isomer is the isomer where the non-hydrogen substituent at C3 of the [2.2.1] azabicyclic compound is projected toward the larger of the two remaining bridges. The exo isomer is the isomer where the non-hydrogen substituent at C3 of the [2.2.1] azabicyclic compound is projected toward the smaller of the two remaining bridges. Thus, there can be four separate isomers: exo-4(R), exo-4(S), endo-4(R), and endo-4(S). Some embodiments of compounds of Formula I for when Azabicyclo is II include racemic mixtures where R₂ is absent (k₂ is 0) or is at C2 or C6; or Azabicyclo II has the exo-4(S) stereochemistry and R₂ has any definition discussed herein and is bonded at any carbon discussed herein.

The compounds of Formula I (Azabicyclo III) have asymmetric center(s) on the [2.2.1] azabicyclic ring at C1, C4 and C5. The scope of this invention includes racemic mixtures and the separate stereoisomers of Formula I being (1R,4R,5S), (1R,4R,5R), (1S,4S,5R), (1S,4S,5S): 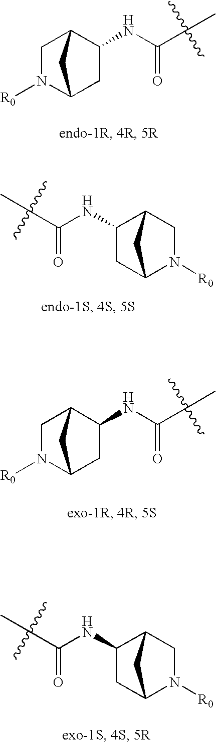
The endo isomer is the isomer where the non-hydrogen substituent at C5 of the [2.2.1] azabicyclic compound is projected toward the larger of the two remaining bridges. The exo isomer is the isomer where the non-hydrogen substituent at C5 of the [2.2.1] azabicyclic compound is projected toward the smaller of the two remaining bridges. Thus, there can be four separate isomers: exo-(1R,4R,5S), exo-(1S,4S,5R), endo-(1S,4S,5S), endo-(1R,4R,5R). Another group of compounds of Formula I includes R₂₋₃ is absent, or is present and either at C3 or bonds to any carbon with sufficient valancy.

The compounds of Formula I (Azabicyclo IV) have asymmetric center(s) on the [2.2.1] azabicyclic ring at C1, C4 and C6. The scope of this invention includes racemic mixtures and the separate stereoisomers of Formula I being exo-(1S,4R,6S), exo-(1R,4S,6R), endo-(1S,4R,6R), and endo-(1R,4S,6S): 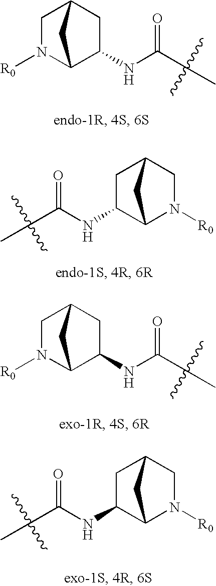
The endo isomer is the isomer where the non-hydrogen substituent at C6 of the [2.2.1] azabicyclic compound is projected toward the larger of the two remaining bridges. The exo isomer is the isomer where the non-hydrogen substituent at C6 of the [2.2.1] azabicyclic compound is projected toward the smaller of the two remaining bridges. Thus, there can be four separate isomers: exo-(1S,4R,6S), exo-(1R,4S,6R), endo-(1S,4R,6R), and endo-(1R,4S,6S). Another group of compounds of Formula I includes R₂₋₃ is H, or is other than H and bonded at C3 or is bonded to any carbon with sufficient valancy.

The compounds of Formula I have asymmetric center(s) on the [3.2.1] azabicyclic ring at C3 and C5. The scope of this invention includes the separate stereoisomers of Formula I being endo-3S, 5R, endo-3R, 5S, exo-3R, 5R, exo-3S, 5S: 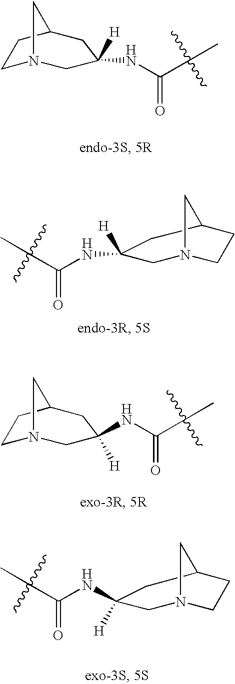
Another group of compounds of Formula I (Azabicyclo V) includes compounds where Azabicyclo V moiety has the stereochemistry of 3R, 5R, or is a racemic mixture and the moiety is either not substituted with R₂ (each is absent) or has one to two substituents being at either C2 and/or C4. When the moiety is substituted, the preferred substituents for substitution at C2 are alkyl, haloalkyl, substituted alkyl, cycloalkyl, or aryl; and for substitution at C4 are F, Cl, Br, I, alkyl, haloalkyl, substituted alkyl, cycloalkyl, or aryl.

The compounds of Formula I (Azabicyclo is VI) have asymmetric centers on the [3.2.2] azabicyclic ring with one center being at C3 when R₂ is absent. The scope of this invention includes racemic mixtures and the separate stereoisomers of Formula I being 3(S) and 3(R): 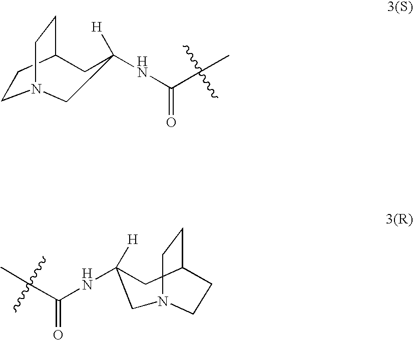
Another group of compounds of Formula I (Azabicyclo VI) includes compounds where Azabicyclo VI moiety is either not substituted with R₂ (each is absent) or has one to two substituents with one being at either C2 or C4 or when two are present, one being at each C2 and C4. When the moiety is substituted, the preferred substituents for substitution at C2 are alkyl, haloalkyl, substituted alkyl, cycloalkyl, or aryl; and for substitution at C4 are F, Cl, Br, I, alkyl, haloalkyl, substituted alkyl, cycloalkyl, or aryl.

Stereoselective syntheses and/or subjecting the reaction product to appropriate purification steps produce substantially enantiomerically pure materials. Suitable stereoselective synthetic procedures for producing enantiomerically pure materials are well known in the art, as are procedures for purifying racemic mixtures into enantiomerically pure fractions.

The compounds of the present invention having the specified stereochemistry above have different levels of activity and that for a given set of values for the variable substitutuents one isomer may be preferred over the other isomers. Although it is desirable that the stereochemical purity be as high as possible, absolute purity is not required. It is preferred to carry out stereoselective syntheses and/or to subject the reaction product to appropriate purification steps so as to produce substantially enantiomerically pure materials. Suitable stereoselective synthetic procedures for producing enantiomerically pure materials are well known in the art, as are procedures for purifying racemic mixtures into enantiomerically pure fractions.

In another aspect, the invention provides an alpha 7 nAChR full agonist of the present invention can also be administered in combination with other agents when treating symptoms associated with infection, inflammation, cancer, or diabetes. For treating these diseases or conditions, a medicament can be prepared comprising a compound of formula I. The same medicament or separate medicament(s), can be prepared comprising any one of the following: an antibacterial; antiviral agent; at least one or more anticancer agent(s) and/or antiemetic agent(s); or at least one agent to treat diabetes. For example, the alpha 7 nAChR full agonist can be co-administered with an antibacterial or antiviral agent, as one medicament or as two separate medicament, to treat an infection, for example, but not limiting, rhinitis. The alpha 7 nAChR full agonist can also be co-administered with anticancer agent(s) and/or antiemetic agent(s) when the disease or condition being treated is cancer, so there could be one medicament or separate medicaments for each agent: one medicament for the alpha 7 nAChR full agonist, at least one medicament for at least one anticancer agent, and at least one medicament for at least one antiemetic agent. And, the alpha 7 nAChR full agonist can be co-administered with at least one agent or more to treat diabetes in one medicament or as separate medicaments. One of ordinary skill in the art of using these other agents knows what is generally used for these other agents and, therefore, a list of those other agents does not need to be repeated herein.

In a combination therapy, the alpha 7 nAChR full agonist and the other agent(s) can be administered simultaneously or at separate intervals. When administered simultaneously, the alpha 7 nAChR full agonist and the other agent(s) can be incorporated into a single pharmaceutical composition, e.g., a pharmaceutical combination therapy composition. Alternatively, more than one, e.g., two or more separate compositions, i.e., one containing an alpha 7 nAChR full agonist and the other containing, for example, the antibacterial agent, can be administered.

In another aspect, the invention provides pharmaceutical compositions comprising an alpha 7 nAChR full agonist according to the invention and a pharmaceutically acceptable carrier or diluent and optionally other adjuvants. Acceptable carriers, diluents, and adjuvants are any of those commercially used in the art, in particular, those used in pharmaceutical compositions comprising, for example but not limitation, an antibacterial agent. Accordingly, such carriers, diluents, and adjuvants need not be repeated here.

These compositions may be formulated with common excipients, diluents or carriers, and compressed into tablets, or formulated elixirs or solutions for convenient oral administration or administered by intramuscular intravenous routes. The compounds can be administered rectally, topically, orally, sublingually, or parenterally and maybe formulated as sustained relief dosage forms and the like.

When separately administered, therapeutically effective amounts of compositions containing and alpha 7 nAChR full agonist and other agent(s) are administered on a different schedule. One may be administered before the other as long as the time between the two administrations falls within a therapeutically effective interval. A therapeutically effective interval is a period of time beginning when one of either (a) the alpha 7 nAChR full agonist, or (b) the other agent(s) is administered to a mammal and ending at the limit of the beneficial effect in the treatment of the disease or condition to be treated from the combination of (a) and (b). The methods of administration of the alpha 7 nAChR full agonist and the other agent(s) may vary. Thus, either agent or both agents may be administered rectally, topically, orally, sublingually, or parenterally.

The amount of therapeutically effective alpha 7 nAChR full agonist that is administered and the dosage regimen for treating a disease or condition with the compounds and/or compositions of this invention depends on a variety of factors, including the age, weight, sex and medical condition of the subject, the severity of the disease, the route and frequency of administration, and the particular compound(s) employed, and thus may vary widely. The compositions contain well know carriers and excipients in addition to a therapeutically effective amount of alpha 7 nAChR full agonist. The pharmaceutical compositions may contain the alpha 7 nAChR full agonist in the range of about 0.001 to 100 mg/kg/day for an adult, preferably in the range of about 0.1 to 50 mg/kg/day for an adult. A total daily dose of about 1 to 1000 mg of a compound of Formula I may be appropriate for an adult. The daily dose can be administered in one to four doses per day. These compositions may be formulated with common excipients, diluents or carriers, and compressed into tablets, or formulated elixirs or solutions for convenient oral administration or administered by intramuscular intravenous routes. The alpha 7 nAChR full agonist can be administered rectally, topically, orally, sublingually, or parenterally and maybe formulated as sustained relief dosage forms and the like.

The combined administration of the alpha 7 nAChR full agonist and the other agent(s) is expected to require less of the generally-prescribed dose for either agent when used alone and or is expected to result in less frequent administration of either or both agents. The skilled clinician may in fact learn that behavioral problems are secondary to the cognitive problems and can be treated with lower dosages of the other agent(s). Determining such dosages and routes of administration should be a routine determination by one skilled in the art of treating patients with the diseases or conditions discussed herein.

Further aspects and embodiments of the invention may become apparent to those skilled in the art from a review of the following detailed description, taken in conjunction with the examples and the appended claims. While the invention is susceptible of embodiments in various forms, described hereafter are specific embodiments of the invention with the understanding that the present disclosure is intended as illustrative, and is not intended to limit the invention to the specific embodiments described herein.

### DETAILED DESCRIPTION OF THE INVENTION

Surprisingly, we have found that α7 nAChR full agonists administered to a mammal in need thereof provide symptomatic relief by decreasing levels of tumor narcrosis factor alpha (TNF-α), and/or by stimulating vascular angiogenesis.

The present invention claims any compound that is a full agonists to an α7 nAChR or α7 nAChR full agonists, described either herein or elsewhere and in particular, and by way of example but not limitation, some α7 nAChR full agonists are the compounds of Formula I as described herein.

The present invention claims any compound that is a full agonist relative to nicotine of an α7 Nicotinic Acetylcholine Receptor (nAChR), or α7 nAChR full agonists, described either herein or elsewhere and in particular, and by way of example and not limitation some α7 nAChR full agonists include compounds of Formula I as described herein. The α7 nAChR full agonists are administered in combination with psychostimulants and/or monoamine reuptake inhibitors. Alpha 7 nAChR full agonists within the scope of the present invention include compounds of Formula I:
Azabicyclo-N(R₁)—C(═X)—W Formula I
wherein Azabicyclo is

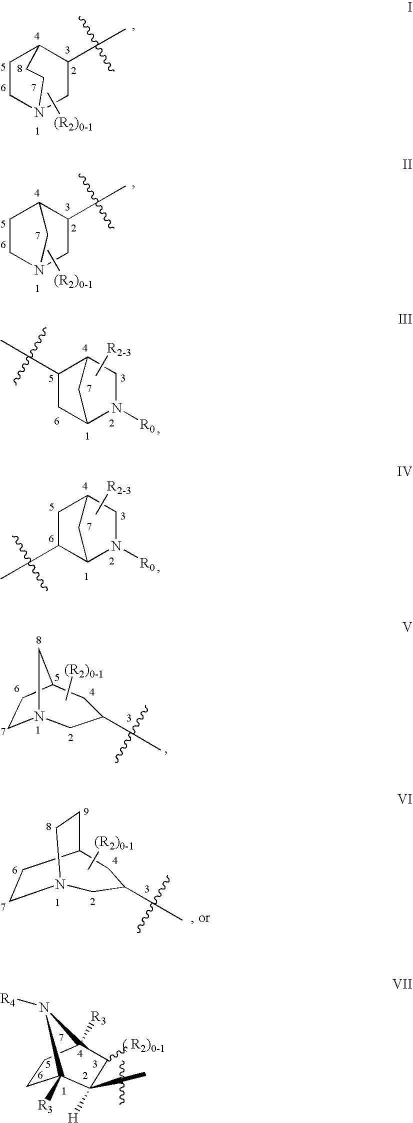

X is O, or S;

R₀ is H, lower alkyl, substituted lower alkyl, or lower haloalkyl;

Each R₁ is H, alkyl, cycloalkyl, haloalkyl, substituted phenyl, or substituted naphthyl;

Each R₂ is independently F, Cl, Br, I, alkyl, substituted alkyl, haloalkyl, cycloalkyl, aryl, or R₂ is absent;

R₂₋₃ is H, F, Cl, Br, I, alkyl, haloalkyl, substituted alkyl, cycloalkyl, or aryl;

Each R₃ is independently H, alkyl, or substituted alkyl;

R₄ is H, alkyl, an amino protecting group, or an alkyl group having 1-3 substituents selected from F, Cl, Br, I, —OH, —CN, —NH₂, —NH(alkyl), or —N(alkyl)₂;

Lower alkyl is both straight- and branched-chain moieties having from 1-4 carbon atoms;

Lower haloalkyl is lower alkyl having 1 to (2n+1) substituent(s) independently selected from F, Cl, Br, or I where n is the maximum number of carbon atoms in the moiety;

Lower substituted alkyl is lower alkyl having 0-3 substituents independently selected from F, Cl, Br, or I and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Alkyl is both straight- and branched-chain moieties having from 1-6 carbon atoms;

Haloalkyl is alkyl having 1 to (2n+1) substituent(s) independently selected from F, Cl, Br, or I where n is the maximum number of carbon atoms in the moiety;

Substituted alkyl is alkyl having 0-3 substituents independently selected from F, Cl, Br, or I and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Alkenyl is straight- and branched-chain moieties having from 2-6 carbon atoms and having at least one carbon-carbon double bond;

Haloalkenyl is alkenyl having 1 to (2n−1) substituent(s) independently selected from F, Cl, Br, or I where n is the maximum number of carbon atoms in the moiety;

Substituted alkenyl is alkenyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Alkynyl is straight- and branched-chained moieties having from 2-6 carbon atoms and having at least one carbon-carbon triple bond;

Haloalkynyl is alkynyl having 1 to (2n−3) substituent(s) independently selected from F, Cl, Br, or I where n is the maximum number of carbon atoms in the moiety;

Substituted alkynyl is alkynyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Cycloalkyl is a cyclic alkyl moiety having from 3-6 carbon atoms;

Halocycloalkyl is cycloalkyl having 1-4 substituents independently selected from F, or Cl;

Substituted cycloalkyl is cycloalkyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Heterocycloalkyl is a cyclic moiety having 4-7 atoms with 1-2 atoms within the ring being —S—, —N(R₁₀)—, or —O—;

Haloheterocycloalkyl is heterocycloalkyl having 1-4 substituents independently selected from F, or Cl;

Substituted heterocycloalkyl is heterocycloalkyl having 0-3 substituents independently selected from F, or Cl, and further having 1 substituent selected from R₅, R₆, —CN, —NO₂, —OR₈, —SR₈, —N(R₈)₂, —C(O)R₈, —C(O)OR₈, —C(S)R₈, —C(O)N(R₈)₂, —NR₈C(O)N(R₈)₂, —NR₈C(O)R₈, —S(O)R₈, —S(O)₂R₈, —OS(O)₂R₈, —S(O)₂N(R₈)₂, —NR₈S(O)₂R₈, phenyl, or phenyl having 1 substituent selected from R₉ and further having 0-3 substituents independently selected from F, Cl, Br, or I;

Lactam heterocycloalkyl is a cyclic moiety having from 4-7 atoms with one atom being only nitrogen with the bond to the lactam heterocycloalkyl thru said atom being only nitrogen and having a ═O on a carbon adjacent to said nitrogen, and having up to 1 additional ring atom being oxygen, sulfur, or nitrogen and further having 0-2 substituents selected from F, Cl, Br, I, or R₇ where valency allows;

Aryl is phenyl, substituted phenyl, naphthyl, or substituted naphthyl;

Substituted phenyl is a phenyl either having 1-4 substituents independently selected from F, Cl, Br, or I, or having 1 substituent selected from R₁₁ and 0-3 substituents independently selected from F, Cl, Br, or I;

Substituted naphthyl is a naphthalene moiety either having 1-4 substituents independently selected from F, Cl, Br, or I, or having 1 substituent selected from R₁₁ and 0-3 substituents independently selected from F, Cl, Br, or I, where the substitution can be independently on either only one ring or both rings of said naphthalene moiety;

Substituted phenoxy is a phenoxy either having 1-3 substituents independently selected from F, Cl, Br, or I, or having 1 substituent selected from R₁₁ and 0-2 substituents independently selected from F, Cl, Br, or I;

R₅ is 5-membered heteroaromatic mono-cyclic moieties containing within the ring 1-3 heteroatoms independently selected from the group consisting of —O—, ═N—, —N(R₁₀)—, and —S—, and having 0-1 substituent selected from R₉ and further having 0-3 substituents independently selected from F, Cl, Br, or I, or R₅ is 9-membered fused-ring moieties having a 6-membered ring fused to a 5-membered ring and having the formula 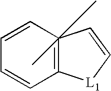
wherein L₁ is O, S, or NR₁₀, 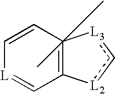
wherein L is CR₁₂ or N, L₂ and L₃ are independently selected from CR₁₂, C(R₁₂)₂, O, S, N, or NR₁₀, provided that both L₂ and L₃ are not simultaneously O, simultaneously S, or simultaneously O and S, or 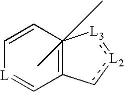
wherein L is CR₁₂ or N, and L₂ and L₃ are independently selected from CR₁₂, O, S, N, or NR₁₀, and each 9-membered fused-ring moiety having 0-1 substituent selected from R₉ and further having 0-3 substituent(s) independently selected from F, Cl, Br, or I, wherein the R₅ moiety attaches to other substituents as defined in formula I at any position as valency allows;

R₆ is 6-membered heteroaromatic mono-cyclic moieties containing within the ring 1-3 heteroatoms selected from ═N— and having 0-1 substituent selected from R₉ and 0-3 substituent(s) independently selected from F, Cl, Br, or I, or R₆ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₉ and 0-3 substituent(s) independently selected from F, Cl, Br, or I, wherein the R₆ moiety attaches to other substituents as defined in formula I at any position as valency allows;

R₇ is alkyl, substituted alkyl, haloalkyl, —OR₁₁, —CN, —NO₂, —N(R₈)₂;

Each R₈ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;

R₉ is alkyl, cycloalkyl, heterocycloalkyl, haloalkyl, halocycloalkyl, haloheterocycloalkyl, —OR₁₄, —SR₁₄, —N(R₁₄)₂, —C(O)R₁₄, —C(O)N(R₁₄)₂, —CN, —NR₁₄C(O)R₁₄, —S(O)₂N(R₁₄)₂, —NR₁₄S(O)₂R₁₄, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;

R₁₀ is H, alkyl, haloalkyl, substituted alkyl, cycloalkyl, halocycloalkyl, substituted cycloalkyl, phenyl, or phenyl having 1 substituent selected from R₇ and further having 0-3 substituents independently selected from F, Cl, Br, or I;

Each R₁₁ is independently H, alkyl, cycloalkyl, heterocycloalkyl, haloalkyl, halocycloalkyl, or haloheterocycloalkyl;

Each R₁₂ is independently H, F, Cl, Br, I, alkyl, cycloalkyl, heterocycloalkyl, haloalkyl, halocycloalkyl, haloheterocycloalkyl, substituted alkyl, substituted cycloalkyl, substituted heterocycloalkyl, —CN, —NO₂, —OR₁₄, —SR₁₄, —N(R₁₄)₂, —C(O)R₁₄, —C(O)N(R₁₄)₂, —NR₁₄C(O)R₁₄, —S(O)₂N(R₁₄)₂, —NR₁₄S(O)₂R₁₄, or a bond;

R₁₃ is —OR₁₄, —SR₁₄, —N(R₁₄)₂, —C(O)R₁₄, —C(O)N(R₁₄)₂, —CN, —CF₃, —NR₁₄C(O)R₁₄, —S(O)₂N(R₁₄)₂, —NR₁₄S(O)₂R₁₄, or —NO₂;

Each R₁₄ is independently H, alkyl, cycloalkyl, heterocycloalkyl, haloalkyl, halocycloalkyl, or haloheterocycloalkyl;

wherein W is (A):

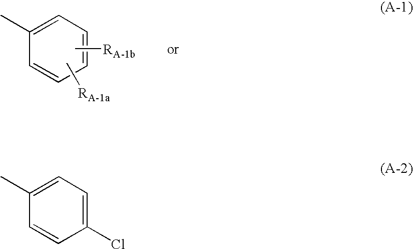

wherein R_{A-1a} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, —R₅, R₆, —OR_{A-3}, —OR_{A-4}, —SR_{A-3}, F, Cl, Br, I, —N(R_{A-3})₂, —N(R_{A-5})₂, —C(O)R_{A-3}, —C(O)R_{A-5}, —CN, —C(O)N(R_{A-3})₂, —C(O)N(R_{A-6})₂, —NR_{A-3}C(O)R_{A-3}, —S(O)R_{A-3}, —OS(O)₂R_{A-3}, —NR_{A-3}S(O)₂R_{A-3}, —NO₂, and —N(H)C(O)N(H)R_{A-3};

R_{A-1b} is —O—R_{A-3}, —S—R_{A-3}, —S(O)—R_{A-3}, —C(O)—R_{A-7}, and alkyl substituted on the ω carbon with R_{A-7} where said ω carbon is determined by counting the longest carbon chain of the alkyl moiety with the C-1 carbon being the carbon attached to the phenyl ring attached to the core molecule and the ω carbon being the carbon furthest from said C-1 carbon;

Each R_{A-3} is independently selected from H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

R_{A-4} is selected from cycloalkyl, halocycloalkyl, substituted cycloalkyl, heterocycloalkyl, haloheterocycloalkyl, or substituted heterocycloalkyl;

Each R_{A-5} is independently selected from cycloalkyl, halocycloalkyl, substituted cycloalkyl, heterocycloalkyl, haloheterocycloalkyl, substituted heterocycloalkyl, R₅, R₆, phenyl, or substituted phenyl;

Each R_{A-6} is independently selected from alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

R_{A-7} is selected from aryl, R₅, or R₆;

wherein W is (B):

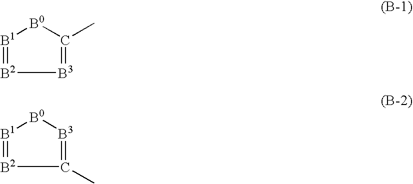

wherein B⁰ is —O—, —S—, or —N(R_{B-0})—;

B¹ and B² are independently selected from ═N—, or ═C(R_{B-1})—;

B³ is ═N—, or ═CH—, provided that when both B¹ and B² are ═C(R_{B-1})— and B³ is ═CH—, only one ═C(R_{B-1})— can be ═CH—, and further provided that when B⁰ is —O—, B² is ═C(R_{B-1})— and B³ is ═C(H)—, B¹ cannot be ═N—,

R_{B-0} is H, alkyl, cycloalkyl, heterocycloalkyl, haloalkyl, halocycloalkyl, haloheterocycloalkyl, substituted alkyl, limited substituted alkyl, substituted cycloalkyl, substituted heterocycloalkyl, or aryl, and provided that when B is (B-2) and B³ is ═N— and B⁰ is N(R_{B-0}), R_{B-0} cannot be phenyl or substituted phenyl;

R_{B-1} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, limited substituted alkyl, limited substituted alkenyl, limited substituted alkynyl, aryl, —OR_{B-2}, —OR_{B-3}, —SR_{B-2}, —SR_{B-3}, F, Cl, Br, I, —N(R_{B-2})₂, —N(R_{B-3})₂, —C(O)R_{B-2}, —C(O)R_{B-3}, —C(O)N(R_{B-2})₂, —C(O)N(R_{B-3})₂, —CN, —NR_{B-2}C(O)R_{B-4}, —S(O)₂N(R_{B-2})₂, —OS(O)₂R_{B-4}, —S(O)₂R_{B-2}, —S(O)₂R_{B-3}, —NR_{B-2}S(O)₂R_{B-2}, —N(H)C(O)N(H)R_{B-2}, —NO₂, R₅, and R₆;

Limited substituted alkyl is alkyl having 0-3 substituents independently selected from F, Cl, Br, or I, and further having 1 substituent on either only the ω carbon and selected from —OR_{B-4}, —SR_{B-4}, —N(R_{B-4})₂, —C(O)R_{B-4}, —NO₂, —C(O)N(R_{B-4})₂, —CN, —NR_{B-2}C(O)R_{B-4}, —S(O)₂N(R_{B-2})₂, or —NR_{B-2}S(O)₂R_{B-2}, or on any carbon with sufficient valency but not on the ω carbon and selected from —R₅, —R₆, —OR_{B-2}, —SR_{B-2}, —N(R_{B-2})₂, —C(O)R_{B-2}, —NO₂, —C(O)N(R_{B-2})₂, —CN, —NR_{B-2}C(O)R_{B-2}, —S(O)₂N(R_{B-2})₂, —NR_{B-2}S(O)₂R_{B-2}, phenyl, or substituted phenyl;

Limited substituted alkenyl is alkenyl having 0-3 substituents independently selected from F, Cl, Br, or I, and further having 1 substituent on either only the ω carbon and selected from —OR_{B-4}, —SR_{B-4}, —N(R_{B-4})₂, —C(O)R_{B-4}, —NO₂, —C(O)N(R_{B-4})₂, —CN, —NR_{B-2}C(O)R_{B-4}, —S(O)₂N(R_{B-2})₂, or —NR_{B-2}S(O)₂R_{B-2}, or on any carbon with sufficient valency but not on the ω carbon and selected from —R₅, —R₆, —OR_{B-2}, —SR_{B-2}, —N(R_{B-2})₂, —C(O)R_{B-2}, —NO₂, —C(O)N(R_{B-2})₂, —CN, —NR_{B-2}C(O)R_{B-2}, —S(O)₂N(R_{B-2})₂, —NR_{B-2}S(O)₂R_{B-2}, phenyl, or substituted phenyl;

Limited substituted alkynyl is alkynyl having 0-3 substituents independently selected from F, Cl, Br, or I, and further having 1 substituent on either only the ω carbon and selected from —OR_{B-4}, —SR_{B-4}, —N(R_{B-4})₂, —C(O)R_{B-4}, —NO₂, —C(O)N(R_{B-4})₂, —CN, —NR_{B-2}C(O)R_{B-4}, —S(O)₂N(R_{B-2})₂, or —NR_{B-2}S(O)₂R_{B-2}, or on any carbon with sufficient valency but not on the ω carbon and selected from —R₅, —R₆, —OR_{B-2}, —SR_{B-2}, —N(R_{B-2})₂, —C(O)R_{B-2}, —NO₂, —C(O)N(R_{B-2})₂, —CN, —NR_{B-2}C(O)R_{B-2}, —S(O)₂N(R_{B-2})₂, —NR_{B-2}S(O)₂R_{B-2}, phenyl, or substituted phenyl;

Each R_{B-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

Each R_{B-3} is independently H, alkyl, haloalkyl, limited substituted alkyl, cycloalkyl, halocycloalkyl, substituted cycloalkyl, heterocycloalkyl, haloheterocycloalkyl, substituted heterocycloalkyl;

R_{B-4} is independently H, alkyl, cycloalkyl, heterocycloalkyl, haloalkyl, halocycloalkyl, or haloheterocycloalkyl;

wherein W is (C):

(C) is a six-membered heterocyclic ring system having 1-2 nitrogen atoms or a 10-membered bicyclic-six-six-fused-ring system having up to two nitrogen atoms within either or both rings, provided that no nitrogen is at a bridge of the bicyclic-six-six-fused-ring system, and further having 1-2 substitutents independently selected from R_{C-1};

Each R_{C-1} is independently H, F, Cl, Br, I, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, lactam heterocycloalkyl, phenyl, substituted phenyl, —NO₂, —CN, —OR_{C-2}, —SR_{C-2}, —SOR_{C-2}, —SO₂R_{C-2}, —NR_{C-2}C(O)R_{C-3}, —NR_{C-2}C(O)R_{C-2}, —NR_{C-2}C(O)R_{C-4}, —N(R_{C-2})₂, —C(O)R_{C-2}, —C(O)₂R_{C-2}, —C(O)N(R_{C-2})₂, —SCN, —S(O)N(R_{C-2})₂, —S(O)₂N(R_{C-2})₂, —NR_{C-2}S(O)₂R_{C-2}, R₅, or R₆;

Each R_{C-2} is independently H, alkyl, cycloalkyl, heterocycloalkyl, alkyl substituted with 1 substituent selected from R_{C-5}, cycloalkyl substituted with 1 substituent selected from R_{C-5}, heterocycloalkyl substituted with 1 substituent selected from R_{C-5}, haloalkyl, halocycloalkyl, haloheterocycloalkyl, phenyl, or substituted phenyl;

Each R_{C-3} is independently H, alkyl, or substituted alkyl;

R_{C-4} is H, alkyl, an amino protecting group, or an alkyl group having 1-3 substituents selected from F, Cl, Br, I, —OH, —CN, —NH₂, —NH(alkyl), or —N(alkyl)₂;

R_{C-5} is —CN, —CF₃, —NO₂, —OR_{C-6}, —SR_{C-6}, —N(R_{C-6})₂, —C(O)R_{C-6}, —SOR_{C-6}, —SO₂R_{C-6}, —C(O)N(R_{C-6})₂, —NR_{C-6}C(O)R_{C-6}, —S(O)₂N(R_{C-6})₂, or —NR_{C-6}S(O)₂R_{C-6};

Each R_{C-6} is independently H, alkyl, cycloalkyl, heterocycloalkyl, haloalkyl, halocycloalkyl, or haloheterocycloalkyl;

wherein W is (D): 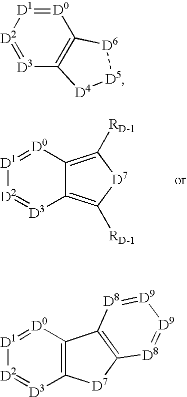
provided that the bond between the —C(═X)— group and the W group may be attached at any available carbon atom within the D group as provided in R_{D-1}, R_{D-3}, and R_{D-4};

D⁰, D¹, D², and D³ are N or C(R_{D-1}) provided that up to one of D⁰, D¹, D², or D³ is N and the others are C(R_{D-1}), further provided that when the core molecule is attached at D² and D⁰ or D¹ is N, D³ is C(H), and further provided that there is only one attachment to the core molecule;

D⁴ - - - D⁵ - - - D⁶ is selected from N(R_{D-2})—C(R_{D-3})═C(R_{D-3}), N═C(R_{D-3})—C(R_{D-4})₂, C(R_{D-3})═C(R_{D-3})—N(R_{D-2}), C(R_{D-3})₂—N(R_{D-2})—C(R_{D-3})₂, C(R_{D-4})₂—C(R_{D-3})═N, N(R_{D-2})—C(R_{D-3})₂—C(R_{D-3})₂, C(R_{D-3})₂—C(R_{D-3})₂—N(R_{D-2}), O—C(R_{D-3})═C(R_{D-3}), O—C(R_{D-3})₂—C(R_{D-3})₂, C(R_{D-3})₂—O—C(R_{D-3})₂, C(R_{D-3})═C(R_{D-3})—O, C(R_{D-3})₂—C(R_{D-3})₂—O, S—C(R_{D-3})═C(R_{D-3}), S—C(R_{D-3})₂—C(R_{D-3})₂, C(R_{D-3})₂—S—C(R_{D-3})₂, C(R_{D-3})═C(R_{D-3})—S, or C(R_{D-3})₂—C(R_{D-3})₂—S;

provided that when C(X) is attached to W at D² and D⁶ is O, N(R_{D-2}), or S, D⁴ - - - D⁵ is not CH═CH;

and further provided that when C(X) is attached to W at D² and D⁴ is O, N(R_{D-2}), or S, D⁵ - - - D⁶ is not CH═CH;

Each R_{D-1} is independently H, F, Br, I, Cl, —CN, —CF₃, —OR_{D-5}, —SR_{D-5}, —N(R_{D-5})₂, or a bond to —C(X)— provided that only one of R_{D-1}, R_{D-3}, and R_{D-4} is said bond;

Each R_{D-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, or R₆;

Each R_{D-3} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-10})₂, —SR_{D-10}, —S(O)₂R_{D-10}, —C(O)R_{D-12}, —CO₂R_{D-10}, aryl, R₅, R₆, or a bond to —C(X)— provided that only one of R_{D-1}, R_{D-3}, and R_{D-4} is said bond;

Each R_{D-4} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-11})₂, —SR_{D-10}, —CO₂R_{D-10}, aryl, R₅, R₆, or a bond to —C(X)— provided that only one of R_{D-1}, R_{D-3}, and R_{D-4} is said bond;

Each R_{D-5} is independently H, C₁₋₃ alkyl, or C₂₋₄ alkenyl;

D⁷ is O, S, or N(R_{D-2});

D⁸ and D⁹ are C(R_{D-1}), provided that when the molecule is attached to the phenyl moiety at D⁹, D⁸ is CH;

Each R_{D-10} is H, alkyl, cycloalkyl, haloalkyl, substituted phenyl, or substituted naphthyl;

Each R_{D-11} is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;

R_{D-12} is H, alkyl, substituted alkyl, cycloalkyl, haloalkyl, heterocycloalkyl, substituted heterocycloalkyl, substituted phenyl, or substituted naphthyl;

wherein W is (E):

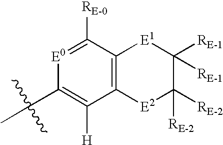

E⁰ is CH or N;

R_{E-0} is H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, R₅, R₆, —OR_{E-3}, —OR_{E-4}, —SR_{E-3}, —SR_{E-5}S, —N(R_{E-3})₂, —NR_{E-3}R_{E-6}, —N(R_{E-6})₂, —C(O)R_{E-3}, —CN, —C(O)N(R_{E-3})₂, —NR_{E-3}C(O)R_{E-3}, —S(O)R_{E-3}, —S(O)R_{E-5}, —OS(O)₂R_{E-3}, —NR_{E-3}S(O)₂R_{E-3}, —NO₂, or —N(H)C(O)N(H)R_{E-3};

E¹ is O, CR_{E-1-1}, or C(R_{E-1-1})₂, provided that when E¹ is CR_{E-1-1}, one R_{E-1} is a bond to CR_{E-1-1}, and further provided that at least one of E¹ or E2 is O;

Each R_{E-1-1} is independently H, F, Br, Cl, CN, alkyl, haloalkyl, substituted alkyl, alkynyl, cycloalkyl, —OR_{E}, or —N(R_{E})₂, provided that at least one R_{E-1-1} is H when E¹ is C(R_{E-1-1})₂;

Each R_{E-1} is independently H, alkyl, substituted alkyl, haloalkyl, cycloalkyl, heterocycloalkyl, or a bond to E¹ provided that E¹ is CR_{E-1-1};

E² is O, CR_{E-2-2}, or C(R_{E-2-2})₂, provided that when E² is CR_{E-2-2}, one R_{E-2} is a bond to CR_{E-2-2}, and further provided that at least one of E¹ or E² is O;

Each R_{E-2-2} is independently H, F, Br, Cl, CN, alkyl, haloalkyl, substituted alkyl, alkynyl, cycloalkyl, —OR_{E}, or —N(R_{E})₂, provided that at least one R_{E-2-2} is H when E² is C(R_{E-2-2})₂;

Each R_{E-2} is independently H, alkyl, substituted alkyl, haloalkyl, cycloalkyl, heterocycloalkyl, or a bond to E² provided that E² is CR_{E-2-2};

Each R_{E} is independently H, alkyl, cycloalkyl, heterocycloalkyl, haloalkyl, halocycloalkyl, or haloheterocycloalkyl;

Each R_{E-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I or substituted phenyl;

R_{E-4} is H, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;

Each R_{E-5} is independently H, haloalkyl, substituted alkyl, cycloalkyl, halocycloalkyl, substituted cycloalkyl, heterocycloalkyl, haloheterocycloalkyl, substituted heterocycloalkyl, R₅, or R₆;

Each R_{E-6} is independently alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I;

wherein W is (F):

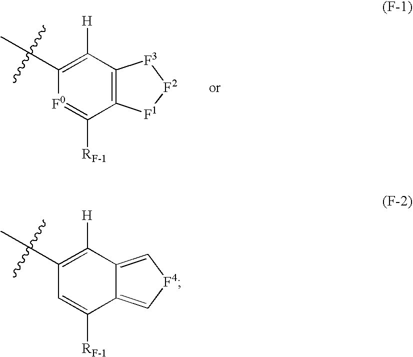

F⁰ is C(H) wherein F¹ - - - F² - - - F³ is selected from O—C(R_{F-2})═N, O—C(R_{F-3})(R_{F-2})—N(R_{F-4}), O—C(R_{F-3})(R_{F-2})—S, O—N═C(R_{F-3}), O—C(R_{F-2})(R_{F-5})—O , O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), C(R_{F-3})(R_{F-6})—C(R_{F-2})(R_{F-6})—C(R_{F-3})(R_{F-6}), or C(R_{F-3})₂—C(R_{F-2})(R_{F-3})—C(R_{F-3})₂;

F⁰ is N wherein F¹ - - - F² - - - F³ is selected from O—C(R_{F-2})═N, O—C(R_{F-3})(R_{F-2})—N(R_{F-4}), O—C(R_{F-3})(R_{F-2})—S, O—N═C(R_{F-3}) O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), C(R_{F-3})═C(R_{F-2})—C(R_{F-3})₂, or C(R_{F-3})₂—C(R_{F-2})(R_{F-3})—C(R_{F-3})₂;

F⁴ is N(R_{F-7}), O, or S;

R_{F-1} is H, F, Cl, Br, I, —CN, —CF₃, —OR_{F-8}, —SR_{F-8}, or —N(R_{F-8})₂;

R_{F-2} is H, F, alkyl, haloalkyl, substituted alkyl, lactam heterocycloalkyl, phenoxy, substituted phenoxy, R₅, R₆, —N(R_{F-4})-aryl, —N(R_{F-4})-substituted phenyl, —N(R_{F-4})-substituted naphthyl, —O-substituted phenyl, —O-substituted naphthyl, —S-substituted phenyl, —S-substituted naphthyl, or alkyl substituted on the ω carbon with R_{F-9} where said ω carbon is determined by counting the longest carbon chain of the alkyl moiety with the C-1 carbon being the carbon attached to W and the ω carbon being the carbon furthest, e.g., separated by the greatest number of carbon atoms in the chain, from said C-1 carbon;

R_{F-3} is H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{F-8}, —C(O)N(R_{F-8})₂, —NHR_{F-8}, —NR_{F-8}COR_{F-8}, —N(R_{F-8})₂, —SR_{F-8}, —C(O)R_{F-8}, —CO₂R_{F-8}, aryl, R₅, or R₆;

R_{F-4} is H, or alkyl;

Each R_{F-5} is independently F, Br, Cl, I, alkyl, substituted alkyl, haloalkyl, alkenyl, substituted alkenyl, haloalkenyl, alkynyl, substituted alkynyl, haloalkynyl, —CN, —CF₃, —OR_{F-8}, —C(O)NH₂, —NHR_{F-8}, —SR_{F-8}, —CO₂R_{F-8}, aryl, phenoxy, substituted phenoxy, heteroaryl, —N(R_{F-4})-aryl, or —O-substituted aryl;

One of R_{F-6} is H, alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆, and each of the other two R_{F-6} is independently selected from alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆;

R_{F-7} is H, alkyl, haloalkyl, substituted alkyl, cycloalkyl, halocycloalkyl, substituted cycloalkyl, phenyl, or phenyl having 1 substituent selected from R₉ and further having 0-3 substituents independently selected from F, Cl, Br, or I;

R_{F-8} is H, alkyl, substituted alkyl, cycloalkyl, haloalkyl, heterocycloalkyl, substituted heterocycloalkyl, substituted phenyl, or substituted naphthyl;

R_{F-9} is aryl, R₅, or R₆;

wherein W is (G):

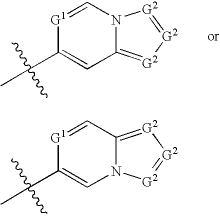

G¹ is N or CH;

Each G² is N or C(R_{G-1}), provided that no more than one G² is N;

Each R_{G-1} is independently H, alkyl, substituted alkyl, haloalkyl, alkenyl, substituted alkenyl, haloalkenyl, alkynyl, substituted alkynyl, haloalkynyl, —CN, —NO₂, F, Br, Cl, I, —C(O)N(R_{G-3})₂, —N(R_{G-3})₂, —SR_{G-6}, —S(O)₂R_{G-6}, —OR_{G-6}, —C(O)R_{G-6}, —CO₂R_{G-6}, aryl, R₅, R₆, or two R_{G-1} on adjacent carbon atoms may combine for W to be a 6-5-6 fused-tricyclic-heteroaromatic-ring system optionally substituted on the newly formed ring where valency allows with 1-2 substitutents independently selected from F, Cl, Br, I, and R_{G-2};

R_{G-2} is alkyl, alkenyl, alkynyl, cycloalkyl, heterocycloalkyl, haloalkyl, haloalkenyl, haloalkynyl, halocycloalkyl, haloheterocycloalkyl, —OR_{G-8}, —SR_{G-8}, —S(O)₂R_{G-8}, —S(O)R_{G-8}, —OS(O)₂R_{G-8}, —N(R_{G-8})₂, —C(O)R_{G-8}, —C(S)R_{G-8}, —C(O)OR_{G-8}, —CN, —C(O)N(R_{G-8})₂, —NR_{G-8}C(O)R_{G-8}, —S(O)₂N(R_{G-8})₂, —NR_{G-8}S(O)₂R_{G-8}, —NO₂, —N(R_{G-8})C(O)N(R_{G-8})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R_{G-7}, naphthyl, or naphthyl having 0-4 substituents independently selected from F, Cl, Br, I, or R_{G-7};

provided that when G² adjacent to the bridge N is C(R_{G-1}) and the other G² are CH, that R_{G-1} is other than H, F, Cl, I, alkyl, substituted alkyl or alkynyl;

Each R_{G-3} is independently H, alkyl, cycloalkyl, heterocycloalkyl, alkyl substituted with 1 substituent selected from R_{G-4}, cycloalkyl substituted with 1 substituent selected from R_{G-4}, heterocycloalkyl substituted with 1 substituent selected from R_{G-4}, haloalkyl, halocycloalkyl, haloheterocycloalkyl, phenyl, or substituted phenyl;

R_{G-4} is —OR_{G-5}, —SR_{G-5}, —N(R_{G-5})₂, —C(O)R_{G-5}, —SOR_{G-5}, —SO₂R_{G-5}, —C(O)N(R_{G-5})₂, —CN, —CF₃, —NR_{G-5}C(O)R_{G-5}, —S(O)₂N(R_{G-5})₂, —NR_{G-5}S(O)₂R_{G-5}, or —NO₂;

Each R_{G-5} is independently H, alkyl, cycloalkyl, heterocycloalkyl, haloalkyl, halocycloalkyl, or haloheterocycloalkyl;

R_{G-6} is H, alkyl, haloalkyl, substituted alkyl, cycloalkyl, halocycloalkyl, substituted cycloalkyl, phenyl, or phenyl having 0-4 substituents independently selected from F, Cl, Br, I, and R_{G-7};

R_{G-7} is alkyl, substituted alkyl, haloalkyl, —OR_{G-5}, —CN, —NO₂, —N(R_{G-3})₂;

Each R_{G-8} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R_{G-7};

wherein W is (H)

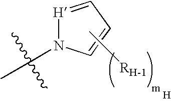

H′ is N or CH;

Each R_{H-1} is independently F, Cl, Br, I, —CN, —NO₂, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, lactam heterocyclcoalkyl, aryl, R₅, R₆, —OR₈, —SR₈, —SOR₈, —SO₂R₈, —SCN, —S(O)N(R₈)₂, —S(O)₂N(R₈)₂, —C(O)R₈, —C(O)₂R₈, —C(O)N(R₈)₂, C(R₈)═N—OR₈, —NC(O)R₅, —NC(O)R_{H-3}, —NC(O)R₆, —N(R₈)₂, —NR₈C(O)R₈, —NR₈S(O)₂R₈, or two R_{H-1} on adjacent carbon atoms may fuse to form a 6-membered ring to give a 5-6 fused, bicyclic moiety where the 6-membered ring is optionally substituted with 1-3 substitutents selected from R_{H-2};

m_{H} is 0, 1, or 2;

R_{H-2} is alkyl, alkenyl, alkynyl, cycloalkyl, heterocycloalkyl, haloalkyl, haloalkenyl, haloalkynyl, halocycloalkyl, haloheterocycloalkyl, —OR_{H-3}, —SR_{H-3}, —S(O)₂R_{H-3}, —S(O)R_{H-3}, —OS(O)₂R_{H-3}, —N(R_{H-3})₂, —C(O)R_{H-3}, —C(S)R_{H-3}, —C(O)OR_{H-3}, —CN, —C(O)N(R_{H-3})₂, —NR_{H-3}C(O)R_{H-3}, —S(O)₂N(R_{H-3})₂, —NR_{H-3}S(O)₂R_{H-3}, —NO₂, —N(R_{H-3})C(O)N(R_{H-3})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R₇, naphthyl, naphthyl having 0-4 substituents independently selected from F, Cl, Br, I, or R₇, or two R_{H-2} on adjacent carbon atoms may combine to form a three-ring-fused-5-6-6 system optionally substituted with up to 3 substituents independently selected from Br, Cl, F, I, —CN, —NO₂, —CF₃, —N(R_{H-3})₂, —N(R_{H-3})C(O)R_{H-3}, alkyl, alkenyl, and alkynyl;

Each R_{H-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R₇;

or pharmaceutical composition, pharmaceutically acceptable salt, racemic mixture, or pure enantiomer thereof.

Abbreviations which are well known to one of ordinary skill in the art may be used (e.g., “Ph” for phenyl, “Me” for methyl, “Et” for ethyl, “h” or “hr” for hour or hours, “min” for minute or minutes, and “rt” for room temperature).

All temperatures are in degrees Centigrade.

Room temperature is within the range of 15-25 degrees Celsius.

AChR refers to acetylcholine receptor.

nAChR refers to nicotinic acetylcholine receptor.

Pre-senile dementia is also known as mild cognitive impairment.

5HT₃R refers to the serotonin-type 3 receptor.

α-btx refers to α-bungarotoxin.

FLIPR refers to a device marketed by Molecular Devices, Inc. designed to precisely measure cellular fluorescence in a high throughput whole-cell assay. (Schroeder et. al., J. Biomolecular Screening, 1(2), p 75-80, 1996).

TLC refers to thin-layer chromatography.

HPLC refers to high pressure liquid chromatography.

MeOH refers to methanol.

EtOH refers to ethanol.

IPA refers to isopropyl alcohol.

THF refers to tetrahydrofuran.

DMSO refers to dimethylsulfoxide.

DMF refers to N,N-dimethylformamide.

EtOAc refers to ethyl acetate.

TMS refers to tetramethylsilane.

TEA refers to triethylamine.

DIEA refers to N,N-diisopropylethylamine.

MLA refers to methyllycaconitine.

Ether refers to diethyl ether.

HATU refers to O-(7-azabenzotriazol-1-yl)-N,N,N′, N′-tetramethyluronium hexafluorophosphate.

CDI refers to carbonyl diimidazole.

NMO refers to N-methylmorpholine-N-oxide.

TPAP refers to tetrapropylammonium perruthenate.

Na₂SO₄ refers to sodium sulfate.

K₂CO₃ refers to potassium carbonate.

MgSO₄ refers to magnesium sulfate.

When Na₂SO₄, K₂CO₃, or MgSO₄ is used as a drying agent, it is anhydrous.

Halogen is F, Cl, Br, or I.

The carbon atom content of various hydrocarbon-containing moieties is indicated by a prefix designating the minimum and maximum number of carbon atoms in the moiety, i.e., the prefix Cᵢ₋ⱼ indicates a moiety of the integer “i” to the integer “j” carbon atoms, inclusive. Thus, for example, C₁₋₆ alkyl refers to alkyl of one to six carbon atoms.

Non-inclusive examples of moieties that fall within the definition of R₅ and R₆ include, but are not limited to, thienyl, benzothienyl, pyridyl, thiazolyl, quinolyl, pyrazinyl, pyrimidyl, imidazolyl, furanyl, benzofuranyl, benzothiazolyl, isothiazolyl, benzisothiazolyl, benzisoxazolyl, benzimidazolyl, indolyl, benzoxazolyl, pyrazolyl, triazolyl, tetrazolyl, isoxazolyl, oxazolyl, pyrrolyl, isoquinolinyl, cinnolinyl, indazolyl, indolizinyl, phthalazinyl, pydridazinyl, triazinyl, isoindolyl, purinyl, oxadiazolyl, furazanyl, benzofurazanyl, benzothiophenyl, benzothiazolyl, quinazolinyl, quinoxalinyl, naphthridinyl, and furopyridinyl.

Non-inclusive examples of heterocycloalkyl include, but are not limited to, tetrahydrofurano, tetrahydropyrano, morpholino, pyrrolidino, piperidino, piperazine, azetidino, azetidinono, oxindolo, dihydroimidazolo, and pyrrolidinono

Some of the amines described herein require the use of an amine-protecting group to ensure functionalization of the desired nitrogen. One of ordinary skill in the art would appreciate where, within the synthetic scheme to use said protecting group. Amino protecting group includes, but is not limited to, carbobenzyloxy (CBz), tert butoxy carbonyl (BOC) and the like. Examples of other suitable amino protecting groups are known to person skilled in the art and can be found in “Protective Groups in Organic synthesis,” 3rd Edition, authored by Theodora Greene and Peter Wuts.

Alkyl substituted on an Co carbon with R_{A-7} is determined by counting the longest carbon chain of the alkyl moiety with the C-1 carbon being the carbon attached to the W moiety and the ω carbon being the carbon furthest, e.g., separated by the greatest number of carbon atoms in the chain, from said C-1 carbon. Therefore, when determining the ω carbon, the C-1 carbon will be the carbon attached, as valency allows, to the W moiety and the Co carbon will be the carbon furthest from said C-1 carbon.

The core molecule is Azabicyclo-N(R₁)—C(═X)—:

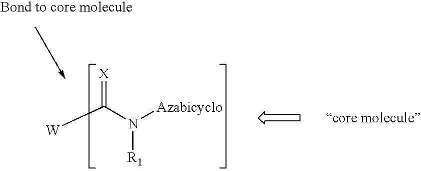

Mammal denotes human and other mammals.

Brine refers to an aqueous saturated sodium chloride solution.

Equ means molar equivalents.

IR refers to infrared spectroscopy.

Lv refers to leaving groups within a molecule, including Cl, OH, or mixed anhydride.

NMR refers to nuclear (proton) magnetic resonance spectroscopy, chemical shifts are reported in ppm (δ) downfield from TMS.

MS refers to mass spectrometry expressed as m/e or mass/charge unit. HRMS refers to high resolution mass spectrometry expressed as m/e or mass/charge unit. [M+H]⁺ refers to an ion composed of the parent plus a proton. [M−H]⁻ refers to an ion composed of the parent minus a proton. [M+Na]⁺ refers to an ion composed of the parent plus a sodium ion. [M+K]⁺ refers to an ion composed of the parent plus a potassium ion. EI refers to electron impact. ESI refers to electrospray ionization. CI refers to chemical ionization. FAB refers to fast atom bombardment.

Alpha-7 nAChR full agonists within the present invention may be in the form of pharmaceutically acceptable salts. The term “pharmaceutically acceptable salts” refers to salts prepared from pharmaceutically acceptable non-toxic bases including inorganic bases and organic bases, and salts prepared from inorganic acids, and organic acids. Salts derived from inorganic bases include aluminum, ammonium, calcium, ferric, ferrous, lithium, magnesium, potassium, sodium, zinc, and the like. Salts derived from pharmaceutically acceptable organic non-toxic bases include salts of primary, secondary, and tertiary amines, substituted amines including naturally occurring substituted amines, cyclic amines, such as arginine, betaine, caffeine, choline, N, N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like. Salts derived from inorganic acids include salts of hydrochloric acid, hydrobromic acid, hydroiodic acid, sulfuric acid, phosphoric acid, phosphorous acid and the like. Salts derived from pharmaceutically acceptable organic non-toxic acids include salts of C₁₋₆ alkyl carboxylic acids, di-carboxylic acids, and tri-carboxylic acids such as acetic acid, propionic acid, fumaric acid, succinic acid, tartaric acid, maleic acid, adipic acid, and citric acid, and aryl and alkyl sulfonic acids such as toluene sulfonic acids and the like.

By the term “effective amount” of a compound as provided herein is meant a nontoxic but sufficient amount of the compound(s) to provide the desired therapeutic effect. As pointed out below, the exact amount required will vary from subject to subject, depending on the species, age, and general condition of the subject, the severity of the disease that is being treated, the particular compound(s) used, the mode of administration, and the like. Thus, it is not possible to specify an exact “effective amount.” However, an appropriate effective amount may be determined by one of ordinary skill in the art using only routine experimentation.

In addition to the compound(s) of Formula I, the compositions use may also comprise one or more non-toxic, pharmaceutically acceptable carrier materials or excipients. A generally recognized compendium of such methods and ingredients is Remington's Pharmaceutical Sciences by E. W. Martin (Mark Publ. Co., 15th Ed., 1975). The term “carrier” material or “excipient” herein means any substance, not itself a therapeutic agent, used as a carrier and/or diluent and/or adjuvant, or vehicle for delivery of a therapeutic agent to a subject or added to a pharmaceutical composition to improve its handling or storage properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition. Acceptable excipients include lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinyl-pyrrolidone, and/or polyvinyl alcohol, and then tableted or encapsulated for convenient administration. Such capsules or tablets may contain a controlled-release formulation as may be provided in a dispersion of active compound in hydroxypropyl-methyl cellulose, or other methods known to those skilled in the art. For oral administration, the pharmaceutical composition may be in the form of, for example, a tablet, capsule, suspension or liquid. If desired, other active ingredients may be included in the composition.

In addition to the oral dosing, noted above, the compositions of the present invention may be administered by any suitable route, e.g., parenterally, bucal, intravaginal, and rectal, in the form of a pharmaceutical composition adapted to such a route, and in a dose effective for the treatment intended. Such routes of administration are well known to those skilled in the art. The compositions may, for example, be administered parenterally, e.g., intravascularly, intraperitoneally, subcutaneously, or intramuscularly. For parenteral administration, saline solution, dextrose solution, or water may be used as a suitable carrier. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, EtOH, corn oil, cottonseed oil, peanut oil, sesame oil, benzyl alcohol, sodium chloride, and/or various buffers. Other adjuvants and modes of administration are well and widely known in the pharmaceutical art.

The serotonin type 3 receptor (5HT₃R) is a member of a superfamily of ligand-gated ion channels, which includes the muscle and neuronal nAChR, the glycine receptor, and the γ-aminobutyric acid type A receptor. Like the other members of this receptor superfamily, the 5HT₃R exhibits a large degree of sequence homology with α7 nAChR but functionally the two ligand-gated ion channels are very different. For example, α7 nAChR is rapidly inactivated, is highly permeable to calcium and is activated by acetylcholine and nicotine. On the other hand, 5HT₃R is inactivated slowly, is relatively impermeable to calcium and is activated by serotonin. These experiments suggest that the α7 nAChR and 5HT₃R proteins have some degree of homology, but function very differently. Indeed the pharmacology of the channels is very different. For example, Ondansetron, a highly selective 5HT₃R antagonist, has little activity at the α7 nAChR. The converse is also true. For example, GTS-21, a highly selective α7 nAChR full agonist, has little activity at the 5HT₃R.

α7 nAChR is a ligand-gated Ca⁺⁺ channel formed by a homopentamer of α7 subunits. Previous studies have established that α-bungarotoxin (α-btx) binds selectively to this homopetameric, α7 nAChR subtype, and that α7 nAChR has a high affinity binding site for both α-btx and methyllycaconitine (MLA). α7 nAChR is expressed at high levels in the hippocampus, ventral tegmental area and ascending cholinergic projections from nucleus basilis to thalamocortical areas. α7 nAChR full agonists increase neurotransmitter release, and increase cognition, arousal, attention, learning and memory.

The α7 nAChR is one receptor system that has proved to be a difficult target for testing. Native α7 nAChR is not routinely able to be stably expressed in most mammalian cell lines (Cooper and Millar, J. Neurochem., 1997, 68(5):2140-51). Another feature that makes functional assays of α7 nAChR challenging is that the receptor is rapidly (100 milliseconds) inactivated. This rapid inactivation greatly limits the functional assays that can be used to measure channel activity.

Recently, Eisele et al. has indicated that a chimeric receptor formed between the N-terminal ligand binding domain of the α7 nAChR (Eisele et al., Nature, 366(6454), p 479-83, 1993), and the pore forming C-terminal domain of the 5-HT₃ receptor expressed well in Xenopus oocytes while retaining nicotinic agonist sensitivity. Eisele et al. used the N-terminus of the avian (chick) form of the α7 nAChR receptor and the C-terminus of the mouse form of the 5-HT₃ gene. However, under physiological conditions the α7 nAChR is a calcium channel while the 5-HT₃R is a sodium and potassium channel. Indeed, Eisele et al. teaches that the chicken α7 nAChR/mouse 5-HT₃R behaves quite differently than the native α7 nAChR with the pore element not conducting calcium but actually being blocked by calcium ions.

WO 00/73431 A2 reports on assay conditions under which the 5-HT₃R can be made to conduct calcium. This assay may be used to screen for agonist activity at this receptor. FLIPR is designed to read the fluorescent signal from each well of a 96 or 384 well plate as fast as twice a second for up to 30 minutes. This assay may be used to accurately measure the functional pharmacology of α7 nAChR and 5HT₃R. To conduct such an assay, one uses cell lines that expressed functional forms of the α7 nAChR using the α7/5-HT₃ channel as the drug target and cell lines that expressed functional 5HT₃R. In both cases, the ligand-gated ion channel was expressed in SH-EP1 cells. Both ion channels can produce robust signal in the FLIPR assay.

TNF-α is a pro-inflammatory cytokine secreted by a variety of cells, including monocytes and macrophages, in response to many inflammatory stimuli (e.g., lipopolysaccharide—LPS) or external cellular stresses (e.g., osmotic shock and peroxide). Elevated levels of TNF-α over basal levels have been implicated in mediating or exacerbating a number of diseases or conditions involving inflammation, pain, cancer, and diabetes. TNF-α is upstream in the, cytokine cascade of inflammation. By decreasing levels of TNF-α, not only are levels of TNF-α minimized, but also elevated levels of other inflammatory and proinflammatory cytokines, such as IL-1, IL-6, and IL-8. TNF-α plays a role in head trauma, stroke, and ischemia. Shohami et al., J. Cereb. Blood Flow Metab., 14, 615 (1994), TNF-α promotes the infiltration of other cytokines (IL-1beta, IL-6) and also chemokines, which promote neutrophil infiltration into the infarct area. TNF-α plays a role in promoting certain viral life cycles and disease states associated with them; for instance, TNF-α secreted by monocytes induced elevated levels of HIV expression in a chronically infected T cell clone. Clouse et al., J. Immunol., 142, 431 (1989); Lahdevirte et al., Am. J. Med. 85, 289 (1988). TNF-α is associated with the HIV mediated states of cachexia due to cancer and muscle degradation.

TNF-α plays a role in pancreatic beta cell destruction and diabetes. Yoon J W, and Jun H S, Diabetologia, 44(3), 271-285 (2001). Pancreatic beta cells produce insulin which helps mediate blood-glucose homeostasis. Deterioration of pancreatic beta cells often accompanies type I diabetes. Pancreatic beta cell functional abnormalities may occur in patients with type II diabetes. Type II diabetes is characterized by a functional resistance to insulin. Further, type II diabetes is also often accompanied by elevated levels of plasma glucagon and increased rates of hepatic glucose production.

In rheumatoid arthritis, TNF-α induces synoviocytes and chondrocytes to produce collagenase and neutral proteases, which lead to tissue destruction within the arthritic joints. In a model of arthritis (collagen-induced arthritis (CIA) in rats and mice), intra-articular administration of TNF-α either prior to or after the induction of CIA led to an accelerated onset of arthritis and a more severe course of the disease. Brahn et al., Lymphokine Cytokine Res., 11, 253 (1992); and Cooper, Clin. Exp. Immunol., 898, 244 (1992). By reducing TNF-α levels, the resulting levels of synoviocytes and chondrocytes are also reduced to prevent or minimize the effects of rheumatoid arthritis.

Alpha 7 nAChR full agonists are useful to treat, or used to prepare a medicament used to treat, diseases or conditions where a mammal receives symptomatic relief from the decrease of levels of TNF-α; these diseases or conditions include, but are not limited to, any one or more or combination of the following: rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome (ARDS); Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, or HIV-3; CMV; influenza, adenovirus, a herpes virus (including HSV-1, HSV-2); herpes zoster; multiple myeloma; acute and chronic myelogenous leukemia; cancer-associated cachexia; pancreatic beta cell destruction; type I or type II diabetes.

Some nicotinic receptors regulate vascular angiogenesis; for example, the binding of nicotine to the alpha-7 nAChR stimulates DNA synthesis and proliferation of vascular endothelial cells. Villablanca, supra. The present invention includes alpha-7 nAChR full agonists that are also useful to treat, or are used to prepare a medicament to treat, diseases or conditions where a mammal receives symptomatic relief from the stimulation of vascular angiogenesis; these diseases or conditions include, but not limited to, any one or more of the following: wound healing (healing burns, and wounds in general including from surgery), bone fracture healing, ischemic heart disease, and stable angina pectoris.

The key step in the preparation of this class of compounds is the coupling of the Azabicyclo moiety with the requisite acid chloride (Lv=Cl), mixed anhydride (e.g., Lv=diphenyl phosphoryl, bis(2-oxo-3-oxazolidinyl)phosphinyl, or acyloxy of the general formula of O—C(O)—R_{Lv}, where R_{Lv} includes phenyl or t-butyl), or carboxylic acid (Lv=OH) in the presence of an activating reagent. Suitable activating reagents are well known in the art, for examples see Kiso, Y., Yajima, H. “Peptides” pp. 39-91, San Diego, Calif., Academic Press, (1995), and include, but are not limited to, agents such as carbodiimides, phosphonium and uronium salts (such as HATU).

Compounds of Formula I can be prepared as shown in Scheme 1. The key step in the preparation of this class of compounds is the coupling of an azabicyclic moiety with the requisite acid chloride (Lv=Cl), mixed anhydride (e.g., Lv=diphenyl phosphoryl, bis(2-oxo-3-oxazolidinyl)phosphinyl, or acyloxy of the general formula of O—C(O)—R_{Lv}, where R_{Lv} includes phenyl or t-butyl), or carboxylic acid (Lv=OH) in the presence of an activating reagent. Suitable activating reagents are well known in the art, for examples see Kiso, Y., Yajima, H. “Peptides” pp. 39-91, San Diego, Calif., Academic Press, (1995), and include, but are not limited to, agents such as carbodiimides, phosphonium and uronium salts (such as HATU).

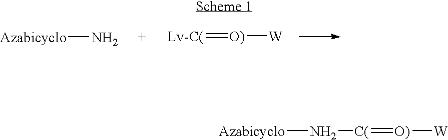

Generally, the carboxylic acid is activated with a uronium salt, preferably HATU (see J. Am. Chem. Soc., 4397 (1993)), in the presence of the Azabicyclico moiety and a base such as DIEA in DMF to afford the desired amides. Alternatively, the carboxylic acid is converted to the acyl azide by using DPPA; the appropriate amine precursor is added to a solution of the appropriate anhydride or azide to give the desired final compounds. In some cases, the ester (Lv being OMe or OEt) may be reacted directly with the amine precursor in refluxing methanol or ethanol to give the compounds of Formula I.

Certain 6-substituted-[2.2.2]-3-amines (Azabicyclo I) are known in the art. The preparation of compounds where R₂ is present is described in Acta Pol. Pharm. 179-85 (1981). Alternatively, the 6-substituted-[2.2.2]-3-amine can be prepared by reduction of an oxime or an imine of the corresponding 6-substituted-3-quinuclidinone by methods known to one of ordinary skill in the art (see J. Labelled Compds. Radiopharm., 53-60 (1995), J. Med. Chem. 988-995, (1998), Synth. Commun. 1895-1911 (1992), Synth. Commun. 2009-2015 (1996)). Alternatively, the 6-substituted-[2.2.2]-3-amine can be prepared from a 6-substituted-3-hydroxyquinuclidine by Mitsunobu reaction followed by deprotection as described in Synth. Commun. 1895-1911 (1995). Alternatively, the 6-substituted-[2.2.2]-3-amine can be prepared by conversion of a 6-substituted-3-hydroxyquinuclidine into the corresponding mesylate or tosylate, followed by displacement with sodium azide and reduction as described in J. Med. Chem. 587-593 (1975). 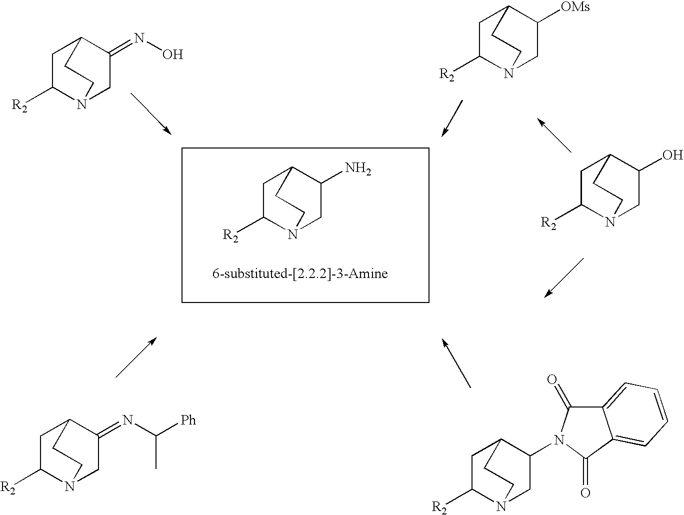
The oximes can be prepared by treatment of the 3-quinuclidinones with hydroxylamine hydrochloride in the presence of base. The imines can be prepared by treatment of the 3-quinuclidinones with a primary amine under dehydrating conditions. The 3-hydroxyquinuclidines can be prepared by reduction of the 3-quinuclidinones. The 6-substituted-3-quinuclidinones can be prepared by known procedures (see J. Gen. Chem. Russia 3791-3795, (1963), J. Chem. Soc. Perkin Trans. I 409-420 (1991), J. Org. Chem. 3982-3996 (2000)).

One of ordinary skill in the art will recognize that the methods described for the reaction of the unsubstituted 3-amino-1-azabicyclo[2.2.1]heptane (R₂=absent) are equally applicable to substituted compounds (R₂≠H). For where Azabicyclo is II, compounds where R₂ is present can be prepared from appropriately substituted nitro alcohols using procedures described in Tetrahedron (1997), 53, p. 11121 as shown below. Methods to synthesize nitro alcohols are well known in the art (see J. Am. Chem. Soc. (1947), 69, p 2608). The scheme below is a modification of the synthesis of exo-3-amino-1-azabicyclo[2.2.1]heptane as the bis(hydro para-toluenesulfonate) salt, described in detail herein, to show how to obtain these amine precursors. The desired salt can be made using standard procedures.

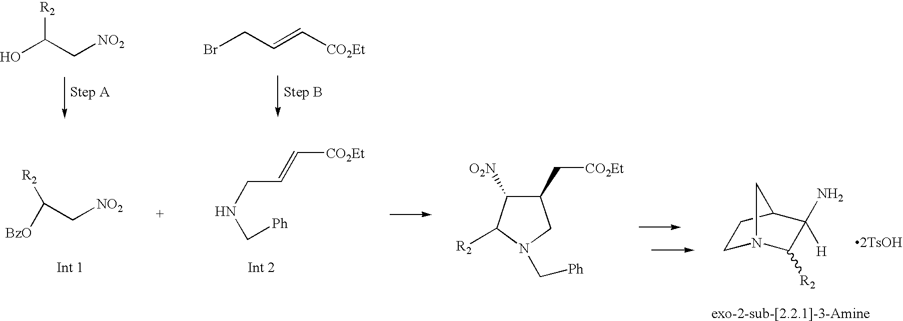

Compounds for Azabicyclo II where R₂ is present can also be prepared by modification of intermediates described in the synthesis of exo-3-amino-1-azabicyclo[2.2.1]heptane as the bis(hydro para-toluenesulfonate) salt, described in detail herein. For example, Int 6 can be oxidized to the aldehyde and treated with an organometallic reagent to provide Int 20 using procedures described in Tetrahedron (1999), 55, p 13899. Int 20 can be converted into the amine using methods described for the synthesis of exo-3-amino-1-azabicyclo[2.2.1]heptane as the bis(hydro para-toluenesulfonate) salt. Once the amine is obtained, the desired salt can be made using standard procedures. 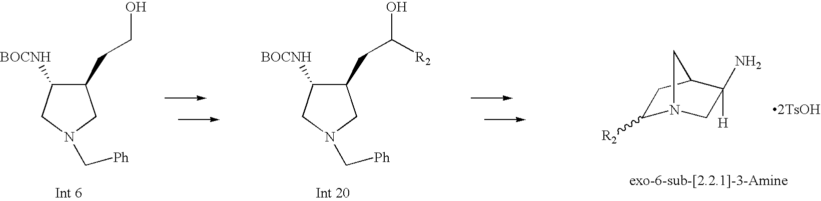
The schemes used are for making exo-3-amino-1-azabicyclo[2.2.1]heptane. However, the modifications discussed are applicable to make the endo isomer also.

There are several methods by which the amine precursor for Azabicyclo III and Azabicyclo IV can be obtained: 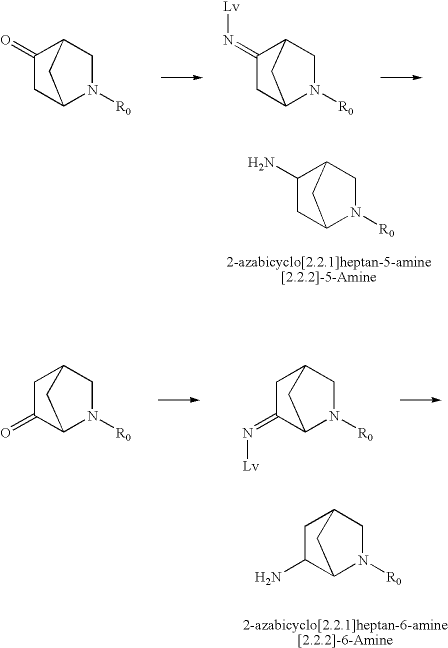
where Lv can be —CH₂Ph, —CH(Me)Ph, —OH, —OMe, or —OCH₂Ph. The respective amine precursors for Azabicyclo III and Azabicyclo IV can be prepared by reduction of an oxime or an imine of the corresponding N-2-azabicyclo[2.2.1]-heptanone by methods known to one skilled in the art (see J. Labelled Compds. Radiopharm., 53-60 (1995), J. Med. Chem. 988-995, (1998), Synth. Commun. 1895-1911 (1992), Synth. Commun. 2009-2015 (1996)). The oximes can be prepared by treatment of the N-2-azabicyclo[2.2.1]heptanones with hydroxylamine hydrochloride in the presence of a base. The imines can be prepared by treatment of the N-2-azabicyclo[2.2.1]-heptanones with a primary amine under dehydrating conditions. The N-2-azabicyclo[2.2.1]heptanones can be prepared by known procedures (see Tet. Lett. 1419-1422 (1999), J. Med. Chem. 2184-2191 (1992), J. Med. Chem. 706-720 (2000), J. Org. Chem., 4602-4616 (1995)).

The exo- and endo-1-azabicyclo[3.2.1]octan-3-amines are prepared from 1-azabicyclic[3.2.1]octan-3-one (Thill, B. P., Aaron, H. S., J. Org. Chem., 4376-4380 (1968)) according to the general procedure as discussed in Lewin, A. H., et al., J. Med. Chem., 988-995 (1998).

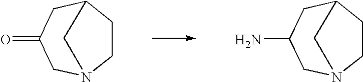

One of ordinary skill in the art will also recognize that the methods described for the reaction of the unsubstituted 1-azabicyclo[3.2.1]octan-3-amine or 1-azabicyclo[3.2.2]nonan-3-amine (R₂=absent) are equally applicable to substituted compounds (R₂ present). The R₂ substituent may be introduced as known to one skilled in the art through standard alkylation chemistry. Exposure of 1-azabicyclo[3.2.1]octan-3-one or 1-azabicyclo[3.2.2]nonan-3-one to a hindered base such as LDA (lithium diisopropylamide) in a solvent such as THF or ether between 0° C. to −78° C. followed by the addition of an alkylating agent (R₂Lv, where Lv=Cl, Br, I, OTs, etc.) will, after being allowed to warm to about 0° C. to rt followed by an aqueous workup, provide the desired compound as a mixture of isomers. Chromatographic resolution (flash, HPLC, or chiral HPLC) will provided the desired purified alkylated ketones. From there, formation of the oxime and subsequent reduction will provide the desired endo or exo isomers.

### Amines

Preparation of N-(2S,3R)-2-methyl-1-azabicyclo[2.2.2]octan-3-amine dihydrochloride (2S-methyl-2.2.2-Amine): See, e.g., US 20020042428 A1.

Preparation of the 1-azabicyclo-2.2.1Amines:

### Synthesis of exo-3-amino-1-azabicyclo[2.2.1]heptane as the bis(hydro para-toluenesulfonate) salt (exo-[2.2.1]-Amine)

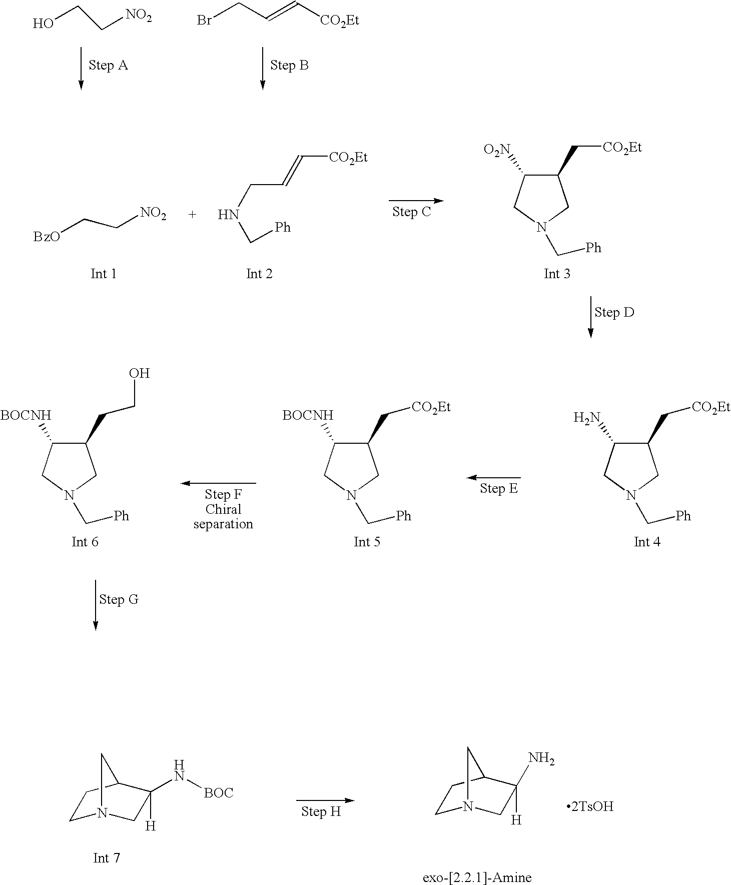 Step A. Preparation of 2-(benzoyloxy)-1-nitroethane (Int 1).

Benzoyl chloride (14.9 mL, 128 mmol) is added to a stirred solution of nitroethanol (9.2 mL, 128 mmol) in dry benzene (120 mL). The solution is refluxed for 24 hr and then concentrated in vacuo. The crude product is purified by flash chromatography on silica gel. Elution with hexanes-EtOAc (80:20) affords Int 1 as a white solid (68% yield): ¹H NMR (CDCl₃) δ 8.0, 7.6, 7.4, 4.9, 4.8.

Step B. Preparation of ethyl E-4-(benzylamino)-2-butenoate (Int 2).

Ethyl E-4-bromo-2-butenoate (10 mL, 56 mmol, tech grade) is added to a stirred solution of benzylamine (16 mL, 146 mmol) in CH₂Cl₂ (200 mL) at rt. The reaction mixture stirs for 15 min, and is diluted with ether (1 L). The mixture is washed with saturated aqueous NaHCO₃ solution (3×) and water, dried (Na₂SO₄), filtered and concentrated in vacuo. The residue is purified by flash chromatography on silica gel. Elution with hexanes-EtOAc (70:30) affords Int 2 as a clear oil (62% yield): ¹H NMR (CDCl₃) δ 7.4-7.2, 7.0, 6.0, 4.2, 3.8, 3.4, 2.1-1.8, 1.3.

Step C. Preparation of trans-4-nitro-1-(phenylmethyl)-3-pyrrolidineacetic acid ethyl ester (Int 3).

A solution of Int 1 (6.81 g, 34.9 mmol) and Int 2 (7.65 g, 34.9 mmol) in EtOH (70 mL) stirs at rt for 15 h and is then concentrated in vacuo. The residue is diluted with ether (100 mL) and saturated aqueous NaHCO₃ solution (100 mL). The organic layer is separated and dried (Na₂SO₄), filtered and concentrated in vacuo. The crude product is purified by flash chromatography on silica gel. Elution with hexanes-EtOAc (85:15) affords Int 3 as a clear oil (76% yield): ¹H NMR (CDCl₃) δ 7.4-7.3, 4.8-4.7, 4.1, 3.8-3.6, 3.3-3.0, 2.7-2.6, 2.4-2.3, 1.2.

Step D. Preparation of trans-4-amino-1-(phenylmethyl)-3-pyrrolidineacetic acid ethyl ester (Int 4).

A mixture of Int 3 (3.28 g, 11.2 mmol) and RaNi (1.5 g) in EtOH (100 mL) is placed in a Parr bottle and hydrogenated for 4 h under an atmosphere of hydrogen (46 psi) at rt. The mixture is filtered through a pad of Celite, and the solvent is removed in vacuo to afford Int 4 as a clear oil (100% yield): ¹H NMR (300 MHz, CDCl₃) δ 7.3-7.2, 4.1, 3.6, 3.2, 3.0-2.9, 2.8, 2.8-2.6, 2.6-2.4, 2.30-2.2, 1.2.

Step E. Preparation of trans-4-(1,1-dimethylethoxycarbonylamido)-1-(phenylmethyl)-3-pyrrolidineacetic acid ethyl ester (Int 5).

Di-tert-butyldicarbonate (3.67 g, 16.8 mmol) is added to a stirred solution of Int 4 (2.94 g, 11.2 mmol) in CH₂Cl₂ (30 mL) cooled in an ice bath. The reaction is allowed to warm to rt and stirred overnight. The mixture is concentrated in vacuo. The crude product is purified by flash chromatography on silica gel. Elution with hexanes-EtOAc (80:20) affords Int 5 as a white solid (77% yield): ¹H NMR (300 MHz, CDCl₃) δ 7.4-7.2, 5.1-4.9, 4.1, 4.0-3.8, 3.6, 3.2-3.0, 2.8-2.6, 2.5-2.4, 2.3-2.1, 1.4, 1.3.

Step F. Preparation of trans (tert-butoxycarbonylamino)-4-(2-hydroxyethyl)-1-(N-phenylmethyl) pyrrolidine (Int 6).

LiAlH₄ powder (627 mg, 16.5 mmol) is added in small portions to a stirred solution of Int 5 (3.0 g, 8.3 mmol) in anhydrous THF (125 mL) in a −5° C. bath. The mixture is stirred for 20 min in a −5° C. bath, then quenched by the sequential addition of water (0.6 mL), 15% (w/v) aqueous NaOH (0.6 mL) and water (1.8 mL). Excess anhydrous K₂CO₃ is added, and the mixture is stirred for 1 h, then filtered. The filtrate is concentrated in vacuo. The residue is purified by flash chromatography on silica gel. Elution with EtOAc affords Int 6 as a white solid (94% yield): ¹H NMR (CDCl₃) δ 7.4-7.3, 5.3-5.2, 4.1-4.0, 3.9-3.7, 3.3-3.2, 2.8-2.7, 2.3-2.1, 1.7, 1.5.

Int 6 is a racemic mixture that can be resolved via chromatography using a Diacel chiral pack AD column. From the two enantiomers thus obtained, the (+)-enantiomer, [α]²⁵_{D}+35 (c 1.0, MeOH), gives rise to the corresponding enantiomerically pure exo-4-S final compounds, whereas the (−)-enantiomer, [α]²⁵_{D}−34 (c 0.98, MeOH), gives rise to enantiomerically pure exo-4-R final compounds. The methods described herein use the (+)-enantiomer of Int 6 to obtain the enantiomerically pure exo-4-S final compounds. However, the methods used are equally applicable to the (−)-enantiomer of Int 6, making non-critical changes to the methods provided herein to obtain the enantiomerically pure exo-4-R final compounds.

Step G. Preparation of exo 3-(tert-butoxycarbonylamino)-1-azabicyclo[2.2.1]heptane (Int 7).

TEA (8.0 g, 78.9 mml) is added to a stirred solution of Int 6 (2.5 g, 7.8 mmol) in CH₂Cl₂ (50 mL), and the reaction is cooled in an ice-water bath. CH₃SO₂Cl (5.5 g, 47.8 mmol) is then added dropwise, and the mixture is stirred for 10 min in an ice-water bath. The resulting yellow mixture is diluted with saturated aqueous NaHCO₃ solution, extracted with CH₂Cl₂ several times until no product remains in the aqueous layer by TLC. The organic layers are combined, washed with brine, dried (Na₂SO₄), and concentrated in vacuo. The residue is dissolved in EtOH (85 mL) and is heated to reflux for 16 h. The reaction mixture is allowed to cool to rt, transferred to a Parr bottle and treated with 10% Pd/C catalyst (1.25 g). The bottle is placed under an atmosphere of hydrogen (53 psi) for 16 h. The mixture is filtered through Celite, and fresh catalyst (10% Pd/C, 1.25 g) is added. Hydrogenolysis continues overnight. The process is repeated three more times until the hydrogenolysis is complete. The final mixture is filtered through Celite and concentrated in vacuo. The residue is purified by flash chromatography on silica gel. Elution with CHCl₃-MeOH—NH₄OH (90:9.5:0.5) affords Int 7 as a white solid (46% yield): ¹H NMR (CDCl₃) δ 5.6-5.5, 3.8-3.7, 3.3-3.2, 2.8-2.7, 2.0-1.8, 1.7-1.5, 1.5.

Step H. Preparation of exo-3-amino-1-azabicyclo[2.2.1]heptane bis(hydro-para-toluenesulfonate).

Para-toluenesulfonic acid monohydrate (1.46 g, 7.68 mmol) is added to a stirred solution of Int 7 (770 mg, 3.63 mmol) in EtOH (50 mL). The reaction mixture is heated to reflux for 10 h, followed by cooling to rt. The precipitate is collected by vacuum filtration and washed with cold EtOH to give exo-[2.2.1]-Amine as a white solid (84% yield): ¹H NMR (CD₃OD) δ 7.7, 7.3, 3.9-3.7, 3.7-3.3, 3.2, 2.4, 2.3-2.2, 1.9-1.8.

### Synthesis of endo-3-amino-1-azabicyclo[2.2.1]heptane as the bis(hydro para-toluenesulfonate) salt (endo-[2.2.1]-Amine)

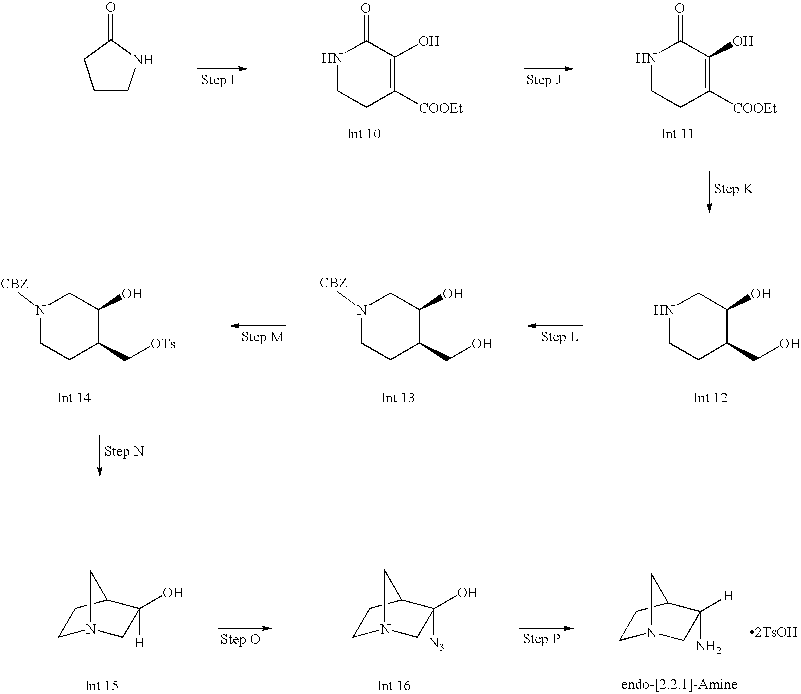 Step I. Preparation of ethyl 5-hydroxy-6-oxo-1,2,3,6-tetrahydropyridine-4-carboxylate (Int 10).

Absolute EtOH (92.0 mL, 1.58 mol) is added to a mechanically stirred suspension of potassium ethoxide (33.2 g, 395 mmol) in dry toluene (0.470 L). When the mixture is homogeneous, 2-pyrrolidinone (33.6 g, 395 mmol) is added, and then a solution of diethyl oxalate (53.1 mL, 390 mmol) in toluene (98 mL) is added via an addition funnel. After complete addition, toluene (118 mL) and EtOH (78 mL) are added sequentially. The mixture is heated to reflux for 18 h. The mixture is cooled to rt and aqueous HCl (150 mL of a 6.0 M solution) is added. The mixture is mechanically stirred for 15 min. The aqueous layer is extracted with CH₂Cl₂, and the combined organic layers are dried (MgSO₄), filtered and concentrated in vacuo to a yellow residue. The residue is recrystallized from EtOAc to afford Int 10 as a yellow solid (38% yield): ¹H NMR (CDCl₃) δ 11.4, 7.4, 4.3, 3.4, 2.6, 1.3.

Step J. Preparation of ethyl cis-3-hydroxy-2-oxopiperidine-4-carboxylate (Int 11).

A mixture of Int 10 (15 g, 81 mmol) and 5% rhodium on carbon (2.0 g) in glacial acetic acid is placed under an atmosphere of hydrogen (52 psi). The mixture is shaken for 72 h. The mixture is filtered through Celite, and the filtrate is concentrated in vacuo to afford Int 11 as a white solid (98% yield): ¹H NMR (CDCl₃) δ 6.3, 4.2, 4.0-3.8, 3.4, 3.3-3.2, 2.2, 1.3.

Step K. Preparation of cis-4-(hydroxymethyl)piperidin-3-ol (Int 12).

Int 11 (3.7 g, 19.9 mmol) as a solid is added in small portions to a stirred solution of LiAlH₄ in THF (80 mL of a 1.0 M solution) in an ice-water bath. The mixture is warmed to rt, and then the reaction is heated to reflux for 48 h. The mixture is cooled in an ice-water bath before water (3.0 mL, 170 mmol) is added dropwise, followed by the sequential addition of NaOH (3.0 mL of a 15% (w/v) solution) and water (9.0 mL, 500 mmol). Excess K₂CO₃ is added, and the mixture is stirred vigorously for 15 min. The mixture is filtered, and the filtrate is concentrated in vacuo to afford Int 12 as a yellow powder (70% yield): ¹H NMR (DMSO-d₆) δ 4.3, 4.1, 3.7, 3.5-3.2, 2.9-2.7, 2.5-2.3, 1.5, 1.3.

Step L. Preparation of benzyl cis-3-hydroxy-4-(hydroxymethyl)piperidine-1-carboxylate (Int 13).

N-(benzyloxy carbonyloxy)succinimide (3.04 g, 12.2 mmol) is added to a stirred solution of Int 12 (1.6 g, 12.2 mmol) in saturated aqueous NaHCO₃ (15 mL) at rt. The mixture is stirred at rt for 18 h. The organic and aqueous layers are separated. The aqueous layer is extracted with ether (3×). The combined organic layers are dried (K₂CO₃), filtered and concentrated in vacuo to afford Int 13 as a yellow oil (99% yield): ¹H NMR (CDCl₃) δ 7.4-7.3, 5.2, 4.3, 4.1, 3.8-3.7, 3.0-2.8, 2.1, 1.9-1.7, 1.4.

Step M. Preparation of benzyl cis-3-hydroxy-4-[(4-methylphenyl)sulfonyl oxymethyl]piperidine-1-carboxylate (Int 14).

Para-toluenesulfonyl chloride (1.0 g, 5.3 mmol) is added to a stirred solution of Int 13 (3.6 g, 5.3 mmol) in pyridine (10 mL) in a −15° C. bath. The mixture is stirred for 4 h, followed by addition of HCl (4.5 mL of a 6.0 M solution). CH₂Cl₂ (5 mL) is added. The organic and aqueous layers are separated. The aqueous layer is extracted with CH₂Cl₂. The combined organic layers are washed with brine, dried (MgSO₄), filtered and concentrated in vacuo to afford Int 14 as a colorless oil (78% yield): ¹H NMR (CDCl₃) δ 7.8, 7.4-7.2, 5.1, 4.3-4.2, 4.1, 3.9-3.8, 2.9-2.7, 2.4, 1.9, 1.6-1.3.

Step N. Preparation of exo-1-azabicyclo[2.2.1]heptan-3-ol (Int 15).

A mixture of Int 14 (3.6 g, 8.6 mmol) and 10% Pd/C catalyst (500 mg) in EtOH (50 mL) is placed under an atmosphere of hydrogen. The mixture is shaken for 16 h. The mixture is filtered through Celite. Solid NaHCO₃ (1.1 g, 13 mmol) is added to the filtrate, and the mixture is heated in an oil bath at 50° C. for 5 h. The solvent is removed in vacuo. The residue is dissolved in saturated aqueous K₂CO₃ solution. Continuous extraction of the aqueous layer using a liquid-liquid extraction apparatus (18 h), followed by drying the organic layer over anhydrous K₂CO₃ and removal of the solvent in vacuo affords Int 15 as a white solid (91% yield): ¹H NMR δ 3.8, 3.0-2.8, 2.6-2.5, 2.4-2.3, 1.7, 1.1.

Step O. Preparation of endo-3-azido-1-azabicyclo[2.2.1]heptane (Int 16).

To a mixture of Int 15 (1.0 g, 8.9 mmol) and triphenyl phosphine (3.0 g, 11.5 mmol) in toluene-THF (50 mL, 3:2) in an ice-water bath are added sequentially a solution of hydrazoic acid in toluene (15 mL of ca. 2 M solution) and a solution of diethyl azadicarboxylate (1.8 mL, 11.5 mmol) in toluene (20 mL). The mixture is allowed to warm to rt and stir for 18 h. The mixture is extracted with aqueous 1.0M HCl solution. The aqueous layer is extracted with EtOAc, and the combined organic layers are discarded. The pH of the aqueous layer is adjusted to 9 with 50% aqueous NaOH solution. The aqueous layer is extracted with CH₂Cl₂ (3×), and the combined organic layers are washed with brine, dried (Na₂SO₄), filtered and concentrated in vacuo. The crude product is purified by flash chromatography on silica gel. Elution with CHCl₃-MeOH—NH₄OH (92:7:1) affords Int 16 as a colorless oil (41% yield): ¹H NMR (CDCl₃) δ 4.1, 3.2, 2.8, 2.7-2.5, 2.2, 1.9, 1.5.

Step P. Preparation of endo-3-amino-1-azabicyclo[2.2.1]heptane bis(hydro-para-toluenesulfonate).

A mixture of Int 16 (250 mg, 1.8 mmol) and 10% Pd/C catalyst (12 mg) in EtOH (10 mL) is placed under an atmosphere of hydrogen (15 psi). The mixture is stirred for 1 h at rt. The mixture is filtered through Celite, and the filtrate is concentrated in vacuo. The residue is dissolved in EtOH (10 mL) and para-toluenesulfonic acid monohydrate (690 mg, 3.7 mmol) is added. The mixture is stirred for 30 min, and the precipitate is filtered. The precipitate is washed sequentially with cold EtOH and ether. The precipitate is dried in vacuo to afford endo-[2.2.1]-Amine as a white solid (85% yield): ¹H NMR (CD₃OD) δ 7.7, 7.3, 4.2, 3.9, 3.6-3.4, 3.3-3.2, 2.4, 2.3, 2.1.

Preparation of exo-tert-butyl (1S, 2R, 4R)-(+)-2-amino-7-azabicyclo[2.2.1]heptane-7-carboxylate (7-aza-[2.2.1]-Amine):

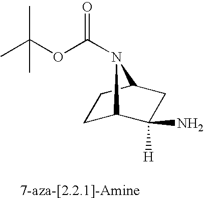

Preparation of methyl-3-bromo-propiolate:

Methyl propiolate (52 ml, 0.583 mole) is combined with recrystallized N-bromo-succinimide (120 g, 0.674 mole) in 1,700 ml acetone under nitrogen. The solution is treated with silver nitrate (9.9 g, 0.0583 mole) neat in a single lot and the reaction is stirred 6 h at RT. The acetone is removed under reduced pressure (25° C., bath temperature) to provide a gray slurry. The slurry is washed with 2×200 ml hexane, the gray solid is removed by filtration, and the filtrate is concentrated in vacuo to provide 95 g of a pale yellow oily residue. The crude material was distilled via short path under reduced pressure (65° C., about 25 mm Hg) into a dry ice/acetone cooled receiver to give 83.7 g (88%) of methyl-3-bromo-propiolate as a pale yellow oil. Anal. calc'd for C₄H₃BrO₂: C, 29.48; H, 1.86. Found: C, 29.09; H, 1.97.

Preparation of 7-tert-butyl 2-methyl 3-bromo-7-azabicyclo[2.2.1]hepta-2,5-diene-2,7-dicarboxylate.

Methyl-3-bromo-propiolate (83.7 g, 0.513 mole) is added to N-t-butyloxy-pyrrole (430 ml, 2.57 mole) under nitrogen. The dark mixture is warmed in a 90° C. bath for 30 h, is cooled, and the bulk of the excess N-t-butyloxy-pyrrole is removed in vacuo using a dry ice/acetone condenser. The dark oily residue is chromatographed over 1 kg silica gel (230-400 mesh) eluting with 0-15% EtOAc/hexane. The appropriate fractions are combined and concentrated to afford 97 g (57%) of 7-tert-butyl 2-methyl 3-bromo-7-azabicyclo[2.2.1]hepta-2,5-diene-2,7-dicarboxylate as a dark yellow oil. HRMS (FAB) calc'd for C₁₃H₁₆BrNO₄+H: 330.0341, found 330.0335 (M+H)⁺.

Preparation of (+/−) Endo-7-tert-butyl 2-methyl 7-azabicyclo[2.2.1]heptane-2,7-dicarboxylate.

7-tert-Butyl 2-methyl 3-bromo-7-azabicyclo[2.2.1]hepta-2,5-diene-2,7-dicarboxylate (97 g, 0.294 mole) is added to 10% Pd/C (6.8 g) in 900 ml absolute EtOH in a PARR bottle. The suspension is diluted with a solution of NaHCO₃ (25 g, 0.301 mole) in 250 ml water and the mixture is hydrogenated at 50 PSI for 2.5 h. The catalyst is removed by filtration, is washed with fresh EtOH, and the filtrate is concentrated in vacuo to give a residue. The residue is partitioned between 1×200 ml saturated NaHCO₃ and CH₂Cl₂ (4×100 ml). The combined organic layer is dried (1:1 K₂CO₃/MgSO₄) and concentrated in vacuo to afford 72.8 g (98%) of (+/−) endo-7-tert-butyl 2-methyl 7-azabicyclo[2.2.1]heptane-2,7-dicarboxylate. MS (EI) for C₁₄H₂₂O₄, m/z: 255 (M)⁺.

Preparation of (+/−) exo-7-(tert-butoxycarbonyl)-7-azabicyclo[2.2.1]heptane-2-carboxylic acid.

(+/−)Endo-7-tert-butyl 2-methyl 7-azabicyclo[2.2.1]heptane-2,7-dicarboxylate (72.8 g, 0.285 mole) is dissolved in 1000 ml dry MeOH in a dried flask under nitrogen. The solution is treated with solid NaOMe (38.5 g, 0.713 mole) neat, in a single lot and the reaction is warmed to reflux for 4 h. The mixture is cooled to 0° C., is treated with 400 ml water, and the reaction is stirred 1 h as it warms to RT. The mixture is concentrated in vacuo to about 400 ml and the pH of the aqueous residue is adjusted to 4.5 with 12N HCl. The precipitate is collected and dried. The tan, slightly tacky solid is washed with 2×100 ml 60% ether in hexane and is dried to provide 47 g (68%) of exo-7-(tert-butoxycarbonyl)-7-azabicyclo[2.2.1]heptane-2-carboxylic acid as an off-white powder. HRMS (FAB) calc'd for C₁₂H₁₉NO₄+H: 242.1392, found 242.1390 (M+H)⁺.

Preparation of (+/−) exo-tert-butyl 2-{[(benzyloxy)carbonyl]amino}-7-azabicyclo[2.2.1]heptane-7-carboxylate.

(+/−)Exo-7-(tert-butoxycarbonyl)-7-azabicyclo[2.2.1]heptane-2-carboxylic acid (32.5 g, 0.135 mole) is combined with TEA (24.4 ml, 0.175 mole) in 560 ml dry toluene in a dry flask under nitrogen. The solution is treated drop-wise with diphenylphosphoryl azide (37.7 ml, 0.175 mole), and is allowed to stir for 20 min at RT. The mixture is treated with benzyl alcohol (18.1 ml, 0.175 mole), and the reaction is stirred overnight at 50° C. The mixture is cooled, is extracted successively with 2×250 ml 5% citric acid, 2×200 ml water, 2×200 ml saturated sodium bicarbonate, and 2×100 ml saturated NaCl. The organic layer is dried (MgSO₄) and concentrated in vacuo to an amber oil. The crude material was chromatographed over 800 g silica gel (230-400 mesh), eluting with 15-50% EtOAc/hexane. The appropriate fractions are combined and concentrated to give 44 g (94%) of (+/−) exo-tert-butyl 2-{[(benzyloxy)carbonyl]amino}-7-azabicyclo[2.2.1]heptane-7-carboxylate as a pale oil. ¹H NMR (CDCl₃) δ 1.29-1.60, 1.44, 1.62-2.01, 3.76-3.88, 4.10, 4.24, 5.10, 7.36 ppm.

Preparation of exo-tert-butyl (1S, 2R, 4R)-(+)-2{[(benzyloxy)carbonyl]amino}-7-azabicyclo[2.2.1]heptane-7-carboxylate and exo-tert-butyl (1R, 2S, 4S)-(−)-2{[(benzyloxy)carbonyl]amino}-7-azabicyclo[2.2.1]heptane-7-carboxylate.

The isolated (+/−) exo-tert-butyl 2-{[(benzyloxy)carbonyl]amino}-7-azabicyclo[2.2.1]heptane-7-carboxylate is resolved via preparative chiral HPLC (50×500 mm Chiralcel OJ column, 30 deg. C., 70 mL/min. 10/90 (v/v) isopropanol/heptane). The resolution affords 10.5 g of exo-tert-butyl (1S, 2R, 4R)-(+)-2{[(benzyloxy)carbonyl]amino}-7-azabicyclo[2.2.1]heptane-7-carboxylate and 15.5 g of exo-tert-butyl-(1R, 2S, 4S)(−)-2{[(benzyloxy)carbonyl]amino }-7-azabicyclo[2.2.1]heptane-7-carboxylate.

The 2R enantiomer is triturated with 12 ml ether followed by 12 ml hexane (to remove lingering diastereo and enantiomeric impurities) and is dried to afford 9.5 g (43%) of purified exo-tert-butyl (1S, 2R, 4R)-(+)-2{[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵_{D}=22, (c 0.42, chloroform).

The 2S enantiomer is triturated with 20 ml ether followed by 20 ml hexane to give 14 g (64%) of purified exo-tert-butyl (1R, 2S, 4S)-(−)-2{[(benzyloxy)carbonyl]amino}-7-azabicyclo[2.2.1]heptane-7-carboxylate with 99% enantiomeric excess. MS (EI) for C₁₉H₂₆N₂O₄, m/z: 346 (M)⁺. [α]²⁵_{D}=−23, (c 0.39, chloroform).

Preparation of exo-tert-butyl-(1S, 2R, 4R)-(+)-2-amino-7-azabicyclo[2.2.1]heptane-7-carboxylate (7-aza-[2.2.1]-Amine).

Exo-tert-butyl (1S, 2R, 4R)-(+)-2{[(benzyloxy)carbonyl]amino}-7-azabicyclo[2.2.1]heptane-7-carboxylate (9.5 g, 27.4 mmol) is combined with 950 mg 10% Pd/C in 75 ml absolute EtOH in a 500 ml Parr bottle. The reaction mixture is hydrogenated at 50 PSI for 3 h, the catalyst is removed by filtration, and the filter cake was washed with MeOH. The filtrate is concentrated in vacuo to give 6.4 g of a residue. The crude material is chromatographed over 200 g silica gel (230-400 mesh) eluting with 7% CH₃OH/CHCl₃ containing 1% conc. NH₄OH. The appropriate fractions are combined and concentrated to give 5.61 g (96%) of exo-tert-butyl-(1S, 2R, 4R)-(+)-2-amino-7-azabicyclo[2.2.1]heptane-7-carboxylate as a pale oil. MS (EI) for C₁₁H₂₀N₂O₂, m/z: 212 (M)+. [α]²⁵D=9, (c 0.67, chloroform).

Preparation of 1-azabicyclo[3.2.1]octan-3-amine:

Preparation of the 3R,5R-[3.2.1]-Amine:

(3S)-1-[(S)-1-Phenethyl]-5-oxo-3-pyrrolidine-carboxylic acid:

According to the literature procedure (Nielsen et al. J. Med. Chem 1990, 70-77), a mixture of itaconic acid (123.17 g, 946.7 mmol) and (S)-(−)-α-methyl benzylamine (122.0 mL, 946.4 mmol) were heated (neat) in a 160° C. oil bath for 4 h. Upon cooling, MeOH (˜200 mL) was added and the resulting solid collected by filtration. The solid was treated with EtOH (˜700 mL) and warmed using a steam bath until ˜450 mL solvent remained. After cooling to rt, the solid was collected and dried to afford 83.2 g as a white crystalline solid: [α]²⁵_{D}=−80 (c 0.97, DMSO). MS (EI) m/z 233 (M⁺).

The lack of a resonance 3.59 indicates a single diastereomer. The other diastereomer can be retrieved from the initial MeOH triturant. Attempts to crystallize this material generally led to small quantities of (3RS)-1-[(S)-1-phenethyl]-5-oxo-3-pyrrolidine-carboxylic acid.

(3S)-1-[(S)-1-Phenethyl]-3-(hydroxymethyl)pyrrolidine:

A suspension (3S)-1-[(S)-1-phenethyl]-5-oxo-3-pyrrolidine-carboxylic acid (82.30 g, 352.8 mmol) in Et₂O (200 mL) was added in small portions to a slurry of LiAlH₄ (17.41 g, 458.6 mmol) in Et₂O (700 mL). The mixture began to reflux during the addition. The addition funnel containing the suspension was rinsed with Et₂O (2×50 mL), and the mixture was heated in a 50° C. oil bath for an additional 2 h and first allowed to cool to rt and then further cooled using an ice bath. The mixture was carefully treated with H₂O (62 mL). The resulting precipitate was filtered, rinsed with Et₂O, and discarded. The filtrate was concentrated to a yellow oil. When EtOAc was added to the oil, a solid began to form. Hexane was then added and removed by filtration and dried to afford 43.3 g as a white solid. [α]²⁵_{D}=−71 (c 0.94, CHCl₃). MS (EI) m/z 205 (M⁺).

(3R)-1-[(S)-1-Phenethyl]-3-(cyanomethyl)pyrrolidine:

A solution of (3S)-1-[(S)-1-phenethyl]-3-(hydroxymethyl)pyrrolidine (42.75 g, 208.23 mmol) in chloroform (350 mL) was heated to reflux under N₂. The solution was treated with a solution of thionyl chloride (41.8 mL, 573 mmol) in chloroform (40 mL) dropwise over 45 min. The mixture stirred for an additional 30 min, was cooled and concentrated. The residue was diluted with H₂O (˜200 mL), 1 N NaOH was added until a pH˜8 (pH paper). A small portion (˜50 mL) of sat. NaHCO₃ was added and the basic mixture was extracted with EtOAc (3×400 mL), washed with brine, dried (MgSO₄), filtered and concentrated to give 46.51 g of a red-orange oil for (3S)-1-[(S)-1-phenethyl]-3-(chloromethyl)pyrrolidine: R_{ƒ}: 0.50 (EtOAc-hexane 1:1); MS (ESI+) m/z 224.2 (MH⁺). The chloride (46.35 g, 208.0 mmol) was transferred to a flask, dimethyl sulfoxide (200 mL) was added, and the solution was treated with NaCN (17.84 g, 363.9 mmol). The mixture was heated under N₂ in a 100° C. oil bath overnight and was cooled. The brown mixture was poured into H₂O (300 mL) and extracted with EtOAc (1000 mL in portions). The combined organic layer was washed with H₂O (6×˜50 mL), brine (˜100 mL), dried (MgSO₄), filtered and concentrated to give 40.61 g as an orange-red oil: R_{ƒ}: 0.40 (EtOAc-PhCH₃ 1:1). MS (ESI+) for m/z 215.2 (M+H⁺).

(3R)-Methyl 1-[(S)-1-phenylethly]pyrrolidine-3-acetate:

Acetyl chloride (270 mL, 3.8 mol) was carefully added to a flask containing chilled (0° C.) methanol (1100 mL). After the addition was complete, the acidic solution stirred for 45 min (0° C.) and then (3R)-1-[(S)-1-phenethyl]-3-(cyanomethyl)pyrrolidine (40.50 g, 189.0 mmol) in methanol (200 mL) was added. The ice bath was removed and the mixture stirred for 100 h at rt. The resulting suspension was concentrated. Water (˜600 mL) was added, the mixture stirred for 45 min and then the pH was adjusted (made basic) through the addition of ˜700 mL sat. aq. NaHCO₃. The mixture was extracted with EtOAc (3×300 mL). The combined organics were washed with brine, dried (MgSO₄), filtered through celite and concentrated to give 36.86 g as an orange-red oil. MS (ESI+) m/z 248.2 (M+H⁺).

(5R)-1-Azabicyclo[3.2.1]octan-3-one hydrochloride:

A solution of (3R)-methyl 1-[(S)-1-phenylethly]pyrrolidine-3-acetate (25.72 g, 104.0 mmol) in THF (265 mL) was cooled under N₂ in a CO₂/acetone bath. Next, ICH₂Cl (22.7 mL, 312.0 mmol) was added, and the mixture stirred for 30 min. A solution of 2.0M lithium diisopropylamide (heptane/THF/ethylbenzene, 156 mL, 312 mmol) was added slowly over 30 min. The internal temperature reached a maximum of −40° C. during this addition. After 1 h, sat. NH₄Cl (100 mL) was added and the mixture was allowed to warm to rt. The organic layer was separated, dried (MgSO₄), filtered and concentrated. The resulting red-brown foam was chromatographed (300 g SiO₂, CHCl₃-MeOH—NH₄OH (89:10:1) followed by CHCl₃-MeOH (3:1). The product fractions were pooled and concentrated to afford (5R)-3-oxo-1-[(1S)-1-phenylethyl]-1-azoniabicyclo[3.2.1]octane chloride (10.12 g) as a tan foam (MS (ESI+) m/z 230.1 (M+H⁺). This foam (10.1 g, 38 mmol) was taken up in MeOH (500 mL), 10% Pd(C) (3.0 g) added and the mixture was hydrogenated (45 psi) overnight. The mixture was filtered and re-subjected to the reduction conditions (9.1 g, 10% Pd/C, 50 psi). After 5 h, TLC indicated the consumption of the (5R)-3-oxo-1-[(1S)-1-phenylethyl]-1-azoniabicyclo[3.2.1]octane chloride. The mixture was filtered, concentrated and triturated (minimal iPrOH) to give 3.73 g in two crops, as an off-white solid: [α]²⁵_{D}=33 (c 0.97, DMSO). MS (EI) m/z 125 (M⁺).

(3R,5R)-1-azabicyclo[3.2.1]octan-3-amine dihydrochloride:

To a flask containing (5R)-1-azabicyclo[3.2.1]octan-3-one hydrochloride (3.64 g, 22.6 mmol), hydroxylamine hydrochloride (2.04 g, 29.4 mmol), and ethanol (130 mL) was added sodium acetate trihydrate (9.23 g, 67.8 mmol). The mixture stirred for 3 h and was filtered and concentrated. The resulting white solid was taken up in n-propanol (100 mL) and sodium (˜13.6 g, 618 mmol) was added over 20-25 portions. The reaction spontaneously began to reflux, and the reaction was heated in an oil bath (100° C.). The addition was complete in ˜20 min and the mixture had solidified after ˜40 min. The oil bath was removed and n-propanol (2×25 mL) was added dissolving the remaining sodium metal. The mixture was carefully quenched through the dropwise addition of H₂O (100 mL). Saturated aq. NaCl (20 mL) was added, and the layers were separated. The organic layer was dried (MgSO₄), filtered, treated with freshly prepared MeOH/HCl, and concentrated. The resulting solid was triturated with 30 mL EtOH, filtered and dried in vaccuo to afford 3.51 g as a white solid: [α]²⁵_{D}=−3 (c 0.94, DMSO). MS (FAB) m/z 127 (MH⁺).

Preparation of endo-1-azabicyclo[3.2.1]octan-3-amine dihydrochloride (endo-[3.2.1]-Amine):

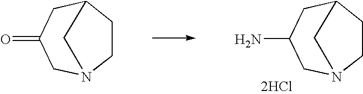

A mixture of 1-azabicyclo[3.2.1]octan-3-one hydrochloride (2.80 g, 17.3 mmol), ethanol (25 mL), and hydroxylamine hydrochloride (1.56 g, 22.4 mmol) is treated with sodium acetate trihydrate (7.07 g, 51.2 mmol). The mixture is stirred for 3 h and evaporated in vacuo. The residue is diluted with CH₂Cl₂, treated with charcoal, filtered and evaporated. The resulting oxime (3.1 mmol) is treated with acetic acid (30 mL) and hydrogenated at 50 psi over PtO₂ (50 mg) for 12 h. The mixture is then filtered and evaporated. The residue is taken up in a minimal amount of water (6 mL) and the pH is adjusted to >12 using solid NaOH. The mixture is then extracted with ethyl acetate (4×25 mL), dried (MgSO₄), filtered, treated with ethereal HCl, and evaporated to give the give endo-[3.2.1]-Amine.

Preparation of the 3.2.2 Amines:

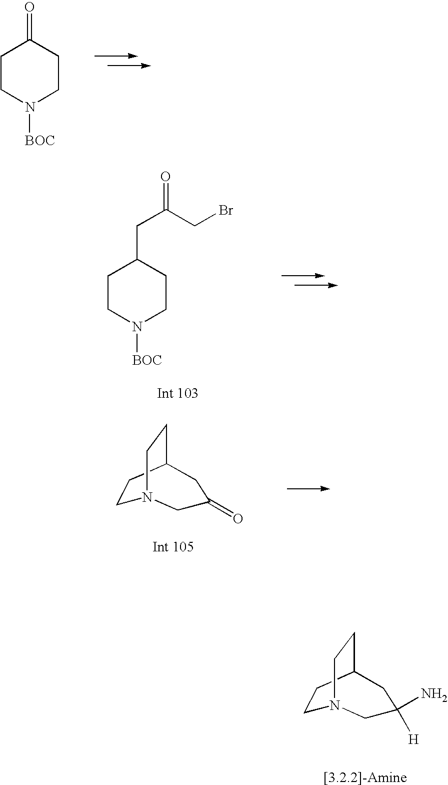

tert-Butyl 4-(2-oxopropylidene)piperidine-1-carboxylate (Int 101):

Sodium hydride (60% oil dispersion, 2.01 g, 50.2 mmol) is washed with pentane (3×) and suspended in dry THF (40 mL). The solution is cooled to 0° C. before diethyl (2-oxopropyl)phosphonate (9.75 g, 50.2 mmol) is added dropwise. After complete addition, the solution is warmed to rt and stirred for 30 min. tert-Butyl 4-oxo-1-piperidinecarboxylate (5.0 g, 25.1 mmol) is added in portions over 10 min, followed by stirring at rt for 2 h. A saturated aqueous solution of ammonium chloride is added, followed by dilution with ether. The organic layer is extracted with water. The organic layer is dried (MgSO₄), filtered and concentrated to a yellow oil. The crude product is purified by flash chromatography on silica gel. Elution with hexanes-ether (60:40) gave 4.5 g (75%) of Int 101 as a white solid: ¹H NMR (CDCl₃) δ 6.2, 3.5, 3.4, 2.9, 2.3, 2.2, 1.5.

Preparation of tert-butyl 4-(2-oxopropyl)piperidine-1-carboxylate (Int 102):

A mixture of Int 101 (4.5 g, 19 mmol) and 10% palladium on activated carbon (450 mg) in EtOH (150 mL) is placed in a Parr bottle and hydrogenated for 5 h at 50 psi. The mixture is filtered through Celite, and the filtrate is concentrated in vacuo to afford 4.3 g (94%) of Int 102 as a clear oil: ¹H NMR (CDCl₃) δ 4.1, 2.8, 2.4, 2.2, 2.0, 1.7, 1.5, 1.1.

tert-Butyl 4-(3-bromo-2-oxopropyl)piperidine-1-carboxylate (Int 103):

To a stirred solution lithium hexamethyldisilylamide in THF (20. 0 mL, 1.0 M) in a −78° C. bath is added chlorotrimethylsilane (11.0 mL, 86.4 mmol) dropwise. The mixture is stirred at −78° C. for 20 min, followed by addition of Int 102 (3.21 g, 13.3 mmol) in a solution of THF (50 mL) dropwise. After complete addition, the mixture is stirred at −78° C. for 30 min. The mixture is warmed to 0° C. in an ice-water bath and phenyltrimethylammonium tribromide (5.25 g, 14.0 mmol) is added. The mixture is stirred in an ice-bath for 30 min, followed by the addition of water and ether. The aqueous layer is washed with ether, and the combined organic layers are washed with saturated aqueous sodium thiosulfate solution. The organic layer is dried (MgSO₄), filtered and concentrated in vacuo to afford a yellow oil. The crude product is purified by flash chromatography on silica gel. Elution with hexanes-ether (60:40) gave 2.2 g (52%) of Int 103 as a lt. yellow oil: ¹H NMR (CDCl₃) δ 4.2-4.1, 3.9, 2.8, 2.7, 2.6, 2.1-2.0, 1.7, 1.5, 1.2-1.1.2.

1-Bromo-3-piperidin-4-ylacetone trifluoroacetate (Int 104):

To a stirred solution of Int 103 (2.2 g, 6.9 mmol) in CH₂Cl₂ (30 mL) in an ice-water bath is added trifluoroacetic acid (10 mL, 130 mmol). The mixture is stirred at 0° C. for 30 min. The volatiles are removed in vacuo to afford 2.0 g (87%) of Int 104 as a yellow residue: MS (ESI) for C₈H₁₅BrNO [M+H] m/e 220.

1-Azabicyclo[3.2.2]nonan-3-one (Int 105):

To a stirred solution of DIEA (13 mL) in acetoniltrile (680 mL) at reflux temperature is added a solution of Int 104 (2.0 g, 6.0 mmol) in acetonitrile (125 mL) over a 4 h period via syringe pump. The mixture is kept at reflux temperature overnight. The mixture is concentrated in vacuo and the remaining residue is partitioned between a saturated aqueous potassium carbonate solution and CHCl₃-MeOH (90:10). The aqueous layer is extracted with CHCl₃-MeOH (90:10), and the combined organic layers are dried (MgSO₄), filtered and concentrated in vacuo to a brown oil. The crude product is purified by flash chromatography on silica gel. Elution with CHCl₃-MeOH—NH₄OH (95:4.5:0.5) gives 600 mg (72%) of Int 105 as a clear solid: ¹H NMR (CDCl₃) δ 3.7, 3.3-3.2, 3.1-3.0, 2.7, 2.3, 2.0-1.8.

1-Azabicyclo[3.2.2]nonan-3-amine bis(4-methylbenzenesulfonate) ([3.2.2]-Amine):

To a stirred mixture of Int 105 (330 mg, 2.4 mmol) and sodium acetate.trihydrate (670 mg, 4.8 mmol) in EtOH (6.0 mL) is added hydroxylamine.hydrochloride (200 mg, 2.8 mmol). The mixture is stirred at rt for 10 h. The mixture is filtered and the filtrate is concentrated in vacuo to a yellow solid. To a solution of the solid (350 mg, 2.3 mmol) in n-propanol (30 mL) at reflux temperature is added sodium metal (2.0 g, 87 mmol) in small portions over 30 min. Heating at reflux is continued for 2 h. The solution is cooled to rt and brine is added. The mixture is extracted with n-propanol, and the combined organic layers are concentrated in vacuo. The residue is taken up in CHCl₃ and the remaining solids are filtered. The filtrate is dried (MgSO₄), filtered and concentrated in vacuo to a clear solid. To a stirred solution of the solid (320 mg, 2.3 mmol) in EtOH (4 mL) is added p-toluenesulfonic acid monohydrate (875 mg, 4.6 mmol). The solution is warmed in a water bath to 45° C. for 30 min, followed by concentration of the solvent to afford 710 mg (62%) of [3.2.2]-Amine as a white solid: ¹H NMR (CD₃OD) δ 7.7, 7.3, 4.1-3.9, 3.6-3.4, 2.6-2.5, 2.4, 2.2-2.1, 2.1-2.0, 1.9.

Resolution of Stereoisomers:

The amine can be coupled to form the appropriate amides or thioamides as a racemic mixture. The racemic mixture can then be resolved by chromatography using chiral columns or chiral HPLC, techniques widely known in the art, to provide the requisite resolved enantiomers 3(R) and 3(S) of said amides.

Coupling procedures using the Azabicyclo moieties discussed herein with various W moieties discussed herein to prepare compounds of formula I are discussed in the following, all of which are incorporated herein by reference: U.S. Pat. No. 6,492,386; U.S. Pat. No. 6,500,840; U.S. Pat. No. 6,562,816; US 2003/0045540A1; US 2003/0055043A1; US 2003/0069296A1; US 2003/0073707A1; US 2003/015089A1; US 2003/0130305A1; US 2003/0153595A1; WO 03/037896; WO 03/40147; WO 03/070728; WO 03/070731; WO 03/070732. Although the compounds made therein may be for one specific Azabicyclo moiety, the procedures discussed, or slight non-critical changes thereof, can be used to make the compounds of formula I.

The intermediates providing the W of formula I either are commercially available or prepared using known procedures, making non-critical changes.

Compounds of Formula I where W is (D) are made using the coupling procedures discussed herein and in the literature, making non-critical changes to obtain the desired compounds. The following intermediates to provide W as (D) of formula I are for exemplification only and are not intended to limit the scope of the present invention. Other intermediates within the scope of the present invention can be obtained using known procedures or by making slight modifications to known procedures.

Intermediate D1: furo[2,3-c]pyridine-5-carboxylic acid

There are many routes for obtaining the carboxylic acid including the preparation of the acid discussed herein and also from hydrolyzing the ester, the preparation of which is discussed in U.S. Pat. No. 6,265,580. n-Butyl furo[2,3-c]pyridine-5-carboxylate is hydrolyzed to the corresponding carboxylate salt on treatment with sodium or potassium hydroxide in aqueous methanol or acetonitrile-methanol mixtures. Acidification to pH 2.5-3.5 generates the carboxylic acid, which is isolated as a solid. The free base can also be prepared directly from n-butyl furo[2,3-c]pyridine-5-carboxylate by direct condensation using at least 1.5 molar equivalents of (R)-3-aminoquinuclidine and heating in ethanol or n-butyl alcohol.

2-Chloro-3-pyridinol (20.0 g, 0.154 mole), NaHCO₃ (19.5 g, 0.232 mole, 1.5 equ), and 150 mL of water are placed in a flask. The flask is placed in an oil bath at 90° C., and after 5 min, 37% aqueous formaldehyde (40.5 mL, 0.541 mole, 3.5 equ) is added in six unequal doses in the following order: 12 mL, 3×8 mL, then 2.2 mL all at 90-min intervals and then the final 2.3 mL after the reaction stirs for 15 h at 90° C. The reaction is stirred at 90° C. for another 4 h and then cooled by placing the flask in an ice bath. The pH of the reaction is then adjusted to 1 using 6N HCl. The reaction is stirred for 1.5 h in an ice bath allowing an undesired solid to form. The undesired solid is removed by filtration, and the filtrate is extracted seven times with EtOAc. The combined organic extracts are concentrated in vacuo, toluene is added to the flask and removed in vacuo to azeotrope water, and then CH₂Cl₂ is added and removed in vacuo to obtain 2-chloro-6-(hydroxymethyl)-3-pyridinol (I-1-D) as a pale yellow solid (81% yield) sufficiently pure for subsequent reaction. MS (EI) for C₆H₆ClNO₂, m/z: 159 (M)⁺.

I-1-D (11.6 g, 72.7 mmol) and NaHCO₃ (18.3 g, 218 mmol) are added to 200 mL H₂O. The mixture is stirred until homogeneous, the flask is placed in an ice bath, iodine (19.4 g, 76.3 mmol) is added, and the reaction is stirred over the weekend at rt. The pH of the mixture is adjusted to 3 with 2N NaHSO₄, and the mixture is extracted with 4×50 mL EtOAc. The combined organic layer is dried (MgSO₄), is filtered, and the filtrate is concentrated in vacuo to a yellow solid. The crude solid is washed with EtOAc to provide 2-chloro-6-(hydroxymethyl)-4-iodo-3-pyridinol (I-2-D) as an off-white solid (62% yield), and the filtrate is concentrated to a small volume and is chromatographed over 250 g silica gel (230-400 mesh) eluting with 2.5:4.5:4:0.1 EtOAc/CH₂Cl₂/hexane/acetic acid to afford additional pure I-2-D (12% yield). MS (EI) for C₆H₅ClINO₂, m/z: 285(M)⁺.

I-2-D (13.9 g, 48.6 mmol) is combined with trimethylsilylacetylene (9.6 mL, 68 mmol), bis(triphenylphosphine) palladium dichloride (1.02 g, 1.46 mmol) and cuprous iodide (139 mg, 0.73 mmol) in 80 mL CHCl₃/40 mL THF under N₂. TEA (21 mL, 151 mmol) is added, and the reaction is stirred 3 h at rt and is diluted with 200 mL CHCl₃. The mixture is washed with 2×150 mL 5% HCl and the combined aqueous layers are extracted with 2×50 mL CHCl₃. The combined organic layer is washed with 100 mL 50% saturated NaCl, is dried (MgSO₄), and concentrated in vacuo to an amber oil. The crude material is chromatographed over 350 g silica gel (230-400 mesh), eluting with 35% EtOAc/hexane to afford 2-chloro-6-(hydroxymethyl)-4-[(trimethylsilyl)ethynyl]-3-pyridinol (I-3-D) as a golden solid (92% yield). MS (EI) for C₁₁H₁₄ClNO₂Si, m/z: 255(M)⁺.

I-3-D (7.9 g, 31.2 mmol) and cuprous iodide (297 mg, 1.6 mmol) in 60 mL EtOH/60 mL TEA are added to a flask. The reaction is placed in an oil bath at 70° C. for 3.5 h, is cooled to rt, and concentrated in vacuo. The residue is partitioned between 100 mL 5% HCl and CH₂Cl₂ (4×50 mL). The combined organic layer is dried (MgSO₄), filtered, and concentrated in vacuo to give 6.5 g of a crude amber solid. The crude material is chromatographed over 300 g silica gel (230-400 mesh) eluting with 30-40% EtOAc/hexane. Two sets of fractions with two different desired compounds are identified by TLC/UV. The two compounds eluted separately. The early-eluting pool of fractions is combined and concentrated to afford [7-chloro-2-(trimethylsilyl)furo[2,3-c]pyridin-5-yl]methanol (I-5-D) as a white solid (46% yield). The later-eluting pool of fractions is combined and concentrated to provide (7-chlorofuro[2,3-c]pyridin-5-yl)methanol (I-4-D) as a white solid (27% yield). MS (EI) for C₈H₆ClNO₂, m/z: 183 (M)⁺ for I-4-D. HRMS (FAB) calculated for C₁₁H₁₄ClNO₂Si m/z: 255.0482, found 255.0481 for I-5-D.

I-5-D (1.05 g, 4.1 mmol) and 10% Pd/C catalyst (1.05 g) are placed in 20 mL absolute EtOH. Cyclohexene (4 mL, 40.1 mmol) is added, and the reaction is refluxed for 2.5 h, and then filtered through celite. The filter cake is washed with 1:1 EtOH/CH₂Cl₂, and the filtrate is concentrated to a pale yellow solid. The residue is partitioned between 40 mL saturated NaHCO₃ and extracted with CH₂Cl₂ (4×20 mL). The combined organic layer is dried (MgSO₄), filtered, and then concentrated in vacuo to a pale oil (1.04 g). The pale oil is chromatographed over 50 g silica gel (230-400 mesh) eluting with 50-70% EtOAc/hexane to afford 5-hydroxymethyl-2-trimethylsilyl-furo[2,3-c]pyridine (I-14-D) as a white solid (90% yield). MS (EI) for C₁₁H₁₅NO₂Si, m/z: 221(M)⁺.

I-14-D (770 mg, 3.48 mmol) is dissolved in 10 mL MeOH. 2N NaOH (3 mL, 6 mmol) is added, and the reaction is stirred for 1.5 h at rt. The solution is concentrated in vacuo to a residue. Water (20 mL) is added to the residue and extracted with 4×10 mL CH₂Cl₂. The combined organic layer is dried (K₂CO₃), filtered, and concentrated in vacuo to afford furo[2,3-c]pyridin-5-yl methanol (I-16-D) as a white solid (90% yield). Analysis calculated for C₈H₇NO₂: C, 64.42; H, 4.73; N, 9.39. Found: C, 64.60; H, 4.56; N, 9.44.

Alternatively, I-3-D is used to obtain I-16-D with fewer steps: I-3-D (44.6 g, 174.4 mmol) is combined with cuprous iodide (1.66 g, 8.72 mmol) and diisopropylamine (44 ml, 300 mmol) in 300 ml methanol under nitrogen. The reaction is warmed to 45-50° C. for 6 h, is cooled to rt and treated with 100 ml saturated NaHCO₃ followed by 100 ml 2N NaOH. The dark mixture is stirred overnight, filtered through celite, the volatiles removed in vacuo and the residue is partitioned between 1×500 ml water and 4×200 ml CH₂Cl₂ (some filtrations is required to effect good separation). The combined organic layer is dried (MgSO₄) and concentrated in vacuo to afford I-4-D (25.25 g, 79%) as a pale orange solid. Anal. Calcd for C₈H₆ClNO₂: C, 52.34; H, 3.29; N, 7.63. Found: C, 52.27; H, 3.23; N, 7.57.

I-4-D (32.0 g, 174 mmol) is combined with zinc powder (34.2 g, 523 mmol) in absolute EtOH (900 mL), using an overhead stirrer. The mixture is heated to 70° C., HCl (87.2 mL, 1.05 mol) is added slowly drop-wise, and the mixture is heated to reflux for 1 h. The mixture is cooled slightly, filtered to remove the metallic zinc and concentrated to near-dryness. The yellow oil is diluted with H₂O (150 mL) and EtOAc (950 mL) and is treated slowly drop-wise with 20% Na₂CO₃ (310 mL) as the mixture is warmed to reflux. The vigorously stirred (using overhead stirrer) mixture is refluxed for 1 h, cooled slightly and the organics removed via cannula under reduced pressure. Additional EtOAc (600 mL) is added, the mixture is heated to reflux for 1 h, cooled slightly and the organics removed as above. More EtOAc (600 mL) is added, the mixture is stirred at rt overnight then heated to reflux for 1 h, cooled slightly and most of the organics removed as above. The remaining mixture is filtered through celite, rinsed with EtOAc until no additional product elutes, and the layers separated. The aqueous layer is further extracted with EtOAc (2×400 mL). The combined organics are dried (MgSO₄) and concentrated to a dark yellow solid (23.6 g). The crude material is chromatographed over 900 g slurry-packed silica gel, eluting with 60% EtOAc/hexane (3 L), 70% EtOAc/hexane (2 L), and finally 100% EtOAc. The appropriate fractions are combined and concentrated in vacuo to afford I-16-D (19.5 g, 75%) as a white solid. Anal. Calcd for C₈H₇NO₂: C, 64.42; H, 4.73; N, 9.39; Found: C, 64.60; H, 4.56; N, 9.44.

Oxalyl chloride (685 μL, 7.8 mmol) is dissolved in 30 mL CH₂Cl₂ in a dry flask under N₂. The flask is placed in a dry-ice/acetone bath, DMSO (1.11 mL, 15.6 mmol) in 5 mL CH₂Cl₂ is added drop-wise, and the mixture is stirred for 20 min. I-16-D (1.0 g, 6.7 mmol) in 10 mL CH₂Cl₂ is added, and the reaction is stirred 30 min at −78° C. TEA (4.7 mL, 33.5 mmol) is added, the reaction is allowed to warm to rt, is stirred 1 h, and washed with 25 mL saturated NaHCO₃. The organic layer is dried (K₂CO₃), filtered, and concentrated in vacuo to an orange solid. The crude material is chromatographed over 50 g silica gel (230-400 mesh) eluting with 33% EtOAc/hexane to provide furo[2,3-c]pyridine-5-carbaldehyde (I-17-D) as a white solid (86% yield). MS (EI) for C₈H₅NO₂, m/z: 147 (M)⁺.

I-17-D (850 mg, 5.8 mmol) is dissolved in 10 mL DMSO. KH₂PO₄ (221 mg, 1.6 mmol) in 3 mL H₂O is added and then NaClO₂ (920 mg, 8.2 mmol) in 7 mL H₂O is added, and the reaction is stirred 3 h at rt. The reaction is diluted with 25 mL water, the pH is adjusted to 10 with 2N NaOH, and the mixture is extracted with 3×20 mL ether. The combined ether layer is discarded. The pH of the aqueous layer is adjusted to 3.5 with 10% aqueous HCl and is extracted with 13×10 mL 10% MeOH/CH₂Cl₂. The MeOH/CH₂Cl₂ organic layer is dried (Na₂SO₄), filtered, and concentrated in vacuo to a pale oil. The residual DMSO is removed under a stream of N₂ at rt to provide a white paste. The paste is dissolved in MeOH and concentrated to dryness. The white solid is washed with ether and dried to afford crude furo[2,3-c]pyridine-5-carboxylic acid (I-18-D) (94% yield). MS (ESI) for C₈H₅NO₃, 162.8 (M−H)⁻.

Intermediate D2: Furo[3,2-c]pyridine-6-carboxylic acid

3-Bromofuran (8.99 mL, 100.0 mmol) is dissolved in DMF (8.5 mL), cooled to 0° C., treated dropwise with POCl₃ (9.79 mL, 105.0 mmol), stirred for 1 h at RT and then heated to 80° C. for 2 h. The mixture is cooled to RT, poured over ice (1 kg) and neutralized to pH 9 with solid K₂CO₃. The mixture is stirred for 1 h, extracted with Et₂O (3×500 mL), dried (K₂CO₃) and concentrated to a dark brown oil. The crude material is chromatographed over 600 g slurry-packed silica gel, eluting with 6% EtOAc/hexane (4 L), 8% EtOAc/hexane (2 L), 10% EtOAc/hexane (1 L), and finally 20% EtOAc/hexane. The appropriate fractions are combined and concentrated in vacuo to afford 14.22 g (81%) of 3-bromo-2-furaldehyde as a yellow oil. MS (EI) m/z: 174 (M⁺).

3-Bromo-2-furaldehyde (14.22 g, 81.3 mmol) is combined with ethylene glycol (6.55 mL, 117.4 mmol) and para-toluene sulfonic acid monohydrate (772 mg, 4.06 mmol) in benzene (200 mL) and heated to reflux with a Dean-Stark trap for 5 h. Additional ethylene glycol (1.64 mL, 29.41 mmol) and benzene (150 mL) are added and the solution is heated for an additional 2 h. The mixture is cooled to RT, treated with saturated NaHCO₃ and stirred for 0.5 h. The layers are separated and the organics are dried (Na₂SO₄) and concentrated to a brown oil (18.8 g). The crude material is chromatographed over 700 g slurry-packed silica gel, eluting with 15% EtOAc/hexane. The appropriate fractions are combined and concentrated in vacuo to afford 16.45 g (92%) of 2-(3-bromo-2-furyl)-1,3-dioxolane as a yellow-orange oil. MS (EI) m/z: 218 (M⁺).

2-(3-Bromo-2-furyl)-1,3-dioxolane (438 mg, 2.0 mmol) is dissolved in Et₂O (5 mL) in a dry flask under nitrogen, cooled to −78° C., treated dropwise with tert-butyllithium (2.59 mL, 4.4 mmol) and stirred for 1 h. DMF (178 μL, 2.3 mmol) in Et₂O (2 mL) is added dropwise, the mixture stirred for 4 h at −78° C., then treated with oxalic acid dihydrate (504 mg, 4.0 mmol) followed by water (2 mL). The cooling bath is removed and the mixture allowed to warm to RT over 1 h. The mixture is diluted with water (20 mL) and EtOAc (20 mL), the layers are separated and the aqueous layer extracted with EtOAc (1×20 mL). The organics are dried (Na₂SO₄) and concentrated to a yellow oil. The crude material is chromatographed over 12 g slurry-packed silica gel, eluting with 15% EtOAc/hexane. The appropriate fractions are combined and concentrated in vacuo to afford 228 mg (68%) of 2-(1,3-dioxolan-2-yl)-3-furaldehyde as a pale yellow oil. MS (EI) m/z: 168 (M⁺).

2-(1,3-Dioxolan-2-yl)-3-furaldehyde (2.91 g, 17.31 mmol) is combined with formic acid (17 mL, 451 mmol) and water (4.25 mL) and stirred at RT for 18 h. The mixture is slowly transferred into a solution of NaHCO₃ (45 g, 541 mmol) in water (600 mL), then strirred for 0.5 h. EtOAc (200 mL) is added, the layers separated and the aqueous layer extracted with EtOAc (2×200 mL). The combined organics are dried (Na₂SO₄) and concentrated to a yellow oil (3.28 g). The crude material is chromatographed over 90 g slurry-packed silica gel, eluting with 20% EtOAc/hexane. The appropriate fractions are combined and concentrated to afford 2.45 g of furan-2,3-dicarbaldehyde slightly contaminated with ethylene glycol diformate as a yellow oil. ¹H NMR (CDCl₃): δ 7.00 (d, J=2 Hz, 1 H), 7.67 (d, J=2 Hz, 1 H), 10.07 (s, 1 H), 10.49 (s, 1 H) ppm.

Methyl (acetylamino)(dimethoxyphosphoryl)acetate (2.34 g, 9.8 mmol) is dissolved in CHCl₃ (40 mL), treated with DBU (1.46 mL, 9.8 mmol), stirred for 5 min then added dropwise to a 0° C. solution of furan-2,3-dicarbaldehyde (1.65 g, 8.9 mmol) in CHCl₃ (80 mL). The mixture is stirred for 2.5 h as the cooling bath expires then 5.5 h at RT and finally 24 h at 50° C. The mixture is concentrated in vacuo to a yellow oily-solid (6.66 g). The crude material is chromatographed over a standard 100 g slurry-packed silica gel, eluting with 65% EtOAc/hexane. The appropriate fractions are combined and concentrated in vacuo to afford 1.30 g (82%) of methyl furo[3,2-c]pyridine-6-carboxylate as a yellow solid. MS (EI) m/z: 177 (M⁺).

Methyl furo[3,2-c]pyridine-6-carboxylate (1.55 g, 8.74 mmol) is dissolved in MeOH (30 mL) and H₂O (15 mL), treated with 3 N NaOH (6.4 mL) and stirred at RT for 7 h. The mixture is concentrated to dryness, dissolved in H₂O (10 mL) and acidified to pH 2 with concentrated HCl. The solution is concentrated to dryness, suspended in a smaller amount of water (7 mL) and the resulting solid collected via filtration (lot A). The filtrate is concentrated, triturated with water (3 mL) and the resulting solid collected via filtration (lot B). The filtrate from lot B is concentrated and carried on without further purification as an acid/salt mixture (lot C). Both lots A and B are dried in a vacuum oven at 50° C. for 18 h to afford 690 mg (48%) for lot A and 591 mg (42%) for lot B of furo[3,2-c]pyridine-6-carboxylic acid as yellow solids. MS (CI) m/z: 164 (M+H⁺).

Intermediate D3: 7-Chlorofuro[2,3-c]pyridine-5-carboxylic acid

Oxalyl chloride (3.1 mL, 35 mmol) is dissolved in 200 mL CH₂Cl₂ in a dried flask under N₂. The flask is placed in a dry-ice/acetone bath at −78° C., DMSO (4.95 mL, 70 mmol) in 10 mL CH₂Cl₂ is added drop-wise, and the mixture is stirred for 20 min. (7-Chlorofuro[2,3-c]pyridin-5-yl)methanol (I-4-D) (5.5 g, 30 mmol) in 10 mL CH₂Cl₂ is added, and the reaction is stirred 30 min at −78° C. TEA (21.3 mL, 153 mmol) is then added. The reaction is stirred 30 min in the dry-ice/acetone bath, an ice bath replaces the dry-ice/acetone bath, and the reaction is stirred 1 h and is washed with 100 mL 1:1 saturated NaCl/NaHCO₃. The organic layer is dried (K₂CO₃), filtered, and concentrated in vacuo to afford 7-chlorofuro[2,3-c]pyridine-5-carbaldehyde (I-6-D as a pale yellow solid (97% yield). MS (EI) for C₈H₄ClNO₂ m/z: 181 (M)⁺.

I-6-D (3.0 g, 16.5 mmol) is dissolved in 40 mL DMSO. KH₂PO₄ (561 mg, 4.1 mmol) in 6.5 mL H₂O is added and then NaClO₂ (2.6 g, 23.1 mmol) in 24 mL H₂O is added, and the reaction is stirred overnight at rt. The reaction is diluted with 200 mL H₂O, the pH is adjusted to 9 with 2N NaOH, and any remaining aldehyde is extracted into 3×50 mL ether. The pH of the aqueous layer is adjusted to 3 with 10% aqueous HCl and is extracted with 4×50 mL EtOAc. The combined organic layer is dried (MgSO₄), filtered, and concentrated in vacuo to a white solid. The solid is washed with ether and dried to afford 7-chlorofuro[2,3-c]pyridine-5-carboxylic acid (I-7-D) (55% yield). MS (CI) for C₈H₄ClNO₃, m/z: 198 (M+H).

Intermediate D4: 2,3-Dihydrofuro[2,3-c]pyridine-5-carboxylic acid

I-7-D (980 mg, 4.98 mmol) is dissolved in 75 mL MeOH containing 500 mg 20% palladium hydroxide on carbon in a 250 mL Parr shaker bottle. The reaction mixture is hydrogenated at 20 PSI for 24 h. The catalyst is removed by filtration and the filtrate is concentrated in vacuo to a white solid. The solid is dissolved in MeOH and is loaded onto 20 mL Dowex 50W-X2 ion exchange resin (hydrogen form) which had been prewashed with MeOH. The column is eluted with 50 mL MeOH followed by 150 mL 5% TEA in MeOH to afford 2,3-dihydrofuro[2,3-c]pyridine-5-carboxylic acid (I-8-D) (74% yield). HRMS (FAB) calculated for C₈H₇NO₃+H: 166.0504, found 166.0498 (M+H).

Intermediate D5: 3,3-Dimethyl-2,3-dihydrofuro[2,3-c]pyridine-5-carboxylic acid

2-Chloro-6-(hydroxymethyl)-4-iodo-3-pyridinol (I-2-D) (6.3 g, 22 mmol) is dissolved in 30 mL DMF in a dry flask under N₂. The flask is placed in an ice bath, and 60% sodium hydride in mineral oil (880 mg, 22 mmol) is added. The reaction is stirred 30 min while the flask is kept in an ice bath. The ice bath is removed for 30 min and then the flask is placed back into the ice bath to cool the reaction. 3-Bromo-2-methylpropene (23.1 mmol) is added, and the reaction is stirred overnight at rt. The reaction is diluted with 150 mL EtOAc and is washed with 4×50 mL 50% saturated 1:1 NaCl/NaHCO₃. The organic layer is dried (Na₂SO₄), filtered, and then concentrated in vacuo to a pale oil which is crystallized from hexanes to afford (6-chloro-4-iodo-5-[(2-methyl-2-propenyl)oxy]-2-pyridinyl)methanol (I-19-D) (86% yield). HRMS (FAB) calculated for C₁₀H₁₁ClINO₂+H: 339.9603, found 339.9604 (M+H).

I-19-D (6.3 g, 18.9 mmol), sodium formate (1.49 g, 21.8 mmol), TEA (8 mL, 57.2 mmol), palladium acetate (202 mg, 0.9 mmol) and tetra (n-butyl)ammonium chloride (5.25 g, 18.9 mmol) are added to 30 mL DMF in a dry flask under N₂. The reaction is warmed to 60° C. for 5 h, is poured into 150 mL EtOAc, and is washed with 4×50 mL 50% saturated 1:1 NaCl/NaHCO₃. The organic layer is dried (Na₂SO₄), filtered, and concentrated in vacuo to a pale oil. The crude material is chromatographed over 40 g silica gel (Biotage), eluting with 30% EtOAc/hexane to afford (7-chloro-3,3-dimethyl-2,3-dihydrofuro[2,3-c]pyridin-5-yl)methanol (I-20-D) (54% yield). MS (EI) for C₁₀H₁₂ClNO₂, m/z: 213 (M)⁺.

I-20-D (2.11 g, 9.9 mmol) and 600 mg 10% Pd/C catalyst are placed in 30 mL EtOH in a 250 mL Parr shaker bottle. 2N NaOH (5 mL, 10 mmol) is then added and the mixture is hydrogenated at 20 PSI for 2.5 h. The catalyst is removed by filtration, and the filtrate is concentrated in vacuo to an aqueous residue. Saturated NaHCO₃ (20 mL) is added to the residue and extracted with 4×20 mL CH₂Cl₂. The combined organic layer is dried (K₂CO₃), filtered, and concentrated in vacuo to afford (3,3-dimethyl-2,3-dihydrofuro[2,3-c]pyridin-5-yl)methanol (I-21-D) (92% yield). MS (EI) for C₁₀H₁₃NO₂, m/z: 179 (M)⁺.

Oxalyl chloride (869 μL, 9.9 mmol) is dissolved in 50 mL CH₂Cl₂ in a dry flask under N₂. The flask is placed in a dry-ice/acetone bath at −78° C., DMSO (1.41 mL, 19.8 mmol) in 5 mL CH₂Cl₂ is added drop-wise, and the mixture is stirred for 20 min. I-21-D (1.53 g, 8.5 mmol) in 5 mL CH₂Cl₂ is then added, and the reaction is stirred 30 min at −78° C. TEA (5.9 mL, 42.5 mmol) is added and the reaction is stirred 20 min at −78° C. The dry-ice/acetone bath is removed, the reaction is stirred 1 h, and the reaction is washed with 25 mL saturated NaHCO₃. The organic layer is dried (K₂CO₃), filtered, and then concentrated in vacuo to an orange solid. The crude material is chromatographed over 40 g silica gel (Biotage) eluting with 25% EtOAc/hexane to afford 3,3-dimethyl-2,3-dihydrofuro[2,3-c]pyridine-5-carbaldehyde (I-22-D) (92% yield). MS (EI) for C₁₀H₁₁NO₂, m/z: 177 (M)⁺.

I-22-D (1.35 g, 7.62 mmol) is dissolved in 40 mL THF, 20 mL t-butanol, and 20 mL H₂O. KH₂PO₄ (3.11 g, 22.9 mmol) and NaClO₂ (2.58 g, 22.9 mmol) are added, and the reaction is stirred over the weekend at rt. The reaction is concentrated in vacuo to a residue. The residue is partitioned between 20 mL water and CH₂Cl₂ (2×50 mL). The combined organic layer is dried (Na₂SO₄), filtered, and then concentrated in vacuo to afford crude 3,3-dimethyl-2,3-dihydrofuro[2,3-c]pyridine-5-carboxylic acid (I-23-D) (99% yield). HRMS (FAB) calculated for C₁₀H₁₁NO₃+H: 194.0817, found 194.0808 (M+H).

Intermediate D6: 2-Methylfuro[2,3-c]pyridine-5-carboxylic acid

2-Chloro-6-(hydroxymethyl)-4-iodo-3-pyridinol (I-2-D) (4.6 g, 16 mmol), propargyl trimethylsilane (2 g, 17.8 mmol), bis(triphenylphosphine) palladium dichloride (156 mg, 0.21 mmol), cuprous iodide (122 mg, 0.64 mmol), and piperidine (3.52 mL, 26.6 mmol) are added to 25 mL DMF in a dry flask under N₂. The mixture is warmed to 45° C. for 7 h, is stirred overnight at rt, and is diluted with 150 mL EtOAc. The mixture is washed with 4×50 mL 50% saturated 1:1 NaCl/NaHCO₃. The organic layer is dried (Na₂SO₄), filtered, and then concentrated in vacuo to an amber oil. The crude material is chromatographed over 40 g silica gel (230-400 mesh) eluting with 35% EtOAc/hexane to afford (7-chloro-2-methylfuro[2,3-c]pyridin-5-yl)methanol (I-24-D) (44% yield). MS (CI) for C₉H₈ClNO₂, m/z: 198 (M+H).

I-24-D (2.0 g, 10.8 mmol) is added to 500 mg 10% Pd/C catalyst in 25 mL EtOH in a 250 mL Parr shaker bottle. 2N NaOH (6 mL, 12 mmol) is added, and the reaction is hydrogenated at 20 PSI for 6 h. The catalyst is removed by filtration, and the filtrate is concentrated in vacuo to an aqueous residue. The residue is partitioned between 50 mL 50% saturated NaCl and 30 mL CH₂Cl₂. The organic layer is dried (K₂CO₃), filtered, and then concentrated in vacuo to afford (2-methylfuro[2,3-c]pyridin-5-yl)methanol (I-25-D) (77% yield). MS (CI) for C₉H₉NO₂, m/z: 164 (M+H).

Oxalyl chloride (784 μL, 8.9 mmol) is dissolved in 25 mL CH₂Cl₂ in a dry flask under N₂. The flask is placed in a dry-ice/acetone bath at −78° C., and DMSO (1.26 mL, 17.8 mmol) in 5 mL CH₂Cl₂ is added. The mixture is stirred for 20 min and I-25-D (1.53 g, 8.5 mmol) in 5 mL CH₂Cl₂ is added. The reaction is stirred 1 h, TEA (5.9 mL, 42.5 mmol) is added, and the reaction is stirred 30 min at −78° C. The flask is placed in an ice bath, and the reaction is stirred 1 h. The reaction is washed with 50 mL saturated NaHCO₃. The organic layer is dried (K₂CO₃), filtered, and then concentrated in vacuo to a tan solid. The crude material is chromatographed over 40 g silica gel (Biotage) eluting with 25% EtOAc/hexane to afford 2-methylfuro[2,3-c]pyridine-5-carbaldehyde (I-26-D) (99% yield). MS (EI) for C₉H₇NO₂, m/z: 161 (M)⁺.

I-26-D (1.15 g, 7.1 mmol) is dissolved in 40 mL THF, 20 mL t-butanol, and 20 mL H₂O. 2-Methyl-2-butene (6.5 mL, 57.4 mmol) is added, and then KH₂PO₄ (3.11 g, 22.9 mmol) and NaClO₂ (2.58 g, 22.9 mmol) are added. The reaction is stirred 6 h at rt. The reaction is concentrated in vacuo. Water (20 ml) is added to the residue, a white solid remained. The white solid is collected, washed with water and then with ether, and is dried to afford 2-methylfuro[2,3-c]pyridine-5-carboxylic acid (I-27-D) (70% yield). MS (EI) for C₉H₇NO₃, m/z: 177 (M)⁺.

Intermediate D7: 3-Methylfuro[2,3-c]pyridine-5-carboxylic acid

2-Chloro-6-(hydroxymethyl)-4-iodo-3-pyridinol (I-2-D) (7.14 g, 25.0 mmol) is dissolved in DMF (50 mL) in a dry flask under N₂, sodium hydride (60% dispersion in mineral oil) (1.0 g, 25.0 mmol) is added, and the reaction is stirred for 1 h at rt. Allyl bromide (2.38 mL, 27.5 mmol) is added, and the reaction mixture is stirred 48 h at rt. The mixture is diluted with EtOAc (50 mL) and washed 4×25 mL of a 50% saturated solution of 1:1 NaCl/NaHCO₃. The organic layer is dried (MgSO₄), filtered and concentrated in vacuo to a white solid. The solid is washed with hexane and dried to afford 3-(allyloxy)-2-chloro-6-(hydroxymethyl)-4-iodopyridine (I-50-D) as a white solid (68% yield). MS (EI) for C₉H₉ClINO₂, m/z: 325 (M)⁺.

I-50-D (5.51 g, 16.9 mmol) is suspended in benzene (30 mL) in a dry flask under N₂. Azo(bis)isobutyryl nitrile (289 mg, 1.8 mmol) is added, the mixture is rapidly heated to reflux, and tributyltin hydride (4.91 mL, 18.2 mmol) in benzene (10 mL) is added. The solution is refluxed for 1.5 h, allowed to cool to rt and concentrated in vacuo. The resulting residue is chromatographed over 125 g slurry-packed silica gel, eluting with a gradient of EtOAc/hexane (20% -60%) to afford (7-chloro-3-methyl-2,3-dihydrofuro[2,3-c]pyridin-5-yl)methanol (I-51-D) as a white solid (89% yield). MS (ESI) for C₉H₁₀ClNO₂+H, m/z: 200.1 (M+H).

I-51-D (3.00 g, 15.0 mmol) is added to 20% palladium hydroxide on carbon (800 mg) and 2N NaOH (9.2 mL, 18.2 mmol) in a Parr shaker bottle. The mixture is hydrogenated at 20 PSI for 3 h, is filtered through celite and concentrated in vacuo to a residue. The resulting residue is partitioned between H₂O (50 mL) and CH₂Cl₂ (4×30 mL). The combined organic layer is dried (MgSO₄), filtered, and concentrated to a colorless oil which solidified upon standing to afford 2.50 g (greater than 100% yield) of (3-methyl-2,3-dihydrofuro[2,3-c]pyridin-5-yl)methanol (I-52-D) as a white crystalline solid. MS (EI) for C₉H₁₁NO₂, m/z: 165 (M)⁺.

I-52-D (2.48 g, 15.03 mmol) is dissolved in pyridine (15 mL), and acetic anhydride (4.18 mL, 45.09 mmol) is added and stirred for 16 h at rt under N₂. The reaction is concentrated in vacuo, and the residue is diluted with EtOAc (75 mL), washed with 50% saturated NaHCO₃ (4×30 mL), and dried (MgSO₄). The organic layer is filtered and concentrated in vacuo to afford (3-methyl-2,3-dihydrofuro[2,3-c]pyridin-5-yl)methyl acetate (I-53-D) as a colorless oil (92% yield). MS (EI) for C₁₁H₁₃NO₃, m/z: 207 (M)⁺.

I-53-D (2.85 g, 13.8 mmol) is dissolved in dioxane (100 mL), 2,3,5,6-tertachlorobenzoquinone (3.72 g, 15.1 mmol) is added, and the reaction is heated to reflux for 17 h. The reaction is concentrated in vacuo. The resulting brown solid is washed with 1:1 EtOAc/ether (50 mL), and the insoluble material filtered off. The filtrate is concentrated to a brown solid, dissolved in MeOH (50 mL), treated with 2N NaOH (16 mL, 32 mmol), and stirred at rt for 1 h. The mixture is concentrated to dryness, dissolved in 1N NaOH (75 mL), and extracted with CH₂Cl₂ (4×50 mL). The combined organic layer is dried (K₂CO₃), filtered, and concentrated to a white solid (2.0 g). The crude material is adsorbed onto silica gel (4 g) and chromatographed over a standard 40 g Biotage column, eluting with 90% EtOAc/hexane to afford (3-methylfuro[2,3-c]pyridin-5-yl)methanol (I-54-D) as a white solid (84% yield). MS (EI) for C₉H₉NO₂, m/z: 163 (M)⁺.

Oxalyl chloride (1.16 mL, 13.2 mmol) is added to CH₂Cl₂ (30 mL) in a dry flask under N₂ and in a dry-ice/acetone bath at −78° C. DMSO (18.80 mL, 26.5 mmol) is slowly added. The solution is stirred for 20 min, and I-54-D (1.88 g, 11.5 mmol) is added. The mixture is stirred for 1 h at −78° C., then 30 min at 0-5° C. The material is washed with saturated NaHCO₃ (75 mL), dried (K₂CO₃), filtered, and concentrated in vacuo to a yellow solid (3.23 g). The crude material is adsorbed onto silica gel (6 g) and chromatographed over a standard 40 g Biotage column, eluting with 25% EtOAc/hexane to afford 3-methylfuro[2,3-c]pyridine-5-carbaldehyde (I-55-D) as a white solid (72% yield). MS (EI) for C₉H₇NO₂, m/z: 161 (M)⁺.

I-55-D (1.33 g, 8.28 mmol) is dissolved in THF (50 mL), tert-butylalcohol (25 mL) and H₂O (25 mL), under N₂, and NaClO₂ (2.81 g, 24.84 mmol) and KH₂PO₄ (2.25 g, 16.56 mmol) are added. The reaction mixture is stirred overnight at rt, concentrated to dryness, dissolved in 50% saturated brine (60 mL) and extracted with ether (3×). TLC of extracts indicates acid as well as residual aldehyde, so the organic and aqueous layers are combined and basified to pH 10 with NH₄OH. The layers are separated and the residual aldehyde extracted with additional ether. The aqueous layer is acidified to pH 3 with concentrated HCl, then extracted with CH₂Cl₂ (4×). Large amounts of acid remained in the aqueous layer, so the aqueous layer is concentrated to dryness. The solid is triturated with CHCl₃ (4×), and then 10% MeOH/CH₂Cl₂ (4×) to extract much of the acid into the supernatant. The combined organic layer is dried (Na₂SO₄), filtered, and concentrated to a tan solid (1.69 g, greater than 100% isolated yield). The solid is diluted with CHCl₃ and is heated to reflux for 3 h. The flask is removed from heat, allowed to cool slightly, then filtered. The filtrate is concentrated to a tan solid (1.02 g). The solid is triturated with ether, filtered and dried to afford 3-methylfuro[2,3-c]pyridine-5-carboxylic acid (I-56-D) as a light tan solid (51% yield). MS (CI) for C₉H₇NO₃, m/z: 178 (M+H).

Intermediate D8: 3-Ethylfuro[2,3-c]pyridine-5-carboxylic acid

From 1-chloro-2-butene and 2-chloro-6-(hydroxymethyl)-4-iodo-3-pyridinol (I-2-D), the corresponding 3-ethylfuro[2,3-c]pyridine-5-carboxylic acid (I-60-D) was prepared. HRMS (FAB) calculated for C₁₀H₉NO₃+H: 192.0661, found 192.0659 (M+H).

Intermediate D10: Furo[2,3-b]pyridine-2-carboxylic

Ethyl glycolate (35.5 mL, 375 mmol) is slowly added (over 20 min) to a slurry of NaOH (15.8 g, 394 mmol) in 1,2-dimethoxyethane (400 mL) under N₂ with the flask being in an ice bath. The mixture is allowed to warm to rt, is stirred for 30 min, and ethyl 2-chloronicotinate (27.84 g, 150 mmol) in 1,2-dimethoxyethane (50 mL) is added over 10 minutes. The reaction is warmed to 65° C. for 15 h in an oil bath. The mixture is concentrated to dryness, the residue is dissolved in H₂O (500 mL), washed with hexane (500 mL), acidified to pH 3 with 5% HCl, and extracted with CHCl₃ (4×400 mL). The combined organic layer is dried (MgSO₄), filtered, and concentrated to a yellow solid. The solid is suspended in ether (200 mL) and heated on a steam bath until concentrated to a volume of 40 mL. The material is allowed to crystallize overnight, then filtered to afford ethyl 3-hydroxyfuro[2,3-b]pyridine-2-carboxylate (I-40-D) as a pale orange solid (41% yield). Additional material is obtained by concentrating the filtrate. Recrystallization in ether a second time afforded I-40-D as a pale yellow solid (7.3% yield). MS (EI) for C₁₀H₉NO₄, m/z: 207 (M)⁺.

I-40-D (207 mg, 1.0 mmol) is added to TEA (139 μL, 1.0 mmol) in CH₂Cl₂ (5 mL) at rt and 2-[N,N-bis(trifluoromethylsulfonyl)amino]-5-chloropyridine (393 mg, 1.0 mmol) is added. The solution is stirred for 1 h at rt, diluted with EtOAc (25 mL) and washed with 50% saturated brine (2×15 mL). The organic layer is dried (Na₂SO₄), filtered, and concentrated to a yellow oil which solidified upon standing. The crude material is adsorbed onto silica gel (1.2 g) and chromatographed over 25 g slurry-packed silica gel, eluting with 20% EtOAc/hexane to afford ethyl 3-([(trifluoromethyl)sulfonyl]oxy)furo[2,3-b]pyridine-2-carboxylate (I-41-D) as a white crystalline solid (98% yield). Analysis calculated for C₁₁H₈F₃NO₆S: C, 38.94; H, 2.38; N, 4.13, found: C, 38.84; H, 2.29; N, 4.11.

I-41-D (1.36 g, 4.0 mmol) is added to 10% Pd/C catalyst (68 mg) and NaHCO₃ (336 mg, 4.0 mmol) in EtOH (100 mL)/H₂O (5 mL) in a 250 mL Parr shaker bottle. The mixture is hydrogenated at 10 PSI for 5 h, filtered and concentrated to a residue. The residue is partitioned between 50% saturated NaHCO₃ (80 mL) and EtOAc (80 mL). The organic layer is dried (Na₂SO₄), filtered, and concentrated in vacuo to a colorless oil which solidified upon standing (793 mg). The crude material is chromatographed over 40 g slurry-packed silica gel, eluting with 25% EtOAc/hexane to afford ethyl furo[2,3-b]pyridine-2-carboxylate (I-42-D) as a white solid (90% yield). MS (EI) for C₁₀H₉NO₃, m/z: 191 (M)⁺.

I-42-D (758 mg, 3.96 mmol) is dissolved in MeOH (20 mL) and lithium hydroxide monohydrate (366 mg, 8.7 mmol) in 6 mL H₂O is added under N₂. The reaction is stirred at rt for 2 h, concentrated to near-dryness, diluted with H₂O (5 mL) and acidified to pH 3 with 10% HCl. The resulting solid is collected by filtration, washed with additional water and dried to afford furo[2,3-b]pyridine-2-carboxylic acid (I-43-D) as a white solid (97% yield). MS (EI) for C₈H₅NO₃, m/z: 163 (M)⁺.

Intermediate D11: 3-Isopropylfuro[2,3-c]pyridine-5-carboxylic acid

3-Isopropylfuro[2,3-c]pyridine-5-carboxylic acid (I-70-D) is obtained starting with 1-chloro-3-methyl-2-butene and 2-chloro-6-(hydroxymethyl)-4-iodo-3-pyridinol (I-2-D), using the method described for Intermediate C7, making non-critical changes. HRMS (FAB) calculated for C₁₁H₁₁NO₃+H: 206.0817, found 206.0817 (M+H)⁺.

Intermediate D12: Thieno[2,3-b]pyridine-2-carboxylic acid

THF (200 mL) in a dry flask under N₂ is chilled by placing the flask in a dry-ice/acetone bath at −78° C. Butyllithium (125 mL, 200 mmol) is added drop-wise, followed by the drop-wise addition of iodobenzene (11.19 mL, 100 mmol) in THF (10 mL). The solution is allowed to stir for 30 min at −78° C. Diisopropylamine (0.70 mL, 5 mmol) in THF (3 mL) and 2-chloropyridine (9.46 mL, 100 mmol) in THF (30 mL) are added successively in a drop-wise manner, and the solution is stirred for 1 h at −40° C. Formyl piperidine (11.1 mL, 100 mmol) in THF (25 mL) is added drop-wise, and the solution is stirred for 1 h at −40° C. The reaction is quenched with 40 mL 6N HCl, diluted with 250 mL ether, and a small amount of sodium thiosulfate solution is added to remove the iodine color. The solution is neutralized with saturated NaHCO₃, filtered, and extracted with ether (3×150 mL). The combined organic layer is dried (Na₂SO₄), filtered, and concentrated in vacuo. The crude material is chromatographed over 600 g slurry-packed silica, eluting with 20% EtOAc/hexane to afford 2-chloronicotinaldehyde (I-90-D) as a pale orange solid (54% yield). MS (EI) for C₆H₄ClNO, m/z: 141 (M)⁺.

I-90-D (1.41 g, 10.01 mmol) is dissolved in DMF (10 mL) and H₂O (1 mL) under N₂. K₂CO₃ (1.56 g, 11.27 mmol) and methyl thioglycolate (1.00 mL, 11.25 mmol) are added portionwise. The reaction is stirred at 35° C. for 24 h, quenched with cold H₂O (75 mL), and placed in an ice bath to enhance precipitation. The precipitate is isolated by filtration, affording methyl-thieno[2,3-b]pyridine-2-carboxylate (I-101-D) as an orange powder (40% yield). MS (EI) for C₉H₇NO₂S, m/z: 193 (M)⁺.

I-101-D (0.700 g, 3.63 mmol) is dissolved in MeOH (15 mL) and 3 mL H₂O. 2N NaOH (1.82 mL, 3.63 mmol) is added drop-wise, and the reaction is stirred at rt for 24 h. The reaction is concentrated in vacuo, and H₂O (40 mL) is added to dissolve the residue. The resulting solution is acidified to pH 4 using concentrated HCl, and the precipitate is isolated by filtration, yielding thieno[2,3-b]pyridine-2-carboxylic acid (I-102-D) as a white powder (85% yield). MS (EI) for C₈H₅NO₂S, m/z: 179 (M)⁺.

Intermediate D13: Thieno[2,3-b]pyridine-5-carboxylic acid

2-Nitrothiophene (33.76 g, 261.4 mmol) is suspended in concentrated HCl (175 mL) and heated to 50° C. Stannous chloride (118.05 g, 523.2 mmol) is added portionwise, maintaining the reaction temperature between 45-50° C. with an ice bath, that is removed after the addition. The solution is allowed to cool slowly to 30° C. over an hour. The solution is then cooled in an ice bath and filtered. The cake is washed with concentrated HCl (20 mL), dried in a stream of air, and washed with ether (50 mL) to afford the hexachlorostannate salt of 2-aminothiophene as a brown solid (26% yield).

3,3-Dimethyl-2-formyl propionitrile sodium (3.33 g, 20.2 mmol) can readily be prepared from the method described by Bertz, S. H., et al., J. Org. Chem., 47, 2216-2217 (1982). 3,3-Dimethyl-2-formyl propionitrile sodium is dissolved in MeOH (40 mL), and concentrated HCl (4 mL) and the hexachlorostannate salt of 2-aminothiophene (10.04 g, 19.1 mmol) in MeOH (130 mL) is slowly added drop-wise to the mixture. Following addition, the mixture is heated to reflux in an oil bath (80° C.) for 4 h, and then MeOH (10 mL) and concentrated HCl (10 mL) are added. The reaction continued refluxing for another 20 h. The solution is cooled to rt, and the reaction is concentrated in vacuo. The purple residue is dissolved in H₂O (60 mL), and the slurry is filtered. The cake is pulverized and stirred vigorously with 5% MeOH/CHCl₃ (105 mL) while heating to 55° C. The mixture is cooled and filtered, and the organic layer is concentrated to a green oil. The crude material is chromatographed over 130 g slurry-packed silica, eluting with 30% EtOAc/hexane to afford thieno[2,3-b]pyridine-5-carbonitrile (I-105-D) as a pale yellow solid (24% yield). HRMS (FAB) calculated for C₈H₄N₂S+H: 161.0173, found 161.0173 (M+H).

NaOH (0.138 g, 3.45 mmol) is added to a solution of I-105-D (0.503 g, 3.14 mmol) dissolved in 70% EtOH/H₂O (12 mL). The mixture is heated to reflux at 100° C. for 3 h. The reaction is concentrated in vacuo, and the residue is dissolved in H₂O (8 mL) and neutralized with concentrated HCl. The slurry is filtered and rinsed with ether. An initial NMR of the isolated material indicates the presence of the carboxamide intermediate, so the material is suspended in 1M NaOH (6 mL) and stirred overnight. Water (10 mL) is added, the solution is extracted with ether (3×10 mL), and the mixture is neutralized with concentrated HCl. The slurry is filtered and rinsed with ether, affording of thieno[2,3-b]pyridine-5-carboxylic acid (I-106-D) as an off-white solid (48% yield). MS (EI) for C₈H₅NO₂S, m/z: 179 (M)⁺.

Intermediate D14: Thieno[2,3-b]pyridine-6-carboxylic acid

2-Nitrothiophene (12.9 g, 99.9 mmol) is dissolved in concentrated HCl (200 mL) and stirred vigorously at 30° C. Granular tin (25 g, 210 mmol) is slowly added portionwise. When the tin is completely dissolved, zinc chloride (6.1 g, 44.7 mmol) in EtOH (70 mL) is added drop-wise, the mixture is heated to 85° C. and malondialdehyde diethyl acetal (24 mL, 100 mmol) in EtOH (30 mL) is added. The solution continued stirring at 85° C. for 1 h, and is quenched by pouring over ice (100 g). The mixture is adjusted to pH 10 with NH₄OH, and the resulting slurry is carefully filtered through celite overnight. The liquor is extracted with CHCl₃ (3×300 mL), and the combined organic layer is dried (MgSO₄), filtered, and concentrated to a brown oil. The crude material is chromatographed over 250 g slurry-packed silica, eluting with 35% EtOAc/hexane to give thieno[2,3-b]pyridine (I-110-D) as an orange oil (26% yield). MS (EI) for C₇H₅NS, m/z: 135 (M)⁺.

I-110-D (3.47 g, 25.7 mmol) is dissolved in acetic acid (12 mL) and heated to 85° C. 30% Hydrogen peroxide (9 mL) is added drop-wise and the solution is allowed to stir overnight. The reaction is allowed to cool to rt and quenched with paraformaldehyde until a peroxide test proved negative using starch-iodine paper. The solution is diluted with H₂O (100 mL) and neutralized with NaHCO₃, then extracted repeatedly with CHCl₃ (12×80 mL, 6×50 mL). The combined organic layer is dried (Na₂SO₄), filtered, and concentrated to a brown solid. The crude material is chromatographed over 70 g slurry-packed silica eluting with 3.5% MeOH/CH₂Cl₂ to afford thieno[2,3-b]pyridine-7-oxide (I-111-D) as a pale yellow solid (22% yield). MS (EI) for C₇H₅NOS m/z: 151 (M)⁺.

A 0.5M solution of I-111-D (5 mL, 2.5 mmol) in CH₂Cl₂ is diluted with 8 mL of CH₂Cl₂ under N₂. Dimethyl carbamyl chloride (0.27 mL, 2.9 mmol) is added drop-wise, followed by the addition of trimethylsilyl cyanide (0.388 mL, 2.9 mmol) via syringe. The reaction is allowed to stir for 9 days and is quenched with 10% K₂CO₃ (10 mL). The layers are allowed to separate, the organic layer is isolated and dried (K₂CO₃), filtered, and concentrated to a brown solid. The crude material is chromatographed over 25 g slurry-packed silica, eluting with 35% EtOAc/hexane to afford thieno[2,3-b]pyridine-6-carbonitrile (I-112-D) as a pale yellow solid (100% yield). Analysis calculated for C₈H₄N₂S: C, 59.98; H, 2.52; N, 17.49, found: C, 59.91; H, 2.57; N, 17.43.

NaOH (398 mg, 9.95 mmol) is added portionwise to a solution of I-112-D (674 mg, 4.2 mmol) in 70% EtOH/H₂O (20 mL). The solution is heated to reflux at 100° C. for 24 h, and the reaction is concentrated in vacuo. The residue is dissolved in H₂O (15 mL) and washed with ether (3×10 mL). Concentrated HCl is used to adjust the pH to 3.5, creating a precipitate. The slurry is filtered, giving thieno[2,3-b]pyridine-6-carboxylic acid (I-113-D) as a white solid (45% yield). MS (EI) for C₈H₅NO₂S, m/z: 179(M)⁺.

Intermediate D15: Thieno[2,3-c]pyridine-2-carboxylic acid

THF (200 mL) is chilled to −70° C. in a dry flask under N₂, and N-butyllithium (24.4 mL, 55.0 mmol) is added drop-wise. The reaction is placed in an ice bath and DIA (7.71 mL, 55.0 mmol) in THF (20 mL) is added drop-wise. The solution is again chilled to −70° C., and 3-chloropyridine (4.75 mL, 50.0 mmol) in THF (20 mL) is added drop-wise. The reaction is allowed to stir for 4 h at −70° C. and ethyl formate (4.44 mL, 55.0 mmol) in THF (20 mL) is added. The reaction is stirred for an additional 3 h at −70° C. and quenched with H₂O (500 mL). The layers are allowed to separate, and the aqueous layer is extracted with EtOAc (3×250 mL). The combined organic layer is dried (MgSO₄), filtered, and concentrated to a dark brown solid. The crude material is chromatographed over 250 g slurry-packed silica, eluting with 50% EtOAc/hexane to give 3-chloroisonicotinaldehyde (I-120-D) as an off-white solid (55% yield). MS (EI) for C₆H₄ClNO, m/z: 141 (M)⁺.

I-120-D (2.12 g, 14.9 mmol) is dissolved in DMF (75 mL) with a small amount of H₂O (7.5 mL). Methyl thioglycolate (1.67 mL, 18.7 mmol) and K₂CO₃ (2.59 g, 18.7 mmol) are added portionwise, and the mixture is stirred at 45° C. for 24 h. The reaction is quenched with cold H₂O (200 mL) and extracted with EtOAc (3×150 mL). The combined organic layer is washed with 50% NaCl solution (3×150 mL), dried (MgSO₄), filtered, and concentrated to an orange solid. The crude material is chromatographed over 40 g slurry-packed silica, eluting with 50% EtOAc/hexane to afford ethyl thieno[2,3-c]pyridine-2-carboxylate (I-121-D) as a pale yellow solid (22% yield).

I-121-D (577 mg, 2.99 mmol) is combined with 2M NaOH (1.5 mL, 3.0 mmol) in MeOH (15 mL) and H₂O (1.5 mL). The reaction is stirred at rt for 24 h. The reaction is concentrated in vacuo and the residue is dissolved in H₂O (75 mL). Concentrated HCl is used to acidify the solution to pH 3. The slurry is filtered, washed with H₂O and ether, and dried, affording thieno[2,3-c]pyridine-2-carboxylic acid (I-122-D) as an off-white solid (38% yield). HRMS (FAB) calculated for C₈H₅NO₂S+H: 180.0119, found 180.0119 (M+H).

Intermediate D16: Thieno[3,2-b]pyridine-2-carboxylic acid

3-Chloropyridine (9.5 mL. 99.9 mmol) is dissolved in acetic acid (35 mL) and heated to 98° C. 30% Hydrogen peroxide (28 mL) is added drop-wise, and the reaction stirred for 5 h at 98° C. The reaction is cooled and paraformaldehyde is added so that a negative peroxide test is achieved using starch-iodine paper. The solution is concentrated in vacuo and the crude paste is chromatographed over 600 g slurry-packed silica eluting with 4 L of 2% MeOH/CH₂Cl₂, 2 L of 4% MeOH/CH₂Cl₂, and finally 1 L of 10% MeOH/CH₂Cl₂ to afford 3-chloropyridine 1-oxide (I-125-D) as a pale oil (100% yield).

A 2M solution of I-125-D (10 mL, 20 mmol) is combined with an additional 90 mL of CH₂Cl₂. Dimethylcarbamoyl chloride (2.03 mL, 22.0 mmol) is added drop-wise, followed by the addition of trimethyl silylcyanide (2.93 mL, 22.0 mmol) via syringe. The reaction is stirred at rt for 10 days and is quenched with 10% K₂CO₃ (100 mL). The layers are allowed to separate, and the organic layer is dried (K₂CO₃), filtered, and concentrated to an orange solid. The crude material is chromatographed over 160 g slurry-packed silica eluting with 40% EtOAc/hexane to yield 3-chloropyridine-2-carbonitrile (I-126-D) as a white solid (59% yield). MS (EI) for C₆H₃ClN₂, m/z 138 (M)⁺.

I-126-D (1.01 g, 7.29 mmol) and K₂CO₃ (1.10 g, 7.96 mmol) are added to DMF (10 mL) and H₂O (1 mL). Methyl thioglycolate (0.709 mL, 7.93 mmol) is added drop-wise, and the solution is heated to 40° C. and stirred for 3 h. The reaction is quenched with cold H₂O (70 mL) and placed on ice to enhance precipitation. The slurry is filtered and the cake is dissolved in CHCl₃. This organic solution is dried (MgSO₄), filtered, and concentrated, affording methyl 3-aminothieno[3,2-b]pyridine-2-carboxylate (I-127-D) as a yellow solid (84% yield). HRMS (FAB) calculated for C₉H₈N₂O₂S+H: 209.0385, found 209.0383 (M+H).

I-127-D (0.919 g, 4.42 mmol) is dissolved in 50% hypophosphorous acid (35 mL) and chilled in an ice bath. Sodium nitrite (0.61 g, 8.84 mmol) is dissolved in a minimal amount of H₂O and added drop-wise to the previous solution, and the reaction is stirred for 3 h in an ice bath. 3M NaOH is used to adjust the pH to 7.9, and the solution is extracted with EtOAc (3×100 mL). The combined organic layer is dried (MgSO₄), filtered, and concentrated to afford methyl thieno[3,2-b]pyridine-2-carboxylate (I-128-D) as a yellow solid (44% yield). MS (EI) for C₉H₇NO₂S, m/z: 193 (M)⁺.

2M NaOH (0.8 mL, 1.6 mmol) and I-128-D (300 mg, 1.55 mmol) are added to MeOH (8 mL) and H₂O (1 mL) and is stirred for 24 h. The reaction is concentrated in vacuo, and the residue is dissolved with H₂O (5 mL). 5% HCl is used to adjust the pH to 3.5, creating a precipitate. The slurry is filtered and washed with ether, affording thieno[3,2-b]pyridine-2-carboxylic acid (I-129-D) as a brown solid (67% yield). HRMS (FAB) calculated for C₈H₅NO₂S+H: 180.0119, found 180.0121 (M+H).

Intermediate D17: Thieno[3,2-b]pyridine-6-carboxylic acid

Methyl 3-aminothiophene-2-carboxylate (1.52 g, 9.68 mmol) is dissolved in 2M NaOH (10 mL, 20 mmol) and heated to reflux in a 115° C. oil bath for 30 min. The mixture is cooled to rt, placed in an ice bath, and carefully acidified with concentrated HCl. The slurry is filtered and rinsed with H₂O (25 mL). The cake is then dissolved in acetone (50 mL), dried (MgSO₄), filtered, and concentrated to a thick paste. The crude material is dissolved in 1-propanol (25 mL), and oxalic acid (0.90 g, 10.0 mmol) is added portionwise. The mixture is heated at 38° C. for 45 min, cooled to rt, and diluted with ether. The precipitate is isolated via filtration, and washed with ether, affording 3-amino-thiophene oxalate (I-135-D) as a fluffy white solid (70% yield). HRMS (FAB) calculated for C₄H₅NS+H: 100.0221, found 100.0229 (M+H).

3,3-Dimethyl-2-formyl propionitrile sodium (5.38 g, 32.6 mmol) is dissolved in MeOH (60 mL) with concentrated HCl (6 mL). I-135-D (6.16 g, 32.6 mmol) is suspended in MeOH (200 mL) and added drop-wise to the acidic solution. The mixture is heated to reflux at 80° C. for 5 h when an additional 20 mL concentrated HCl and 20 mL H₂O are added; the mixture continues refluxing for another 12 h. The mixture is concentrated in vacuo, and the residue is dissolved with cold H₂O (100 mL). The resulting precipitate is filtered off and dried, giving thieno[3,2-b]pyridine-6-carbonitrile (I-136-D) as a brown solid (44% yield). HRMS (FAB) calculated for C₈H₄N₂S+H: 161.0173, found 161.0170 (M+H).

I-136-D (1.99 g, 12.5 mmol) is dissolved in 70% EtOH/H₂O (20 mL), and NaOH (0.52 g, 13.0 mmol) is added portionwise. The mixture is heated at 100° C. for 15 h and then allowed to cool to rt. The mixture is concentrated in vacuo. The residue is dissolved in cold H₂O (30 mL), and the solution is rinsed with ether (3×10 mL). The pH is adjusted to 3.5 with concentrated HCl to precipitate the desired product that is removed by filtration to give thieno[3,2-b]pyridine-6-carboxylic acid (I-137-D) as a tan solid (77% yield). HRMS (FAB) calculated for C₈H₅NO₂S+H: 180.0119, found 180.0118 (M+H).

Intermediate D18: Thieno[3,2-c]pyridine-2-carboxylic acid

4-Chloropyridine hydrochloride (15 g, 99.9 mmol) is free-based by stirring in 1000 mL 1:1 saturated NaHCO₃/ether for 1 h. The layers are allowed to separate, the aqueous layer is extracted with ether (2×175 mL), and the combined organic layer is dried (MgSO₄), filtered, and concentrated to an oil. THF (300 mL) is chilled to −70° C. in a dry flask. N-butyllithium (105.1 mL, 168.2 mmol) is added drop-wise, and the mixture is placed in an ice bath. Diisopropylamine (23.6 mL. 168.4 mmol) in THF (50 mL) is added drop-wise, the yellow solution is stirred for 30 min, and the reaction is cooled to −70° C. The free-based 4-chloropyridine oil (9.55 g, 84.1 mmol) is dissolved in THF (50 mL) and added drop-wise to the chilled yellow solution, that turned dark red after the addition. The reaction is stirred at −70° C. for 2 h. Ethyl formate (13.6 mL, 168.3 mmol) in THF (25 mL) is then added drop-wise to the dark solution at −70° C. After 2 hours, the reaction is warmed to −10° C. and quenched with water (450 mL). The layers are allowed to separate, and the aqueous layer is extracted with ether (3×200 mL). The combined organic layer is dried (MgSO₄), filtered, and concentrated in vacuo to an oil. The crude material is chromatographed over 320 g slurry-packed silica eluting with 30% EtOAc/hexane to afford 4-chloropyridine-3-carboxaldehyde (I-140-D) an orange oil which solidified under vacuum to an orange solid (21% yield).

I-140-D (2.53 g, 17.9 mmol) is dissolved in DMF (20 mL) and H₂O (2 mL). K₂CO₃ (2.97 g, 21.5 mmol) and methyl thioglycolate (1.92 mL, 21.5 mmol) are added portionwise. The reaction is stirred at 45° C. for 24 h, then quenched with cold H₂O (100 mL), and the flask is placed on ice to enhance precipitation. The precipitate is isolated by filtration and dried, affording methyl thieno[3,2-c]pyridine-2-carboxylate (I-141-D) as a white solid (92% yield). MS (EI) for C₉H₇NO₂S, m/z: 193 (M)⁺.

I-141-D (2.65 g, 13.7 mmol) is dissolved in MeOH (70 mL) and H₂O (5 mL). 2N NaOH (6.86 mL, 13.7 mmol) is added drop-wise, and the reaction is stirred at rt for 24 h. The reaction is concentrated in vacuo, and H₂O (150 mL) is added to dissolve the residue. The resulting salt solution is acidified to pH 3.5 using concentrated HCl, and the precipitate is isolated by filtration and dried, affording thieno[3,2-c]pyridine-2-carboxylic acid (I-142-D) as a white powder (57% yield). HRMS (FAB) calculated for C₈H₅NO₂S+H: 180.0119, found 180.0124 (M+H).

Intermediate D19: Thieno[2,3-c]pyridine-5-carboxylic acid

Glyoxylic acid monohydrate (20.3 g, 221 mmol) and benzyl carbamate (30.6 g, 202 mmol) are added to ether (200 mL). The solution is allowed to stir for 24 h at rt. The resulting thick precipitate is filtered, and the residue is washed with ether, affording ([(benzyloxy)carbonyl]amino)(hydroxy)acetic acid (I-150-D) as a white solid (47% yield). MS (CI) for C₁₀H₁₁NO₅+H m/z: 226 (M+H).

I-150-D (11.6 g, 51.5 mmol) is dissolved in absolute MeOH (120 mL) and chilled in an ice bath. Concentrated sulfuric acid (2.0 mL) is carefully added drop-wise. The ice bath is allowed to expire as the solution stirred for 2 days. The reaction is quenched by pouring onto a mixture of 500 g ice with saturated NaHCO₃ solution (400 mL). The solution is extracted with EtOAc (3×300 mL), and the combined organic layer is dried (MgSO₄), filtered, and concentrated to a pale oil that crystallized upon standing, giving methyl([(benzyloxy)carbonyl]amino)(methoxy)-acetate (I-151-D) as a white solid (94% yield). Analysis calculated for C₁₂H₁₅NO₅: C, 56.91; H, 5.97; N, 5.53, found: C, 56.99; H, 6.02; N, 5.60.

I-151-D (11.76 g, 46.4 mmol) is dissolved in toluene (50 mL) under N₂ and heated to 70° C. Phosphorous trichloride (23.2 mL, 46.4 mmol) is added drop-wise via syringe, and the solution is stirred for 18 h at 70° C. Trimethyl phosphite (5.47 mL, 46.4 mmol) is then added drop-wise, and stirring continued for an additional 2 h at 70° C. The mixture is concentrated in vacuo to an oil, and the crude material is dissolved in EtOAc (100 mL) and washed with saturated NaHCO₃ (3×50 mL). The organic layer is dried (Na₂SO₄), filtered, and concentrated to a volume of 30 mL. This remaining solution is stirred vigorously while hexane is added until a precipitate formed. The precipitated solid is removed by filtration, affording methyl ([(benzyloxy)carbonyl]amino) (dimethoxyphosphoryl)acetate (I-152-D) as a white solid (84% yield). MS (EI) for C₁₃H₁₈NO₇P, m/z: 331 (M)⁺.

I-152-D (12.65 g, 38.2 mmol) and acetic anhydride (9.02 mL, 95.5 mmol) in MeOH (100 mL) were added to a Parr flask. The solution is hydrogenated with 10% Pd/C catalyst (0.640 g) at 45 PSI for 3 h. The catalyst is filtered off, and the filtrate is concentrated in vacuo to an oil. The oil is placed under reduced pressure and solidified as the reduced pressure is applied. The white residue is dissolved in a small amount of EtOAc and stirred vigorously while pentane is added until a precipitate began to form. The precipitate is removed by filtration to give methyl (acetylamino)(dimethoxyphosphoryl)acetate (I-153-D) as a white powder (87% yield). MS (CI) for C₇H₁₄NO₆P, m/z: 240 (M+H).

2,3-Thiophene dicarboxaldehyde (1.40 g, 9.99 mmol) is dissolved in CH₂Cl₂ (100 mL) and the flask is placed in an ice bath. I-152-D (2.63 g, 11.0 mmol) is dissolved in CH₂Cl₂ (50 mL), 1,8-diazabicyclo[5.4.0]undec-7-ene (1.65 mL, 11.0 mmol) is added, and this solution is added drop-wise to the chilled thiophene solution. The reaction mixture is stirred for 1 h while the flask is in an ice bath and then over night at rt. The reaction is concentrated in vacuo, and the crude material is chromatographed over 300 g slurry-packed silica eluting with 50% EtOAc/hexane. The fractions were collected in two different groups to obtain the desired compounds. Each group of fractions is combined and concentrated separately. The first group of fractions affords methyl thieno[2,3-c]pyridine-5-carboxylate (I-154-D) as a white solid (41% yield), and the second group of fractions affords methyl thieno[3,2-c]pyridine-6-carboxylate (I-155-D) as a yellow solid (38% yield). MS (EI) for I-154-D for C₉H₇NO₂S, m/z: 193 (M)⁺. MS (EI) for I-155-D for C₉H₇NO₂S, m/z: 193 (M)⁺.

I-154-D (736 mg, 3.8 mmol) is dissolved in MeOH (16 mL) with water (2 mL). 2M NaOH (2.0 mL, 4.0 mmol) is added drop-wise and the solution stirred at rt. After 2 days (complete disappearance of ester by TLC), the reaction is concentrated in vacuo. The residue is dissolved in H₂O (12 mL), and the pH is adjusted to 3.5 with 10% HCl. The precipitated solid is removed by filtration, and the solid is rinsed with ether, affording thieno[2,3-c]pyridine-5-carboxylic acid (I-156-D) as a white solid (58% yield). HRMS (FAB) calculated for C₈H₅NO₂S+H: 180.0119, found 180.0123 (M+H).

Intermediate D20: Thieno[3,2-c]pyridine-6-carboxylic acid

Methyl thieno[3,2-c]pyridine-6-carboxylate (I-155-D) (678 mg, 3.5 mmol) is dissolved in MeOH (16 mL) and H₂O (2 mL). 2M NaOH (1.8 mL, 3.6 mmol) is added drop-wise, and the solution stirred at rt. After 2 days (complete disappearance of ester by TLC), the solution is concentrated in vacuo. The residue is dissolved in H₂O (12 mL), and the pH is adjusted to 3.5 with 10% HCl. The precipitated solid is removed by filtration, and the solid is rinsed with ether, affording thieno[3,2-c]pyridine-6-carboxylic acid (I-160-D) as a white solid (43% yield). HRMS (FAB) calculated for C₈H₅NO₂S+H: 180.0119, found 180.0123 (M+H).

Intermediate D21: 1H-Pyrrolo[2,3-c]pyridine-5-carboxylic acid

2,4-Lutidine (51.4 mL, 0.445 mole) is added drop-wise to 250 mL fuming sulfuric acid in a flask under N₂ in an ice bath. The solution is treated portionwise with potassium nitrate (89.9 g, 0.889 mole) over a 15 min period. The reaction is stirred 1 h in an ice bath, 2 h at rt, is gradually warmed in a 100° C. oil bath for 5 h, and then in a 130° C. oil bath for 4 h. The mixture is cooled, is poured into 1000 mL ice, and the mixture is neutralized with NaHCO₃ (1,100 g, 13.1 mole). The precipitated Na₂SO₄ is removed by filtration, the solid is washed with 500 mL H₂O and the filtrate is extracted with 4×500 mL ether. The combined organic layer is dried (MgSO₄) and is concentrated in vacuo to a yellow oil (50 g). The crude oil is distilled under vacuum to provide three fractions: 16 g recovered 2,4-lutidine (85° C.), 16 g 2,4-dimethyl-3-nitro-pyridine (I-169-D) contaminated with 25% 2,4-dimethyl-5-nitro-pyridine (135-145° C.), and 16 g 2,4-dimethyl-5-nitro-pyridine (I-170-D) contaminated with 2,4-dimethyl-3-nitropyridine (145-153° C.). ¹H NMR of C169 (CDCl₃) δ 2.33, 2.54, 7.10, 8.43 ppm. ¹H NMR of C170 (CDCl₃) δ 2.61, 2.62, 7.16, 9.05 ppm.

I-170-D/I-169-D (75:25) (5.64 g, 37 mmol) is combined with benzeneselenic anhydride (8.2 g, 22.8 mmol) in 300 mL dioxane in a flask under N₂. The reaction is warmed to reflux for 10 h, is cooled, and is concentrated to a dark yellow oil. The oil is chromatographed over 250 g silica gel (230-400 mesh) eluting with 15% EtOAc/hexane to afford 2-formyl-4-methyl-5-nitropyridine (I-171-D) (66% yield). HRMS (EI) calculated for C₇H₆N₂O₃: 166.0378, found 166.0383 (M⁺).

I-171-D (1.15 g, 6.9 mmol), p-toluene sulfonic acid (41 mg, 0.22 mmol), and ethylene glycol (1.41 mL, 25 mmol) are added to 25 mL toluene in a flask equipped with a Dean-Starke trap. The reaction is warmed to reflux for 2 h, is cooled to rt, and is concentrated in vacuo to an oily residue. The crude oil is chromatographed over 40 g silica gel (Biotage), eluting with 20% EtOAc/hexane to afford 2-(1,3-dioxolan-2-yl)-4-methyl-5-nitropyridine (I-172-D) (90% yield). MS (EI) for C₉H₁₀N₂O₄, m/z: 210 (M)⁺.

I-172-D (1.3 g, 6.2 mmol) and DMF dimethyl acetal (1.12 mL, 8.4 mmol) are added to 15 mL DMF under N₂. The reaction is warmed to 90° C. for 3 h, is cooled, and the reaction is concentrated in vacuo. The residue is combined with 1.25 g 5% Pd/BaSO₄ in 20 mL EtOH in a 250 mL Parr shaker bottle and the mixture is hydrogenated at ambient pressure until uptake ceased. The catalyst is removed by filtration, and the filtrate is combined with 500 mg 10% Pd/C catalyst in a 250 mL Parr shaker bottle. The mixture is hydrogenated at ambient pressure for 1 h. No additional hydrogen uptake is observed. The catalyst is removed by filtration, and the filtrate is concentrated in vacuo to a tan solid. The crude material is chromatographed over 50 g silica gel (230-400 mesh), eluting with 7% MeOH/CH₂Cl₂. The appropriate fractions are combined and concentrated to afford 5-(1,3-dioxolan-2-yl)-1H-pyrrolo[2,3-c]pyridine (I-173-D) (69% yield). MS for C₁₀H₁₀N₂O₂, (EI) m/z: 190 (M)⁺.

I-1730-D (800 mg, 4.21 mmol) is dissolved in 44 mL 10% aqueous acetonitrile. p-Toluene sulfonic acid (630 mg, 3.3 mmol) is added, and the mixture is heated to reflux for 5 h. The mixture is cooled to rt, is concentrated in vacuo, and the resultant residue is diluted with 15 mL saturated NaHCO₃. A pale yellow solid is collected, washed with water, and is dried to afford 1H-pyrrolo[2,3-c]pyridine-5-carbaldehyde (I-174-D) (81% yield). HRMS (FAB) calculated for C₈H₆N₂O+H: 147.0558, found 147.0564 (M+H).

I-174-D (500 mg, 3.42 mmol) is dissolved in 1.5 mL formic acid. The solution is cooled in an ice bath, 30% aqueous hydrogen peroxide (722 μL, 6.8 mmol) is added drop-wise, and the reaction is stirred 1 h in an ice bath, and allowed to stand overnight at 5° C. The mixture is diluted with H₂O, the solid is collected, washed with H₂O and is dried to give 522 mg of an off-white solid. The formate salt is added to 7 mL H₂O, 3 mL 2N NaOH is added, and the pH is adjusted to 3 with 5% aqueous HCl. The precipitate is collected and is dried to afford 1H-pyrrolo[2,3-c]pyridine-5-carboxylic acid (I-176-D) (67% yield). HRMS (FAB) calculated for C₈H₆N₂O₂+H: 163.0508, found 163.0507 (M+H).

Intermediate D22: 1-Methyl-pyrrolo[2.3-c]pyridine-5-carboxylic acid

5-(1,3-Dioxolan-2-yl)-1H-pyrrolo[2,3-c]pyridine (I-173-D) (1.05 g, 5.52 mmol) is dissolved in 20 mL THF in a dried flask under N₂. 60% Sodium hydride (243 mg, 6.07 mmol) is added, the reaction is stirred 30 min, methyl iodide (360 μL, 5.8 mmol) is added, and the reaction is stirred overnight at rt. The reaction is concentrated in vacuo and the residue is partitioned between 10 mL saturated NaCl and CH₂Cl₂ (4×10 mL). The combined organic layer is dried (K₂CO₃) and is concentrated in vacuo to a tan paste. The crude material is chromatographed over 50 g silica gel (230-400 mesh) eluting with 5% MeOH/CH₂Cl₂. The appropriate fractions are combined and concentrated to afford 5-(1,3-dioxolan-2-yl)-1-methyl-1H-pyrrolo[2,3-c]pyridine (I-175-D) (86% yield). HRMS (FAB) calculated for C₁₁H₁₂N₂O₂+H: 205.0977, found 205.0983.

I-175-D (920 mg, 4.5 mmol) is dissolved in 25 mL 10% aqueous acetonitrile in a flask. p-Toluene sulfonic acid (630 mg, 3.3 mmol) is added, and the mixture is heated to 90° C. for 8 h. The mixture is cooled to rt, concentrated in vacuo, and the residue is partitioned between 15 mL saturated NaHCO₃ and CH₂Cl₂ (4×10 mL). The combined organic layer is dried (K₂CO₃) and is concentrated in vacuo to afford 1-methyl-pyrrolo[2,3-c]pyridine-5-carbaldehyde (I-177-D) (99% yield). HRMS (FAB) calculated for C₉H₈N₂O+H: 161.0715, found 161.0711.

I-177-D (690 mg, 4.3 mmol) is dissolved in 2 mL formic acid. The solution is cooled in an ice bath, 30% aqueous hydrogen peroxide (970 μL, 8.6 mmol) is added drop-wise, and the reaction is stirred 1 h in an ice bath, and allowed to stand overnight at 5° C. The mixture is concentrated to dryness, is suspended in H₂O, and the pH is adjusted to 7 with 2N NaOH. The mixture is concentrated to dryness, is dissolved in MeOH, and is passed over 15 mL 50W-X2 ion exchange resin (hydrogen form) eluting with 200 mL MeOH followed by 200 mL 5% Et₃N/MeOH. The basic wash is concentrated to dryness to afford 1-methyl-pyrrolo[2,3-c]pyridine-5-carboxylic acid (I-178-D) (78% yield). HRMS (FAB) calculated for C₉H₈N₂O₂+H: 177.0664, found 177.0672 (M+H).

Intermediate D23: 3-Bromofuro[2,3-c]pyridine-5-carboxylic acid

Furo[2,3-c]pyridin-5-ylmethyl acetate (5.17 g, 27.05 mmol) is dissolved in CH₂Cl₂ (130 mL), layered with saturated NaHCO₃ (220 mL), treated with Br₂ (8.36 mL, 162.3 mmol) and stirred very slowly for 4.5 h at rt. The mixture is stirred vigorously for 30 min, is diluted with CH₂Cl₂ (100 mL) and the layers separated. The aqueous layer is extracted with CH₂Cl₂ (2×100 mL) and the combined organics are concentrated to a small volume under a stream of nitrogen. The solution is diluted with EtOH (200 mL), treated with K₂CO₃ (22.13 g, 160.1 mmol) and stirred for 2.5 days at rt. The mixture is concentrated to dryness, partitioned between 50% saturated NaCl (200 mL) and CH₂Cl₂ (5×200 mL), dried (Na₂SO₄) and concentrated in vacuo to a yellow solid (6.07 g). The crude material is adsorbed onto silica gel (12 g) and chromatographed over 250 g slurry-packed silica gel, eluting with a gradient of 50% EtOAc/hexane to 100% EtOAc. The appropriate fractions are combined and concentrated in vacuo to afford 5.02 g (81%) of (3-bromofuro[2,3-c]pyridin-5-yl)methanol as a white solid. MS (EI) m/z: 227 (M⁺).

Oxalyl chloride (1.77 mL, 20.1 mmol) is combined with CH₂Cl₂ (60 mL) in a dried flask under nitrogen, cooled to −78° C., treated dropwise with DMSO (2.86 mL, 40.25 mmol) and stirred for 20 min. The cooled solution is treated drop-wise with a solution of (3-bromofuro[2,3-c]pyridin-5-yl)methanol (4.0 mg, 17.5 mmol) in THF (50 mL), stirred for 1 h, then treated drop-wise with Et₃N (12.2 mL, 87.5 mmol). The mixture is stirred for 30 min at −78° C., then 30 min at 0° C. The mixture is washed with saturated NaHCO₃ (120 mL) and the organics dried (K₂CO₃) and concentrated in vacuo to a dark yellow solid (3.91 g). The crude material is chromatographed over 150 g slurry-packed silica gel, eluting with 30% EtOAc/hexane. The appropriate fractions are combined and concentrated in vacuo to afford 3.93 g (99%) of 3-bromofuro[2,3-c]pyridine-5-carbaldehyde as a white solid. MS (EI) m/z: 225 (M⁺).

3-Bromofuro[2,3-c]pyridine-5-carbaldehyde (3.26 g, 14.42 mmol) is dissolved in THF (100 mL)/t-BuOH (50 mL)/H₂O (50 mL), treated with a single portion of NaOCl₂ (4.89 g, 43.3 mmol) and KH₂PO₄ (3.92 g, 28.8 mmol) and stirred at rt for 18 h. The white solid is collected via filtration and the filtrate is concentrated in vacuo to dryness. The residue is suspended in water (25 mL), acidified to pH 2 with concentrated HCl and the resulting solid collected via filtration. The collected solids are dried in a vacuum oven at 50° C. for 18 h and combined to afford 3.52 g (99%) of 3-bromofuro[2,3-c]pyridine-5-carboxylic acid as a white solid. MS (EI) m/z: 241 (M⁺).

Intermediate D24: 3-Chlorofuro[2,3-c]pyridine-5-carboxylic acid

Furo[2,3-c]pyridin-5-ylmethanol (7.70 g, 51.63 mmol) is dissolved in pyridine (45 mL), treated with acetic anhydride (14.36 mL, 154.9 mmol) and stirred for 18 h at rt. The pyridine is removed in vacuo and the resulting residue dissolved in EtOAc (200 mL), washed with 50% saturated sodium bicarbonate (4×90 mL), dried (MgSO₄) and concentrated in vacuo to afford 9.32 g (94%) of furo[2,3-c]pyridin-5-ylmethyl acetate as a yellow oil. MS (EI) m/z: 191 (M⁺), 277, 148, 119, 118, 86, 84, 77, 63, 51, 50.

Furo[2,3-c]pyridin-5-ylmethyl acetate (956 mg, 5 mmol) is dissolved in CH₂Cl₂ (40 mL) and cooled to 0° C. Chlorine gas is bubbled through the solution for 15 min, the cooling bath is immediately removed and the mixture stirred for 2 h. The mixture is re-cooled to 0° C., saturated with chlorine gas, the cooling bath removed and the solution warmed to rt. The solution is layered with saturated NaHCO₃ (20 mL), stirred gently for 2 h then stirred vigorously for 15 min. The mixture is diluted with saturated NaHCO₃ (50 mL), extracted with CH₂Cl₂ (1×40 mL then 1×20 mL), dried (K₂CO₃) and concentrated to a volume of 20 mL under a stream of nitrogen. The solution is diluted with EtOH (35 mL), treated with K₂CO₃ (4.09 g, 29.6 mmol) and stirred for 18 h at rt. Water (7 mL) is added and the mixture stirred for 2 days. The mixture is concentrated to dryness, partitioned between 50% saturated NaCl (50 mL) and CH₂Cl₂ (4×50 mL), dried (K₂CO₃) and concentrated in vacuo to a brown solid (833 mg). The crude material is chromatographed over a standard 40 g Biotage column, eluting with 50% EtOAc/hexane. The appropriate fractions are combined and concentrated to afford 624 mg (68%) of (3-chlorofuro[2,3-c]pyridin-5-yl)methanol as a yellow oil. ¹H NMR (DMSO-d₆): δ 4.69, 5.56, 7.69, 8.55, 8.93 ppm.

Oxalyl chloride (231 μL, 2.6 mmol) is combined with CH₂Cl₂ (10 mL), cooled to −78° C., treated dropwise with DMSO (373 μL, 5.3 mmol) and stirred for 20 min. The cooled solution is treated dropwise with a solution of (3-chlorofuro[2,3-c]pyridin-5-yl)methanol (420 mg, 2.3 mmol) in THF (5 mL)/CH₂Cl₂ (5 mL), stirred for 1 h, then treated dropwise with Et₃N (1.59 mL, 11.45 mmol). The mixture is stirred for 30 min at −78° C., then 30 min at 0° C. The mixture is washed with saturated NaHCO₃ (20 mL) and the organics dried (K₂CO₃) and concentrated in vacuo to a yellow solid (410 mg). The crude material is chromatographed over 20 g slurry-packed silica gel, eluting with 15% EtOAc/hexane. The appropriate fractions are combined and concentrated in vacuo to afford 322 mg (77%) of 3-chlorofuro[2,3-c]pyridine-5-carbaldehyde as a white solid. ¹H NMR (CDCl₃): δ 7.89, 8.33, 9.02, 10.18 ppm.

3-Chlorofuro[2,3-c]pyridine-5-carbaldehyde (317 mg, 1.74 mmol) is dissolved in THF (10 mL)/t-BuOH (5 mL)/H₂O (5 mL), treated with a single portion of sodium chlorite (592 mg, 5.24 mmol) and KH₂PO₄ (473 mg, 3.48 mmol) and stirred at rt for 18 h. The reaction mixture is concentrated in vacuo to dryness, suspended in water (10 mL), acidified to pH 3.5 with concentrated HCl and stirred at rt for 2 h. The resulting solid is filtered, washed with water and dried in a vacuum oven at 40° C. for 18 h to afford 364 mg of 3-chlorofuro[2,3-c]pyridine-5-carboxylic acid as a white solid. MS (EI) m/z: 197 (M⁺).

Intermediate D25: Benzothieno[3,2-c]pyridine-3-carboxylic acid

N-butyl lithium (150.6 ml, 241 mmol) is added dropwise to ether (100 ml) at −20° C. under N₂. 3-Bromothianaphthene (10.5 ml, 80.3 mmol) is dissolved in ether (50 ml) and also added dropwise to the chilled solution, stirring cold for 0.5 h. DMF (16.3 ml, 210 mmol) is dissolved in ether (75 ml) and added dropwise, and the solution stirred an additional 15 h at −20° C. The reaction is quenched onto ice (300 g) in 10% H₂SO₄ (200 ml) and stirred until both layers turn yellow in color. The resulting slurry is filtered, and the cake is allowed to dry in the air stream, affording 1-benzothiophene-2,3-dicarbaldehyde (I-180-D) as a yellow solid (60% yield). HRMS (FAB) calculated for C₁₀H₆O₂S+H: 191.0167, found 191.0172 (M+H).

1-Benzothiophene-2,3-dicarbaldehyde (I-180-D) (1.91 g, 10.0 mmol) is dissolved in CH₂Cl₂ (100 ml) and chilled in an ice bath. Methyl (acetylamino)(dimethoxyphosphoryl) acetate (I-152-D) (2.63 g, 11.0 mmol) is dissolved in CH₂Cl₂ (50 ml) and added to 1,8-diazabicyclo[5.4.0]undec-7-ene (1.65 ml, 11.0 mmol), stirring for 5 minutes. This solution is added dropwise to the chilled thiophene solution. The reaction mixture is stirred in the ice bath for 1 h and then over night at rt. The reaction is concentrated in vacuo and the crude material is chromatographed over 500 g slurry-packed silica eluting with 50% ethyl acetate/hexane to afford methyl benzothieno[3,2-c]pyridine-3-carboxylate (I-181-D) as a white solid (73% yield). MS for C₁₃H₉NO₂S, (EI) m/z: 243 (M)⁺.

I-181-D (1.43 g, 5.87 mmol) is dissolved in MeOH (25 ml) with H₂O (3 ml). 2M NaOH (3.0 ml, 6.0 mmol) is added dropwise and the solution stirred at rt. After 4 days (complete disappearance of ester by TLC), the reaction is concentrated in vacuo. The residue is dissolved in H₂O (5 ml) and the pH is adjusted to 3 with 10% HCl. The solution is stirred over night before precipitation is complete. The slurry is filtered and the cake is rinsed with ether, giving a 100% yield of benzothieno[3,2-c]pyridine-3-carboxylic acid (I-182-D)as a white solid. HRMS (FAB) calculated for C₁₂H₇NO₂S+H 230.0276, found 230.0275 (M+H).

Intermediate D26: Thieno[3,4-c]pyridine-6-carboxylic acid

3,4-Dibromothiophene (12.5 ml, 113 mmol) is combined with CuCN (30.4 g, 339 mmol) in DMF (40 ml) in a dry flask under nitrogen utilizing an over-head stirrer. The reaction is allowed to reflux at 180° C. for 5 h. The dark mixture is then poured into a solution of FeCl₃ (113.6 g, 700 mmol) in 1.7M HCl (200 ml) and heated at 65° C. for 0.5 h, again using the over-head stirrer. The reaction is cooled to rt and extracted with CH₂Cl₂ (7×300 ml). Each extract is washed individually with 200 ml each 6M HCl (2×), water, saturated NaHCO₃, and water. The organics are then combined, dried (MgSO₄), filtered, and concentrated, affording 10.49 g (69%) of 3,4-dicyanothiophene as a fluffy tan solid. HRMS (EI) calcd for C₆H₂N₂S: 133.9939, found 133.9929 (M⁺).

3,4-Dicyanothiophene (5.0 g, 37.2 mmol) is suspended in benzene (150 ml) in a dry flask under nitrogen utilizing an over-head stirrer. Diisobutyl aluminum hydride (1.0M in toluene) (82.0 ml, 82.0 mmol) is added dropwise, and the reaction stirred at rt for 2 h. The reaction is then carefully quenched with MeOH (5 ml) and poured onto 30% H₂SO₄ (60 ml) with ice (200 g). The slurry is stirred until all lumps are dissolved, and the layers are allowed to separate. The aqueous layer is extracted with Et₂O (4×200 ml), and the combined organics are dried (MgSO₄), filtered, and adsorbed onto silica. The crude material is chromatographed over 225 g slurry-packed silica, eluting with 40% EtOAc/hexane. The appropriate fractions are combined and concentrated to afford 1.88 g (36%) of 3,4-thiophene dicarboxaldehyde as a pale yellow solid. MS (EI) m/z: 140 (M⁺).

3,4-Thiophene dicarboxaldehyde (1.0 g, 7.13 mmol) is dissolved in CH₂Cl₂ (40 ml) and chilled to 0° C. Methyl (acetylamino)(dimethoxyphosphoryl)acetate (1.88 g, 7.85 mmol) is dissolved in CH₂Cl₂ (30 ml) and combined with DBU (1.1 ml, 7.85 mmol). This solution is added dropwise to the chilled thiophene solution after stirring for 5 min. The reaction mixture is stirred at 0° C. for 1 h and then overnight at rt. The volatiles are removed in vacuo and the crude material is chromatographed over 68 g slurry-packed silica eluting with 70% EtOAc/hexane. The appropriate fractions are combined and concentrated to yield 2.09 g of the carbinol intermediate as a white foam. The intermediate is dissolved in CHCl₃ (50 ml) and treated with DBU (1.32 ml, 8.8 mmol) and trifluoracetic anhydride (1.24 ml, 8.8 mmol) in a drop-wise fashion. The reaction is stirred overnight at rt and is then quenched with saturated NaHCO₃ solution (50 ml). The layers are separated, and the aqueous layer is extracted with CHCl₃ (2×50 ml). The combined organics are dried (MgSO₄), filtered, and concentrated to a yellow oil. This oil is chromatographed over 50 g slurry-packed silica, eluting with 90% EtOAc/hexane. The appropriate fractions are combined and concentrated to afford 1.2 g (88%) of methyl thieno[3,4-c]pyridine-6-carboxylate as a yellow solid. MS (EI) m/z: 193 (M⁺).

Methyl thieno[3,4-c]pyridine-6-carboxylate (250 mg, 1.3 mmol) is dissolved in MeOH (7 ml) and water (1 ml). 2M NaOH (0.72 ml, 1.43 mmol) is added drop-wise. The reaction is stirred overnight at rt and is monitored by TLC. The volatiles are removed in vacuo and the residue is dissolved in water (2 ml). 10% HCl is used to adjust the pH to 3, and the reaction again stirred overnight at rt. The aqueous solution is extracted repeatedly with EtOAc (20×10 ml). The combined organics are dried (MgSO₄), filtered, and concentrated to a yellow solid. The amount of isolated product via extraction is minimal (67 mg), so the aqueous layer is concentrated and found to contain the majority of product. Extraction of the solid aqueous residue with EtOAc provided 225 mg (97%) of thieno[3,4-c]pyridine-6-carboxylic acid as a yellow solid. MS (EI) m/z: 179 (M⁺).

Intermediate D27: Benzofuran-5-carboxylic acid

1-(2,3-Dihydrobenzofuran-5-yl)ethanone is made using a procedure, making non-critical changes, as described in Dunn, J. P.; Ackerman, N. A.; Tomolois, A. J. J. Med. Chem. 1986, 29, 2326. Similar yield (82%) and similar purity (95%) are obtained. ¹H NMR (400 MHz, CDCl₃) δ 7.89, 7.83, 6.84, 4.70, 3.29, 2.58.

A mixture of 1-(2,3-dihydrobenzofuran-5-yl)ethanone (4.0 g, 25 mmol) and sodium hypochlorite [160 mL of a 6.0% aqueous solution, (Clorox brand of bleach)] at 55° C. is stirred for 1 h. The mixture (now homogeneous) is cooled to rt and solid sodium bisulfite is added until a clear color persists. Hydrochloric acid (80 mL of a 1.0 N aqueous solution) is added, followed by extraction with EtOAc. The organic layer is washed with brine, dried (MgSO₄), filtered, and concentrated in vacuo to afford 3.93 g (97%) of 2,3-dihydrobenzofuran-5-carboxylic acid as a white solid. ¹H NMR (400 MHz, CDCl₃) δ 11.0-10.3, 8.00, 6.87, 4.72, 3.31.

To a stirred solution of 2,3-dihydrobenzofuran-5-carboxylic acid (3.96 g, 24.1 mmol) in MeOH (200 mL) is added concentrated sulfuric acid (0.5 mL). The mixture is heated to reflux for 24 h. The mixture is cooled to rt, followed by the addition of solid sodium bicarbonate. The reaction mixture is concentrated in vacuo, and the remaining residue is partitioned between EtOAc and water. The aqueous layer is extracted with EtOAc, and the combined organic layers are dried (MgSO₄), filtered and concentrated in vacuo to afford 4.22 g (98%) of methyl 2,3-dihydrobenzofuran-5-carboxylate as a white solid. ¹H NMR (400 MHz, CDCl₃) δ 7.93-7.89, 6.82, 4.69, 3.86, 3.28.

To a stirred solution of methyl 2,3-dihydrobenzofuran-5-carboxylate (4.2 g, 24 mmol) in anhydrous p-dioxane (150 mL) under argon atmosphere is added 2,3-dichloro-5,6-dicyano-1,4-benzoquinone (6.42 g, 28 mmol). The mixture is heated to reflux for 24 h, followed by cooling to rt. The reaction mixture is partitioned between ether and ½ saturated aqueous sodium carbonate solution. The organic layer is extracted several times with ½ saturated aqueous sodium carbonate solution. The organic layer is washed with water, dried (MgSO₄), filtered, and concentrated in vacuo to give a mixture (92%) of recovered starting material methyl 2,3-dihydrobenzofuran-5-carboxylate and methyl benzofuran-5-carboxylate in a ratio of 1:3. The crude product is purified by preparative HPLC using a Chiralcel OJ column. Elution with heptane-iso-propyl alcohol, (80:20, flow rate=70 mL/min) gives 0.75 g (18%) of methyl 2,3-dihydrobenzofuran-5-carboxylate as a white solid and 2.5 g (61%) of methyl benzofuran-5-carboxylate as a white solid. ¹H NMR for methyl benzofuran-5-carboxylate (400 MHz, CDCl₃) δ 8.40, 8.07, 7.73, 7.57, 6.89, 3.99.

A stirred mixture of methyl benzofuran-5-carboxylate (1.3 g, 7.38 mmol) in MeOH (51 mL) and sodium hydroxide (41 mL of a 5% aqueous solution) is heated to 65° C. for 4 h. The mixture is cooled to rt, and MeOH was removed in vacuo. The remaining aqueous layer is extracted with CH₂Cl₂. The CH₂Cl₂ layer is discarded, and the aqueous layer is acidified to pH=1 with concentrated hydrochloric acid. The aqueous layer is extracted with CHCl₃. The organic layer is washed with water, dried (MgSO₄), filtered and concentrated in vacuo to afford 1.2 g (98%) of benzofuran-5-carboxylic acid as a white solid. ¹H NMR (400 MHz, DMSO-d₆) δ 12.9, 8.30, 8.11, 7.92, 7.69, 7.09.

Compounds of Formula I where W is (E) are made using the coupling procedures discussed herein and in cited references, making non-critical changes to obtain the desired compounds. The following intermediates to provide W of formula I are for exemplification only and are not intended to limit the scope of the present invention. Other intermediates within the scope of the present invention can be obtained using known procedures or by making slight modifications to known procedures.

It will be apparent to those skilled in the art that the requisite carboxylic acids can be obtained through synthesis via literature procedures or through the slight modification thereof. For example, compounds of Formula I where E⁰ is N and E¹ and E² are O, can be obtained as follows: 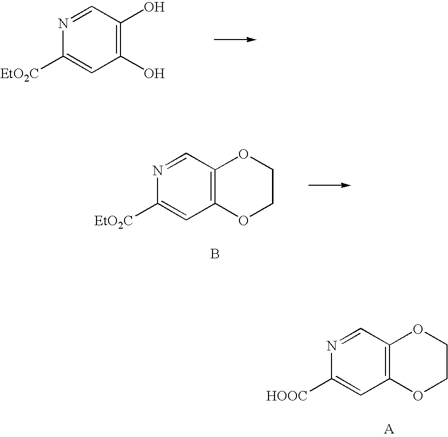
Acid A can be prepared from ethyl 4,5-dihydroxypyridine-2-carboxylate (see Z. Naturfirsch, 34b, 1729-1736, 1979). Alkylation with 1,2-dibromoethane gives B. Saponification of B with aqueous NaOH would provide the requisite carboxylic acid A. The resulting acid is coupled with an Azabicyclo using conditions described herein.

Substituents can be introduced for R_{E-1} or R_{E-2} where E⁰ is CH and E¹ and E² are each Oais described in Taniguchi, Eiji, et al., Biosci. Biotech. Biochem., 56 (4), 630-635, 1992. See also Henning, R.; Lattrell, R.; Gerhards, H. J.; Leven, M.; J. Med. Chem.; 30; 5; 1987; 814-819. This is also applicable to make the final compounds where E⁰ is N, starting with ethyl 4,5-dihydroxypyridine-2-carboxylate to obtain the ester intermediate which could be saponified:

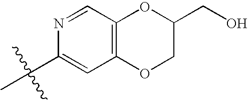

Furthermore, where E⁰ is N, the compounds where one R_{E-1} is a bond to CR_{E-1-1} or where one R_{E-2} is a bond to CR_{E-2-2}, the compounds can be obtained using methods described herein for E⁰ is CH, making non-critical changes. Moreover, where at least one R_{E-1} and/or at least one R_{E-2} is other than H and is not a bond, the compounds can be obtained using methods described herein for where E⁰ is CH.

Compounds where E⁰ is N, only one of E¹ or E² is O, R_{E-0} is other than H, and one of R_{E-1} or R_{E-2} is a bond, can be obtained as discussed herein using procedures for where E⁰ is CH. For example, 2-chloro-6-(hydroxymethyl)-4-vinylpyridin-3-ol could be converted into (8-chloro-2-methyl-2H-pyrano[2,3-c]pyridin-6-yl)methanol using the procedures discussed herein. The alcohol could be oxidized to the corresponding carboxylic acid:

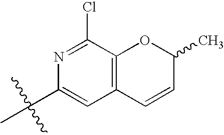

Similarly, (8-chloro-2H-pyrano[2,3-c]pyridin-6-yl)methanol can be oxidized to give 8-chloro-2H-pyrano[2,3-c]pyridin-6-carboxylic acid:

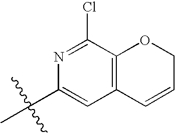

Some specific examples are provided for exemplification and are not intended to limit the scope of the present invention:

Intermediate E1: 2,3-Dihydro-1,4-benzodioxine-6-carboxylic acid

A suspension of calcium ethoxide (816 mg, 6.3 mmol), butene oxide (5.2 mL, 93 mmol) and 2,4-diiodophenol (2.17 g, 6.3 mmol) is heated in a sealed flask at 80° C. for 18 h. The reaction mixture is allowed to cool, poured into 1N HCl and extracted three times with CH₂Cl₂. The combined organic extracts are dried (Na₂SO₄), filtered and concentrated in vacuo. The resulting material is purified by column chromatography (two columns, step gradient of 30-40-50% CH₂Cl₂ in hexanes) to give 1-(2,4-diiodophenoxy)butan-2-ol as a clear oil (1.73 g, 67%).¹H NMR (400 MHz, CDCl₃) δ 8.04, 7.56, 6.57, 4.03, 3.9, 3.84, 2.42, 1.65, 1.04.

A solution of 1-(2,4-diiodophenoxy)butan-2-ol (1.27 g, 3.0) in pyridine (12 mL) is degassed by repeatedly evacuating the flask then filling with N₂. Sodium hydride (60% suspension, 153 mg, 3.8 mmol) is added and the resulting mixture is stirred for 15 min. Copper (I) chloride (15 mg, 0.15 mmol) is added, and the resulting mixture is heated at 80° C. for 2 h. The reaction is allowed to cool, poured into 1M HCl and extracted three times with CH₂Cl₂. The combined organic extracts are dried (Na₂SO₄), filtered and concentrated in vacuo. The resulting material is purified by column chromatography (10% CH₂Cl₂ in hexanes) to give 2-ethyl-7-iodo-2,3-dihydro-1,4-benzodioxine as a clear oil (493 mg, 57%). ¹H NMR (400 MHz, CDCl₃) δ 7.20, 7.10, 6.61, 4.22, 4.01, 3.85, 1.7, 1.6, 1.06.

A solution of 2-ethyl-7-iodo-2,3-dihydro-1,4-benzodioxine (486 mg, 1.68 mmol) in DMF (3 mL) is degassed by repeatedly evacuating the flask and filling with N₂. Zn(CN)₂ (117 mg, 1.0 mmol), and Pd(PPh₃)₄ (97 mg, 0.084 mmol) are added, and the resulting solution is degassed, and is then heated to 80° C. for 1.5 h. The reaction is allowed to cool, poured into water and extracted two times with ether. The combined organic extracts are dried (Na₂SO₄), filtered and concentrated in vacuo. The resulting material is purified by column chromatography (step gradient, 25-50% CH₂Cl₂ in hexanes) to give 3-ethyl-2,3-dihydro-1,4-benzodioxine-6-carbonitrile as a clear oil (296 mg, 92%). ¹H NMR (400 MHz, CDCl₃) δ 7.16, 7.13, 6.91, 4.31, 4.05, 3.93, 1.7, 1.6, 1.08.

KOH (218 mg, 3.9 mmol) is added to a mixture of 3-ethyl-2,3-dihydro-1,4-benzodioxine-6-carbonitrile (247 mg, 1.3 mmol), ethanol (3 mL) and water (1 mL). The resulting mixture is heated to 80° C. for 24 hours. The reaction is allowed to cool, diluted with water (2 mL) and acidified to pH<2 with concentrated HCl. The resulting solid is filtered, washed with water and dried at 60° C. under vacuum to give 3-ethyl-2,3-dihydro-1,4-benzodioxine-6-carboxylic acid as a white solid (249 mg, 92%). ¹H NMR (400 MHz, DMSO-d₆) δ 12.66, 7.43, 7.37, 6.95, 4.38, 4.10, 3.95, 1.64, 1.01.

Intermediate E2: 2-(Phenoxymethyl)-2,3-dihydro-1,4-benzodioxine-6-carboxylic acid

6-Bromo-2,3-dihydro-1,4-benzodioxin-2-yl)methanol is prepared according to literature reports for 6-fluoro-2,3-dihydro-benzo-1,4-dioxin-2-yl)-methanol. See Henning, R.; Lattrell, R.; Gerhards, H. J.; Leven, M.; J. Med. Chem.; 30; 5; 1987; 814-819. The intermediate is obtained in 70% yield as a solid: ¹H NMR (400 MHz, CDCl₃) δ 7.08, 7.00, 6.81, 4.25-4.40, 4.10-4.20, 3.85-4.00, 1.95; MS (EI) m/z 244 (M⁺).

A mixture of (6-bromo-2,3-dihydro-1,4-benzodioxin-2-yl)methanol (3.94 g, 16.1 mmol) and DMF (35 mL) at rt is treated with a 60% dispersion of NaH in mineral oil (0.706 g, 17.7 mmol). After 15 min, the mixture is treated with benzyl bromide (2.10 mL, 17.7 mmol). After 2 h, the mixture is poured into H₂O and extracted with EtOAc (2×125 mL). The combined organics are washed with H₂O (3×100 mL), brine, dried (MgSO₄), filtered, and concentrated. The resulting oil is adsorbed onto SiO₂ and chromatographed (Biotage 40M+SIM, 5% EtOAc/Hexane). The product fractions are pooled and concentrated to give an oil which solidified (upon standing) to give 3.91 g (73%) of 2-[(benzyloxy)methyl]-6-bromo-2,3-dihydro-1,4-benzodioxine: ¹H NMR (400 MHz, CDCl₃) δ 7.30-7.45, 7.06, 6.99, 6.81, 4.60-4.70, 4.30-4.40, 4.05-4.15, 3.65-3.85; MS (EI) m/z 244 (M⁺).

A mixture of 2-[(benzyloxy)methyl]-6-bromo-2,3-dihydro-1,4-benzodioxine (3.63 g, 10.8 mmol) in THF (60, mL) is cooled in a CO₂/acetone bath under N₂. A solution of t-butyl lithium in pentane (1.3 M, 17.5 mL, 22.8 mmol) is added. After 5 min, CO₂ (g) is bubbled through the mixture and the mixture is warmed to rt. A solution of HCl in methanol is added and the mixture concentrated. The residue is extracted between NaOH (1 N) and EtOAc. The organic layer is discarded. The pH of the aqueous layer is adjusted to ˜4 and is extracted with EtOAc (2×100 mL). The combined organics are washed with H₂O (3×100 mL), brine, dried (MgSO₄), filtered, and concentrated. The resulting oil is chromatographed (Biotage 40M, 2% MeOH/CH₂Cl₂). The product fractions are pooled and concentrated to an give oil 1.66 g (51%) of 2-(phenoxymethyl)-2,3-dihydro-1,4-benzodioxine-6-carboxylic acid.

Intermediate E3: 3-[(Benzyloxy)methyl]-2,3-dihydro-1.4-benzodioxine-6-carboxylic acid

(R) and (S)-(7-Bromo-2,3-dihydro-benzo-1,4-dioxin-2-yl)-methanol are prepared according to the literature example. The racemic mixture is obtained starting with racemic epichlorohydrin. See Aiba, Y.; Hasegawa, et al., Bioorg. Med. Chem. Lett.; 11; 20; 2001; 2783-2786.

A mixture of 7-bromo-2,3-dihydro-1,4-benzodioxin-2-yl)methanol (2.73 g, 11.1 mmol) and DMF (25 mL) at 0° C. is treated with a 60% dispersion of NaH in mineral oil (0.49 g, 12.3 mmol). After 15 min, the mixture is treated with benzyl bromide (1.46 mL, 12.37 mmol). After 2 h, the mixture is poured into H₂O and extracted with EtOAc (2×125 mL). The combined organic layers are washed with H₂O (3×100 mL), brine, dried (MgSO₄), filtered, and concentrated. The resulting oil is adsorbed onto SiO₂ and chromatographed (Biotage 40M+SIM, 5% EtOAc/Hexane). The product fractions are pooled and concentrated to provide an oil, which solidified (upon standing) to give 3.48 g (93%) of 2-[(benzyloxy)methyl]-7-bromo-2,3-dihydro-1,4-benzodioxine.

A mixture of 2-[(benzyloxy)methyl]-7-bromo-2,3-dihydro-1,4-benzodioxine (3.35 g, 10.0 mmol) in THF (60, mL) is cooled in a CO₂/acetone bath under N₂. A solution of t-butyl lithium in pentane (1.7 M, 6.0 mL, 10.2 mmol) is added. After 5 min, CO₂ (g) is bubbled through the mixture and the mixture is warmed to rt. A solution of HCl in methanol is added and the mixture concentrated. The residue is chromatographed (Biotage 40M, 3% MeOH/CH₂Cl₂). The product fractions are pooled and concentrated to give 1.19 g (40%) of 3-[(benzyloxy)methyl]-2,3-dihydro-1,4-benzodioxine-6-carboxylic acid as an oil.

Intermediate E4: (3S)-3-[(Benzyloxy)methyl]-2,3-dihydro-1,4-benzodioxine-6-carboxyl acid

Intermediate E4 is obtained following the procedures discussed for Intermediate E3, making non-critical changes, and starting with [(2S)-7-bromo-2,3-dihydro-1,4-benzodioxin-2-yl]methanol

Intermediate E5: (3R) 3-[(Benzyloxy)methyl]-2,3-dihydro-1,4-benzodioxine-6-carboxylic acid

Intermediate E5 is obtained following the procedures discussed for Intermediate E3, making non-critical changes, and starting with (3R)-3-[(benzyloxy)methyl]-2,3-dihydro-1,4-benzodioxine-6-carboxylic acid.

Intermediate E6: (3S)-3-(Phenoxymethyl)-2,3-dihydro-1,4-benzodioxine-6-carboxylic acid

A mixture of [(2S)-7-bromo-2,3-dihydro-1,4-benzodioxin-2-yl]methanol (2.26 g, 9.20 mmol), phenol (0.87 g, 9.2 mmol), triphenylphosphine (2.42 g, 9.20 mmol) and THF (80 mL) is cooled in a 0° C. bath under N₂. Diethylazodicarboxylate (1.50 ml, 9.5 mmol) is added, and the mixture is allowed to warm to rt overnight. The mixture is adsorbed onto SiO₂ and chromatographed (Biotage 40S+SIM, (1:19) EtOAc:hexane). The product fractions are pooled and concentrated to afford 1.45 g (49%) of (2S)-7-bromo-2-(phenoxymethyl)-2,3-dihydro-1,4-benzodioxine as a clear oil.

Intermediate E7: (3R)-3-(Phenoxymethyl)-2,3-dihydro-1,4-benzodioxine-6-carboxylic acid

A mixture of [(2R)-7-bromo-2,3-dihydro-1,4-benzodioxin-2-yl]methanol (0.648 g, 2.64 mmol), phenol (0.248 g, 2.64 mmol), triphenylphosphine (0.692 g, 2.64 mmol) and THF (26 mL) is cooled in a 0° C. bath under N₂. Diethylazodicarboxylate (0.42 ml, 2.7 mmol) is added and the mixture allowed to warm to rt overnight. The mixture is concentrated, partitioned between EtOAc and H₂O, the organic layer dried (MgSO₄), adsorbed onto SiO₂, and chromatographed (Biotage 40S+SIM, (1:19) EtOAc:hexane). The product fractions are pooled and concentrated to afford 0.315 g (37%) of (2R)-7-bromo-2-(phenoxymethyl)-2,3-dihydro-1,4-benzodioxine as an oil. A solution of this oil (0.280 g, 0.87 mmol) and THF (30 ml) is cooled in a CO₂ (s)/acetone bath under N₂. To this is added a solution of tert-butyl lithium in pentane (1.7 M, 1.10 ml, 1.9 mmol). After stirring for 5 min, CO₂ (g) is bubbled through the solution for an additional 10 min. The mixture is treated with MeOH/HCl and allowed to warm to rt. The mixture is concentrated, and the residue is chromatographed (Biotage 40S, (1:499) MeOH:CH₂Cl₂). The product fractions are pooled and concentrated to afford 0.103 g (41%) of (3R)-3-(phenoxymethyl)-2,3-dihydro-1,4-benzodioxine-6-carboxylic acid as a solid.

Intermediate E8: 2,3-Dihydro-1,4-dioxino[2,3-c]pyridine-7-carboxylic acid

To a stirred solution of 4,5-hydroxypyridine-2-carboxylic acid [see: Kenichi Mochida, et al. J. Antibiot. 1987, 182] (800 mg, 4.18 mmol) in MeOH (30 mL) is added concentrated sulfuric acid (1 mL). The mixture is heated to reflux for 2 days. The mixture is cooled to rt, followed by addition of solid sodium bicarbonate. The mixture is diluted with water and the precipitate is filtered and dried to give 527 mg (75%) of methyl 4,5-dihydroxypyridine-2-carboxylate: ¹H NMR (400 MHz, MeOH-d₄) δ 7.68, 7.24, 3.97.

To a stirred solution of methyl 4,5-dihydroxypyridine-2-carboxylate (348 mg, 2.06 mmol) in DMF (20 mL) is added solid K₂CO₃ (3.1 g, 22 mmol) and 1,2-dibromoethane (386 μL, 4.5 mmol). The mixture is heated at 115° C. for 2 h. DMF is removed in vacuo, the residue is partitioned between water and EtOAc. The aqueous layer is again extracted with EtOAc. The combined organic layers are dried (MgSO₄) and concentrated in vacuo to give a yellow solid for methyl 2,3-dihydro-1,4-dioxino[2,3-c]pyridine-7-carboxylate (348 mg, 86%): ¹H NMR (400 MHz, CDCl₃) δ 8.29, 7.71, 4.39, 3.99.

To a stirred solution of methyl 2,3-dihydro-1,4-dioxino[2,3-c]pyridine-7-carboxylate (300 mg, 1.54 mmol) in MeOH (10 mL) is added NaOH (10 mL of a 5% aqueous solution). The mixture is heated to reflux for 3 h, followed by cooling to rt. The methanol is removed in vacuo and the remaining aqueous layer is acidified to pH=5 with 1N HCl, extracted with CH₂Cl₂ continuously for 2 days. The organic layer is concentrated to a white solid (245 mg, 88%) for 2,3-dihydro-1,4-dioxino[2,3-c]pyridine-7-carboxylic acid: ¹H NMR (400 MHz, DMSO-d₆) δ 13-12, 8.21, 7.52, 4.39.

Intermediate E9: Chromane-6-carboxylic acid

A mixture of chromene (see: Chatterjea, J. Indian Chem. Soc. 1959, 35, 78.) (5.00 g, 37.8 mmol) and 10% palladium on activated carbon (250 mg) in glacial acetic acid (100 mL) is placed in a Parr bottle. The mixture is shaken under an atmosphere of hydrogen (45 psi) for 3 h at rt. The mixture is filtered through Celite and the filtrate is concentrated in vacuo to afford 5.00 g (98%) of chromane as light yellow oil: ¹H NMR (400 MHz, CDCl₃) δ 7.15-7.05, 6.89, 6.80, 4.23, 2.84, 2.08-2.02.

To a stirred solution of acetyl chloride (4.78 mL, 67.1 mmol) in dry CH₂Cl₂ (20 mL) in a −10° C. bath is added aluminum trichloride (4.76 g, 35.7 mmol) in small portions. The mixture is stirred for 15 min until the solution became homogeneous. The solution is added via canula to a separate solution of chromane (4.79 g, 35.7 mmol) in CH₂Cl₂ (30 mL) all at −10° C. After complete addition, the solution is stirred at −10° C. for 30 min. The solution is poured over a mixture of crushed ice and concentrated HCl. The mixture is extracted with CH₂Cl₂. The combined organic layers are washed with brine, dried (MgSO₄), filtered and concentrated in vacuo. The remaining residue is purified via crystallization from hexanes to give 4.0 g (64%) of 1-(3,4-dihydro-2H-chromen-6-yl)ethanone as a white solid. ¹H NMR (400 MHz, CDCl₃) δ7.76-7.73, 6.75, 4.27, 2.86, 2.57, 2.09-2.03.

A mixture of 1-(3,4-dihydro-2H-chromen-6-yl)ethanone (3.80 g, 22.0 mmol) and sodium hypochlorite [150 mL of a 6.0% aqueous solution, (Clorox brand of bleach)] in a 55° C. oil bath is stirred for 2 h. The mixture (now homogeneous) is cooled to rt and solid sodium bisulfite is added until a clear color persisted. HCl (ca 15 mL of a 6.0 M aqueous solution) is added, followed by extraction with EtOAc. The organic layer is washed with brine, dried (MgSO₄), filtered, and concentrated in vacuo to afford 3.10 g (82%) of chromane-6-carboxylic acid as a white solid. ¹H NMR (400 MHz, DMSO-d₆) δ 12.55, 7.67, 7.6, 6.79, 4.20, 2.77, 1.96-1.90.

Intermediate E10: Chromane-7-carboxylic acid

To a stirred solution of methyl 4-formyl-3-hydroxybenzoate [see: Harayama, Chem. Pharm. Bull. 1994, 2170] (0.8 g, 4.1 mmol) and anhydrous K₂CO₃ (1.1 g, 8.0 mmol) in acetone (12 mL) is added allyl bromide (0.70 mL, 8.1 mmol). The mixture is heated in a 48° C. oil bath for 2 h. The reaction mixture is cooled to rt and filtered. The mother liquor is concentrated in vacuo to a brown oil. The crude product is purified by flash chromatography on SiO₂. Elution with hexanes-EtOAc (85:15) gives 0.85 g (49%) of methyl 3-(allyloxy)-4-formylbenzoate as a clear solid: ¹H NMR (400 MHz, CDCl₃) δ 10.6, 7.9, 7.7, 6.1, 5.5, 5.4, 4.8, 4.0.

Sodium hydride [220 mg (60% oil dispersion), 5.4 mmol], is washed with pentane (3×) and is suspended in THF (12 mL) in a 0° C. ice bath. Methyl triphenylphosphonium bromide (1.7 g, 4.7 mmol) is added. The suspension is allowed to warm to rt and stir for 30 min. A solution of methyl 3-(allyloxy)-4-formylbenzoate (0.85 g, 3.8 mmol) in THF (5 mL) is added via canula. The mixture is stirred at rt for 2 h. The mixture is diluted with EtOAc and washed with brine. The organic layer is dried with MgSO₄, filtered and concentrated in vacuo to a yellow residue. The crude product is triturated with hexanes, filtered and dried in vacuo to a clear oil for methyl 3-(allyloxy)-4-vinylbenzoate (680 mg, 81%): ¹H NMR (400 MHz, CDCl₃) δ 7.65-7.54, 7.13, 6.13, 5.88, 5.49-5.29, 4.65, 3.93.

To a stirred solution of methyl 3-(allyloxy)-4-vinylbenzoate (0.67 g, 3.1 mmol) in CH₂Cl₂ (20 mL) at rt is added benzylidene-bis(tricyclohexylphosphine)-dichlororuthenium (63 mg, 0.076 mmol). The mixture is stirred at rt for 2 h. The reaction mixture is concentrated in vacuo to a dark residue. The crude product is purified by flash chromatography on SiO₂. Elution with hexanes-EtOAc (95:5) gives 372 mg (64%) of methyl 2H-chromene-7-carboxylate as a clear oil: ¹H NMR (400 MHz, CDCl₃) δ 7.56, 7.46, 7.01, 6.46, 5.91, 4.89, 3.91.

A mixture of methyl 2H-chromene-7-carboxylate (372 mg, 1.96 mmol) and 10% Pd/C (25 mg) in methanol (15 mL) is stirred under 1 atm of hydrogen at rt for 3 h. The mixture is filtered through Celite and the filtrate is concentrated to a yellow residue. The crude product is purified by flash chromatography on SiO₂. Elution with hexanes-EtOAc (95:5) gives 140 mg (37%) of methyl chromane-7-carboxylate as a clear oil: ¹H NMR (400 MHz, CDCl₃) δ 7.51, 7.47, 7.10, 4.23, 3.91, 2.85, 2.04.

To a stirred solution of methyl chromane-7-carboxylate (140 mg, 0.73 mmol) in MeOH (5 mL) is added NaOH (5 mL of a 5% aqueous solution). The mixture is heated in a 85° C. oil bath for 3 h, followed by cooling to rt. The methanol is removed in vacuo and the remaining aqueous layer is acidified to pH=1 with concentrated HCl, extracted with EtOAc (3×). The combined organic layers are dried (MgSO₄) and concentrated to a white solid for chromane-7-carboxylic acid (130 mg, 100%): ¹H NMR (400 MHz, DMSO-d₆) δ 13-12, 7.37, 7.24, 7.16, 4.16, 2.79, 1.92.

Intermediate E11: 2H-chromene-6-carboxylic acid

To a stirred solution of ethyl 3-formyl-4-hydroxybenzoate [see: Skattebol, Acta. Chemica. Scandinavica 1999, 53, 258] (1.9 g, 10.0 mmol) and anhydrous K₂CO₃ (2.7 g, 19.5 mmol) in acetone (30 mL) is added allyl bromide (1.7 mL, 19.8 mmol). The mixture is heated in a 60° C. oil bath for 2 h. The mixture is cooled to rt, filtered and concentrated in vacuo to afford 2.1 g (92%) of ethyl 4-(allyloxy)-3-formylbenzoate as a white solid: ¹H NMR (400 MHz, CDCl₃) δ 10.5, 8.5, 8.2, 7.1, 6.1, 5.5, 5.4, 4.8, 4.4, 1.4.

To a stirred suspension of sodium hydride [588 mg (60% oil dispersion), 15 mmol), which had been previously washed with pentane (3×), in THF (30 mL) in a 0° C. ice bath is added methyl triphenylphosphonium bromide (4.6 g, 13 mmol). The suspension is allowed to warm to rt and stir for 30 min. A solution of ethyl 4-(allyloxy)-3-formylbenzoate (2.3 g, 9.8 mmol) in THF (10 mL) is added via canula. The mixture is stirred at rt 2 h. The mixture is diluted with EtOAc and washed with brine. The organic layer is dried of MgSO₄, filtered and concentrated in vacuo to a yellow residue. The crude product is purified by flash chromatography on SiO₂. Elution with hexanes-EtOAc (95:5) gives 1.8 g (79%) of ethyl 4-(allyloxy)-3-vinylbenzoate as a clear oil: ¹H NMR (400 MHz, CDCl₃) δ 8.2, 7.9, 7.1, 6.9, 6.1, 5.9, 5.5, 5.3, 4.7, 4.4, 1.4.

To a stirred solution of ethyl 4-(allyloxy)-3-vinylbenzoate (1.8 g, 7.7 mmol) in CH₂Cl₂ (40 mL) at rt is added benzylidene-bis(tricyclohexylphosphine)-dichlororuthenium (127 mg, 0.15 mmol). The mixture is stirred at rt for 2.5 h. The reaction mixture is concentrated in vacuo to a dark residue. The crude product is purified by flash chromatography on SiO₂. Elution with hexanes-EtOAc (95:5) gives 1.3 g (80%) of ethyl 2H-chromene-6-carboxylate as a clear oil: ¹H NMR (400 MHz, CDCl₃) δ 7.8, 7.7, 6.8, 6.4, 5.8, 4.9, 4.4, 1.4.

To a stirred solution of ethyl 2H-chromene-6-carboxylate in MeOH (80 mL) is added NaOH (40 mL of a 5% aqueous solution). The mixture is heated in a 60° C. oil bath for 30 min, followed by cooling to rt. The methanol is removed in vacuo and the remaining aqueous layer is acidified to pH=1 with concentrated HCl. The solid precipitate is filtered and washed with water to afford 130 mg (13%) of 2H-chromene-6-carboxylic acid as a white solid: ¹H NMR (400 MHz, CDCl₃) δ 12-11, 7.9, 7.7, 6.8, 6.5, 5.8, 5.0.

Intermediate E12: 2-Methyl-2H-chromene-6-carboxylic acid

To a stirred solution of lithium bis(trimethylsilyl)amide (1.0 M solution in tetrahydrofuran) (8 mL) in a 0° C. ice bath is added methyl triphenylphonium bromide (1.92 g, 5.38 mmol). The mixture is allowed to warm to rt and stir for 10 min. A solution of methyl 3-formyl-4-hydroxybenzoate (200 mg, 1.11 mmol) in THF (3 mL) is added to the above solution. The mixture is stirred at rt for 5 h. The reaction mixture is acidified to pH=5 with 1N HCl, and extracted with ether (3×). The combined organic layers are washed with brine, dried (MgSO₄), filtered and concentrated to a yellow oil. The crude product is purified by chromatography on SiO₂. Elution with hexanes-EtOAc (80:20) gives 130 mg (66%) of methyl 4-hydroxy-3-vinylbenzoate as a white solid: ¹H NMR (400 MHz, CDCl₃) δ 8.12, 7.86, 6.93, 6.85, 5.84, 5.50, 5.46, 3.92.

To a stirred solution of methyl 4-hydroxy-3-vinylbenzoate (410 mg, 2.3 mmol), triphenylphosphine (787 mg, 3.0 mmol), 3-buten-2-ol (260 μL, 3.0 mmol) in THF (15 mL) at 0° C. is added a solution of diethyl azadicarboxylate (472 μL, 3.0 mmol) in THF (5 mL). The mixture is allowed to warm to rt and stir overnight. The mixture is concentrated in vacuo and the residue is purified by chromatography on SiO₂. Elution with hexanes-EtOAc (95:5) gives 371 mg (69%) of methyl 3-formyl-4-[(1-methylprop-2-enyl)oxy]benzoate as a clear oil: ¹H NMR (400 MHz, CDCl₃) δ 8.18, 7.89, 7.08, 6.90, 5.94, 5.86, 5.36-5.30, 4.93, 3.91, 1.51.

To a stirred solution of methyl 3-formyl-4-[(1-methylprop-2-enyl)oxy]-benzoate (370 mg, 1.59 mmol) in CH₂Cl₂ (8 mL) at rt is added benzylidene-bis(tricyclohexylphosphine)dichlororuthenium (56 mg, 0.068 mmol). The mixture is stirred at rt overnight. The reaction mixture is concentrated in vacuo to a dark residue. The crude product is purified by flash chromatography on SiO₂. Elution with hexanes-EtOAc (95:5) gives 225 mg (69%) of methyl 2-methyl-2H-chromene-6-carboxylate as a clear oil: ¹H NMR (400 MHz, CDCl₃) δ 7.82, 7.68, 6.79, 6.41, 5.71, 5.11, 3.89, 1.48.

To a stirred solution of methyl 2-methyl-2H-chromene-6-carboxylate (225 mg, 1.10 mmol) in MeOH (5 mL) is added NaOH (5 mL of a 5% aqueous solution). The mixture is heated in a 60° C. oil bath for 40 min, followed by cooling to rt. The methanol is removed in vacuo and the remaining aqueous layer is acidified to pH=5 with 1N HCl. The solution is extracted with EtOAc (2×), washed with brine, dried (MgSO₄) and concentrated in vacuo to afford 209 mg (100%) of 2-methyl-2H-chromene-6-carboxylic acid as a yellow oil: ¹H NMR (400 MHz, DMSO-d6) δ 13-12, 7.68, 7.65, 6.80, 6.53, 5.85, 5.10, 1.37.

Intermediate E13: 3,4-Dihydro-2H-pyrano[2,3-c]pyridine-6-carboxylic acid

2-Chloro-3-pyridinol (20.0 g, 0.154 mole and NaHCO₃ (19.5 g, 0.232 mole, 1.5 equ) are dissolved in 150 ml of water. The reaction mixture is placed in an oil bath at 90° C. and after 5 min is treated with 37% aqueous formaldehyde (40.5 ml, 0.541 mole, 3.5 equ) which is added in six unequal doses; 12 ml initially, 3×8 ml followed by 1×2.2 ml all at 90 min intervals with the final 2.3 ml added after maintaining at 90° C. overnight (15 h). After stirring in the 90° C. bath for an additional 4 h, the flask is placed in ice bath, and the contents are treated with 100 ml of crushed ice, acidified with 39 ml of 6 N HCl to pH 1, and the precipitated material is stirred for 1.5 h in an ice bath. The undesired solid is removed by filtration, and the filtrate is extracted seven times with EtOAc. The combined organic extracts are concentrated at reduced pressure, treated with toluene, reconcentrated on rotary evaporator to azeotrope most of the water, suspended in CH₂Cl₂ and reconcentrated again at reduced pressure to obtain 19.9 g (81%) of 2-chloro-6-(hydroxymethyl)-3-pyridinol as a pale yellow solid sufficiently pure for subsequent reaction. MS for C₆H₆ClNO₂: m/z: 159 (M)⁺.

2-Chloro-6-(hydroxymethyl)-3-pyridinol (11.6 g, 72.7 mmol) and NaHCO₃ (18.3 g, 218 mmol) are dissolved in 200 ml water in a flask. The mixture is stirred until homogeneous, is cooled in an ice bath, is treated with iodine (19.4 g, 76.3 mmol), and is stirred over 60 h at rt as the cooling bath expired. The pH of the mixture is adjusted to 3 with 2N NaHSO₄, and the mixture is extracted with 4×50 ml EtOAc. The combined organic layer is dried (MgSO₄) and is concentrated in vacuo to a yellow solid. The crude solid is washed with EtOAc to provide 12.9 g (62%) of 2-chloro-6-(hydroxymethyl)-4-iodo-3-pyridinol as an off-white solid. The filtrate is concentrated to a small volume and is chromatographed over 250 g SiO₂ (230-400 mesh) eluting with EtOAc/CH₂Cl₂/hexane/acetic acid 2.5:4.5:4:0.1. The appropriate fractions are combined and concentrated to afford an additional 2.4 g (12%) of pure 2-chloro-6-(hydroxymethyl)-4-iodo-3-pyridinol. MS for C₆H₅ClINO₂, m/z: 285 (M)⁺.

2-Chloro-6-(hydroxymethyl)-4-iodopyridin-3-ol (5.7 g, 20 mmol) is combined with bis (triphenylphosphine) palladium dichloride (1.12 g, 1.6 mmol) in 50 ml DMF under nitrogen. The mixture is treated with tetravinyl tin, is warmed to 60° C. for 6 h followed by 50° C. for 18 h, and at rt for 72 h. The mixture is diluted with 250 ml EtOAc and is extracted with 4×100 ml 2:1:1 water/saturated NaCl/saturated NaHCO₃. The organic layer is dried (MgSO₄) and is concentrated in vacuo to a yellow oil. The crude material is chromatographed over 200 g SiO₂ (230-400 mesh) eluting with 37% EtOAc/hexane. The appropriate fractions are combined and concentrated to afford 1.45 g (39%) of 2-chloro-6-(hydroxymethyl)-4-vinylpyridin-3-ol as a pale yellow solid. MS for C₈H₈ClNO₂ (EI) m/z: 185 (M)⁺.

2-Chloro-6-(hydroxymethyl)-4-vinylpyridin-3-ol (1.35 g, 7.8 mmol) is dissolved in 12 ml DMF in a dry flask under nitrogen. The yellow solution is treated with 60% sodium hydride (312 mg, 7.8 mmol), is stirred 30 min, and is treated with allyl bromide (744 μL, 8.6 mmol). The reaction is stirred 6 h at RT, is diluted with 50 ml EtOAc, and is washed with 4×25 ml 2:1:1 water/sat'd NaCl/sat'd NaHCO₃. The organic layer is dried (MgSO₄) and is concentrated in vacuo to a yellow oil. The crude material is chromatographed over 50 g SiO₂ (230-400 mesh) eluting with 30% EtOAc/hexane. The appropriate fractions are combined and concentrated to give 1.43 g (81%) of [5-(allyloxy)-6-chloro-4-vinylpyridin-2-yl]methanol as a white solid. MS for C₁₁H₁₂ClNO₂ (EI) m/z: 225 (M)⁺.

[5-(Allyloxy)-6-chloro-4-vinylpyridin-2-yl]methanol (225 mg, 1.0 mmol) is combined with bis (tricyclohexylphosphine) benzylidene ruthenium (IV) dichloride (16.5 mg, 0.02 mmol) in 5 ml CH₂Cl₂ and the reaction is stirred 4 h at RT. The volatiles are removed in vacuo and the residue is chromatographed over 15 g SiO₂ (230-400 mesh) eluting with 40% EtOAc/hexane. The appropriate fractions are combined and concentrated to give 175 mg (89%) of (8-chloro-2H-pyrano[2,3-c]pyridin-6-yl)methanol as a tan solid. MS for C₉H₈ClNO₂ (EI) m/z: 197 (M)⁺.

(8-Chloro-2H-pyrano[2,3-c]pyridin-6-yl)methanol (988 mg, 5.0 mmol) is combined with 100 mg 10% Pd/C in 25 ml EtOH containing 3 ml (6 mmol) of 2N aqueous NaOH in a 250 ml PARR shaker bottle. The reaction is hydrogenated at 50 PSI for 48 h, the catalyst is removed by filtration, and the filtrate is concentrated to dryness. The mixture is partitioned between 1×10 ml 1:1 saturated NaCl/conc. NH₄OH and 4×10 ml CH₂Cl₂ and the combined organic layer is dried (K₂CO₃). The mixture is concentrated in vacuo to give 730 mg (89%) of 3,4-dihydro-2H-pyrano[2,3-c]pyridin-6-ylmethanol as an off-white solid. HRMS (FAB) calcd for C₉H₁₁NO₂+H: 166.0868, found 166.0868 (M+H)⁺.

Oxalyl chloride (452 μL, 5.1 mmol) is dissolved in 15 ml CH₂Cl₂ under nitrogen at −78° C. The solution is treated drop-wise with DMSO (729 μL, 10.3 mmol) in 5 ml CH₂Cl₂ and the mixture is stirred 30 min at −78° C. 3,4-Dihydro-2H-pyrano[2,3-c]pyridin-6-ylmethanol (731 mg, 4.4 mmol) is added drop-wise to the reaction mixture in 5 ml CH₂Cl₂ and the reaction is stirred 30 min at −78° C. The mixture is treated with TEA (3.08 ml, 22.1 mmol), is stirred 30 min at −78° C. and 2 h at 0° C. The mixture is washed with 1×10 ml saturated NaHCO₃, is dried (K₂CO₃), and is concentrated in vacuo. The crude intermediate is chromatographed over 25 g SiO₂ (230-400 mesh) eluting with 35% EtOAc/hexane. The appropriate fractions are combined and concentrated to give 685 mg (95%) of the aldehyde as an off-white solid.

The aldehyde (685 mg, 4.2 mmol) is combined with NaClO₂ (80%, 1.42 g, 12.6 mmol) and KH₂PO₄ in 15 ml THF/7 ml t-BuOH/7 ml water and the reaction is stirred overnight under a stream of nitrogen. The reaction is concentrated to dryness in vacuo and the residue is dissolved in 10 ml water. The pH of the mixture is adjusted to 5 with 12 N HCl, the white solid is collected, washed with water, and is dried in vacuo at 50° C. to afford 565 mg (82%) of 3,4-dihydro-2H-pyrano[2,3-c]pyridine-6-carboxylic acid as a white solid. HRMS (FAB) calcd for C₉H₉NO₃+H: 180.0661, found 180.0652 (M+H)⁺.

Compounds of Formula I where W is (F) are made using the coupling procedures discussed herein and in cited references, making non-critical changes to obtain the desired compounds. The following intermediates to provide W of formula I are for exemplification only and are not intended to limit the scope of the present invention. Other intermediates within the scope of the present invention can be obtained using known procedures or by making slight modifications to known procedures.

Intermediate F1: 1,3-Benzoxazole-6-carboxylic acid

A mixture of 4-amino-3-hydroxybenzoic acid (250 mg, 1.63 mmol) and trimethyl orthoformate (500 μL, 4.57 mmol) is heated in an oil bath at 100° C. for 2 h. The mixture is cooled to rt and diluted with MeOH. The resulting solution is filtered through a pad of Celite, and the filtrate is concentrated in vacuo to give Intermediate F1 as a brown solid (237 mg, 89%): ¹H NMR (DMSO-d₆) δ 13.2, 8.9, 8.3, 8.0, 7.9.

Intermediate F2: 2-Methyl-1,3-benzoxazole-6-carboxylic acid

A mixture of 4-amino-3-hydroxybenzoic acid (500 mg, 3.7 mmol) and trimethyl orthoacetate (1.0 mL, 7.9 mmol) is heated in an oil bath to 100° C. for 2 h. The mixture is cooled to rt and diluted with MeOH. The resulting solution is filtered through a pad of Celite, and the filtrate is concentrated in vacuo to give Intermediate F2 as an off-white solid (266 mg, 46%): ¹H NMR (DMSO-d₆) δ 13.1, 8.2, 8.0, 7.7, 2.7.

Intermediate F3: 1,3-Benzoxazole-5-carboxylic acid

A mixture of 4-amino-3-hydroxybenzoic acid (1.0 g, 6.5 mmol) and trimethyl orthoformate (2.0 mL, 18.3 mmol) is heated in an oil bath at 100° C. for 30 h. The mixture is cooled to rt and diluted with MeOH. The resulting solution is filtered through a pad of Celite, and the filtrate is concentrated in vacuo to give Intermediate F3 as a brown solid (290 mg, 27%): ¹H NMR (DMSO-d₆) δ 13.0, 8.9, 8.3, 8.1, 7.9.

Intermediate F4: 2-Methyl-1,3-benzoxazole-5-carboxylic acid

A mixture of 4-amino-3-hydroxybenzoic acid (480 mg, 3.1 mmol) and trimethyl orthoacetate (1.0 mL, 7.9 mmol) is heated in an oil bath to 107° C. for 2 h. The mixture is cooled to rt and diluted with MeOH. The resulting solution is filtered through a pad of silica gel and the filtrate is concentrated in vacuo to give Intermediate F4 as an orange solid (490 mg, 88%): ¹H NMR (DMSO-d₆) δ 13.0, 8.2, 8.0, 7.8, 2.7.

Intermediate F5: 5-Indancarboxylic acid

To a stirred 6% aqueous sodium hypochlorite solution in an oil bath to 55° C. is added 1-indane-5-yl-ethanone (1.0 g, 6.2 mmol). The solution is stirred at 55° C. for 2 h, followed by cooling to rt. Solid sodium bisulfite is added until the solution became clear. The mixture is diluted with water, followed by aqueous hydrochloric acid (6.0 M). The solid that forms is filtered and washed several times with water. The solid is dried under high vacuum at 60° C. for 5 h to afford Intermediate F5 as a white solid (0.96 g, 95%): ¹H NMR (CDCl₃) δ 8.0, 7.9, 7.3, 3.0, 2.1.

Intermediate F6: [1,3]Oxazolo[5,4-c]pyridine-6-carboxylic acid

2-Chloro-3-pyridinol (20.0 g, 0.154 mole), NaHCO₃ (19.5 g, 0.232 mole, 1.5 equ), and 150 mL of water are placed in a flask. The flask is placed in an oil bath at 90° C., and after 5 minutes, 37% aqueous formaldehyde (40.5 mL, 0.541 mole, 3.5 equ) is added in six unequal doses in the following order: 12 mL, 3×8 mL, then 2.2 mL all at 90-minute intervals and then the final 2.3 mL after the reaction had stirred for 15 h at 90° C. The reaction is stirred at 90° C. for another 4 h and then is cooled by placing the flask in an ice bath. The pH of the reaction is then adjusted to 1 using 6N HCl. The reaction is stirred for 1.5 h in an ice bath allowing an undesired solid to form. The undesired solid is removed by filtration, and the filtrate is extracted seven times with EtOAc. The combined organic extracts are concentrated in vacuo, toluene is added to the flask and removed in vacuo to azeotrope water, and then CH₂Cl₂ is added and removed in vacuo to obtain 2-chloro-6-(hydroxymethyl)-3-pyridinol (I-10-F) as a pale yellow solid (81% yield) sufficiently pure for subsequent reaction. MS (EI) for C₆H₆ClNO₂, m/z: 159(M)⁺.

I-10-F (11.6 g, 72.7 mmol) and NaHCO₃ (18.3 g, 218 mmol) are added to 200 mL water. The mixture is stirred until homogeneous, the flask is placed in an ice bath, iodine (19.4 g, 76.3 mmol) is added, and the reaction is stirred over the weekend at rt. The pH of the mixture is adjusted to 3 with 2N NaHSO₄, and the mixture is extracted with 4×50 mL EtOAc. The combined organic layer is dried (MgSO₄), is filtered, and the filtrate is concentrated in vacuo to a yellow solid. The crude solid is washed with EtOAc to provide 2-chloro-6-(hydroxymethyl)-4-iodo-3-pyridinol (I-12-F as an off-white solid (62% yield), and the filtrate is concentrated to a small volume and is chromatographed over 250 g silica gel (230-400 mesh) eluting with 2.5:4.5:4:0.1 EtOAc/CH₂Cl₂/hexane/acetic acid. The desire fractions are combined and concentrated to afford an additional pure I-12-F (12% yield). MS (EI) for C₆H₅ClINO₂, m/z: 285(M)⁺.

4-(Benzylamino)-2-chloro-6-(hydroxymethyl)-3-pyridinol (I-13-F) may be produced by amination of 2-chloro-6-(hydroxymethyl)-4-iodo-3-pyridinol (I-12-F) with benzylamine under palladium catalysis. Amination of aryl iodides with primary amines such as benzylamine under palladium catalysis is generally described in a review by B. H. Yang and S. L. Buchwald in J. Organomet. Chem., 576, 125-146, 1999 and in greater detail in the references therein.

I-13-F may be oxidized to 4-(benzylamino)-2-chloro-3-hydroxypyridine-6-carboxaldehyde (I-14-F) under a wide variety of conditions (e.g., TPAP and NMO in CH₂Cl₂). I-14-F may be oxidized to produce the corresponding carboxylic acid I-15-F using an oxidizing reagent such as NaClO₂ and KH₂PO₄ in DMSO/H₂O or Ag₂O, or hydrogen peroxide or ruthenium tetroxide.

Removal of the benzyl group and the chloro group of Acid I-15-F may be accomplished by utilizing hydrogen or a hydrogen source (e.g., cyclohexene, cyclohexadiene, ammonium formate, hydrazine, etc.) in the presence of Pd/C or other catalyst, under a variety of conditions and in various solvents, to produce 4-amino-5-hydroxypyridine-2-carboxylic acid (Acid I-16-F).

Cyclocondensation of Acid I-16-F with trimethyl orthoformate in the presence of catalytic para-toluenesulfonic acid may be conducted to produce [1,3]oxazolo[5,4-c]pyridine-6-carboxylic acid.

Intermediate F7: 2-Benzoisothiophene-5-carboxylic acid

Intermediate F7 can be made by the saponification of the methyl ester I-20-E, which can be made pursuant to Wynberg, Hans, et al., Recl. Trav. Chim. Pays-Bas (1968), 87(10), 1006-1010.

Intermediate F8: 1,3-Benzothiazole-5-carboxylic acid

A solution of sodium sulfide.nanohydrate (1.15 g, 4.9 mmol) in methanol-water (ca. 10 mL, 1:1) is warmed on a hot plate. To this solution is added elemental sulfur (150 mg, 4.6 mmol). Heating is continued for 15 min before the solution is poured into a separate solution of 1.0 g (4.6 mmol) of methyl 4-chloro-3-nitrobenzoate (see: Kuene, J. Am. Chem. Soc. 1962, 48, 837.) in MeOH (5.0 mL). The mixture is stirred for 30 min, followed by cooling in a refrigerator overnight. The solid precipitate is filtered, washed with water and methanol, and dried in vacuo at 50° C. to afford 650 mg (65%) of dimethyl 4,4′-dithio-bis-(3-nitrobenzoate) as a yellow solid: ¹H NMR (400 MHz, CDCl₃) δ 9.0, 8.2, 7.9, 4.0.

To a stirred solution of dimethyl 4,4′-dithio-bis-(3-nitrobenzoate) (900 mg, 2.12 mmol) in ethanol is added tin powder (1.91 g, 17.0 mmol). The mixture is heated in a 70° C. oil bath for 30 minutes before 2.8 mL of concentrated hydrochloric acid is added drop-wise. After complete addition, the mixture is stirred for an additional 10 min, followed by cooling to RT. The reaction mixture is filtered and the fitrate is concentrated in vacuo to a solid. The solid is washed with 1.0M aqueous hydrochloric acid and dried in vacuo to afford a yellow solid. The solid (750 mg, 3.42 mmol) is suspended in formic acid (4 mL) in a 100° C. oil bath. Zinc dust (15 mg) is added to the reaction. The mixture is stirred for 10 min, followed by cooling to RT. The mixture is diluted with water and extracted with EtOAc. The organic layer is dried (MgSO₄), filtered and concentrated in vacuo to afford 640 mg (97%) of methyl 1,3-benzothiazole-5-carboxylate as a yellow solid: ¹H NMR (400 MHz, CDCl₃) δ 9.1, 8.9, 8.2, 8.1, 4.0.

To a stirred solution of methyl 1,3-benzothiazole-5-carboxylate (290 mg, 1.5 mmol) in MeOH (20 mL) is added sodium hydroxide (10 mL of a 5% aqueous solution). The mixture is heated in a 65° C. oil bath for 30 min, followed by cooling to RT. The mixture is diluted with water and extracted with hexanes-ether (1:1). The organic layer is discarded and the aqueous layer is acidified with concentrated hydrochloric acid to pH=1. The aqueous layer is extracted with ether. The ethereal layer is dried (MgSO₄), filtered and concentrated in vacuo to a yellow powder for 1,3-benzothiazole-5-carboxylic acid (260 mg, 98%): ¹H NMR (400 MHz, DMSO-d₆) δ 13-12.5, 9.5, 8.6, 8.3, 8.0.

Intermediate F9: 3-Methyl-1,2-benzisoxazole-6-carboxylic acid

3-Hydroxybenzoic acid (13.8 g, 100 mmol) is dissolved in concentrated NH₄OH (200 mL) using an overhead stirrer and is treated slowly dropwise with a solution of iodine (23.4 g, 92 mmol) and KI (18.26 g, 110 mmol) in water (100 mL). The solution is stirred for 1 h at rt and then treated rapidly dropwise with concentrated HCl (180 mL). The white solid is collected via filtration, rinsed with water and dried overnight [by pulling air through the solid] in vacuo to afford 13.05 g (54%) of 3-hydroxy-4-iodobenzoic acid as a tan solid. ¹H NMR (DMSO-d₆): δ 7.13, 7.43, 7.80, 10.71, 12.98 ppm.

3-Hydroxy-4-iodobenzoic acid (12.55 g, 47.5 mmol) is dissolved in MeOH (200 mL), treated slowly dropwise with thionyl chloride (32.3 mL, 442.9 mmol) at rt, then heated to reflux for 20 h. The mixture is concentrated to dryness and partitioned between CH₂Cl₂ (100 mL) and saturated NaHCO₃ (50 mL). Not all of the residue is solubilized, so the mixture is filtered and the solid is washed with a small amount of CH₂Cl₂ and MeOH. The original filtrate and the organic washes are combined, concentrated to dryness, dissolved in 10% MeOH/CH₂Cl₂ (200 mL), diluted with water (50 mL) and the layers separated. The organics are washed with saturated NaHCO₃ (2×50 mL), then water (50 mL), dried (Na₂SO₄) and concentrated to a tan solid. This solid is triturated with CH₂Cl₂ (50 mL) and filtered. The two solids are combined to afford 9.4 g (70%) of methyl 3-hydroxy-4-iodobenzoate as a beige solid. HRMS (FAB) calcd for C₈H₇IO₃+H₁: 278.9520, found 278.9521.

Methyl 3-hydroxy-4-iodobenzoate (5.22 g, 18.8 mmol) is combined with trimethylsilylacetylene (3.71 mL, 26.3 mmol), bis(triphenylphosphine)palladium dichloride (386 mg, 0.55 mmol) and cuprous iodide (54 mg, 0.28 mmol) in THF (20 mL)/CHCl₃ (40 mL) in a dry flask, under nitrogen. TEA (8.14 mL<58.4 mmol) is added and the mixture is heated to 50° C. for 4 h. The mixture is diluted with CHCl₃ (60 mL), washed with 5% HCl (2×40 mL), dried (MgSO₄) and concentrated to a brown paste (8.31 g). The crude material is chromatographed over a standard 90 g Biotage column, eluting with 10% EtOAc/hexane (1 L) followed by 15% EtOAc/hexane (1 L). The appropriate fractions are combined and concentrated to afford 4.22 g (91%) of methyl 3-hydroxy-4-[(trimethylsilyl)ethynyl]benzoate as a yellow solid. HRMS (FAB) calcd for C₁₃H₁₆O₃SI+H₁: 249.0947, found 249.0947.

Methyl 3-hydroxy-4-[(trimethylsilyl)ethynyl]benzoate (540 mg, 2.17 mmole) is combined with 4 ml formic acid under nitrogen. The reaction is warmed to 80° C. for 12 h, is cooled to rt, and the volatiles are removed in vacuo. The black residue is chromatographed over 25 g silica gel (230-400 mesh) eluting with 15% EtOAc/hexane. The appropriate fractions are combined and concentrated to provide 350 mg (83%) of methyl 4-acetyl-3-hydroxybenzoate as a pale yellow solid. ¹H NMR (CDCl₃) δ 2.70, 3.95, 7.54, 7.64, 7.82, 12.10 ppm.

Methyl 4-acetyl-3-hydroxybenzoate (350 mg, 1.8 mmole) is combined with 5 ml absolute EtOH. The solution is treated with hydroxylamine hydrochloride (125 mg, 1.8 mmole) dissolved in 0.9 ml 2N aqueous NaOH, and the reaction is stirred overnight at rt. The volatiles are removed in vacuo and the residue is washed with H₂O, collected, and dried to give 294 mg (78%) of methyl 3-hydroxy-4-[N-hydroxyethanimidoyl]benzoate as a tan solid. MS (EI) m/z: 209 (M⁺).

Methyl 3-hydroxy-4-[N-hydroxyethanimidoyl]benzoate (250 mg, 1.19 mmole) is combined with triphenylphosphine (446 mg, 1.7 mmole) in 14 ml dry THF in a dry flask under nitrogen. The solution is treated slowly dropwise with N,N′-diethylazidodicarboxylate (268 μL, 1.7 mmole) in 10 ml dry THF. The reaction is stirred 4 h at rt. The volatiles are removed in vacuo and the residue is chromatographed over 30 g silica gel (230-400 mesh) eluting with 10% EtOAc/hexane. The appropriate fractions are combined and concentrated to provide 125 mg (55%) of methyl 3-methyl-1,2-benzisoxazole-6-carboxylate slightly contaminated (<10%) with methyl 4-acetyl-3-hydroxybenzoate. ¹H NMR (CDCl₃) δ 2.64, 4.00, 7.70, 8.01, 8.25 ppm.

Methyl 3-methyl-1,2-benzisoxazole-6-carboxylate (170 mg, 0.89 mmole) is dissolved in 6 ml MeOH under nitrogen. The solution is treated with 2N aqueous NaOH (1 ml, 2 mmole) and the mixture is stirred 4 h at rt. The volatiles are removed in vacuo and the residue is dissolved in 4 ml water. The pH of the solution is adjusted to 3 with 10% aqueous HCl, the white precipitate is collected, is washed with water, and is dried to give 144 mg (92%) of 3-methyl-1,2-benzisoxazole-6-carboxylic acid as a white solid. MS m/z (ESI): 176.2 (M−H)⁻.

Intermediate F10: 3-Methyl-1,2-benzisoxazole-5-carboxylic acid

Intermediate F13 is obtained according to the methods discussed for preparing Intermediate F12 starting with 4-hydroxybenzoic acid.

Intermediate F11: 1H-indazole-6-carboxylic acid

To a stirred solution of 3-amino-4-methylbenzoic acid (5.0 g, 33 mmol) in a mixture of water (50 mL) and concentrated hydrochloric acid (15 mL) in an acetone-crushed ice bath is added a solution of sodium nitrite in water (12 mL) dropwise. The solution is stirred for 10 min, followed by the addition of tert-butyl mercaptan (1.8 mL, 16 mmol). The mixture is stirred for 1 h. The solid precipitate is filtered, washed with water and dried in vacuo to obtain 3.85 g (95%) of 3-[(E)-(tert-butylthio)diazenyl]-4-methylbenzoic acid as a tan solid: ¹H NMR (400 MHz, DMSO-d₆) δ 13.2, 7.8, 7.5, 7.3, 2.1, 1.6.

To a stirred solution of potassium tert-butoxide (8.1 g, 73 mmol) in DMSO (30 mL) was added a solution of 3-[(E)-(tert-butylthio)diazenyl]-4-methylbenzoic acid (1.9 g, 7.3 mmol) at RT. The mixture was stirred overnight, followed by the adition of ice water. The aqueous layer was extracted with ethyl acetate. The organic layer was dicarded. The pH of the aqueous layer was adjusted to 4-5 with aqueous 1N HCl. The aqueous layer was extracted with ethyl acetate. The organic layer was washed with brine, dried (MgSO₄), filtered and concentrated in vacuo to afford 800 mg (97%) of 1H-indazole-6-carboxylic acid as a tan solid: ¹H NMR (400 MHz, DMSO-d₆) δ 13.4, 13.0, 8.2, 8.1, 7.9, 7.7.

Compounds of Formula I where W is (G) are made using the coupling procedures discussed herein and in US 20020049225A1 and US 20020042428A1, making non-critical changes to obtain compounds where Azabicyclo is other than I. The following intermediates to provide W of formula I are for exemplification only and are not intended to limit the scope of the present invention. Other intermediates within the scope of the present invention can be obtained using known procedures or by making slight modifications to known procedures.

It will be apparent to those skilled in the art that the requisite carboxylic acids can be synthesized by known procedures, or modification thereof, some of which are described herein. For example, 3-(pyrrolo[1,2-c]pyrimidine)carboxylic acid can be synthesized from the corresponding pyrrole-2-carboxaldehyde by reaction with an isocyanoacetate in the presence of base as described in J. Org. Chem. 1999, 64, 7788 and J. Org. Chem. 1976, 41, 1482 or by methods described in Liebigs Ann. Chem. 1987, 491. Scheme 1G depicts this transformation.

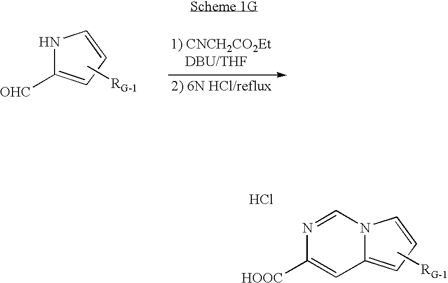

The pyrrolo[1,2-a]pyrazine acid fragment can be prepared using the methods shown in Scheme 2G. The ester intermediate can be prepared using methods described in Dekhane, M.; Potier, P.; Dodd, R. H. Tetrahedron 1993, 49, 8139-46, whereby the requisite pyrrole-2-carboxaldehyde is reacted with aminoester diethylacetal to form the imine. The imine can then be cyclized under acidic conditions to afford the desired bicyclic core. The resulting ester can be hydrolyzed under typical hydrolysis procedures well known in the art to afford the requisite pyrrolo[1,2-a]pyrazine acids.

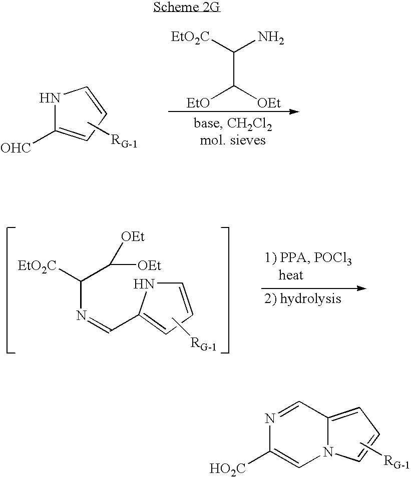

The pyrrole-2-carboxaldehydes can be obtained from commercial sources or can be synthesized by known procedures. For example, pyrrole-2-carboxaldehyde can be converted into 4-halo, 5-halo and 4,5-dihalopyrrole-2-carboxaldehydes as described in Bull. Soc. Chim. Fr. 1973, 351. See Examples 12-22. Alternatively, substituted pyrroles can be converted into pyrrole carboxaldehydes by Vilsmeier formylation using procedures well known in the art (see J. Het. Chem. 1991, 28, 2053, Synth. Commun. 1994, 24, 1389 or Synthesis, 1995, 1480. Scheme 3G depicts these transformations.

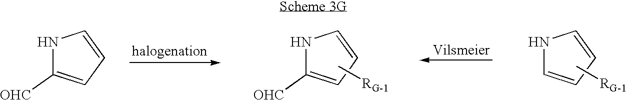

Non-limiting examples of W when W is (G):
Ethyl pyrrolo[1,2-c]pyrimidine-3-carboxylate:

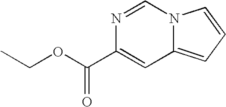

A solution of pyrrole-2-carboxaldehyde (3.6 g, 38.1 mmol) in 40 mL dry THF is added to ethyl isocyanoacetate (4.3 g, 38.1 mmol) and DBU (5.8 g, 38.2 mmol) in 60 mL dry THF. After stirring at RT overnight, the reaction is neutralized with 10% AcOH. The solvent is removed in vacuo. The residue is taken up in EtOAc/H₂O, the aqueous layer is extracted with EtOAc, dried (MgSO₄), filtered and concentrated. The residue is purified by flash chromatography on silica gel eluting with 30-70% EtOAc/hexanes. The carboxylate is obtained (4.45 g, 61%) as an off-white solid. ¹H NMR (400 MHz, CDCl₃) δ 8.86, 8.24, 7.54, 7.01, 6.78, 4.45, 1.44.

The following compounds are made from the corresponding pyrrole-2-carboxaldehydes, making non-critical variations:

Ethyl 7-chloropyrrolo[1,2-c]pyrimidine-3-carboxylate. Yield 25% starting from 5-chloropyrrole-2-carboxaldehyde. ¹H NMR (400 MHz, CDCl₃) δ 8.86, 8.21, 6.91-6.89, 6.80-6.77, 4.50-4.43, 1.47-1.42.

Ethyl 6-chloropyrrolo[1,2-c]pyrimidine-3-carboxylate. Yield 49% starting from 4-chloropyrrole-2-carboxaldehyde. ¹H NMR (400 MHz, CDCl₃) δ 8.76, 8.14, 7.51, 6.72, 4.49-4.42, 1.46-1.41.

Ethyl 6-bromopyrrolo[1,2-c]pyrimidine-3-carboxylate. Yield 9% starting from 4-bromopyrrole-2-carboxaldehyde. ¹H NMR (400 MHz, CDCl₃) δ 8.77, 8.15, 7.55, 6.79, 4.49-4.42, 1.46-1.41.

Pyrrolo[1,2-c]pyrimidine-3-carboxylic acid hydrochloride: 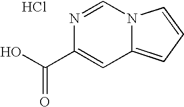
Ethyl pyrrolo[1,2-c]pyrimidine-3-carboxylate (4.1 g, 21.2 mmol) is dissolved/suspended in 100 mL concentrated HCl. The mixture is heated under reflux. After 4 h, the reaction is cooled and the solvent is removed in vacuo. Absolute EtOH is added and the solvent is removed (twice) to afford a yellow-green solid. The solid is triturated with Et₂O and dried to give 4.28 g (100%) of pyrrolo[1,2-c]pyrimidine-3-carboxylic acid as the hydrochloride salt. The solid can be recrystallized from EtOH. ¹H NMR (400 MHz, DMSO) δ 9.24, 8.21, 7.90, 7.06, 6.85.

The following compounds are made from the corresponding ethyl pyrrolo[1,2-c]pyrimidine-3-carboxylates, making non-critical variations:

7-Chloropyrrolo[1,2-c]pyrimidine-3-carboxylic acid hydrochloride. Yield 77%. ¹H NMR (400 MHz, d6-DMSO) δ 9.3, 9.04, 8.25, 7.16-7.14, 6.96-6.94.

6-Chloropyrrolo[1,2-c]pyrimidine-3-carboxylic acid hydrochloride. Yield 95%. ¹H NMR (400 MHz, d6-DMSO) δ 11.15, 9.14, 8.15, 8.04, 6.91.

6-Bromopyrrolo[1,2-c]pyrimidine-3-carboxylic acid hydrochloride. Yield 97%. ¹H NMR (400 MHz, d6-DMSO) δ 10.2, 9.12, 8.15, 8.04, 6.96.

Imidazo[1,5-a]pyridine-7-carboxylic acid:

Methyl nicotinate 1-oxide (Coperet, C.; Adolfsson, H.; Khuong, T-A. V.; Yudin, A. K.; Sharpless, K. B. J. Org. Chem. 1998, 63, 1740-41.) (5.0 g, 32.2 mmol) and dimethylsulfate (3.2 ml, 33.2 mmol) are placed in a 100 ml flask and heated to 65-70° C. for 2 h. Upon cooling a salt precipitates. The resulting precipitate is dissolved in water (12 ml). An oxygen free solution of KCN (2.5 g, 38.7 mmol) in water (9.5 ml) is added dropwise to the mixture with vigorous stirring at 0° C. After stirring for 1 h at 0° C., the mixture is warmed to rt and stirred overnight. The solution is extracted with CH₂Cl₂ (3×25 ml) and the combined organic layers are dried (NaSO₄), filtered, and the solvent removed under vacuum. The resulting solid is purified by silica gel chromatography (EtOAc) to give a yellow solid (4.2 g, 25.9 mmol, 80%) for methyl 2-cyanoisonicotinate. MS (ESI+) for C₈H₆N₂O₂ m/z 163.0 (M+H)⁺.

To a solution of methyl 2-cyanoisonicotinate (4.22 g, 25.9 mmol) and 10% palladium on charcoal (2.8 g, 2.6 mmol) in MeOH (400 ml) was added conc. HCl (7.5 ml). The mixture is hydrogenated at rt and balloon pressure, until no more hydrogen is consumed (about 2 h). The reaction mixture is filtered through a pad of celite and the solvent is removed in vacuum to give a yellow solid (4.5 g, 18.8 mmol, 73%) for methyl 2-(aminomethyl) isonicotinate. This compound is used without further purification. MS (ESI+) for C₈H₁₀N₂O₂ m/z 167.2 (M+H)⁺; HRMS (FAB) calcd for C₈H₁₀N₂O₂+H 167.0820, found 167.0821.

Procedure A:

A mixture of methyl 2-(aminomethyl) isonicotinate (4.3 g, 18.0 mmol) and acetic formic anhydride (which is prepared by heating to 50° C. acetic anhydride (75.0 ml) and formic acid (65.0 ml) for 2 h) is stirred at rt for 1 h. The reaction mixture is heated to 35° C. with an oil bath for 1 h. The reaction mixture is cooled to 0° C. in an ice-bath and neutralized with ammonium hydroxide at such a rate that the temperature did not rise above 5° C. The mixture is extracted with CH₂Cl₂ (3×200 ml) and the combined organic layers are dried (NaSO₄), filtered, and the solvent removed under vacuum. The resulting solid is purified with DOWEX 50WX2-400 ion-exchange resin to give a yellow solid (3.2 g, 18.0 mmol, 100%) for methyl imidazo[1,2-a]pyridin-6-carboxylate. MS (ESI+) for C₉H₈N₂O₂ m/z 177.03 (M+H)⁺.

Procedure B:

Methyl imidazo[1,2-a]pyridin-6-carboxylate (3.2 g, 18.0 mmol) is dissolved in 3N HCl (200 ml) and heated under reflux for 3 h. The solvent is removed under vacuum and the resulting brown solid is recrystallized from H₂O/EtOH/Et₂O to afford a light brown solid (4.3 g, 21.6 mmol, 119%) for imidazo[1,5-a]pyridine-7-carboxylic acid. HRMS (FAB) calcd for C₈H₆N₂O₂+H 163.0508, found 163.0489.

Pyrrolo[1,2-a]pyrazine-3-carboxylic acid hydrochloride:

Procedure E:

Pyrrole-2-carboxaldehyde (recrystallized from EtOAc/hexanes prior to use) (3.67 g, 38.6 mmol) is added to a solution of ethyl 3-ethoxy-O-ethylserinate (7.95 g, 38.6 mmol) in freshly distilled THF or CH₂Cl₂ (100 mL) in an oven dried 250 mL flask. 3 Å activated molecular sieves (approximately ⅓ the volume of the reaction vessel) are added, and the resulting mixture is allowed to stir under nitrogen until the starting pyrrole-2-carboxaldehyde is consumed as determined by ¹H NMR. The reaction mixture is filtered through a pad of celite, and the solvent removed in vacuo to give an orange oil (9.59 g) for ethyl 3-ethoxy-O-ethyl-N-(1H-pyrrol-2-ylmethylene)serinate that is used without purification: MS (ESI+) for C₁₄H₂₂N₂O₄ m/z 282.96 (M+H)⁺.

Procedure F:

To a hot (65° C.) solution of TFA (44 mL, 510 mmol) and phosphorus oxychloride (39.0 g, 140 mmol) is added drop-wise a solution of ethyl 3-ethoxy-O-ethyl-N-(1H-pyrrol-2-ylmethylene)serinate (Dekhane, M; Potier, P; Dodd, R. H. Tetrahedron, 49, 1993, 8139-46.) (9.6 g, 28.0 mmol) in anhydrous 1,2-dichloroethane (200 mL). The black mixture is allowed to stir at 65° C. for 18 h at which point it is cooled to rt and neutralized with sat. NaHCO₃ and solid NaHCO₃ to pH˜9. The phases are separated and the basic phase extracted with EtOAc (4×100 mL). The organic phases are combined, washed with brine, dried (NaSO₄), filtered, and concentrated to give a black oil that is purified with silica gel chromatography (35% EtOAc/heptanes to 50% over several liters) to give a light brown solid for ethyl pyrrolo[1,2-a]pyrazine-3-carboxylate. Yield 24%. HRMS (FAB) calcd for C₁₀H₁₀N₂O₂+H 191.0820, found 191.0823.

Pyrrolo[1,2-a]pyrazine-3-carboxylic acid hydrochloride is prepared from ethyl pyrrolo[1,2-a]pyrazine-3-carboxylate, using Procedure B to give a pale brown solid. Yield 90%. HRMS (FAB) calcd for C₈H₆O₂N₂+H 163.0508, found 163.0513,

Pyrazino[1.2-a]indole-3-carboxylic acid hydrocholoride:

To a suspension of lithium aluminum hydride (10.6 g, 264 mmol) in THF (200 mL) is added dropwise a solution of ethyl indole-2-carboxylate (50.0 g, 256 mmol) in THF (250 mL) over 25 minutes. After 3 h, water (10.6 mL) is carefully added, followed by 15% NaOH (10.6 mL), followed by additional portion of water (31.8 mL). The resulting suspension is dried (Na₂SO₄) and filtered through celite. After concentration under reduced pressure, the white solid (34.0 g) is crystallized from EtOAc/hexanes to give white needles for 1H-indol-2-ylmethanol. Yield 83%. HRMS (FAB) calcd for C₉H₉NO+H 148.0762, found 148.0771.

1H-Indole-2-carbaldehyde is prepared according to Berccalli, E. M., et al, J. Org. Chem. 2000, 65, 8924-32, and crystallized from EtOAc/hexanes to give a yellow/brown plates. Yield 81%. MS (ESI+) for C₉H₇NO m/z 146.1 (M+H)⁺.

Ethyl 3-ethoxy-O-ethyl-N-(1H-indol-2-ylmethylene)serinate is prepared using Procedure E to give an orange oil. Yield 94%. MS (ESI+) for C₁₈H₂₄N₂O₄ m/z 333.8 (M+H)⁺.

Procedure G:

Ethyl 9H-beta-carboline-3-carboxylate and ethyl pyrazino[1,2-a]indole-3-carboxylate are prepared according to Dekhane, M., et al, Tetrahedron, 49, 1993, 8139-46, to give a dark colored solid that is purified with silica gel chromatography (20% to 75% EtOAc/hexanes as the eluent) to give the ethyl 9H-beta-carboline-3-carboxylate as a brown solid (yield 16%) and the ethyl pyrazino[1,2-a]indole-3-carboxylate as a brown soild (yield 35%). Ethyl 9H-beta-carboline-3-carboxylate; MS (ESI+) for C₁₄H₁₂N₂O₂ m/z 241.10 (M+H)⁺; MS (ESI−) for C₁₄H₁₂N₂O₂ m/z 239.15 (M−H)⁻.

Procedure H:

To a solution of ethyl pyrazino[1,2-a]indole-3-carboxylate (0.49 g, 2.0 mmol) in EtOH (30 mL) is added crushed potassium hydroxide (1.1 g, 20.0 mmol) followed by water (30 mL). The resulting dark colored solution is stirred at rt for 40 min and then neutralized with conc. HCl to pH˜2. The acidic mixture is concentrated to dryness to afford pyrazino[1,2-a]indole-3-carboxylic acid hydrochloride. HRMS (FAB) calcd for C₁₂H₈N₂O₂+H 213.0664, found 213.0658.

Compounds of Formula I where W is (H) are made using the coupling procedures discussed herein, making non-critical changes. The following intermediates to provide formula I where W is (H) are for exemplification only and are not intended to limit the scope of the present invention. Other intermediates within the scope of the present invention can be obtained using known procedures or by making slight modifications thereof.

It will be apparent to those skilled in the art that the requisite carboxylic acids or carboxylic acid equivalents for when W is (H) can be obtained through synthesis via literature procedures or through the slight modification thereof. For example, methods to prepare carboxylic acids or carboxylic acid equivalents starting from pyrroles or pyrazoles are known to one of ordinary skill in the art (see J. Org. Chem. 1987, 52, 2319, Tetrahedron Lett. 1999, 40, 2733 and Greene, T. W. and Wuts, P. G. M. “Protective Groups in Organic Synthesis”, 3rd Edition, p. 549, New York:Wiley, (1999)). Several pyrroles and pyrazoles of the Formula W-H are commercially available or can be obtained by methods described in Synthesis 1997, 563, J. Heterocyclic Chem. 1993, 30, 865, Heterocycles 1982, 19, 1223 and J. Org. Chem. 1984, 49, 3239.

### EXAMPLE 1(H)

### N-[(3R)-1-azabicyclo[2.2.2]oct-3-yl]-4-bromo-1H-pyrazole-1-carboxamide hydrochloride

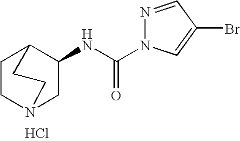

A solution of 4-bromopyrazole (0.52 g, 3.5 mmol) in 30 mL EtOAc is added to excess phosgene (10 mL, 20% solution in toluene) in EtOAc. After complete addition, the solution is refluxed for 1 h, cooled and concentrated in vacuo. EtOAc is added, and the mixture is concentrated again. The residue is treated with 20 mL THF, (R)-(+)-3-aminoquinuclidine dihydrochloride (0.71 g, 3.5 mmol) and excess TEA (5.0 mL, 68.1 mmol). After 60 h, 1N NaOH solution is added. The mixture is extracted with CHCl₃, dried (MgSO₄), filtered and concentrated. The residue is purified by flash chromatography (Biotage 40S, 90:9:1 CHCl₃/MeOH/NH₄OH). Example 1(H) is prepared and recrystallized from MeOH/EtOAc to afford 289 mg (25%) of a white solid. HRMS (FAB) calcd for C₁₁H₁₅BrN₄O+H 299.0508, found 299.0516.

### EXAMPLE 2(H)

### N-[(3R)-1-azabicyclo[2.2.2]oct-3-yl]-4-iodo-1H-pyrazole-1-carboxamide hydrochloride

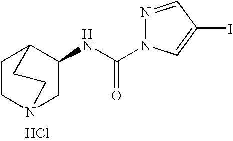

Phenyl chloroformate (0.75 mL, 6.0 mmol) is added dropwise to a solution of 4-iodopyrazole (1.05 g, 5.4 mmol) and TEA (0.9 mL, 6.5 mmol) in 15 mL CH₂Cl₂. The reaction is stirred at RT. After 60 h, water is added. The mixture is extracted with CH₂Cl₂, dried (MgSO₄), filtered and concentrated. Hexane is added and the solvent is removed in vacuo. A white solid forms on standing to provide 1.6 g (95%) of phenyl 4-iodo-1H-pyrazole-1-carboxylate. MS (EI) m/z 315.1 (M⁺).

Phenyl 4-iodo-1H-pyrazole-1-carboxylate (1.6 g, 5.2 mmol) and (R)-(+)-3-aminoquinuclidine dihydrochloride (1.0 g, 5.2 mmol) are suspended in 10 mL DMF. DIEA (2.7 mL, 15.5 mmol) is added dropwise. After 36 h, the solvent is removed and the residue is taken up in 1N NaOH and CHCl₃. The aqueous layer is extracted with CHCl₃, dried (MgSO₄), filtered and concentrated. The residue is purified by chromatography (Biotage 40S, 90:9:1 CHCl₃/MeOH/NH₄OH) to provide 1.66 g (93%) of the product as a white solid. A portion of the material is converted into the hydrochloride salt and recrystallized from MeOH/EtOAc. HRMS (FAB) calcd for C₁₁H₁₅IN₄O+H 347.0370, found 347.0357.

### EXAMPLE 3(H)

### N-[(3R)-1-azabicyclo[2.2.2]oct-3-yl]-4-(2-chlorophenyl)-1H-pyrazole-1-carboxamide hydrochloride

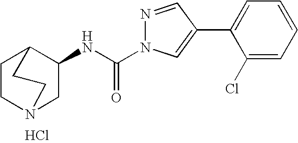

Hydrazine hydrate (0.55 mL, 11.3 mmol) is added to a suspension of 2-chlorophenylmalondialdehyde dissolved in 20 mL EtOH. The mixture is heated under reflux for 3 min, then allowed to stir at RT overnight. The solvent is removed in vacuo to provide 4-(2-chlorophenyl)-1H-pyrazole as a yellow solid. MS (EI) m/z 177.0 (M⁻).

4-Nitrophenyl chloroformate (2.3 g, 11.5 mmol) and 4-(2-chlorophenyl)-1H-pyrazole (2.0 g, 11.0 mmol) are dissolved in 30 mL CH₂Cl₂ and cooled to 0° C. TEA (1.7 mL, 12.0 mmol) is added, and the reaction is allowed to warm to RT. After 30 min, additional 4-nitrophenyl chloroformate (0.25 g) and TEA are added. After 1 h, water is added. The mixture is extracted with CH₂Cl₂, dried (MgSO₄), filtered and concentrated to give a solid. The solid is triturated with hexanes, filtered and dried to provide 1.7 g (45%) of the crude 4-nitrophenyl 4-(2-chlorophenyl)-1H-pyrazole-1-carboxylate.

A portion of 4-nitrophenyl 4-(2-chlorophenyl)-1H-pyrazole-1-carboxylate (0.34 g, 1.0 mmol) and (R)-(+)-3-aminoquinuclidine dihydrochloride (0.22 g, 1.1 mmol) are suspended in 5 mL DMF. TEA (0.4 mL, 3.0 mmol) is added dropwise. After 18 h, 1N NaOH is added, and the solvent is removed under reduced pressure. The residue is taken up in 1N NaOH and CHCl₃. The aqueous layer is extracted with CHCl₃, dried (MgSO₄), filtered and concentrated. The residue is purified by chromatography (Biotage 40S, 90:9:1 CHCl₃/MeOH/NH₄OH). The hydrochloride salt is prepared and recrystallized from MeOH/EtOAc to provide 102 mg (28%) of the product. HRMS (FAB) calcd for C₁₇H₁₉ClN₄O+H 331.1325, found 331.1312.

### EXAMPLE 4(H)

### N-[(3R,5R)-1-azabicyclo[3.2.1]oct-3-yl]-4-iodo-1H-pyrazole-1-carboxamide

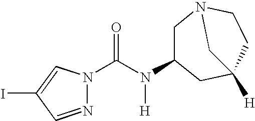

A solution of 4-iodopyrazole (1.05 g, 5.4 mmol) in 15 mL CH₂Cl₂ is treated with TEA (0.90 mL, 6.5 mmol) and phenylchloroformate (0.75 ml, 6.0 mmol). The mixture is stirred for 5 h and treated with H2O (1 mL). The aqueous layer is discarded and the organic dried (MgSO₄). The mixture is filtered, and evaporated to a yellow oil which solidifies upon evaporation from hexane. A portion of this solid (0.628 g, 2.0 mmol) is added to DMF (10 ml) containing (3R,5R)-1-azabicyclo[3.2.1]octan-3-amine dihydrochloride (0.398 g, 2.0 mmol). Diisopropylethyl amine (1.1 mL, 6.0 mmol) is added and the mixture becomes nearly homogeneous. The mixture is extracted between EtOAc and H₂O. The organic layer is washed with H₂O (3×), brine, dried (MgSO₄), and the mixture is evaporated. The resulting material is taken up in hot EtOAc, filtered through celite, and allowed to stand at RT. The resulting solid is collected and dried to afford Example 4(H) (0.142 g, 20%) as a white solid: HRMS (ESI) calcd for C₁₁H₁₅N₄OI (MH+) 347.0370, found 347.0370. Anal. Calcd for C₁₁H₁₅IN₄O: C, 38.17; H, 4.37; N, 16.18. Found: C, 38.43; H, 4.42; N, 16.11.

Materials and Methods for Identifying Binding Constants:

Membrane Preparation. Male Sprague-Dawley rats (300-350 g) are sacrificed by decapitation and the brains (whole brain minus cerebellum) are dissected quickly, weighed and homogenized in 9 volumes/g wet weight of ice-cold 0.32 M sucrose using a rotating pestle on setting 50 (10 up and down strokes). The homogenate is centrifuged at 1,000×g for 10 minutes at 4° C. The supernatant is collected and centrifuged at 20,000×g for 20 minutes at 4° C. The resulting pellet is resuspended to a protein concentration of 1-8 mg/mL. Aliquots of 5 mL homogenate are frozen at −80° C. until needed for the assay. On the day of the assay, aliquots are thawed at room temperature and diluted with Kreb's −20 mM Hepes buffer pH 7.0 (at room temperature) containing 4.16 mM NaHCO₃, 0.44 mM KH₂PO₄, 127 mM NaCl, 5.36 mM KCl, 1.26 mM CaCl₂, and 0.98 mM MgCl₂, so that 25-150 μg protein are added per test tube. Proteins are determined by the Bradford method (Bradford, M. M., Anal. Biochem., 72, 248-254, 1976) using bovine serum albumin as the standard.

Binding Assay. For saturation studies, 0.4 mL homogenate are added to test tubes containing buffer and various concentrations of radioligand, and are incubated in a final volume of 0.5 mL for 1 hour at 25° C. Nonspecific binding was determined in tissues incubated in parallel in the presence of 0.05 ml MLA for a final concentration of 1 μM MLA, added before the radioligand. In competition studies, drugs are added in increasing concentrations to the test tubes before addition of 0.05 ml [³H]-MLA for a final concentration of 3.0 to 4.0 nM [³H]-MLA. The incubations are terminated by rapid vacuum filtration through Whatman GF/B glass filter paper mounted on a 48 well Brandel cell harvester. Filters are pre-soaked in 50 mM Tris HCl pH 7.0-0.05% polyethylenimine. The filters are rapidly washed two times with 5 mL aliquots of cold 0.9% saline and then counted for radioactivity by liquid scintillation spectrometry.

Data Analysis. In competition binding studies, the inhibition constant (Ki) was calculated from the concentration dependent inhibition of [³H]-MLA binding obtained from non-linear regression fitting program according to the Cheng-Prusoff equation (Cheng, Y. C. and Prussoff, W. H., Biochem. Pharmacol., 22, p. 3099-3108, 1973). Hill coefficients were obtained using non-linear regression (GraphPad Prism sigmoidal dose-response with variable slope).

1. A method of treating a mammal in need thereof, wherein the mammal would receive symptomatic relief by decreasing the level of TNF-α comprising administering an alpha 7 nAChR full agonist.

2. The method of claim 1, wherein the symptomatic relief would be to treat the mammal for infection, inflammation, cancer, or diabetes.

3. The method of claim 2, wherein the mammal would optionally receive an antiviral or antibacterial agent for a therapeutically effective interval, and wherein infection or inflammation is caused by rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome; Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, HIV-3; cytomegalovirus; influenza; adenovirus; a herpes virus; or herpes zoster.

4. The method of claim 2, wherein cancer is multiple myeloma; acute and chronic myelogenous leukemia; or cancer-associated cachexia, and where the mammal would optionally receive at least one of an anticancer agent or antiemetic agent for a therapeutically effective interval.

5. The method of claim 2, wherein diabetes is type I and type II diabetes, and wherein the mammal optionally receives at least one agent for the treatment of diabetes for a therapeutically effective interval.

6. The method of claim 2, wherein diabetes is associated with pancreatic beta cell destruction.

7. The method of claim 1, wherein the full agonist is a compound of formula I:
Azabicyclo-N(R₁)—C(═X)—W Formula I
wherein Azabicyclo is 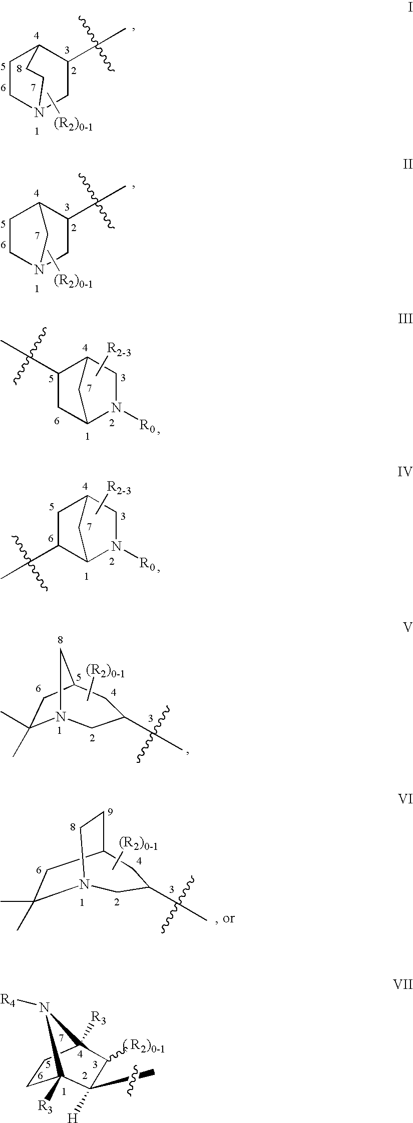
X is O, or S;
R⁰ is H, lower alkyl, substituted lower alkyl, or lower haloalkyl;
Each R₁ is H, alkyl, cycloalkyl, haloalkyl, substituted phenyl, or substituted naphthyl;
Each R₂ is independently F, Cl, Br, I, alkyl, substituted alkyl, haloalkyl, cycloalkyl, aryl, or R₂ is absent;
R₂₋₃ is H, F, Cl, Br, I, alkyl, haloalkyl, substituted alkyl, cycloalkyl, or aryl;
Each R₃ is independently H, alkyl, or substituted alkyl;
R₄ is H, alkyl, an amino protecting group, or an alkyl group having 1-3 substituents selected from F, Cl, Br, I, —OH, —CN, —NH₂, —NH(alkyl), or —N(alkyl)₂;
R₅ is 5-membered heteroaromatic mono-cyclic moieties containing within the ring 1-3 heteroatoms independently selected from the group consisting of —O—, ═N—, —N(R₁₀)—, and —S—, and having 0-1 substituent selected from R₉ and further having 0-3 substituents independently selected from F, Cl, Br, or I, or R₅ is 9-membered fused-ring moieties having a 6-membered ring fused to a 5-membered ring and having the formula 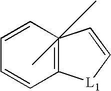
wherein L₁ is O, S, or NR₁₀, 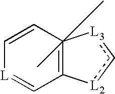
wherein L is CR₁₂ or N, L₂ and L₃ are independently selected from CR₁₂, C(R₁₂)₂, O, S, N, or NR₁₀, provided that both L₂ and L₃ are not simultaneously O, simultaneously S, or simultaneously O and S, or 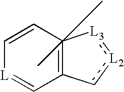
wherein L is CR₁₂ or N, and L₂ and L₃ are independently selected from CR₁₂, O, S, N, or NR₁₀, and each 9-membered fused-ring moiety having 0-1 substituent selected from R₉ and further having 0-3 substituent(s) independently selected from F, Cl, Br, or I, wherein the R₅ moiety attaches to other substituents as defined in formula I at any position as valency allows;
R₆ is 6-membered heteroaromatic mono-cyclic moieties containing within the ring 1-3 heteroatoms selected from ═N— and having 0-1 substituent selected from R₉ and 0-3 substituent(s) independently selected from F, Cl, Br, or I, or R₆ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₉ and 0-3 substituent(s) independently selected from F, Cl, Br, or I, wherein the R₆ moiety attaches to other substituents as defined in formula I at any position as valency allows;
R₇ is alkyl, substituted alkyl, haloalkyl, —OR₁₁, —CN, —NO₂, —N(R₈)₂;
Each R₈ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;
R₉ is alkyl, cycloalkyl, heterocycloalkyl, haloalkyl, halocycloalkyl, haloheterocycloalkyl, —OR₁₄, —SR₁₄, —N(R₁₄)₂, —C(O)R₁₄, —C(O)N(R₁₄)₂, —CN, —NR₁₄C(O)R₁₄, —S(O)₂N(R₁₄)₂, —NR₁₄S(O)₂R₁₄, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
R₁₀ is H, alkyl, haloalkyl, substituted alkyl, cycloalkyl, halocycloalkyl, substituted cycloalkyl, phenyl, or phenyl having 1 substituent selected from R₇ and further having 0-3 substituents independently selected from F, Cl, Br, or I;
Each R₁₁ is independently H, alkyl, cycloalkyl, heterocycloalkyl, haloalkyl, halocycloalkyl, or haloheterocycloalkyl;
Each R₁₂ is independently H, F, Cl, Br, I, alkyl, cycloalkyl, heterocycloalkyl, haloalkyl, halocycloalkyl, haloheterocycloalkyl, substituted alkyl, substituted cycloalkyl, substituted heterocycloalkyl, —CN, —NO₂, —OR₁₄, —SR₁₄, —N(R₁₄)₂, —C(O)R₁₄, —C(O)N(R₁₄)₂, —NR₁₄C(O)R₁₄, —S(O)₂N(R₁₄)₂, —NR₁₄S(O)₂R₁₄, or a bond;
R₁₃ is —OR₁₄, —SR₁₄, —N(R₁₄)₂, —C(O)R₁₄, —C(O)N(R₁₄)₂, —CN, —CF₃, —NR₁₄C(O)R₁₄, —S(O)₂N(R₁₄)₂, —NR₁₄S(O)₂R₁₄, or —NO₂;
Each R₁₄ is independently H, alkyl, cycloalkyl, heterocycloalkyl, haloalkyl, halocycloalkyl, or haloheterocycloalkyl;
wherein W is (A): 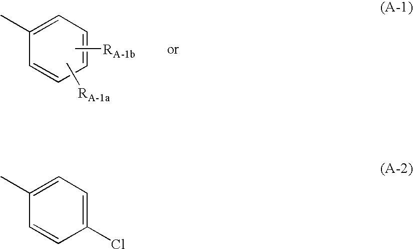
wherein R_{A-1a} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, —R₅, R₆, —OR_{A-3}, —SR_{A-3}, F, Cl, Br, I, —N(R_{A-3})₂, —C(O)R_{A-3}, —CN, —C(O)N(R_{A-3})₂, —NR_{A-3}C(O)R_{A-3}, —S(O)R_{A-3}, —OS(O)₂R_{A-3}, —NR_{A-3}S(O)₂R_{A-3}, —NO₂, and —N(H)C(O)N(H)R_{A-3};
R_{A-1b} is —O—R_{A-3}, —S—R_{A-3}, —S(O)—R_{A-3}, —C(O)—R_{A-7}, and alkyl substituted on the ω carbon with R_{A-7};
Each R_{A-3} is independently selected from H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
R_{A-4} is selected from cycloalkyl, halocycloalkyl, substituted cycloalkyl, heterocycloalkyl, haloheterocycloalkyl, or substituted heterocycloalkyl;
Each R_{A-5} is independently selected from cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{A-6} is independently selected from alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
R_{A-7} is selected from aryl, R₅, or R₆;
wherein W is (B): 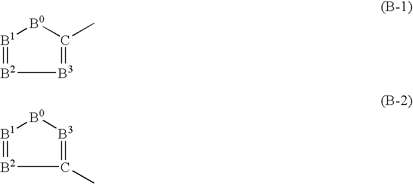
wherein B⁰ is —O—, —S—, or —N(R_{B-0})—;
B¹ and B² are independently selected from ═N—, or ═C(R_{B-1})—;
B³ is ═N—, or ═CH—, provided that when both B¹ and B² are ═C(R_{B-1})— and B³ is ═CH—, only one ═C(R_{B-1})— can be ═CH—, and further provided that when B⁰ is —O—, B² is ═C(R_{B-1})— and B³ is ═C(H)—, B¹ cannot be ═N—,
R_{B-0} is H, alkyl, cycloalkyl, heterocycloalkyl, haloalkyl, halocycloalkyl, haloheterocycloalkyl, substituted alkyl, substituted cycloalkyl, substituted heterocycloalkyl, or aryl, and provided that when B is (B-2) and B³ is ═N— and B⁰ is N(R_{B-0}), R_{B-0} cannot be phenyl or substituted phenyl;
R_{B-1} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, —OR_{B-2}, —SR_{B-2}, F, Cl, Br, I, —N(R_{B-2})₂, —C(O)R_{B-2}, —C(O)N(R_{B-2})₂, —CN, —NR_{B-2}C(O)R_{B-4}, —S(O)₂N(R_{B-2})₂, —OS(O)₂R_{B-4}, —S(O)₂R_{B-2}, —NR_{B-2}S(O)₂R_{B-2}, —N(H)C(O)N(H)R_{B-2}, —NO₂, R₅, and R₆;
Each R_{B-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{B-3} is independently H, alkyl, haloalkyl, limited substituted alkyl, cycloalkyl, halocycloalkyl, substituted cycloalkyl, heterocycloalkyl, haloheterocycloalkyl, substituted heterocycloalkyl;
R_{B-4} is independently H, alkyl, cycloalkyl, heterocycloalkyl, haloalkyl, halocycloalkyl, or haloheterocycloalkyl;
wherein W is (C):
(C) is a six-membered heterocyclic ring system having 1-2 nitrogen atoms or a 10-membered bicyclic-six-six-fused-ring system having up to two nitrogen atoms within either or both rings, provided that no nitrogen is at a bridge of the bicyclic-six-six-fused-ring system, and further having 1-2 substitutents independently selected from R_{C-1};
Each R_{C-1} is independently H, F, Cl, Br, I, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, lactam heterocycloalkyl, phenyl, substituted phenyl, —NO₂, —CN, —OR_{C-2}, —SR_{C-2}, —SOR_{C-2}, —SO₂R_{C-2}, —NR_{C-2}C(O)R_{C-2}, —N(R_{C-2})₂, —C(O)R_{C-2}, —C(O)₂R_{C-2}, —C(O)N(R_{C-2})₂, —SCN, —S(O)N(R_{C-2})₂, —S(O)₂N(R_{C-2})₂, —NR_{C-2}S(O)₂R_{C-2}, R₅, or R₆;
Each R_{C-2} is independently H, alkyl, cycloalkyl, heterocycloalkyl, alkyl substituted with 1 substituent selected from R_{C-5}, cycloalkyl substituted with 1 substituent selected from R_{C-5}, heterocycloalkyl substituted with 1 substituent selected from R_{C-5}, haloalkyl, halocycloalkyl, haloheterocycloalkyl, phenyl, or substituted phenyl;
Each R_{C-3} is independently H, alkyl, or substituted alkyl;
R_{C-4} is H, alkyl, an amino protecting group, or an alkyl group having 1-3 substituents selected from F, Cl, Br, I, —OH, —CN, —NH₂, —NH(alkyl), or —N(alkyl)₂;
R_{C-5} is —CN, —CF₃, —NO₂, —OR_{C-6}, —SR_{C-6}, —N(R_{C-6})₂, —C(O)R_{C-6}, —SOR_{C-6}, —SO₂R_{C-6}, —C(O)N(R_{C-6})₂, —NR_{C-6}C(O)R_{C-6}, —S(O)₂N(R_{C-6})₂, or —NR_{C-6}S(O)₂R_{C-6};
Each R_{C-6} is independently H, alkyl, cycloalkyl, heterocycloalkyl, haloalkyl, halocycloalkyl, or haloheterocycloalkyl;
wherein W is (D): 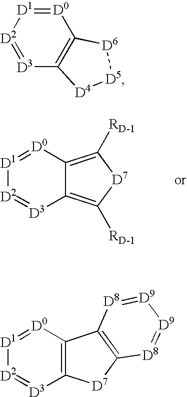
provided that the bond between the —C(═X)— group and the W group may be attached at any available carbon atom within the D group as provided in R_{D-1}, R_{D-3}, and R_{D-4};
D⁰, D¹, D², and D³ are N or C(R_{D-1}) provided that up to one of D⁰, D¹, D², or D³ is N and the others are C(R_{D-1}), further provided that when the core molecule is attached at D² and D⁰ or D¹ is N, D³ is C(H), and further provided that there is only one attachment to the core molecule;
D⁴ - - - D⁵ - - - D¹ is selected from N(R_{D-2})—C(R_{D-3})═C(R_{D-3}), N═C(R_{D-3})—C(R_{D-4})₂, C(R_{D-3})═C(R_{D-3})—N(R_{D-2}), C(R_{D-3})₂—N(R_{D-2})—C(R_{D-3})₂, C(R_{D-4})₂—C(R_{D-3})═N, N(R_{D-2})—C(R_{D-3})₂—C(R_{D-3})₂, C(R_{D-3})₂—C(R_{D-3})₂—N(R_{D-2}), O—C(R_{D-3})═C(R_{D-3}), O—C(R_{D-3})₂—C(R_{D-3})₂, C(R_{D-3})₂—O—C(R_{D-3})₂, C(R_{D-3})═C(R_{D-3})—O, C(R_{D-3})₂—C(R_{D-3})₂—O, S—C(R_{D-3})═C(R_{D-3}), S—C(R_{D-3})₂—C(R_{D-3})₂, C(R_{D-3})₂—S—C(R_{D-3})₂, C(R_{D-3})═C(R_{D-3})—S, or C(R_{D-3})₂—C(R_{D-3})₂—S;
provided that when C(X) is attached to W at D² and D⁶ is O, N(R_{D-2}), or S, D⁴ - - - D⁵ is not CH═CH;
and further provided that when C(X) is attached to W at D² and D⁴ is O, N(R_{D-2}), or S, D⁵ - - - D⁶ is not CH═CH;
Each R_{D-1} is independently H, F, Br, I, Cl, —CN, —CF₃, —OR_{D-5}, —SR_{D-5}, —N(R_{D-5})₂, or a bond to —C(X)— provided that only one of R_{D-1}, R_{D-3}, and R_{D-4} is said bond;
Each R_{D-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, or R₆;
Each R_{D-3} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-10})₂, —SR_{D-10}, —S(O)₂R_{D-10}, —C(O)R_{D-12}, —CO₂R_{D-10}, aryl, R₅, R₆, or a bond to —C(X)— provided that only one of R_{D-1}, R_{D-3}, and R_{D-4} is said bond;
Each R_{D-4} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-11})₂, —SR_{D-10}, —CO₂R_{D-10}, aryl, R₅, R₆, or a bond to —C(X)— provided that only one of R_{D-1}, R_{D-3}, and R_{D-4} is said bond;
Each R_{D-5} is independently H, C₁₋₃ alkyl, or C₂₋₄ alkenyl;
D⁷ is O, S, or N(R_{D-2});
D⁸ and D⁹ are C(R_{D-1}), provided that when the molecule is attached to the phenyl moiety at D⁹, D⁸ is CH;
Each R_{D-10} is H, alkyl, cycloalkyl, haloalkyl, substituted phenyl, or substituted naphthyl;
Each R_{D-11} is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;
R_{D-12} is H, alkyl, substituted alkyl, cycloalkyl, haloalkyl, heterocycloalkyl, substituted heterocycloalkyl, substituted phenyl, or substituted naphthyl;
wherein W is (E): 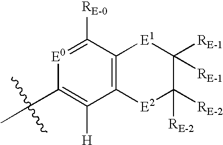
E⁰ is CH or N;
R_{E-0} is H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, R₅, R₆, —OR_{E-3}, —SR_{E-3}, —N(R_{E-3})₂, —C(O)R_{E-3}, —CN, —C(O)N(R_{E-3})₂, —NR_{E-3}C(O)R_{E-3}, —S(O)R_{E-3}, —S(O)R_{E-5}, —OS(O)₂R_{E-3}, —NR_{E-3}S(O)₂R_{E-3}, —NO₂, or —N(H)C(O)N(H)R_{E-3};
E¹ is O, CR_{E-1-1}, or C(R_{E-1-1})₂, provided that when E¹ is CR_{E-1-1}, one R_{E-1} is a bond to CR_{E-1-1}, and further provided that at least one of E¹ or E² is O;
Each R_{E-1-1} is independently H, F, Br, Cl, CN, alkyl, haloalkyl, substituted alkyl, alkynyl, cycloalkyl, —OR_{E}, or —N(R_{E})₂, provided that at least one R_{E-1-1} is H when E¹ is C(R_{E-1-1})₂;
Each R_{E-1} is independently H, alkyl, substituted alkyl, haloalkyl, cycloalkyl, heterocycloalkyl, or a bond to E¹ provided that E¹ is CR_{E-1-1};
E² is O, CR_{E-2-2}, or C(R_{E-2-2})₂, provided that when E² is CR_{E-2-2}, one R_{E-2} is a bond to CR_{E-2-2}, and further provided that at least one of E¹ or E² is O;
Each R_{E-2-2} is independently H, F, Br, Cl, CN, alkyl, haloalkyl, substituted alkyl, alkynyl, cycloalkyl, —OR_{E}, or —N(R_{E})₂, provided that at least one R_{E-2-2} is H when E² is C(R_{E-2-2})₂;
Each R_{E-2} is independently H, alkyl, substituted alkyl, haloalkyl, cycloalkyl, heterocycloalkyl, or a bond to E² provided that E² is CR_{E-2-2};
Each R_{E} is independently H, alkyl, cycloalkyl, heterocycloalkyl, haloalkyl, halocycloalkyl, or haloheterocycloalkyl;
Each R_{E-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I or substituted phenyl;
R_{E-4} is H, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{E-5} is independently H, haloalkyl, substituted alkyl, cycloalkyl, halocycloalkyl, substituted cycloalkyl, heterocycloalkyl, haloheterocycloalkyl, substituted heterocycloalkyl, R₅, or R₆;
Each R_{E-6} is independently alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I;
wherein W is (F): 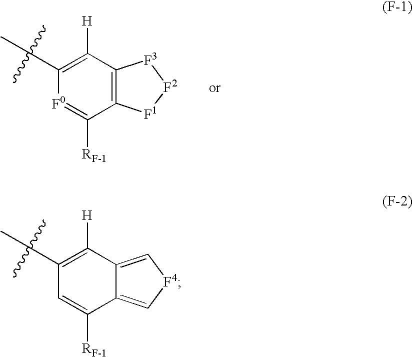
F⁰ is C(H) wherein F¹ - - - F² - - - F³ is selected from O—C(R_{F-2})═N, O—C(R_{F-3})(R_{F-2})—N(R_{F-4}), O—C(R_{F-3})(R_{F-2})—S, O—N═C(R_{F-3}), O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), or C(R_{F-3})₂—C(R_{F-2})(R_{F-3})—C(R_{F-3})₂;
F⁰ is N wherein F¹ - - - F² - - - F³ is selected from O—C(R_{F-2})═N, O—C(R_{F-3})(R_{F-2})—N(R_{F-4}), O—C(R_{F-3})(R_{F-2})—S, O—N═C(R_{F-3}) O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), C(R_{F-3})═C(R_{F-2})—C(R_{F-3})₂, or C(R_{F-3})₂—C(R_{F-2})(R_{F-3})—C(R_{F-3})₂;
F⁴ is N(R_{F-7}), O, or S;
R_{F-1} is H, F, Cl, Br, I, —CN, —CF₃, —OR_{F-8}, —SR_{F-8}, or —N(R_{F-8})₂;
R_{F-2} is H, F, alkyl, haloalkyl, substituted alkyl, lactam heterocycloalkyl, phenoxy, substituted phenoxy, R₅, R₆, —N(R_{F-4})-aryl, —N(R_{F-4})-substituted phenyl, —N(R_{F-4})-substituted naphthyl, —O-substituted phenyl, —O-substituted naphthyl, —S-substituted phenyl, —S-substituted naphthyl, or alkyl substituted on the ω carbon with R_{F-9};
R_{F-3} is H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{F-8}, —C(O)N(R_{F-8})₂, —NHR_{F-8}, —NR_{F-8}COR_{F-8}, —N(R_{F-8})₂, —SR_{F-8}, —C(O)R_{F-8}, —CO₂R_{F-8}, aryl, R₅, or R₆;
R_{F-4} is H, or alkyl;
Each R_{F-5} is independently F, Br, Cl, I, alkyl, substituted alkyl, haloalkyl, alkenyl, substituted alkenyl, haloalkenyl, alkynyl, substituted alkynyl, haloalkynyl, —CN, —CF₃, —OR_{F-8}, —C(O)NH₂, —NHR_{F-8}, —SR_{F-8}, —CO₂R_{F-8}, aryl, phenoxy, substituted phenoxy, heteroaryl, —N(R_{F-4})-aryl, or —O-substituted aryl;
One of R_{F-6} is H, alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆, and each of the other two R_{F-6} is independently selected from alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆;
R_{F-7} is H, alkyl, haloalkyl, substituted alkyl, cycloalkyl, halocycloalkyl, substituted cycloalkyl, phenyl, or phenyl having 1 substituent selected from R₉ and further having 0-3 substituents independently selected from F, Cl, Br, or I;
R_{F-8} is H, alkyl, substituted alkyl, cycloalkyl, haloalkyl, heterocycloalkyl, substituted heterocycloalkyl, substituted phenyl, or substituted naphthyl;
R_{F-9} is aryl, R₅, or R₆;
wherein W is (G): 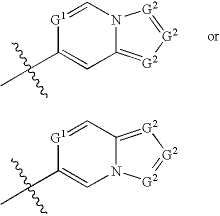
G¹ is N or CH;
Each G² is N or C(R_{G-1}), provided that no more than one G² is N;
Each R_{G-1} is independently H, alkyl, substituted alkyl, haloalkyl, alkenyl, substituted alkenyl, haloalkenyl, alkynyl, substituted alkynyl, haloalkynyl, —CN, —NO₂, F, Br, Cl, I, —C(O)N(R_{G-3})₂, —N(R_{G-3})₂, —SR_{G-6}, —S(O)₂R_{G-6}, —OR_{G-6}, —C(O)R_{G-6}, —CO₂R_{G-6}, aryl, R₅, R₆, or two R_{G-1} on adjacent carbon atoms may combine for W to be a 6-5-6 fused-tricyclic-heteroaromatic-ring system optionally substituted on the newly formed ring where valency allows with 1-2 substitutents independently selected from F, Cl, Br, I, and R_{G-2};
R_{G-2} is alkyl, alkenyl, alkynyl, cycloalkyl, heterocycloalkyl, haloalkyl, haloalkenyl, haloalkynyl, halocycloalkyl, haloheterocycloalkyl, —OR_{G-8}, —SR_{G-8}, —S(O)₂R_{G-8}, —S(O)R_{G-8}, —OS(O)₂R_{G-8}, —N(R_{G-8})₂, —C(O)R_{G-8}, —C(S)R_{G-8}, —C(O)OR_{G-8}, —CN, —C(O)N(R_{G-8})₂, —NR_{G-8}C(O)R_{G-8}, —S(O)₂N(R_{G-8})₂, —NR_{G-8}S(O)₂R_{G-8}, —NO₂, —N(R_{G-8})C(O)N(R_{G-8})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R_{G-7}, naphthyl, or naphthyl having 0-4 substituents independently selected from F, Cl, Br, I, or R_{G-7};
provided that when G² adjacent to the bridge N is C(R_{G-1}) and the other G² are CH, that R_{G-1} is other than H, F, Cl, I, alkyl, substituted alkyl or alkynyl;
Each R_{G-3} is independently H, alkyl, cycloalkyl, heterocycloalkyl, alkyl substituted with 1 substituent selected from R_{G-4}, cycloalkyl substituted with 1 substituent selected from R_{G-4}, heterocycloalkyl substituted with 1 substituent selected from R_{G-4}, haloalkyl, halocycloalkyl, haloheterocycloalkyl, phenyl, or substituted phenyl;
R_{G-4} is —OR_{G-5}, —SR_{G-5}, —N(R_{G-5})₂, —C(O)R_{G-5}, —SOR_{G-5}, —SO₂R_{G-5}, —C(O)N(R_{G-5})₂, —CN, —CF₃, —NR_{G-5}C(O)R_{G-5}, —S(O)₂N(R_{G-5})₂, —NR_{G-5}S(O)₂R_{G-5}, or —NO₂;
Each R_{G-5} is independently H, alkyl, cycloalkyl, heterocycloalkyl, haloalkyl, halocycloalkyl, or haloheterocycloalkyl;
R_{G-6} is H, alkyl, haloalkyl, substituted alkyl, cycloalkyl, halocycloalkyl, substituted cycloalkyl, phenyl, or phenyl having 0-4 substituents independently selected from F, Cl, Br, I, and R_{G-7};
R_{G-7} is alkyl, substituted alkyl, haloalkyl, —OR_{G-5}, —CN, —NO₂, —N(R_{G-3})₂;
Each R_{G-8} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R_{G-7};
wherein W is (H) 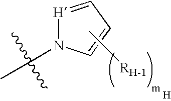
H′ is N or CH;
Each R_{H-1} is independently F, Cl, Br, I, —CN, —NO₂, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, lactam heterocyclcoalkyl, aryl, R₅, R₆, —OR₈, —SR₈, —SOR₈, —SO₂R₈, —SCN, —S(O)N(R₈)₂, —S(O)₂N(R₈)₂, —C(O)R₈, —C(O)₂R₈, —C(O)N(R₈)₂, C(R₈)═N—OR₈, —NC(O)R₅, —NC(O)R_{H-3}, —NC(O)R₆, —N(R₈)₂, —NR₈C(O)R₈, —NR₈S(O)₂R₈, or two R_{H-1} on adjacent carbon atoms may fuse to form a 6-membered ring to give a 5-6 fused, bicyclic moiety where the 6-membered ring is optionally substituted with 1-3 substitutents selected from R_{H-2};
m_{H} is 0, 1, or 2;
R_{H-2} is alkyl, alkenyl, alkynyl, cycloalkyl, heterocycloalkyl, haloalkyl, haloalkenyl, haloalkynyl, halocycloalkyl, haloheterocycloalkyl, —OR_{H-3}, —SR_{H-3}, —S(O)₂R_{H-3}, —S(O)R_{H-3}, —OS(O)₂R_{H-3}, —N(R_{H-3})₂, —C(O)R_{H-3}, —C(S)R_{H-3}, —C(O)OR_{H-3}, —CN, —C(O)N(R_{H-3})₂, —NR_{H-3}C(O)R_{H-3}, —S(O)₂N(R_{H-3})₂, —NR_{H-3}S(O)₂R_{H-3}, —NO₂, —N(R_{H-3})C(O)N(R_{H-3})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R₇, naphthyl, naphthyl having 0-4 substituents independently selected from F, Cl, Br, I, or R₇, or two R_{H-2} on adjacent carbon atoms may combine to form a three-ring-fused-5-6-6 system optionally substituted with up to 3 substituents independently selected from Br, Cl, F, I, —CN, —NO₂, —CF₃, —N(R_{H-3})₂, —N(R_{H-3})C(O)R_{H-3}, alkyl, alkenyl, and alkynyl;
Each R_{H-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R₇;
or pharmaceutically acceptable salt, racemic mixture, or pure enantiomer thereof.

8. The method of claim 7, wherein the symptomatic relief would be to treat the mammal for pain, inflammation, cancer, or diabetes.

9. The method of claim 8, wherein the mammal would optionally receive an antiviral or antibacterial agent for a therapeutically effective interval, and wherein infection or inflammation is caused by rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome; Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, HIV-3; cytomegalovirus; influenza; adenovirus; a herpes virus; or herpes zoster.

10. The method of claim 8, wherein cancer is multiple myeloma; acute and chronic myelogenous leukemia; or cancer-associated cachexia, and where the mammal would optionally receive at least one of an anticancer agent or antiemetic agent for a therapeutically effective interval.

11. The method of claim 8, wherein diabetes is type I and type II diabetes, and wherein the mammal optionally receives at least one agent for the treatment of diabetes for a therapeutically effective interval.

12. The method of claim 8, wherein diabetes is associated with pancreatic beta cell destruction.

13. The method of claim 7, wherein X is O and R₁ is H.

14. The method of claim 13, wherein W is (C), (D), (E), (F), and (G).

15. The method of claim 14, wherein Azabicyclo is I, II, V and VII.

16. The method of claim 15, wherein W is 4-chlorophen-1-yl; dibenzo[b,d]thiophene-2-yl; isoquinoline-3-yl; furo[2,3-c]pyridine-5-yl; furo[3,2-c]pyridine-6; 1,3-benzodioxole-5-yl; 2,3-dihydro-1,4-benzodioxine-6-yl; 2,3-dihydro-1,4-benzodioxine-7-yl; 1,3-benzoxazole-5-yl; thieno[2,3-c]pyridine-5-yl; thieno[3,2-c]pyridine-6-yl; [1]benzothieno[3,2-c]pyridine-3-yl; 1,3-benzothiazole-6-yl; thieno[3,4-c]pyridine-6-yl; 2,3-dihydro-1-benzofuran-5-yl; 1-benzofuran-5-yl; furo[3,2-c]pyridine-6-yl; [1]benzothieno[2,3-c]pyridine-3-yl; dibenzo[b,d]furan-2-yl; 1-benzofuran-6-yl; 2-naphthyl; 1H-indole-6-yl; pyrrolo[1,2-c]pyrimidine-3-yl; 1-benzothiophene-5-yl; 1-benzothiophene-5-yl; 1-benzothiophene-6-yl; pyrrolo[1,2-a]pyrazine-3-yl; 1H-indole-6-yl; pyrazino[1,2-a]indole-3-yl; 1,3-benzothiazole-6-yl; [1]benzofuro[2,3-c]pyridine-3-yl; [1]benzofuro[2,3-c]pyridine-3-yl; 2H-chromene-6-yl; indolizine-6-yl; and [1,3]dioxolo[4,5-c]pyridine-6-yl; any of which is optionally substituted as allowed in claim 15.

17. The method of claim 16, wherein the agonist is
N-[(3R)-1-azabicyclo[2.2.2]oct-3-yl]-4-chlorobenzamide;
N-[(3R)-1-azabicyclo[2.2.2]oct-3-yl]dibenzo[b,d]thiophene-2-carboxamide;
N-[(3R)-1-azabicyclo[2.2.2]oct-3-yl]isoquinoline-3-carboxamide;
N-[(3R)-1-azabicyclo[2.2.2]oct-3-yl]furo[2,3-c]pyridine-5-carboxamide;
N-[(3R)-1-azabicyclo[2.2.2]oct-3-yl]-1,3-benzodioxole-5-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-2,3-dihydro-1,4-benzodioxine-6-carboxamide;
N-[(3R)-1-azabicyclo[2.2.2]oct-3-yl]-3-methylfuro[2,3-c]pyridine-5-carboxamide;
N-[(1S,2R,4R)-7-azabicyclo[2.2.1]hept-2-yl]isoquinoline-3-carboxamide;
N-[(1S,2R,4R)-7-azabicyclo[2.2.1]hept-2-yl]-3-methylfuro[2,3-c]pyridine-5-carboxamide;
N-[(3R)-1-azabicyclo[2.2.2]oct-3-yl]-1,3-benzoxazole-5-carboxamide;
N-[(3R)-1-azabicyclo[2.2.2]oct-3-yl]-2-methyl-1,3-benzoxazole-5-carboxamide;
N-[(1S,2R,4R)-7-azabicyclo[2.2.1]hept-2-yl]thieno[2,3-c]pyridine-5-carboxamide;
N-[(1S,2R,4R)-7-azabicyclo[2.2.1]hept-2-yl]thieno[3,2-c]pyridine-6-carboxamide;
N-[(1S,2R,4R)-7-azabicyclo[2.2.1]hept-2-yl]furo[2,3-c]pyridine-5-carboxamide;
N-[(3R)-1-azabicyclo[2.2.2]oct-3-yl]-3-ethylfuro[2,3-c]pyridine-5-carboxamide;
N-[(3R)-1-azabicyclo[2.2.2]oct-3-yl]-3-isopropylfuro[2,3-c]pyridine-5-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
5-{[(2R)-7-azoniabicyclo[2.2.1]hept-2-ylamino]carbonyl}-3-ethylfuro[2,3-c]pyridin-6-ium dichloride;
5-{[(2R)-7-azoniabicyclo[2.2.1]hept-2-ylamino]carbonyl}-3-isopropylfuro[2,3-c]pyridin-6-ium dichloride;
N-[(3R,4S)-1-azabicyclo[2.2.1]hept-3-yl]furo[2,3-c]pyridine-5-carboxamide;
N-1-azabicyclo[2.2.2]oct-3-yl[1]benzothieno[3,2-c]pyridine-3-carboxamide;
N-[(3R)-1-azabicyclo[2.2.2]oct-3-yl]-1,3-benzothiazole-6-carboxamide;
N-[(3R)-1-azabicyclo[2.2.2]oct-3-yl]-3-chlorofuro[2,3-c]pyridine-5-carboxamide;
N-1-azabicyclo[2.2.2]oct-3-ylfuro[2,3-c]pyridine-5-carboxamide;
N-[(3R)-1-azabicyclo[2.2.2]oct-3-yl]thieno[3,4-c]pyridine-6-carboxamide;
N-[(3R,5R)-1-azabicyclo[3.2.1]oct-3-yl]-3-methylfuro[2,3-c]pyridine-5-carboxamide;
N-[(3R,4S)-1-azabicyclo[2.2.1]hept-3-yl]-3-methylfuro[2,3-c]pyridine-5-carboxamide;
N-[(3R)-1-azabicyclo[2.2.2]oct-3-yl]-2,3-dihydro-1-benzofuran-5-carboxamide;
N-[(3R,4S)-1-azabicyclo[2.2.1]hept-3-yl]thieno[2,3-c]pyridine-5-carboxamide;
N-[(3R)-1-azabicyclo[2.2.2]oct-3-yl]-1-benzofuran-5-carboxamide;
N-[(3R)-1-azabicyclo[2.2.2]oct-3-yl]furo[3,2-c]pyridine-6-carboxamide;
N-[(3R,4S)-1-azabicyclo[2.2.1]hept-3-yl]thieno[3,2-c]pyridine-6-carboxamide;
N-[(3R,4S)-1-azabicyclo[2.2.1]hept-3-yl]3-ethylfuro[2,3-c]pyridine-5-carboxamide;
N-[(3R,4S)-1-azabicyclo[2.2.1]hept-3-yl]3-isopropylfuro[2,3-c]pyridine-5-carboxamide;
N-[(1S,2R,4R)-7-azabicyclo[2.2.1]hept-2-yl]-3-chlorofuro[2,3-c]pyridine-5-carboxamide;
N-[(3R,4S)-1-azabicyclo[2.2.1]hept-3-yl]3-chlorofuro[2,3-c]pyridine-5-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]furo[3,2-c]pyridine-6-carboxamide;
N-[(3R,5R)-1-azabicyclo[3.2.1]oct-3-yl]-4-chlorobenzamide;
N-[(1S,2R,4R)-7-azabicyclo[2.2.1]hept-2-yl]thieno[3,4-c]pyridine-6-carboxamide;
N-[(1S,2R,4R)-7-azabicyclo[2.2.1]hept-2-yl]dibenzo[b,d]thiophene-2-carboxamide;
N-[(3R,4S)-1-azabicyclo[2.2.1]hept-3-yl]-1-benzofuran-5-carboxamide;
N-[(3R)-1-azabicyclo[2.2.2]oct-3-yl][1]benzothieno[2,3-c]pyridine-3-carboxamide;
N-[(1S,2R,4R)-7-azabicyclo[2.2.1]hept-2-yl][1]benzothieno[2,3-c]pyridine-3-carboxamide;
N-[(1S,2R,4R)-7-azabicyclo[2.2.1]hept-2-yl]-1-benzofuran-5-carboxamide;
N-[(1S,2R,4R)-7-azabicyclo[2.2.1]hept-2-yl]dibenzo[b,d]furan-2-carboxamide;
N-[(3R,5R)-1-azabicyclo[3.2.1]oct-3-yl]furo[2,3-c]pyridine-5-carboxamide;
N-[(3R,5R)-1-azabicyclo[3.2.1]oct-3-yl]furo[3,2-c]pyridine-6-carboxamide;
N-[(3R,5R)-1-azabicyclo[3.2.1]oct-3-yl]-1-benzofuran-5-carboxamide;
N-[(3R)-1-azabicyclo[2.2.2]oct-3-yl]-3-bromofuro[2,3-c]pyridine-5-carboxamide;
N-[(1S,2R,4R)-7-azabicyclo[2.2.1]hept-2-yl]-3-bromofuro[2,3-c]pyridine-5-carboxamide;
N-[(3R)-1-azabicyclo[2.2.2]oct-3-yl]-1-benzofuran-6-carboxamide;
N-[(2S,3R)-2-methyl-1-azabicyclo[2.2.2]oct-3-yl]-2-naphthamide;
N-[(3R)-1-azabicyclo[2.2.2]oct-3-yl]pyrrolo[1,2-c]pyrimidine-3-carboxamide;
N-[(3R,5R)-1-azabicyclo[3.2.1]oct-3-yl]thieno[2,3-c]pyridine-5-carboxamide;
N-[(3R,5R)-1-azabicyclo[3.2.1]oct-3-yl]thieno[3,2-c]pyridine-6-carboxamide;
N-[(3R,4S)-1-azabicyclo[2.2.1]hept-3-yl]-1H-indole-6-carboxamide;
N-[(2S,3R)-2-methyl-1-azabicyclo[2.2.2]oct-3-yl]thieno[2,3-c]pyridine-5-carboxamide;
3-methyl-N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]-1-benzofuran-5-carboxamide;
N-[(2S,3R)-2-methyl-1-azabicyclo[2.2.2]oct-3-yl]thieno[3,2-c]pyridine-6-carboxamide;
N-[(2S,3R)-2-methyl-1-azabicyclo[2.2.2]oct-3-yl]pyrrolo[1,2-c]pyrimidine-3-carboxamide;
N-[(2S,3R)-2-methyl-1-azabicyclo[2.2.2]oct-3-yl]-1,3-benzothiazole-6-carboxamide;
N-[(3R,5R)-1-azabicyclo[3.2.1]oct-3-yl]pyrrolo[1,2-c]pyrimidine-3-carboxamide;
N-[(3R)-1-azabicyclo[2.2.2]oct-3-yl]-1-benzothiophene-5-carboxamide;
N-[(1S,2R,4R)-7-azabicyclo[2.2.1]hept-2-yl]pyrrolo[1,2-c]pyrimidine-3-carboxamide;
N-[(3R,4S)-1-azabicyclo[2.2.1]hept-3-yl]pyrrolo[1,2-c]pyrimidine-3-carboxamide;
N-[(3R,4S)-1-azabicyclo[2.2.1]hept-3-yl]-3-bromofuro[2,3-c]pyridine-5-carboxamide;
N-[(3R,4S)-1-azabicyclo[2.2.1]hept-3-yl]-1,3-benzodioxole-5-carboxamide;
N-[(3R)-1-azabicyclo[2.2.2]oct-3-yl]-3-bromo-1-benzofuran-5-carboxamide;
N-[(1S,2R,4R)-7-azabicyclo[2.2.1]hept-2-yl]-3-bromo-1-benzofuran-5-carboxamide;
N-[(3R)-1-azabicyclo[2.2.2]oct-3-yl]-3-bromothieno[2,3-c]pyridine-5-carboxamide;
N-[(1S,2R,4R)-7-azabicyclo[2.2.1]hept-2-yl]-3-bromothieno[2,3-c]pyridine-5-carboxamide;
N-[(3R,4S)-1-azabicyclo[2.2.1]hept-3-yl]-1-benzothiophene-5-carboxamide;
N-[(3S)-1-azabicyclo[2.2.2]oct-3-yl]furo[2,3-c]pyridine-5-carboxamide;
N-[(3R)-1-azabicyclo[2.2.2]oct-3-yl]-3-methyl-1-benzofuran-5-carboxamide;
N-[(1S,2R,4R)-7-azabicyclo[2.2.1]hept-2-yl]-3-methyl-1-benzofuran-5-carboxamide;
N-[(3R)-1-azabicyclo[2.2.2]oct-3-yl]-2-methyl-1-benzofuran-6-carboxamide;
N-[(3R,5R)-1-azabicyclo[3.2.1]oct-3-yl]-1-benzofuran-6-carboxamide;
N-[(2S,3R)-2-methyl-1-azabicyclo[2.2.2]oct-3-yl]-1-benzofuran-6-carboxamide;
N-[(2S,3R)-2-methyl-1-azabicyclo[2.2.2]oct-3-yl]-1-benzothiophene-5-carboxamide;
N-[(3R)-1-azabicyclo[2.2.2]oct-3-yl]-1-benzothiophene-6-carboxamide;
N-[(3R)-1-azabicyclo[2.2.2]oct-3-yl]pyrrolo[1,2-a]pyrazine-3-carboxamide;
N-[(3R,4S)-1-azabicyclo[2.2.1]hept-3-yl]-1-benzothiophene-6-carboxamide;
N-[(3R)-1-azabicyclo[2.2.2]oct-3-yl]-1-methyl-1H-indole-6-carboxamide;
N-[(3S)-1-azabicyclo[2.2.2]oct-3-yl]-1-benzofuran-5-carboxamide;
N-[(3R)-1-azabicyclo[2.2.2]oct-3-yl]-3-isopropyl-1-benzofuran-5-carboxamide;
N-[(1S,2R,4R)-7-azabicyclo[2.2.1]hept-2-yl]-3-isopropyl-1-benzofuran-5-carboxamide;
N-[(3R)-1-azabicyclo[2.2.2]oct-3-yl]-3-ethynylfuro[2,3-c]pyridine-5-carboxamide;
N-[(3R)-1-azabicyclo[2.2.2]oct-3-yl]-1H-indazole-6-carboxamide;
N-[(3R)-1-azabicyclo[2.2.2]oct-3-yl]-2-methyl-1-benzofuran-5-carboxamide;
N-[(1S,2R,4R)-7-azabicyclo[2.2.1]hept-2-yl]-2-methyl-1-benzofuran-5-carboxamide;
N-[(3R)-1-azabicyclo[2.2.2]oct-3-yl]pyrazino[1,2-a]indole-3-carboxamide;
3-bromo-N-[(2S,3R)-2-methyl-1-azabicyclo[2.2.2]oct-3-yl]furo[2,3-c]pyridine-5-carboxamide;
N-[(3R,5R)-1-azabicyclo[3.2.1]oct-3-yl]pyrrolo[1,2-a]pyrazine-3-carboxamide;
N-[(3R)-1-azabicyclo[2.2.2]oct-3-yl]-7-methoxy-2-naphthamide;
N-[(1S,2R,4R)-7-azabicyclo[2.2.1]hept-2-yl]pyrrolo[1,2-a]pyrazine-3-carboxamide;
N-[(3R,5R)-1-azabicyclo[3.2.1]oct-3-yl]-1,3-benzothiazole-6-carboxamide;
N-[(3R,4S)-1-azabicyclo[2.2.1]hept-3-yl]-3-bromo-1-benzofuran-6-carboxamide;
N-[(3R)-1-azabicyclo[2.2.2]oct-3-yl][1]benzofuro[2,3-c]pyridine-3-carboxamide;
N-[(1S,2R,4R)-7-azabicyclo[2.2.1]hept-2-yl][1]benzofuro[2,3-c]pyridine-3-carboxamide;
N-[(3R)-1-azabicyclo[2.2.2]oct-3-yl]-3-ethynyl-1-benzofuran-5-carboxamide;
N-[(1S,2R,4R)-7-azabicyclo[2.2.1]hept-2-yl]-3-ethynyl-1-benzofuran-5-carboxamide;
N-[(3R)-1-azabicyclo[2.2.2]oct-3-yl]-2H-chromene-6-carboxamide;
N-[(3R)-1-azabicyclo[2.2.2]oct-3-yl]-3-prop-1-ynyl-1-benzofuran-5-carboxamide;
N-[(3R)-1-azabicyclo[2.2.2]oct-3-yl]-2-phenyl-1,3-benzodioxole-5-carboxamide;
N-[(3R)-1-azabicyclo[2.2.2]oct-3-yl]-6-bromopyrrolo[1,2-a]pyrazine-3-carboxamide;
N-[(3R)-1-azabicyclo[2.2.2]oct-3-yl]-3-prop-1-ynylfuro[2,3-c]pyridine-5-carboxamide;
N-[(2S,3R)-2-methyl-1-azabicyclo[2.2.2]oct-3-yl]pyrrolo[1,2-a]pyrazine-3-carboxamide;
N-[(3R)-1-azabicyclo[2.2.2]oct-3-yl]indolizine-6-carboxamide;
2-amino-N-[(3R)-1-azabicyclo[2.2.2]oct-3-yl]-1,3-benzothiazole-6-carboxamide;
N-[(3R)-1-azabicyclo[2.2.2]oct-3-yl]-6-ethynylpyrrolo[1,2-a]pyrazine-3-carboxamide;
N-[(3R)-1-azabicyclo[2.2.2]oct-3-yl]-8-methoxy-2-naphthamide;
N-[(2S,3R)-2-methyl-1-azabicyclo[2.2.2]oct-3-yl]indolizine-6-carboxamide;
N-[(3R)-1-azabicyclo[2.2.2]oct-3-yl][1,3]dioxolo[4,5-c]pyridine-6-carboxamide;
N-[(1S,2R,4R)-7-azabicyclo[2.2.1]hept-2-yl][1,3]dioxolo[4,5-c]pyridine-6-carboxamide;
N-[(3R)-1-azabicyclo[2.2.2]oct-3-yl]-3-cyano-1-benzofuran-5-carboxamide;
N-[(3R,4S)-1-azabicyclo[2.2.1]hept-3-yl][1,3]dioxolo[4,5-c]pyridine-6-carboxamide;
N-[(3R)-1-azabicyclo[2.2.2]oct-3-yl]-3-ethyl-2,3-dihydro-1,4-benzodioxine-6-carboxamide;
N-[(3R)-1-azabicyclo[2.2.2]oct-3-yl]-3-ethyl-2,3-dihydro-1,4-benzodioxine-7-carboxamide;
N-[(3R)-1-azabicyclo[2.2.2]oct-3-yl]-7-hydroxy-2-naphthamide;
N-[(1S,2R,4R)-7-azabicyclo[2.2.1]hept-2-yl]-3-ethynylfuro[2,3-c]pyridine-5-carboxamide;
N-[(1S,2R,4R)-7-azabicyclo[2.2.1]hept-2-yl]-6-chloroisoquinoline-3-carboxamide;
N-[(3R)-1-azabicyclo[2.2.2]oct-3-yl]-6-methylisoquinoline-3-carboxamide;
N-[(1S,2R,4R)-7-azabicyclo[2.2.1]hept-2-yl]-6-methylisoquinoline-3-carboxamide;
N-[(3R)-1-azabicyclo[2.2.2]oct-3-yl]-3-cyanofuro[2,3-c]pyridine-5-carboxamide;
N-[(3R)-1-azabicyclo[2.2.2]oct-3-yl]-2-naphthamide; and
N-[(3R)-1-azabicyclo[2.2.2]oct-3-yl]dibenzo[b,d]furan-2-carboxamide;
provided that the agonist is the free base or a pharmaceutically acceptable salt thereof.

18. The method of claim 17, wherein the symptomatic relief would be to treat the mammal for pain, inflammation, cancer, or diabetes.

19. The method of claim 18, wherein the mammal would optionally receive an antiviral or antibacterial agent for a therapeutically effective interval, and wherein infection or inflammation is caused by rheumatoid arthritis; rheumatoid spondylitis; muscle degeneration; osteoporosis; osteoarthritis; psoriasis; contact dermatitis; bone resorption diseases; atherosclerosis; Paget's disease; uveititis; gouty arthritis; inflammatory bowel disease; adult respiratory distress syndrome; Crohn's disease; rhinitis; ulcerative colitis; anaphylaxis; asthma; Reiter's syndrome; tissue rejection of a graft; ischemia reperfusion injury; brain trauma; stroke; multiple sclerosis; cerebral malaria; sepsis; septic shock; toxic shock syndrome; fever and myalgias due to infection; HIV-1, HIV-2, HIV-3; cytomegalovirus; influenza; adenovirus; a herpes virus; or herpes zoster.

20. The method of claim 18, wherein cancer is multiple myeloma; acute and chronic myelogenous leukemia; or cancer-associated cachexia, and where the mammal would optionally receive at least one of an anticancer agent or antiemetic agent for a therapeutically effective interval.

21. The method of claim 18, wherein diabetes is type I and type II diabetes, and wherein the mammal optionally receives at least one agent for the treatment of diabetes for a therapeutically effective interval.

22. The method of claim 18, wherein diabetes is associated with pancreatic beta cell destruction.

23. The method of claim 18, wherein the compound is
N-[(3R)-1-azabicyclo[2.2.2]oct-3-yl]dibenzo[b,d]thiophene-2-carboxamide;
N-1-azabicyclo[2.2.2]oct-3-yl[1]benzothieno[3,2-c]pyridine-3-carboxamide;
N-[(3R)-1-azabicyclo[2.2.2]oct-3-yl][1]benzothieno[2,3-c]pyridine-3-carboxamide; and
N-[(1S,2R,4R)-7-azabicyclo[2.2.1]hept-2-yl][1]benzothieno[2,3-c]pyridine-3-carboxamide;
provided that the agonist is the free base or a pharmaceutically acceptable salt thereof.

24. The method of claim 18, wherein the compound is
N-[(2S,3R)-2-methyl-1-azabicyclo[2.2.2]oct-3-yl]-2-naphthamide;
N-[(3R)-1-azabicyclo[2.2.2]oct-3-yl]-7-methoxy-2-naphthamide;
N-[(3R)-1-azabicyclo[2.2.2]oct-3-yl]-8-methoxy-2-naphthamide;
N-[(3R)-1-azabicyclo[2.2.2]oct-3-yl]-7-hydroxy-2-naphthamide;
N-[(3R)-1-azabicyclo[2.2.2]oct-3-yl]-2-naphthamide;
N-[(3R)-1-azabicyclo[2.2.2]oct-3-yl]isoquinoline-3-carboxamide;
N-[(1S,2R,4R)-7-azabicyclo[2.2.1]hept-2-yl]isoquinoline-3-carboxamide;
N-[(1S,2R,4R)-7-azabicyclo[2.2.1]hept-2-yl]-6-chloroisoquinoline-3-carboxamide;
N-[(3R)-1-azabicyclo[2.2.2]oct-3-yl]-6-methylisoquinoline-3-carboxamide; and
N-[(1S,2R,4R)-7-azabicyclo[2.2.1]hept-2-yl]-6-methylisoquinoline-3-carboxamide;
provided that the agonist is the free base or a pharmaceutically acceptable salt thereof.

25. The method of claim 18, wherein the compound is
N-[(3R)-1-azabicyclo[2.2.2]oct-3-yl]furo[2,3-c]pyridine-5-carboxamide;
N-[(3R)-1-azabicyclo[2.2.2]oct-3-yl]furo[3,2-c]pyridine-6-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-3-methylfuro[2,3-c]pyridine-5-carboxamide;
N-[(1S,2R,4R)-7-azabicyclo[2.2.1]hept-2-yl]-3-methylfuro[2,3-c]pyridine-5-carboxamide;
N-[(1S,2R,4R)-7-azabicyclo[2.2.1]hept-2-yl]furo[2,3-c]pyridine-5-carboxamide;
N-[(3R)-1-azabicyclo[2.2.2]oct-3-yl]-3-ethylfuro[2,3-c]pyridine-5-carboxamide;
N-[(3R)-1-azabicyclo[2.2.2]oct-3-yl]-3-isopropylfuro[2,3-c]pyridine-5-carboxamide;
N-[(3R,4S)-1-azabicyclo[2.2.1]hept-3-yl]furo[2,3-c]pyridine-5-carboxamide;
N-[(3R)-1-azabicyclo[2.2.2]oct-3-yl]-3-chlorofuro[2,3-c]pyridine-5-carboxamide;
N-1-azabicyclo[2.2.2]oct-3-ylfuro[2,3-c]pyridine-5-carboxamide;
N-[(3R,5R)-1-azabicyclo[3.2.1]oct-3-yl]-3-methylfuro[2,3-c]pyridine-5-carboxamide;
N-[(3R,4S)-1-azabicyclo[2.2.1]hept-3-yl]-3-methylfuro[2,3-c]pyridine-5-carboxamide;
N-[(3R,4S)-1-azabicyclo[2.2.1]hept-3-yl]3-ethylfuro[2,3-c]pyridine-5-carboxamide;
N-[(3R,4S)-1-azabicyclo[2.2.1]hept-3-yl]3-isopropylfuro[2,3-c]pyridine-5-carboxamide;
N-[(1S,2R,4R)-7-azabicyclo[2.2.1]hept-2-yl]-3-chlorofuro[2,3-c]pyridine-5-carboxamide;
N-[(3R,4S)-1-azabicyclo[2.2.1]hept-3-yl]3-chlorofuro[2,3-c]pyridine-5-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]furo[3,2-c]pyridine-6-carboxamide;
N-[(3R,5R)-1-azabicyclo[3.2.1]oct-3-yl]furo[3,2-c]pyridine-6-carboxamide;
N-[(3R,5R)-1-azabicyclo[3.2.1]oct-3-yl]furo[2,3-c]pyridine-5-carboxamide;
N-[(3R)-1-azabicyclo[2.2.2]oct-3-yl]-3-bromofuro[2,3-c]pyridine-5-carboxamide;
N-[(1S,2R,4R)-7-azabicyclo[2.2.1]hept-2-yl]-3-bromofuro[2,3-c]pyridine-5-carboxamide;
3-methyl-N-[(2S,3R)-2-methyl-1-azabicyclo[2.2.2]oct-3-yl]furo[2,3-c]pyridine-5-carboxamide;
N-[(3R,4S)-1-azabicyclo[2.2.1]hept-3-yl]-3-bromofuro[2,3-c]pyridine-5-carboxamide;
N-[(3S)-1-azabicyclo[2.2.2]oct-3-yl]furo[2,3-c]pyridine-5-carboxamide;
N-[(3R)-1-azabicyclo[2.2.2]oct-3-yl]-3-ethynylfuro[2,3-c]pyridine-5-carboxamide;
3-bromo-N-[(2S,3R)-2-methyl-1-azabicyclo[2.2.2]oct-3-yl]furo[2,3-c]pyridine-5-carboxamide;
N-[(3R)-1-azabicyclo[2.2.2]oct-3-yl]-3-prop-1-ynylfuro[2,3-c]pyridine-5-carboxamide;
N-[(1S,2R,4R)-7-azabicyclo[2.2.1]hept-2-yl]-3-ethynylfuro[2,3-c]pyridine-5-carboxamide; and
N-[(3R)-1-azabicyclo[2.2.2]oct-3-yl]-3-cyanofuro[2,3-c]pyridine-5-carboxamide;
provided that the agonist is the free base or a pharmaceutically acceptable salt thereof.

26. The method of claim 18, wherein the compound is
N-[(3R)-1-azabicyclo[2.2.2]oct-3-yl]-2,3-dihydro-1-benzofuran-5-carboxamide;
N-[(3R)-1-azabicyclo[2.2.2]oct-3-yl]-1-benzofuran-5-carboxamide;
N-[(3R,4S)-1-azabicyclo[2.2.1]hept-3-yl]-1-benzofuran-5-carboxamide;
N-[(1S,2R,4R)-7-azabicyclo[2.2.1]hept-2-yl]-1-benzofuran-5-carboxamide;
N-[(3R,5R)-1-azabicyclo[3.2.1]oct-3-yl]-1-benzofuran-5-carboxamide;
N-[(3R)-1-azabicyclo[2.2.2]oct-3-yl]-1-benzofuran-6-carboxamide;
N-[(2S,3R)-2-methyl-1-azabicyclo[2.2.2]oct-3-yl]-1-benzofuran-5-carboxamide;
N-[(3R)-1-azabicyclo[2.2.2]oct-3-yl]-3-bromo-1-benzofuran-5-carboxamide;
N-[(1S,2R,4R)-7-azabicyclo[2.2.1]hept-2-yl]-3-bromo-1-benzofuran-5-carboxamide;
N-[(3R)-1-azabicyclo[2.2.2]oct-3-yl]-3-methyl-1-benzofuran-5-carboxamide;
N-[(1S,2R,4R)-7-azabicyclo[2.2.1]hept-2-yl]-3-methyl-1-benzofuran-5-carboxamide;
N-[(3R)-1-azabicyclo[2.2.2]oct-3-yl]-2-methyl-1-benzofuran-6-carboxamide;
N-[(3R,5R)-1-azabicyclo[3.2.1]oct-3-yl]-1-benzofuran-6-carboxamide;
N-[(2S,3R)-2-methyl-1-azabicyclo[2.2.2]oct-3-yl]-1-benzofuran-6-carboxamide;
N-[(3S)-1-azabicyclo[2.2.2]oct-3-yl]-1-benzofuran-5-carboxamide;
N-[(3R)-1-azabicyclo[2.2.2]oct-3-yl]-3-isopropyl-1-benzofuran-5-carboxamide;
N-[(1S,2R,4R)-7-azabicyclo[2.2.1]hept-2-yl]-3-isopropyl-1-benzofuran-5-carboxamide;
N-[(3R)-1-azabicyclo[2.2.2]oct-3-yl]-2-methyl-1-benzofuran-5-carboxamide;
N-[(1S,2R,4R)-7-azabicyclo[2.2.1]hept-2-yl]-2-methyl-1-benzofuran-5-carboxamide;
N-[(3R,4S)-1-azabicyclo[2.2.1]hept-3-yl]-3-bromo-1-benzofuran-6-carboxamide;
N-[(3R)-1-azabicyclo[2.2.2]oct-3-yl]-3-ethynyl-1-benzofuran-5-carboxamide;
N-[(1S,2R,4R)-7-azabicyclo[2.2.1]hept-2-yl]-3-ethynyl-1-benzofuran-5-carboxamide;
N-[(3R)-1-azabicyclo[2.2.2]oct-3-yl]-3-prop-1-ynyl-1-benzofuran-5-carboxamide; and
N-[(3R)-1-azabicyclo[2.2.2]oct-3-yl]-3-cyano-1-benzofuran-5-carboxamide;
provided that the agonist is the free base or a pharmaceutically acceptable salt thereof.

27. The method of claim 18, wherein the compound is
N-[(3R)-1-azabicyclo[2.2.2]oct-3-yl]pyrazino[1,2-a]indole-3-carboxamide;
N-[(3R,4S)-1-azabicyclo[2.2.1]hept-3-yl]-1H-indole-6-carboxamide;
N-[(3R)-1-azabicyclo[2.2.2]oct-3-yl]-1-methyl-1H-indole-6-carboxamide;
N-[(3R)-1-azabicyclo[2.2.2]oct-3-yl]-1-benzothiophene-5-carboxamide;
N-[(3R,4S)-1-azabicyclo[2.2.1]hept-3-yl]-1-benzothiophene-5-carboxamide;
N-[(2S,3R)-2-methyl-1-azabicyclo[2.2.2]oct-3-yl]-1-benzothiophene-5-carboxamide;
N-[(3R)-1-azabicyclo[2.2.2]oct-3-yl]-1-benzothiophene-6-carboxamide; and
N-[(3R,4S)-1-azabicyclo[2.2.1]hept-3-yl]-1-benzothiophene-6-carboxamide;
provided that the agonist is the free base or a pharmaceutically acceptable salt thereof.

28. The method of claim 18, wherein the compound is
N-[(1S,2R,4R)-7-azabicyclo[2.2.1]hept-2-yl]thieno[2,3-c]pyridine-5-carboxamide;
N-[(1S,2R,4R)-7-azabicyclo[2.2.1]hept-2-yl]thieno[3,2-c]pyridine-6-carboxamide;
N-[(1S,2R,4R)-7-azabicyclo[2.2.1]hept-2-yl]thieno[3,4-c]pyridine-6-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
N-[(3R)-1-azabicyclo[2.2.2]oct-3-yl]thieno[3,4-c]pyridine-6-carboxamide;
N-[(3R,4S)-1-azabicyclo[2.2.1]hept-3-yl]thieno[2,3-c]pyridine-5-carboxamide;
N-[(3R,4S)-1-azabicyclo[2.2.1]hept-3-yl]thieno[3,2-c]pyridine-6-carboxamide;
N-[(3R,5R)-1-azabicyclo[3.2.1]oct-3-yl]thieno[2,3-c]pyridine-5-carboxamide;
N-[(3R,5R)-1-azabicyclo[3.2.1]oct-3-yl]thieno[3,2-c]pyridine-6-carboxamide;
N-[(2S,3R)-2-methyl-1-azabicyclo[2.2.2]oct-3-yl]thieno[2,3-c]pyridine-5-carboxamide;
N-[(2S,3R)-2-methyl-1-azabicyclo[2.2.2]oct-3-yl]thieno[3,2-c]pyridine-6-carboxamide;
N-[(3R)-1-azabicyclo[2.2.2]oct-3-yl]-3-bromothieno[2,3-c]pyridine-5-carboxamide; and
N-[(1S,2R,4R)-7-azabicyclo[2.2.1]hept-2-yl]-3-bromothieno[2,3-c]pyridine-5-carboxamide;
provided that the agonist is the free base or a pharmaceutically acceptable salt thereof.

29. The method of claim 18, wherein the compound is
N-[(3R)-1-azabicyclo[2.2.2]oct-3-yl]-1,3-benzothiazole-6-carboxamide;
N-[(2S,3R)-2-methyl-1-azabicyclo[2.2.2]oct-3-yl]-1,3-benzothiazole-6-carboxamide;
N-[(3R,5R)-1-azabicyclo[3.2.1]oct-3-yl]-1,3-benzothiazole-6-carboxamide;
2-amino-N-[(3R)-1-azabicyclo[2.2.2]oct-3-yl]-1,3-benzothiazole-6-carboxamide;
N-[(3R)-1-azabicyclo[2.2.2]oct-3-yl]-1H-indazole-6-carboxamide;
N-[(3R)-1-azabicyclo[2.2.2]oct-3-yl][1,3]dioxolo[4,5-c]pyridine-6-carboxamide; and
N-[(1S,2R,4R)-7-azabicyclo[2.2.1]hept-2-yl][1,3]dioxolo[4,5-c]pyridine-6-carboxamide;
provided that the agonist is the free base or a pharmaceutically acceptable salt thereof.

30. The method of claim 18, wherein the compound is
N-[(3R)-1-azabicyclo[2.2.2]oct-3-yl]pyrrolo[1,2-c]pyrimidine-3-carboxamide;
N-[(3R,5R)-1-azabicyclo[3.2.1]oct-3-yl]pyrrolo[1,2-c]pyrimidine-3-carboxamide;
N-[(1S,2R,4R)-7-azabicyclo[2.2.1]hept-2-yl]pyrrolo[1,2-c]pyrimidine-3-carboxamide;
N-[(3R,4S)-1-azabicyclo[2.2.1]hept-3-yl]pyrrolo[1,2-c]pyrimidine-3-carboxamide;
N-[(2S,3R)-2-methyl-1-azabicyclo[2.2.2]oct-3-yl]pyrrolo[1,2-c]pyrimidine-3-carboxamide;
N-[(3R)-1-azabicyclo[2.2.2]oct-3-yl]pyrrolo[1,2-a]pyrazine-3-carboxamide;
N-[(3R,5R)-1-azabicyclo[3.2.1]oct-3-yl]pyrrolo[1,2-a]pyrazine-3-carboxamide;
N-[(1S,2R,4R)-7-azabicyclo[2.2.1]hept-2-yl]pyrrolo[1,2-a]pyrazine-3-carboxamide;
N-[(3R)-1-azabicyclo[2.2.2]oct-3-yl]-6-bromopyrrolo[1,2-a]pyrazine-3-carboxamide;
N-[(2S,3R)-2-methyl-1-azabicyclo[2.2.2]oct-3-yl]pyrrolo[1,2-a]pyrazine-3-carboxamide;
N-[(3R)-1-azabicyclo[2.2.2]oct-3-yl]-6-ethynylpyrrolo[1,2-a]pyrazine-3-carboxamide;
N-[(3R)-1-azabicyclo[2.2.2]oct-3-yl]indolizine-6-carboxamide; and
N-[(2S,3R)-2-methyl-1-azabicyclo[2.2.2]oct-3-yl]indolizine-6-carboxamide;
provided that the agonist is the free base or a pharmaceutically acceptable salt thereof.

31. A method of treating a mammal in need thereof, wherein the mammal would receive symptomatic relief by stimulating vascular angiogensis comprising the administration of an alpha 7 nAChR full agonist.

32. The method of claim 31, wherein the disease or condition is wound healing, healing bone fracture, ischemic heart disease, or stable angina pectoris.

33. The method of claim 31, wherein the wound is from surgery or burn.

34. The method of claim 31, wherein the full agonist is a compound of formula I:
Azabicyclo-N(R₁)—C(═X)—W Formula I
wherein Azabicyclo is 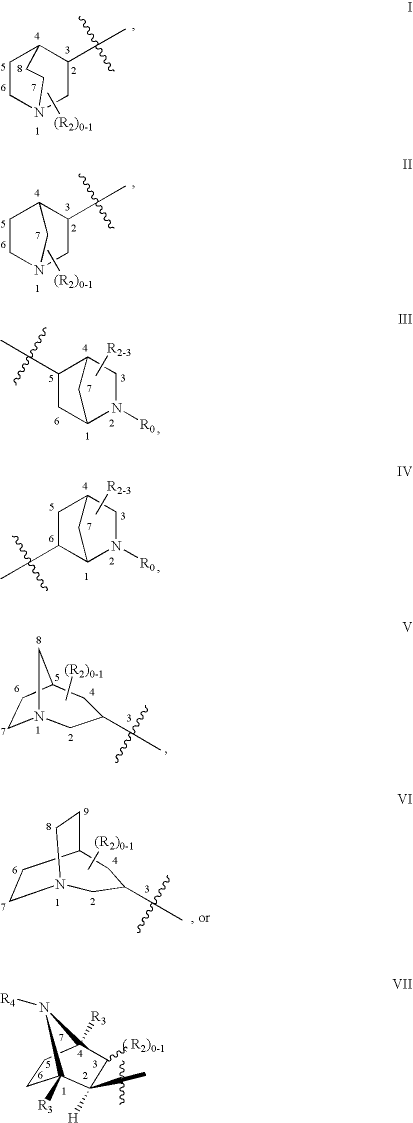
X is O, or S;
R₀ is H, lower alkyl, substituted lower alkyl, or lower haloalkyl;
Each R₁ is H, alkyl, cycloalkyl, haloalkyl, substituted phenyl, or substituted naphthyl;
Each R₂ is independently F, Cl, Br, I, alkyl, substituted alkyl, haloalkyl, cycloalkyl, aryl, or R₂ is absent;
R₂₋₃ is H, F, Cl, Br, I, alkyl, haloalkyl, substituted alkyl, cycloalkyl, or aryl;
Each R₃ is independently H, alkyl, or substituted alkyl;
R₄ is H, alkyl, an amino protecting group, or an alkyl group having 1-3 substituents selected from F, Cl, Br, I, —OH, —CN, —NH₂, —NH(alkyl), or —N(alkyl)₂;
R₅ is 5-membered heteroaromatic mono-cyclic moieties containing within the ring 1-3 heteroatoms independently selected from the group consisting of —O—, ═N—, —N(R₁₀)—, and —S—, and having 0-1 substituent selected from R⁹ and further having 0-3 substituents independently selected from F, Cl, Br, or I, or R₅ is 9-membered fused-ring moieties having a 6-membered ring fused to a 5-membered ring and having the formula 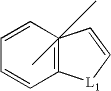
wherein L₁ is O, S, or NR₁₀, 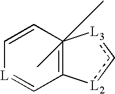
wherein L is CR₁₂ or N, L₂ and L₃ are independently selected from CR₁₂, C(R₁₂)₂, O, S, N, or NR₁₀, provided that both L₂ and L₃ are not simultaneously O, simultaneously S, or simultaneously O and S, or 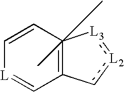
wherein L is CR₁₂ or N, and L₂ and L₃ are independently selected from CR₁₂, O, S, N, or NR₁₀, and each 9-membered fused-ring moiety having 0-1 substituent selected from R₉ and further having 0-3 substituent(s) independently selected from F, Cl, Br, or I, wherein the R₅ moiety attaches to other substituents as defined in formula I at any position as valency allows;
R₆ is 6-membered heteroaromatic mono-cyclic moieties containing within the ring 1-3 heteroatoms selected from ═N— and having 0-1 substituent selected from R₉ and 0-3 substituent(s) independently selected from F, Cl, Br, or I, or R₆ is 10-membered heteroaromatic bi-cyclic moieties containing within one or both rings 1-3 heteroatoms selected from ═N—, including, but not limited to, quinolinyl or isoquinolinyl, each 10-membered fused-ring moiety having 0-1 substituent selected from R₉ and 0-3 substituent(s) independently selected from F, Cl, Br, or I, wherein the R₆ moiety attaches to other substituents as defined in formula I at any position as valency allows;
R₇ is alkyl, substituted alkyl, haloalkyl, —OR₁₁, —CN, —NO₂, —N(R₈)₂;
Each R₈ is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;
R₉ is alkyl, cycloalkyl, heterocycloalkyl, haloalkyl, halocycloalkyl, haloheterocycloalkyl, —OR₁₄, —SR₁₄, —N(R₁₄)₂, —C(O)R₁₄, —C(O)N(R₁₄)₂, —CN, —NR₁₄C(O)R₁₄, —S(O)₂N(R₁₄)₂, —NR₁₄S(O)₂R₁₄, —NO₂, alkyl substituted with 1-4 substituent(s) independently selected from F, Cl, Br, I, or R₁₃, cycloalkyl substituted with 1-4 substituent(s) independently selected from F, Cl, Br, I, or R₁₃, or heterocycloalkyl substituted with 1-4 substituent(s) independently selected from F, Cl, Br, I, or R₁₃;
R₁₀ is H, alkyl, haloalkyl, substituted alkyl, cycloalkyl, halocycloalkyl, substituted cycloalkyl, phenyl, or phenyl having 1 substituent selected from R₇ and further having 0-3 substituents independently selected from F, Cl, Br, or I;
Each R₁₁ is independently H, alkyl, cycloalkyl, heterocycloalkyl, haloalkyl, halocycloalkyl, or haloheterocycloalkyl;
Each R₁₂ is independently H, F, Cl, Br, I, alkyl, cycloalkyl, heterocycloalkyl, haloalkyl, halocycloalkyl, haloheterocycloalkyl, substituted alkyl, substituted cycloalkyl, substituted heterocycloalkyl, —CN, —NO₂, —OR₁₄, —SR₁₄, —N(R₁₄)₂, —C(O)R₁₄, —C(O)N(R₁₄)₂, —NR₁₄C(O)R₁₄, —S(O)₂N(R₁₄)₂, —NR₁₄S(O)₂R₁₄, or a bond;
R₁₃ is —OR₁₄, —SR₁₄, —N(R₁₄)₂, —C(O)R₁₄, —C(O)N(R₁₄)₂, —CN, —CF₃, —NR₁₄C(O)R₁₄, —S(O)₂N(R₁₄)₂, —NR₁₄S(O)₂R₁₄, or —NO₂;
Each R₁₄ is independently H, alkyl, cycloalkyl, heterocycloalkyl, haloalkyl, halocycloalkyl, or haloheterocycloalkyl;
wherein W is (A): 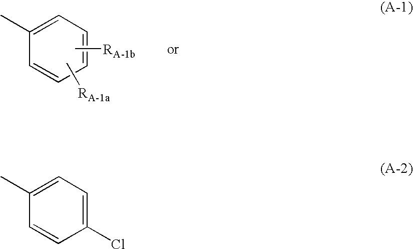
wherein R_{A-1a} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, —R₅, R₆, —OR_{A-3}, —SR_{A-3}, F, Cl, Br, I, —N(R_{A-3})₂, —C(O)R_{A-3}, —CN, —C(O)N(R_{A-3})₂, —NR_{A-3}C(O)R_{A-3}, —S(O)R_{A-3}, —OS(O)₂R_{A-3}, —NR_{A-3}S(O)₂R_{A-3}, —NO₂, and —N(H)C(O)N(H)R_{A-3};
R_{A-1b} is —O—R_{A-3}, —S—R_{A-3}, —S(O)—R_{A-3}, —C(O)—R_{A-7}, and alkyl substituted on the ω carbon with R_{A-7};
Each R_{A-3} is independently selected from H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
R_{A-4} is selected from cycloalkyl, halocycloalkyl, substituted cycloalkyl, heterocycloalkyl, haloheterocycloalkyl, or substituted heterocycloalkyl;
Each R_{A-5} is independently selected from cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{A-6} is independently selected from alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
R_{A-7} is selected from aryl, R₅, or R₆;
wherein W is (B): 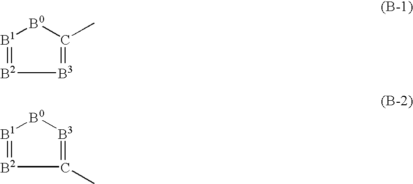
wherein B⁰ is —O—, —S—, or —N(R_{B-0})—;
B¹ and B² are independently selected from ═N—, or ═C(R_{B-1})—;
B³ is ═N—, or ═CH—, provided that when both B¹ and B² are ═C(R_{B-1})— and B³ is ═CH—, only one ═C(R_{B-1})— can be ═CH—, and further provided that when B⁰ is —O—, B² is ═C(R_{B-1})— and B³ is ═C(H)—, B¹ cannot be ═N—,
R_{B-0} is H, alkyl, cycloalkyl, heterocycloalkyl, haloalkyl, halocycloalkyl, haloheterocycloalkyl, substituted alkyl, substituted cycloalkyl, substituted heterocycloalkyl, or aryl, and provided that when B is (B-2) and B³ is ═N— and B⁰ is N(R_{B-0}), R_{B-0} cannot be phenyl or substituted phenyl;
R_{B-1} is H, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, —OR_{B-2}, —SR_{B-2}, F, Cl, Br, I, —N(R_{B-2})₂, —C(O)R_{B-2}, —C(O)N(R_{B-2})₂, —CN, —NR_{B-2}C(O)R_{B-4}, —S(O)₂N(R_{B-2})₂, —OS(O)₂R_{B-4}, —S(O)₂R_{B-2}, —NR_{B-2}S(O)₂R_{B-2}, —N(H)C(O)N(H)R_{B-2}, —NO₂, R₅, and R₆;
Each R_{B-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{B-3} is independently H, alkyl, haloalkyl, limited substituted alkyl, cycloalkyl, halocycloalkyl, substituted cycloalkyl, heterocycloalkyl, haloheterocycloalkyl, substituted heterocycloalkyl;
R_{B-4} is independently H, alkyl, cycloalkyl, heterocycloalkyl, haloalkyl, halocycloalkyl, or haloheterocycloalkyl;
wherein W is (C):
(C) is a six-membered heterocyclic ring system having 1-2 nitrogen atoms or a 10-membered bicyclic-six-six-fused-ring system having up to two nitrogen atoms within either or both rings, provided that no nitrogen is at a bridge of the bicyclic-six-six-fused-ring system, and further having 1-2 substitutents independently selected from R_{C-1};
Each R_{C-1} is independently H, F, Cl, Br, I, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, lactam heterocycloalkyl, phenyl, substituted phenyl, —NO₂, —CN, —OR_{C-2}, —SR_{C-2}, —SOR_{C-2}, —SO₂R_{C-2}, —NR_{C-2}C(O)R_{C-2}, —N(R_{C-2})₂, —C(O)R_{C-2}, —C(O)₂R_{C-2}, —C(O)N(R_{C-2})₂, —SCN, —S(O)N(R_{C-2})₂, —S(O)₂N(R_{C-2})₂, —NR_{C-2}S(O)₂R_{C-2}, R₅, or R₆;
Each R_{C-2} is independently H, alkyl, cycloalkyl, heterocycloalkyl, alkyl substituted with 1 substituent selected from R_{C-5}, cycloalkyl substituted with 1 substituent selected from R_{C-5}, heterocycloalkyl substituted with 1 substituent selected from R_{C-5}, haloalkyl, halocycloalkyl, haloheterocycloalkyl, phenyl, or substituted phenyl;
Each R_{C-3} is independently H, alkyl, or substituted alkyl;
R_{C-4} is H, alkyl, an amino protecting group, or an alkyl group having 1-3 substituents selected from F, Cl, Br, I, —OH, —CN, —NH₂, —NH(alkyl), or —N(alkyl)₂;
R_{C-5} is —CN, —CF₃, —NO₂, —OR_{C-6}, —SR_{C-6}, —N(R_{C-6})₂, —C(O)R_{C-6}, —SOR_{C-6}, —SO₂R_{C-6}, —C(O)N(R_{C-6})₂, —NR_{C-6}C(O)R_{C-6}, —S(O)₂N(R_{C-6})₂, or —NR_{C-6}S(O)₂R_{C-6};
Each R_{C-6} is independently H, alkyl, cycloalkyl, heterocycloalkyl, haloalkyl, halocycloalkyl, or haloheterocycloalkyl;
wherein W is (D): 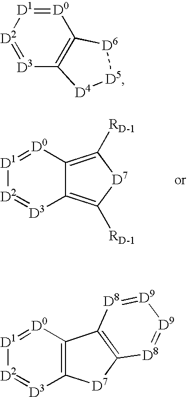
provided that the bond between the —C(═X)— group and the W group may be attached at any available carbon atom within the D group as provided in R_{D-1}, R_{D-3}, and R_{D-4};
D⁰, D¹, D², and D³ are N or C(R_{D-1}) provided that up to one of D⁰, D¹, D², or D³ is N and the others are C(R_{D-1}), further provided that when the core molecule is attached at D² and D⁰ or D¹ is N, D³ is C(H), and further provided that there is only one attachment to the core molecule;
D⁴ - - - D⁵ - - - D⁶ is selected from N(R_{D-2})—C(R_{D-3})═C(R_{D-3}), N═C(R_{D-3})—C(R_{D-4})₂, C(R_{D-3})═C(R_{D-3})—N(R_{D-2}), C(R_{D-3})₂—N(R_{D-2})—C(R_{D-3})₂, C(R_{D-4})₂—C(R_{D-3})═N, N(R_{D-2})—C(R_{D-3})₂—C(R_{D-3})₂, C(R_{D-3})₂—C(R_{D-3})₂—N(R_{D-2}), O—C(R_{D-3})═C(R_{D-3}), O—C(R_{D-3})₂—C(R_{D-3})₂, C(R_{D-3})₂—O—C(R_{D-3})₂, C(R_{D-3})═C(R_{D-3})—O, C(R_{D-3})₂—C(R_{D-3})₂-O, S—C(R_{D-3})═C(R_{D-3}), S—C(R_{D-3})₂—C(R_{D-3})₂, C(R_{D-3})₂—S—C(R_{D-3})₂, C(R_{D-3})═C(R_{D-3})—S, or C(R_{D-3})₂—C(R_{D-3})₂—S;
provided that when C(X) is attached to W at D² and D⁶ is O, N(R_{D-2}), or S, D⁴ - - - D⁵ is not CH═CH;
and further provided that when C(X) is attached to W at D² and D⁴ is O, N(R_{D-2}), or S, D⁵ - - - D⁶ is not CH═CH;
Each R_{D-1} is independently H, F, Br, I, Cl, —CN, —CF₃, —OR_{D-5}, —SR_{D-5}, —N(R_{D-5})₂, or a bond to —C(X)— provided that only one of R_{D-1}, R_{D-3}, and R_{D-4} is said bond;
Each R_{D-2} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, or R₆;
Each R_{D-3} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-10})₂, —SR_{D-10}, —S(O)₂R_{D-10}, —C(O)R_{D-12}, —CO₂R_{D-10}, aryl, R₅, R₆, or a bond to —C(X)— provided that only one of R_{D-1}, R_{D-3}, and R_{D-4} is said bond;
Each R_{D-4} is independently H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{D-10}, —C(O)N(R_{D-11})₂, —NR_{D-10}COR_{D-12}, —N(R_{D-11})₂, —SR_{D-10}, —CO₂R_{D-10}, aryl, R₅, R₆, or a bond to —C(X)— provided that only one of R_{D-1}, R_{D-3}, and R_{D-4} is said bond;
Each R_{D-5} is independently H, C₁₋₃ alkyl, or C₂₋₄ alkenyl;
D⁷ is O, S, or N(R_{D-2});
D⁸ and D⁹ are C(R_{D-1}), provided that when the molecule is attached to the phenyl moiety at D⁹, D⁸ is CH;
Each R_{D-10} is H, alkyl, cycloalkyl, haloalkyl, substituted phenyl, or substituted naphthyl;
Each R_{D-11} is independently H, alkyl, cycloalkyl, heterocycloalkyl, alkyl substituted with 1 substituent selected from R₁₃, cycloalkyl substituted with 1 substituent selected from R₁₃, heterocycloalkyl substituted with 1 substituent selected from R₁₃, haloalkyl, halocycloalkyl, haloheterocycloalkyl, phenyl, or substituted phenyl;
R_{D-12} is H, alkyl, substituted alkyl, cycloalkyl, haloalkyl, heterocycloalkyl, substituted heterocycloalkyl, substituted phenyl, or substituted naphthyl;
wherein W is (E): 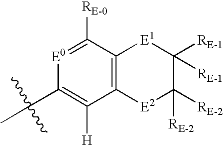
E⁰ is CH or N;
R_{E-0} is H, F, Cl, Br, I, alkyl, alkenyl, alkynyl, cycloalkyl, heterocycloalkyl, haloalkyl, haloalkenyl, haloalkynyl, halocycloalkyl, haloheterocycloalkyl, substituted alkyl, substituted alkenyl, substituted alkynyl, substituted cycloalkyl, substituted heterocycloalkyl, aryl, R₅, R₆, —OR_{E-3}, —SR_{E-3}, —N(R_{E-3})₂, —C(O)R_{E-3}, —CN, —C(O)N(R_{E-3})₂, —NR_{E-3}C(O)R_{E-3}, —S(O)R_{E-3}, —S(O)R_{E-5}, —OS(O)₂R_{E-3}, —NR_{E-3}S(O)₂R_{E-3}, —NO₂, or —N(H)C(O)N(H)R_{E-3};
E¹ is O, CR_{E-1-1}, or C(R_{E-1-1})₂, provided that when E¹ is CR_{E-1-1}, one R_{E-1} is a bond to CR_{E-1-1}, and further provided that at least one of E¹ or E² is O;
Each R_{E-1-1} is independently H, F, Br, Cl, CN, alkyl, haloalkyl, substituted alkyl, alkynyl, cycloalkyl, —OR_{E}, or —N(R_{E})₂, provided that at least one R_{E-1-1} is H when E¹ is C(R_{E-1-1})₂;
Each R_{E-1} is independently H, alkyl, substituted alkyl, haloalkyl, cycloalkyl, heterocycloalkyl, or a bond to E¹ provided that E¹ is CR_{E-1-1};
E² is O, CR_{E-2-2}, or C(R_{E-2-2})₂, provided that when E² is CR_{E-2-2}, one R_{E-2} is a bond to CR_{E-2-2}, and further provided that at least one of E¹ or E² is O;
Each R_{E-2-2} is independently H, F, Br, Cl, CN, alkyl, haloalkyl, substituted alkyl, alkynyl, cycloalkyl, —OR_{E}, or —N(R_{E})₂, provided that at least one R_{E-2-2} is H when E² is C(R_{E-2-2})₂;
Each R_{E-2} is independently H, alkyl, substituted alkyl, haloalkyl, cycloalkyl, heterocycloalkyl, or a bond to E² provided that E² is CR_{E-2-2};
Each R_{E} is independently H, alkyl, cycloalkyl, heterocycloalkyl, haloalkyl, halocycloalkyl, or haloheterocycloalkyl;
Each R_{E-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I or substituted phenyl;
R_{E-4} is H, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or substituted phenyl;
Each R_{E-5} is independently H, haloalkyl, substituted alkyl, cycloalkyl, halocycloalkyl, substituted cycloalkyl, heterocycloalkyl, haloheterocycloalkyl, substituted heterocycloalkyl, R₅, or R₆;
Each R_{E-6} is independently alkyl, haloalkyl, substituted alkyl, cycloalkyl, halocycloalkyl, substituted cycloalkyl, heterocycloalkyl, haloheterocycloalkyl, substituted heterocycloalkyl, R₅, R₆, phenyl, or phenyl having 1 substituent selected from R₉ and further having 0-3 substituents independently selected from F, Cl, Br, or I;
wherein W is (F): 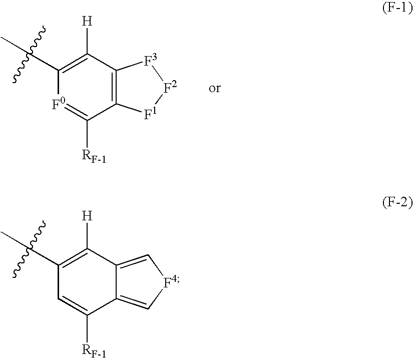
F⁰ is C(H) wherein F¹ - - - F² - - - F³ is selected from O—C(R_{F-2})═N, O—C(R_{F-3})(R_{F-2})—N(R_{F-4}), O—C(R_{F-3})(R_{F-2})—S, O—N═C(R_{F-3}), O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂—O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), or C(R_{F-3})₂—C(R_{F-2})(R_{F-3})—C(R_{F-3})₂;
F⁰ is N wherein F¹ - - - F² - - - F³ is selected from O—C(R_{F-2})═N, O—C(R_{F-3})(R_{F-2})—N(R_{F-4}), O—C(R_{F-3})(R_{F-2})—S, O—N═C(R_{F-3}) O—C(R_{F-2})(R_{F-3})—O, S—C(R_{F-2})═N, S—C(R_{F-3})(R_{F-2})—N(R_{F-4}), S—N═C(R_{F-3}), N═C(R_{F-2})—O, N═C(R_{F-2})—S, N═C(R_{F-2})—N(R_{F-4}), N(R_{F-4})—N═C(R_{F-3}), N(R_{F-4})—C(R_{F-3})(R_{F-2})—O, N(R_{F-4})—C(R_{F-3})(R_{F-2})—S, N(R_{F-4})—C(R_{F-3})(R_{F-2})—N(R_{F-4}), C(R_{F-3})₂-O—N(R_{F-4}), C(R_{F-3})₂—N(R_{F-4})—O, C(R_{F-3})₂—N(R_{F-4})—S, C(R_{F-3})═N—O, C(R_{F-3})═N—S, C(R_{F-3})═N—N(R_{F-4}), C(R_{F-3})═C(R_{F-2})—C(R_{F-3})₂, or C(R_{F-3})₂—C(R_{F-2})(R_{F-3})—C(R_{F-3})₂;
F⁴ is N(R_{F-7}), O, or S;
R_{F-1} is H, F, Cl, Br, I, —CN, —CF₃, —OR_{F-8}, —SR_{F-8}, or —N(R_{F-8})₂;
R_{F-2} is H, F, alkyl, haloalkyl, substituted alkyl, lactam heterocycloalkyl, phenoxy, substituted phenoxy, R₅, R₆, —N(R_{F-4})-aryl, —N(R_{F-4})-substituted phenyl, —N(R_{F-4})-substituted naphthyl, —O-substituted phenyl, —O-substituted naphthyl, —S-substituted phenyl, —S-substituted naphthyl, or alkyl substituted on the ω carbon with R_{F-9};
R_{F-3} is H, F, Br, Cl, I, alkyl, substituted alkyl, haloalkyl, alkenyl, substituted alkenyl, haloalkenyl, alkynyl, substituted alkynyl, haloalkynyl, heterocycloalkyl, substituted heterocycloalkyl, lactam heterocycloalkyl, —CN, —NO₂, —OR_{F-8}, —C(O)N(R_{F-8})₂, —NHR_{F-8}, —NR_{F-8}COR_{F-8}, —N(R_{F-8})₂, —SR_{F-8}, —C(O)R_{F-8}, —CO₂R_{F-8}, aryl, R₅, or R₆;
R_{F-4} is H, or alkyl;
Each R_{F-5} is independently F, Br, Cl, I, alkyl, substituted alkyl, haloalkyl, alkenyl, substituted alkenyl, haloalkenyl, alkynyl, substituted alkynyl, haloalkynyl, —CN, —CF₃, —OR_{F-8}, —C(O)NH₂, —NHR_{F-8}, —SR_{F-8}, —CO₂R_{F-8}, aryl, phenoxy, substituted phenoxy, heteroaryl, —N(R_{F-4})-aryl, or —O-substituted aryl;
One of R_{F-6} is H, alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆, and each of the other two R_{F-6} is independently selected from alkyl, substituted alkyl, haloalkyl, alkenyl, substituted alkenyl, haloalkenyl, alkynyl, substituted alkynyl, haloalkynyl, —CN, F, Br, Cl, I, —OR_{F-8}, —C(O)NH₂, —NHR_{F-8}, —SR_{F-8}, —CO₂R_{F-8}, aryl, R₅, or R₆;
R_{F-7} is H, alkyl, haloalkyl, substituted alkyl, cycloalkyl, halocycloalkyl, substituted cycloalkyl, phenyl, or phenyl having 1 substituent selected from R₉ and further having 0-3 substituents independently selected from F, Cl, Br, or I;
R_{F-8} is H, alkyl, substituted alkyl, cycloalkyl, haloalkyl, heterocycloalkyl, substituted heterocycloalkyl, substituted phenyl, or substituted naphthyl;
R_{F-9} is aryl, R₅, or R₆;
wherein W is (G): 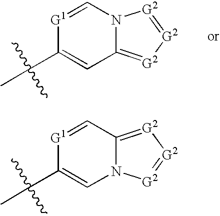
G¹ is N or CH;
Each G² is N or C(R_{G-1}), provided that no more than one G² is N;
Each R_{G-1} is independently H, alkyl, substituted alkyl, haloalkyl, alkenyl, substituted alkenyl, haloalkenyl, alkynyl, substituted alkynyl, haloalkynyl, —CN, —NO₂, F, Br, Cl, I, —C(O)N(R_{G-3})₂, —N(R_{G-3})₂, —SR_{G-6}, —S(O)₂R_{G-6}, —OR_{G-6}, —C(O)R_{G-6}, —CO₂R_{G-6}, aryl, R₅, R₆, or two R_{G-1} on adjacent carbon atoms may combine for W to be a 6-5-6 fused-tricyclic-heteroaromatic-ring system optionally substituted on the newly formed ring where valency allows with 1-2 substitutents independently selected from F, Cl, Br, I, and R_{G-2};
R_{G-2} is alkyl, alkenyl, alkynyl, cycloalkyl, heterocycloalkyl, haloalkyl, haloalkenyl, haloalkynyl, halocycloalkyl, haloheterocycloalkyl, —OR_{G-8}, —SR_{G-8}, —S(O)₂R_{G-8}, —S(O)R_{G-8}, —OS(O)₂R_{G-8}, —N(R_{G-8})₂, —C(O)R_{G-8}, —C(S)R_{G-8}, —C(O)OR_{G-8}, —CN, —C(O)N(R_{G-8})₂, —NR_{G-8}C(O)R_{G-8}, —S(O)₂N(R_{G-8})₂, —NR_{G-8}S(O)₂R_{G-8}, —NO₂, —N(R_{G-8})C(O)N(R_{G-8})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R_{G-7}, naphthyl, or naphthyl having 0-4 substituents independently selected from F, Cl, Br, I, or R_{G-7};
provided that when G² adjacent to the bridge N is C(R_{G-1}) and the other G² are CH, that R_{G-1} is other than H, F, Cl, I, alkyl, substituted alkyl or alkynyl;
Each R_{G-3} is independently H, alkyl, cycloalkyl, heterocycloalkyl, alkyl substituted with 1 substituent selected from R_{G-4}, cycloalkyl substituted with 1 substituent selected from R_{G-4}, heterocycloalkyl substituted with 1 substituent selected from R_{G-4}, haloalkyl, halocycloalkyl, haloheterocycloalkyl, phenyl, or substituted phenyl;
R_{G-4} is —OR_{G-5}, —SR_{G-5}, —N(R_{G-5})₂, —C(O)R_{G-5}, —SOR_{G-5}, —SO₂R_{G-5}, —C(O)N(R_{G-5})₂, —CN, —CF₃, —NR_{G-5}C(O)R_{G-5}, —S(O)₂N(R_{G-5})₂, —NR_{G-5}S(O)₂R_{G-5}, or —NO₂;
Each R_{G-5} is independently H, alkyl, cycloalkyl, heterocycloalkyl, haloalkyl, halocycloalkyl, or haloheterocycloalkyl;
R_{G-6} is H, alkyl, haloalkyl, substituted alkyl, cycloalkyl, halocycloalkyl, substituted cycloalkyl, phenyl, or phenyl having 0-4 substituents independently selected from F, Cl, Br, I, and R_{G-7};
R_{G-7} is alkyl, substituted alkyl, haloalkyl, —OR_{G-5}, —CN, —NO₂, —N(R_{G-3})₂;
Each R_{G-8} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R_{G-7};
wherein W is (H) 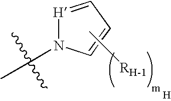
H′ is N or CH;
Each R_{H-1} is independently F, Cl, Br, I, —CN, —NO₂, alkyl, haloalkyl, substituted alkyl, alkenyl, haloalkenyl, substituted alkenyl, alkynyl, haloalkynyl, substituted alkynyl, cycloalkyl, halocycloalkyl, substituted cycloalkyl, heterocycloalkyl, haloheterocycloalkyl, substituted heterocycloalkyl, lactam heterocyclcoalkyl, aryl, R₅, R₆, —OR₈, —SR₈, —SOR₈, —SO₂R₈, —SCN, —S(O)N(R₈)₂, —S(O)₂N(R₈)₂, —C(O)R₈, —C(O)₂R₈, —C(O)N(R₈)₂, C(R₈)═N—OR₈, —NC(O)R₅, —NC(O)R_{H-3}, —NC(O)R₆, —N(R₈)₂, —NR₈C(O)R₈, —NR₈S(O)₂R₈, or two R_{H-1} on adjacent carbon atoms may fuse to form a 6-membered ring to give a 5-6 fused, bicyclic moiety where the 6-membered ring is optionally substituted with 1-3 substitutents selected from R_{H-2};
m_{H} is 0, 1, or 2;
R_{H-2} is alkyl, alkenyl, alkynyl, cycloalkyl, heterocycloalkyl, haloalkyl, haloalkenyl, haloalkynyl, halocycloalkyl, haloheterocycloalkyl, —OR_{H-3}, —SR_{H-3}, —S(O)₂R_{H-3}, —S(O)R_{H-3}, —OS(O)₂R_{H-3}, —N(R_{H-3})₂, —C(O)R_{H-3}, —C(S)R_{H-3}, —C(O)OR_{H-3}, —CN, —C(O)N(R_{H-3})₂, —NR_{H-3}C(O)R_{H-3}, —S(O)₂N(R_{H-3})₂, —NR_{H-3}S(O)₂R_{H-3}, —NO₂, —N(R_{H-3})C(O)N(R_{H-3})₂, substituted alkyl, substituted alkenyl, substituted alkynyl, substituted cycloalkyl, substituted heterocycloalkyl, lactam heterocycloalkyl, phenyl, phenyl having 0-4 substituents independently selected from F, Cl, Br, I and R₇, naphthyl, naphthyl having-g 0-4 suibstituents independently selected from F, Cl, Br, I, or R₇, or two R_{H-2} on adjacent carbon atoms may combine to form a three-ring-fused-5-6-6 system optionally substituted with up to 3 substituents independently selected from Br, Cl, F, I, —CN, —NO₂, —CF₃, —N(R_{H-3})₂, —N(R_{H-3})C(O)R_{H-3}, alkyl, alkenyl, and alkynyl;
Each R_{H-3} is independently H, alkyl, haloalkyl, substituted alkyl, cycloalkyl, halocycloalkyl, substituted cycloalkyl, heterocycloalkyl, haloheterocycloalkyl, substituted heterocycloalkyl, phenyl, or phenyl substituted with 0-4 independently selected from F, Cl, Br, I, or R₇;
or pharmaceutically acceptable salt, racemic mixture, or pure enantiomer thereof.

35. The method of claim 34, wherein the disease or condition is wound healing, healing bone fracture, ischemic heart disease, or stable angina pectoris.

36. The method of claim 34, wherein the wound is from surgery or burn.

37. The method of claim 34, wherein X is O and R₁ is H.

38. The method of claim 37, wherein W is (C), (D), (E), (F), and (G).

39. The method of claim 38, wherein Azabicyclo is I, II, V and VII.

40. The method of claim 39, wherein W is 4-chlorophen-1-yl; dibenzo[b,d]thiophene-2-yl; isoquinoline-3-yl; furo[2,3-c]pyridine-5-yl; furo[3,2-c]pyridine-6; 1,3-benzodioxole-5-yl; 2,3-dihydro-1,4-benzodioxine-6-yl; 2,3-dihydro-1,4-benzodioxine-7-yl; 1,3-benzoxazole-5-yl; thieno[2,3-c]pyridine-5-yl; thieno[3,2-c]pyridine-6-yl; [1]benzothieno[3,2-c]pyridine-3-yl; 1,3-benzothiazole-6-yl; thieno[3,4-c]pyridine-6-yl; 2,3-dihydro-1-benzofuran-5-yl; 1-benzofuran-5-yl; furo[3,2-c]pyridine-6-yl; [1]benzothieno[2,3-c]pyridine-3-yl; dibenzo[b,d]furan-2-yl; 1-benzofuran-6-yl; 2-naphthyl; 1H-indole-6-yl; pyrrolo[1,2-c]pyrimidine-3-yl; 1-benzothiophene-5-yl; 1-benzothiophene-5-yl; 1-benzothiophene-6-yl; pyrrolo[1,2-a]pyrazine-3-yl; 1H-indole-6-yl; pyrazino[1,2-a]indole-3-yl; 1,3-benzothiazole-6-yl; [1]benzofuro[2,3-c]pyridine-3-yl; [1]benzofuro[2,3-c]pyridine-3-yl; 2H-chromene-6-yl; indolizine-6-yl; and [1,3]dioxolo[4,5-c]pyridine-6-yl; any of which is optionally substituted as allowed in claim 39.

41. The method of claim 40, wherein the agonist is
N-[(3R)-1-azabicyclo[2.2.2]oct-3-yl]-4-chlorobenzamide;
N-[(3R)-1-azabicyclo[2.2.2]oct-3-yl]dibenzo[b,d]thiophene-2-carboxamide;
N-[(3R)-1-azabicyclo[2.2.2]oct-3-yl]isoquinoline-3-carboxamide;
N-[(3R)-1-azabicyclo[2.2.2]oct-3-yl]furo[2,3-c]pyridine-5-carboxamide;
N-[(3R)-1-azabicyclo[2.2.2]oct-3-yl]-1,3-benzodioxole-5-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-2,3-dihydro-1,4-benzodioxine-6-carboxamide;
N-[(3R)-1-azabicyclo[2.2.2]oct-3-yl]-3-methylfuro[2,3-c]pyridine-5-carboxamide;
N-[(1S,2R,4R)-7-azabicyclo[2.2.1]hept-2-yl]isoquinoline-3-carboxamide;
N-[(1S,2R,4R)-7-azabicyclo[2.2.1]hept-2-yl]-3-methylfuro[2,3-c]pyridine-5-carboxamide;
N-[(3R)-1-azabicyclo[2.2.2]oct-3-yl]-1,3-benzoxazole-5-carboxamide;
N-[(3R)-1-azabicyclo[2.2.2]oct-3-yl]-2-methyl-1,3-benzoxazole-5-carboxamide;
N-[(1S,2R,4R)-7-azabicyclo[2.2.1]hept-2-yl]thieno[2,3-c]pyridine-5-carboxamide;
N-[(1S,2R,4R)-7-azabicyclo[2.2.1]hept-2-yl]thieno[3,2-c]pyridine-6-carboxamide;
N-[(1S,2R,4R)-7-azabicyclo[2.2.1]hept-2-yl]furo[2,3-c]pyridine-5-carboxamide;
N-[(3R)-1-azabicyclo[2.2.2]oct-3-yl]-3-ethylfuro[2,3-c]pyridine-5-carboxamide;
N-[(3R)-1-azabicyclo[2.2.2]oct-3-yl]-3-isopropylfuro[2,3-c]pyridine-5-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
5-{[(2R)-7-azoniabicyclo[2.2.1]hept-2-ylamino]carbonyl}-3-ethylfuro[2,3-c]pyridin-6-ium dichloride;
5-{[(2R)-7-azoniabicyclo[2.2.1]hept-2-ylamino]carbonyl}-3-isopropylfuro[2,3-c]pyridin-6-ium dichloride;
N-[(3R,4S)-1-azabicyclo[2.2.1]hept-3-yl]furo[2,3-c]pyridine-5-carboxamide;
N-1-azabicyclo[2.2.2]oct-3-yl [1]benzothieno[3,2-c]pyridine-3-carboxamide;
N-[(3R)-1-azabicyclo[2.2.2]oct-3-yl]-1,3-benzothiazole-6-carboxamide;
N-[(3R)-1-azabicyclo[2.2.2]oct-3-yl]-3-chlorofuro[2,3-c]pyridine-5-carboxamide;
N-1-azabicyclo[2.2.2]oct-3-ylfuro[2,3-c]pyridine-5-carboxamide;
N-[(3R)-1-azabicyclo[2.2.2]oct-3-yl]thieno[3,4-c]pyridine-6-carboxamide;
N-[(3R,5R)-1-azabicyclo[3.2.1]oct-3-yl]-3-methylfuro[2,3-c]pyridine-5-carboxamide;
N-[(3R,4S)-1-azabicyclo[2.2.1]hept-3-yl]-3-methylfuro[2,3-c]pyridine-5-carboxamide;
N-[(3R)-1-azabicyclo[2.2.2]oct-3-yl]-2,3-dihydro-1-benzofuran-5-carboxamide;
N-[(3R,4S)-1-azabicyclo[2.2.1]hept-3-yl]thieno[2,3-c]pyridine-5-carboxamide;
N-[(3R)-1-azabicyclo[2.2.2]oct-3-yl]-1-benzofuran-5-carboxamide;
N-[(3R)-1-azabicyclo[2.2.2]oct-3-yl]furo[3,2-c]pyridine-6-carboxamide;
N-[(3R,4S)-1-azabicyclo[2.2.1]hept-3-yl]thieno[3,2-c]pyridine-6-carboxamide;
N-[(3R,4S)-1-azabicyclo[2.2.1]hept-3-yl]3-ethylfuro[2,3-c]pyridine-5-carboxamide;
N-[(3R,4S)-1-azabicyclo[2.2.1]hept-3-yl]3-isopropylfuro[2,3-c]pyridine-5-carboxamide;
N-[(1S,2R,4R)-7-azabicyclo[2.2.1]hept-2-yl]-3-chlorofuro[2,3-c]pyridine-5-carboxamide;
N-[(3R,4S)-1-azabicyclo[2.2.1]hept-3-yl]3-chlorofuro[2,3-c]pyridine-5-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]furo[3,2-c]pyridine-6-carboxamide;
N-[(3R,5R)-1-azabicyclo[3.2.1]oct-3-yl]-4-chlorobenzamide;
N-[(1S,2R,4R)-7-azabicyclo[2.2.1]hept-2-yl]thieno[3,4-c]pyridine-6-carboxamide;
N-[(1S,2R,4R)-7-azabicyclo[2.2.1]hept-2-yl]dibenzo[b,d]thiophene-2-carboxamide;
N-[(3R,4S)-1-azabicyclo[2.2.1]hept-3-yl]-1-benzofuran-5-carboxamide;
N-[(3R)-1-azabicyclo[2.2.2]oct-3-yl][1]benzothieno[2,3-c]pyridine-3-carboxamide;
N-[(1S,2R,4R)-7-azabicyclo[2.2.1]hept-2-yl][1]benzothieno[2,3-c]pyridine-3-carboxamide;
N-[(1S,2R,4R)-7-azabicyclo[2.2.1]hept-2-yl]-1-benzofuran-5-carboxamide;
N-[(1S,2R,4R)-7-azabicyclo[2.2.1]hept-2-yl]dibenzo[b,d]furan-2-carboxamide;
N-[(3R,5R)-1-azabicyclo[3.2.1]oct-3-yl]furo[2,3-c]pyridine-5-carboxamide;
N-[(3R,5R)-1-azabicyclo[3.2.1]oct-3-yl]furo[3,2-c]pyridine-6-carboxamide;
N-[(3R,5R)-1-azabicyclo[3.2.1]oct-3-yl]-1-benzofuran-5-carboxamide;
N-[(3R)-1-azabicyclo[2.2.2]oct-3-yl]-3-bromofuro[2,3-c]pyridine-5-carboxamide;
N-[(1S,2R,4R)-7-azabicyclo[2.2.1]hept-2-yl]-3-bromofuro[2,3-c]pyridine-5-carboxamide;
N-[(3R)-1-azabicyclo[2.2.2]oct-3-yl]-1-benzofuran-6-carboxamide;
N-[(2S,3R)-2-methyl-1-azabicyclo[2.2.2]oct-3-yl]-2-naphthamide;
N-[(3R)-1-azabicyclo[2.2.2]oct-3-yl]pyrrolo[1,2-c]pyrimidine-3-carboxamide;
N-[(3R,5R)-1-azabicyclo[3.2.1]oct-3-yl]thieno[2,3-c]pyridine-5-carboxamide;
N-[(3R,5R)-1-azabicyclo[3.2.1]oct-3-yl]thieno[3,2-c]pyridine-6-carboxamide;
N-[(3R,4S)-1-azabicyclo[2.2.1]hept-3-yl]-1H-indole-6-carboxamide;
N-[(2S,3R)-2-methyl-1-azabicyclo[2.2.2]oct-3-yl]thieno[2,3-c]pyridine-5-carboxamide;
3-methyl-N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]-1-benzofuran-5-carboxamide;
N-[(2S,3R)-2-methyl-1-azabicyclo[2.2.2]oct-3-yl]thieno[3,2-c]pyridine-6-carboxamide;
N-[(2S,3R)-2-methyl-1-azabicyclo[2.2.2]oct-3-yl]pyrrolo[1,2-c]pyrimidine-3-carboxamide;
N-[(2S,3R)-2-methyl-1-azabicyclo[2.2.2]oct-3-yl]-1,3-benzothiazole-6-carboxamide;
N-[(3R,5R)-1-azabicyclo[3.2.1]oct-3-yl]pyrrolo[1,2-c]pyrimidine-3-carboxamide;
N-[(3R)-1-azabicyclo[2.2.2]oct-3-yl]-1-benzothiophene-5-carboxamide;
N-[(1S,2R,4R)-7-azabicyclo[2.2.1]hept-2-yl]pyrrolo[1,2-c]pyrimidine-3-carboxamide;
N-[(3R,4S)-1-azabicyclo[2.2.1]hept-3-yl]pyrrolo[1,2-c]pyrimidine-3-carboxamide;
N-[(3R,4S)-1-azabicyclo[2.2.1]hept-3-yl]-3-bromofuro[2,3-c]pyridine-5-carboxamide;
N-[(3R,4S)-1-azabicyclo[2.2.1]hept-3-yl]-1,3-benzodioxole-5-carboxamide;
N-[(3R)-1-azabicyclo[2.2.2]oct-3-yl]-3-bromo-1-benzofuran-5-carboxamide;
N-[(1S,2R,4R)-7-azabicyclo[2.2.1]hept-2-yl]-3-bromo-1-benzofuran-5-carboxamide;
N-[(3R)-1-azabicyclo[2.2.2]oct-3-yl]-3-bromothieno[2,3-c]pyridine-5-carboxamide;
N-[(1S,2R,4R)-7-azabicyclo[2.2.1]hept-2-yl]-3-bromothieno[2,3-c]pyridine-5-carboxamide;
N-[(3R,4S)-1-azabicyclo[2.2.1]hept-3-yl]-1-benzothiophene-5-carboxamide;
N-[(3S)-1-azabicyclo[2.2.2]oct-3-yl]furo[2,3-c]pyridine-5-carboxamide;
N-[(3R)-1-azabicyclo[2.2.2]oct-3-yl]-3-methyl-1-benzofuran-5-carboxamide;
N-[(1S,2R,4R)-7-azabicyclo[2.2.1]hept-2-yl]-3-methyl-1-benzofuran-5-carboxamide;
N-[(3R)-1-azabicyclo[2.2.2]oct-3-yl]-2-methyl-1-benzofuran-6-carboxamide;
N-[(3R,5R)-1-azabicyclo[3.2.1]oct-3-yl]-1-benzofuran-6-carboxamide;
N-[(2S,3R)-2-methyl-1-azabicyclo[2.2.2]oct-3-yl]-1-benzofuran-6-carboxamide;
N-[(2S,3R)-2-methyl-1-azabicyclo[2.2.2]oct-3-yl]-1-benzothiophene-5-carboxamide;
N-[(3R)-1-azabicyclo[2.2.2]oct-3-yl]-1-benzothiophene-6-carboxamide;
N-[(3R)-1-azabicyclo[2.2.2]oct-3-yl]pyrrolo[1,2-a]pyrazine-3-carboxamide;
N-[(3R,4S)-1-azabicyclo[2.2.1]hept-3-yl]-1-benzothiophene-6-carboxamide;
N-[(3R)-1-azabicyclo[2.2.2]oct-3-yl]-1-methyl-1H-indole-6-carboxamide;
N-[(3S)-1-azabicyclo[2.2.2]oct-3-yl]-1-benzofuran-5-carboxamide;
N-[(3R)-1-azabicyclo[2.2.2]oct-3-yl]-3-isopropyl-1-benzofuran-5-carboxamide;
N-[(1S,2R,4R)-7-azabicyclo[2.2.1]hept-2-yl]-3-isopropyl-1-benzofuran-5-carboxamide;
N-[(3R)-1-azabicyclo[2.2.2]oct-3-yl]-3-ethynylfuro[2,3-c]pyridine-5-carboxamide;
N-[(3R)-1-azabicyclo[2.2.2]oct-3-yl]-1H-indazole-6-carboxamide;
N-[(3R)-1-azabicyclo[2.2.2]oct-3-yl]-2-methyl-1-benzofuran-5-carboxamide;
N-[(1S,2R,4R)-7-azabicyclo[2.2.1]hept-2-yl]-2-methyl-1-benzofuran-5-carboxamide;
N-[(3R)-1-azabicyclo[2.2.2]oct-3-yl]pyrazino[1,2-a]indole-3-carboxamide;
33-bromo-N-[(2S,3R)-2-methyl-1-azabicyclo[2.2.2]oct-3-yl]furo[2,3-c]pyridine-5-carboxamide;
N-[(3R,5R)-1-azabicyclo[3.2.1]oct-3-yl]pyrrolo[1,2-a]pyrazine-3-carboxamide;
N-[(3R)-1-azabicyclo[2.2.2]oct-3-yl]-7-methoxy-2-naphthamide;
N-[(1S,2R,4R)-7-azabicyclo[2.2.1]hept-2-yl]pyrrolo[1,2-a]pyrazine-3-carboxamide;
N-[(3R,5R)-1-azabicyclo[3.2.1]oct-3-yl]-1,3-benzothiazole-6-carboxamide;
N-[(3R,4S)-1-azabicyclo[2.2.1]hept-3-yl]-3-bromo-1-benzofuran-6-carboxamide;
N-[(3R)-1-azabicyclo[2.2.2]oct-3-yl][1]benzofuro[2,3-c]pyridine-3-carboxamide;
N-[(1S,2R,4R)-7-azabicyclo[2.2.1]hept-2-yl][1]benzofuro[2,3-c]pyridine-3-carboxamide;
N-[(3R)-1-azabicyclo[2.2.2]oct-3-yl]-3-ethynyl-1-benzofuran-5-carboxamide;
N-[(1S,2R,4R)-7-azabicyclo[2.2.1]hept-2-yl]-3-ethynyl-1-benzofuran-5-carboxamide;
N-[(3R)-1-azabicyclo[2.2.2]oct-3-yl]-2H-chromene-6-carboxamide;
N-[(3R)-1-azabicyclo[2.2.2]oct-3-yl]-3-prop-1-ynyl-1-benzofuran-5-carboxamide;
N-[(3R)-1-azabicyclo[2.2.2]oct-3-yl]-2-phenyl-1,3-benzodioxole-5-carboxamide;
N-[(3R)-1-azabicyclo[2.2.2]oct-3-yl]-6-bromopyrrolo[1,2-a]pyrazine-3-carboxamide;
N-[(3R)-1-azabicyclo[2.2.2]oct-3-yl]-3-prop-1-ynylfuro[2,3-c]pyridine-5-carboxamide;
N-[(2S,3R)-2-methyl-1-azabicyclo[2.2.2]oct-3-yl]pyrrolo[1,2-a]pyrazine-3-carboxamide;
N-[(3R)-1-azabicyclo[2.2.2]oct-3-yl]indolizine-6-carboxamide;
2-amino-N-[(3R)-1-azabicyclo[2.2.2]oct-3-yl]-1,3-benzothiazole-6-carboxamide;
N-[(3R)-1-azabicyclo[2.2.2]oct-3-yl]-6-ethynylpyrrolo[1,2-a]pyrazine-3-carboxamide;
N-[(3R)-1-azabicyclo[2.2.2]oct-3-yl]-8-methoxy-2-naphthamide;
N-[(2S,3R)-2-methyl-1-azabicyclo[2.2.2]oct-3-yl]indolizine-6-carboxamide;
N-[(3R)-1-azabicyclo[2.2.2]oct-3-yl][1,3]dioxolo[4,5-c]pyridine-6-carboxamide;
N-[(1S,2R,4R)-7-azabicyclo[2.2.1]hept-2-yl][1,3]dioxolo[4,5-c]pyridine-6-carboxamide;
N-[(3R)-1-azabicyclo[2.2.2]oct-3-yl]-3-cyano-1-benzofuran-5-carboxamide;
N-[(3R,4S)-1-azabicyclo[2.2.1]hept-3-yl][1,3]dioxolo[4,5-c]pyridine-6-carboxamide;
N-[(3R)-1-azabicyclo[2.2.2]oct-3-yl]-3-ethyl-2,3-dihydro-1,4-benzodioxine-6-carboxamide;
N-[(3R)-1-azabicyclo[2.2.2]oct-3-yl]-3-ethyl-2,3-dihydro-1,4-benzodioxine-7-carboxamide;
N-[(3R)-1-azabicyclo[2.2.2]oct-3-yl]-7-hydroxy-2-naphthamide;
N-[(1S,2R,4R)-7-azabicyclo[2.2.1]hept-2-yl]-3-ethynylfuro[2,3-c]pyridine-5-carboxamide;
N-[(1S,2R,4R)-7-azabicyclo[2.2.1]hept-2-yl]-6-chloroisoquinoline-3-carboxamide;
N-[(3R)-1-azabicyclo[2.2.2]oct-3-yl]-6-methylisoquinoline-3-carboxamide;
N-[(1S,2R,4R)-7-azabicyclo[2.2.1]hept-2-yl]-6-methylisoquinoline-3-carboxamide;
N-[(3R)-1-azabicyclo[2.2.2]oct-3-yl]-3-cyanofuro[2,3-c]pyridine-5-carboxamide;
N-[(3R)-1-azabicyclo[2.2.2]oct-3-yl]-2-naphthamide; and
N-[(3R)-1-azabicyclo[2.2.2]oct-3-yl]dibenzo[b,d]furan-2-carboxamide;
provided that the agonist is the free base or a pharmaceutically acceptable salt thereof.

42. The method of claim 41, wherein the disease or condition is wound healing, healing bone fracture, ischemic heart disease, or stable angina pectoris.

43. The method of claim 41, wherein the wound is from surgery or burn.

44. The method of claim 41, wherein the compound is
N-[(3R)-1-azabicyclo[2.2.2]oct-3-yl]dibenzo[b,d]thiophene-2-carboxamide;
N-1-azabicyclo[2.2.2]oct-3-yl [1]benzothieno[3,2-c]pyridine-3-carboxamide;
N-[(3R)-1-azabicyclo[2.2.2]oct-3-yl][1]benzothieno[2,3-c]pyridine-3-carboxamide; and
N-[(1S,2R,4R)-7-azabicyclo[2.2.1]hept-2-yl][1]benzothieno[2,3-c]pyridine-3-carboxamide;
provided that the agonist is the free base or a pharmaceutically acceptable salt thereof.

45. The method of claim 41, wherein the compound is
N-[(2S,3R)-2-methyl-1-azabicyclo[2.2.2]oct-3-yl]-2-naphthamide;
N-[(3R)-1-azabicyclo[2.2.2]oct-3-yl]-7-methoxy-2-naphthamide;
N-[(3R)-1-azabicyclo[2.2.2]oct-3-yl]-8-methoxy-2-naphthamide;
N-[(3R)-1-azabicyclo[2.2.2]oct-3-yl]-7-hydroxy-2-naphthamide;
N-[(3R)-1-azabicyclo[2.2.2]oct-3-yl]-2-naphthamide;
N-[(3R)-1-azabicyclo[2.2.2]oct-3-yl]isoquinoline-3-carboxamide;
N-[(1S,2R,4R)-7-azabicyclo[2.2.1]hept-2-yl]isoquinoline-3-carboxamide;
N-[(1S,2R,4R)-7-azabicyclo[2.2.1]hept-2-yl]-6-chloroisoquinoline-3-carboxamide;
N-[(3R)-1-azabicyclo[2.2.2]oct-3-yl]-6-methylisoquinoline-3-carboxamide; and
N-[(1S,2R,4R)-7-azabicyclo[2.2.1]hept-2-yl]-6-methylisoquinoline-3-carboxamide;
provided that the agonist is the free base or a pharmaceutically acceptable salt thereof.

46. The method of claim 41, wherein the compound is
N-[(3R)-1-azabicyclo[2.2.2]oct-3-yl]furo[2,3-c]pyridine-5-carboxamide;
N-[(3R)-1-azabicyclo[2.2.2]oct-3-yl]furo[3,2-c]pyridine-6-carboxamide;
N-[(3R)-1-azabicyclo[2.2.2]oct-3-yl]-2-methylfuro[2,3-c]pyridine-5-carboxamide;
N-[(3R)-1-azabicyclo[2.2.2]oct-3-yl]-3-methylfuro[2,3-c]pyridine-5-carboxamide;
N-[(1S,2R,4R)-7-azabicyclo[2.2.1]hept-2-yl]-3-methylfuro[2,3-c]pyridine-5-carboxamide;
N-[(1S,2R,4R)-7-azabicyclo[2.2.1]hept-2-yl]furo[2,3-c]pyridine-5-carboxamide;
N-[(3R)-1-azabicyclo[2.2.2]oct-3-yl]-3-ethylfuro[2,3-c]pyridine-5-carboxamide;
N-[(3R)-1-azabicyclo[2.2.2]oct-3-yl]-3-isopropylfuro[2,3-c]pyridine-5-carboxamide;
N-[(3R,4S)-1-azabicyclo[2.2.1]hept-3-yl]furo[2,3-c]pyridine-5-carboxamide;
N-[(3R)-1-azabicyclo[2.2.2]oct-3-yl]-3-chlorofuro[2,3-c]pyridine-5-carboxamide;
N-1-azabicyclo[2.2.2]oct-3-ylfuro[2,3-c]pyridine-5-carboxamide;
N-[(3R,5R)-1-azabicyclo[3.2.1]oct-3-yl]-3-methylfuro[2,3-c]pyridine-5-carboxamide;
N-[(3R,4S)-1-azabicyclo[2.2.1]hept-3-yl]-3-methylfuro[2,3-c]pyridine-5-carboxamide;
N-[(3R,4S)-1-azabicyclo[2.2.1]hept-3-yl]3-ethylfuro[2,3-c]pyridine-5-carboxamide;
N-[(3R,4S)-1-azabicyclo[2.2.1]hept-3-yl]3-isopropylfuro[2,3-c]pyridine-5-carboxamide;
N-[(1S,2R,4R)-7-azabicyclo[2.2.1]hept-2-yl]-3-chlorofuro[2,3-c]pyridine-5-carboxamide;
N-[(3R,4S)-1-azabicyclo[2.2.1]hept-3-yl]3-chlorofuro[2,3-c]pyridine-5-carboxamide;
N-[(2S,3R)-2-methyl-1-azabicyclo[2.2.2]oct-3-yl]furo[2,3-c]pyridine-5-carboxamide;
N-[(2S,3R)-2-methyl-1-azabicyclo[2.2.2]oct-3-yl]furo[3,2-c]pyridine-6-carboxamide;
N-[(3R,5R)-1-azabicyclo[3.2.1]oct-3-yl]furo[3,2-c]pyridine-6-carboxamide;
N-[(3R,5R)-1-azabicyclo[3.2.1]oct-3-yl]furo[2,3-c]pyridine-5-carboxamide;
N-[(3R)-1-azabicyclo[2.2.2]oct-3-yl]-3-bromofuro[2,3-c]pyridine-5-carboxamide;
N-[(1S,2R,4R)-7-azabicyclo[2.2.1]hept-2-yl]-3-bromofuro[2,3-c]pyridine-5-carboxamide;
3-methyl-N-[(2S,3R)-2-methyl-1-azabicyclo[2.2.2]oct-3-yl]furo[2,3-c]pyridine-5-carboxamide;
N-[(3R,4S)-1-azabicyclo[2.2.1]hept-3-yl]-3-bromofuro[2,3-c]pyridine-5-carboxamide;
N-[(3S)-1-azabicyclo[2.2.2]oct-3-yl]furo[2,3-c]pyridine-5-carboxamide;
N-[(3R)-1-azabicyclo[2.2.2]oct-3-yl]-3-ethynylfuro[2,3-c]pyridine-5-carboxamide;
3-bromo-N-[(2S,3R)-2-methyl-1-azabicyclo[2.2.2]oct-3-yl]furo[2,3-c]pyridine-5-carboxamide;
N-[(3R)-1-azabicyclo[2.2.2]oct-3-yl]-3-prop-1-ynylfuro[2,3-c]pyridine-5-carboxamide;
N-[(1S,2R,4R)-7-azabicyclo[2.2.1]hept-2-yl]-3-ethynylfuro[2,3-c]pyridine-5-carboxamide; and
N-[(3R)-1-azabicyclo[2.2.2]oct-3-yl]-3-cyanofuro[2,3-c]pyridine-5-carboxamide;
provided that the agonist is the free base or a pharmaceutically acceptable salt thereof.

47. The method of claim 41, wherein the compound is
N-[(3R)-1-azabicyclo[2.2.2]oct-3-yl]-2,3-dihydro-1-benzofuran-5-carboxamide;
N-[(3R)-1-azabicyclo[2.2.2]oct-3-yl]-1-benzofuran-5-carboxamide;
N-[(3R,4S)-1-azabicyclo[2.2.1]hept-3-yl]-1-benzofuran-5-carboxamide;
N-[(1S,2R,4R)-7-azabicyclo[2.2.1]hept-2-yl]-1-benzofuran-5-carboxamide;
N-[(3R,5R)-1-azabicyclo[3.2.1]oct-3-yl]-1-benzofuran-5-carboxamide;
N-[(3R)-1-azabicyclo[2.2.2]oct-3-yl]-1-benzofuran-6-carboxamide;
N-[(2S,3R)-2-methyl-1-azabicyclo[2.2.2]oct-3-yl]-1-benzofuran-5-carboxamide;
N-[(3R)-1-azabicyclo[2.2.2]oct-3-yl]-3-bromo-1-benzofuran-5-carboxamide;
N-[(1S,2R,4R)-7-azabicyclo[2.2.1]hept-2-yl]-3-bromo-1-benzofuran-5-carboxamide;
N-[(3R)-1-azabicyclo[2.2.2]oct-3-yl]-3-methyl-1-benzofuran-5-carboxamide;
N-[(1S,2R,4R)-7-azabicyclo[2.2.1]hept-2-yl]-3-methyl-1-benzofuran-5-carboxamide;
N-[(3R)-1-azabicyclo[2.2.2]oct-3-yl]-2-methyl-1-benzofuran-6-carboxamide;
N-[(3R,5R)-1-azabicyclo[3.2.1]oct-3-yl]-1-benzofuran-6-carboxamide;
N-[(2S,3R)-2-methyl-1-azabicyclo[2.2.2]oct-3-yl]-1-benzofuran-6-carboxamide;
N-[(3S)-1-azabicyclo[2.2.2]oct-3-yl]-1-benzofuran-5-carboxamide;
N-[(3R)-1-azabicyclo[2.2.2]oct-3-yl]-3-isopropyl-1-benzofuran-5-carboxamide;
N-[(1S,2R,4R)-7-azabicyclo[2.2.1]hept-2-yl]-3-isopropyl-1-benzofuran-5-carboxamide;
N-[(3R)-1-azabicyclo[2.2.2]oct-3-yl]-2-methyl-1-benzofuran-5-carboxamide;
N-[(1S,2R,4R)-7-azabicyclo[2.2.1]hept-2-yl]-2-methyl-1-benzofuran-5-carboxamide;
N-[(3R,4S)-1-azabicyclo[2.2.1]hept-3-yl]-3-bromo-1-benzofuran-6-carboxamide;
N-[(3R)-1-azabicyclo[2.2.2]oct-3-yl]-3-ethynyl-1-benzofuran-5-carboxamide;
N-[(1S,2R,4R)-7-azabicyclo[2.2.1]hept-2-yl]-3-ethynyl-1-benzofuran-5-carboxamide;
N-[(3R)-1-azabicyclo[2.2.2]oct-3-yl]-3-prop-1-ynyl-1-benzofuran-5-carboxamide; and
N-[(3R)-1-azabicyclo[2.2.2]oct-3-yl]-3-cyano-1-benzofuran-5-carboxamide;
provided that the agonist is the free base or a pharmaceutically acceptable salt thereof.

48. The method of claim 41, wherein the compound is
N-[(3R)-1-azabicyclo[2.2.2]oct-3-yl]pyrazino[1,2-a]indole-3-carboxamide;
N-[(3R,4S)-1-azabicyclo[2.2.1]hept-3-yl]-1H-indole-6-carboxamide;
N-[(3R)-1-azabicyclo[2.2.2]oct-3-yl]-1-methyl-1H-indole-6-carboxamide;
N-[(3R)-1-azabicyclo[2.2.2]oct-3-yl]-1-benzothiophene-5-carboxamide;
N-[(3R,4S)-1-azabicyclo[2.2.1]hept-3-yl]-1-benzothiophene-5-carboxamide;
N-[(2S,3R)-2-methyl-1-azabicyclo[2.2.2]oct-3-yl]-1-benzothiophene-5-carboxamide;
N-[(3R)-1-azabicyclo[2.2.2]oct-3-yl]-1-benzothiophene-6-carboxamide; and
N-[(3R,4S)-1-azabicyclo[2.2.1]hept-3-yl]-1-benzothiophene-6-carboxamide;
provided that the agonist is the free base or a pharmaceutically acceptable salt thereof.

49. The method of claim 41, wherein the compound is
N-[(1S,2R,4R)-7-azabicyclo[2.2.1]hept-2-yl]thieno[2,3-c]pyridine-5-carboxamide;
N-[(1S,2R,4R)-7-azabicyclo[2.2.1]hept-2-yl]thieno[3,2-c]pyridine-6-carboxamide;
N-[(1S,2R,4R)-7-azabicyclo[2.2.1]hept-2-yl]thieno[3,4-c]pyridine-6-carboxamide;
N-[(3R)-1-azabicyclo[2.2.2]oct-3-yl]thieno[2,3-c]pyridine-5-carboxamide;
N-[(3R)-1-azabicyclo[2.2.2]oct-3-yl]thieno[3,2-c]pyridine-6-carboxamide;
N-[(3R)-1-azabicyclo[2.2.2]oct-3-yl]thieno[3,4-c]pyridine-6-carboxamide;
N-[(3R,4S)-1-azabicyclo[2.2.1]hept-3-yl]thieno[2,3-c]pyridine-5-carboxamide;
N-[(3R,4S)-1-azabicyclo[2.2.1]hept-3-yl]thieno[3,2-c]pyridine-6-carboxamide;
N-[(3R,5R)-1-azabicyclo[3.2.1]oct-3-yl]thieno[2,3-c]pyridine-5-carboxamide;
N-[(3R,5R)-1-azabicyclo[3.2.1]oct-3-yl]thieno[3,2-c]pyridine-6-carboxamide;
N-[(2S,3R)-2-methyl-1-azabicyclo[2.2.2]oct-3-yl]thieno[2,3-c]pyridine-5-carboxamide;
N-[(2S,3R)-2-methyl-1-azabicyclo[2.2.2]oct-3-yl]thieno[3,2-c]pyridine-6-carboxamide;
N-[(3R)-1-azabicyclo[2.2.2]oct-3-yl]-3-bromothieno[2,3-c]pyridine-5-carboxamide; and
N-[(1S,2R,4R)-7-azabicyclo[2.2.1]hept-2-yl]-3-bromothieno[2,3-c]pyridine-5-carboxamide;
provided that the agonist is the free base or a pharmaceutically acceptable salt thereof.

50. The method of claim 41, wherein the compound is
N-[(3R)-1-azabicyclo[2.2.2]oct-3-yl]-1,3-benzothiazole-6-carboxamide;
N-[(2S,3R)-2-methyl-1-azabicyclo[2.2.2]oct-3-yl]-1,3-benzothiazole-6-carboxamide;
N-[(3R,5R)-1-azabicyclo[3.2.1]oct-3-yl]-1,3-benzothiazole-6-carboxamide;
2-amino-N-[(3R)-1-azabicyclo[2.2.2]oct-3-yl]-1,3-benzothiazole-6-carboxamide;
N-[(3R)-1-azabicyclo[2.2.2]oct-3-yl]-1H-indazole-6-carboxamide;
N-[(3R)-1-azabicyclo[2.2.2]oct-3-yl][1,3]dioxolo[4,5-c]pyridine-6-carboxamide; and
N-[(1S,2R,4R)-7-azabicyclo[2.2.1]hept-2-yl][1,3]dioxolo[4,5-c]pyridine-6-carboxamide;
provided that the agonist is the free base or a pharmaceutically acceptable salt thereof.

51. The method of claim 41, wherein the compound is
N-[(3R)-1-azabicyclo[2.2.2]oct-3-yl]pyrrolo[1,2-c]pyrimidine-3-carboxamide;
N-[(3R,5R)-1-azabicyclo[3.2.1]oct-3-yl]pyrrolo[1,2-c]pyrimidine-3-carboxamide;
N-[(1S,2R,4R)-7-azabicyclo[2.2.1]hept-2-yl]pyrrolo[1,2-c]pyrimidine-3-carboxamide;
N-[(3R,4S)-1-azabicyclo[2.2.1]hept-3-yl]pyrrolo[1,2-c]pyrimidine-3-carboxamide;
N-[(2S,3R)-2-methyl-1-azabicyclo[2.2.2]oct-3-yl]pyrrolo[1,2-c]pyrimidine-3-carboxamide;
N-[(3R)-1-azabicyclo[2.2.2]oct-3-yl]pyrrolo[1,2-a]pyrazine-3-carboxamide;
N-[(3R,5R)-1-azabicyclo[3.2.1]oct-3-yl]pyrrolo[1,2-a]pyrazine-3-carboxamide;
N-[(1S,2R,4R)-7-azabicyclo[2.2.1]hept-2-yl]pyrrolo[1,2-a]pyrazine-3-carboxamide;
N-[(3R)-1-azabicyclo[2.2.2]oct-3-yl]-6-bromopyrrolo[1,2-a]pyrazine-3-carboxamide;
N-[(2S,3R)-2-methyl-1-azabicyclo[2.2.2]oct-3-yl]pyrrolo[1,2-a]pyrazine-3-carboxamide;
N-[(3R)-1-azabicyclo[2.2.2]oct-3-yl]-6-ethynylpyrrolo[1,2-a]pyrazine-3-carboxamide;
N-[(3R)-1-azabicyclo[2.2.2]oct-3-yl]indolizine-6-carboxamide; and
N-[(2S,3R)-2-methyl-1-azabicyclo[2.2.2]oct-3-yl]indolizine-6-carboxamide;
provided that the agonist is the free base or a pharmaceutically acceptable salt thereof.
